(19) United States
(12) Patent Application Publication
(10) Pub. No.: US 2022/0267464 A1
(43) Pub. Date: Aug. 25, 2022

(54) FUSION OF AN ANTIBODY BINDING CEA AND 4-1BBL

(71) Applicant: Hoffmann-La Roche Inc., Little Falls, NJ (US)

(72) Inventors: Claudia Ferrara Koller, Zug (CH); Thomas Hofer, Zuerich (CH); Christian Klein, Bonstetten (CH); Ekkehard Moessner, Bonstetten (CH); Christina Claus, Ennetbaden (CH); Ralf Hosse, Mettmenstetten (CH); Bianca Scherer, Urdorf (CH); Pablo Umana, Wollerau (CH)

(73) Assignee: Hoffmann-La Roche Inc., Little Falls, NJ (US)

(21) Appl. No.: 17/644,903

(22) Filed: Dec. 17, 2021

Related U.S. Application Data
(63) Continuation of application No. PCT/EP2020/067582, filed on Jun. 24, 2020.

(30) Foreign Application Priority Data
Jun. 26, 2019 (EP) 19182505.8

(51) Int. Cl. C07K 16/30; A61P 35/00; C07K 14/705

(52) U.S. Cl. CPC C07K 16/3007; A61P 35/00; C07K 14/70578; C07K 2317/24; C07K 2317/522; C07K 2317/33; C07K 2317/526; C07K 2317/55; C07K 2317/524; C07K 2317/31

(57) ABSTRACT

The invention relates to new humanized CEA antibodies and to CEA targeting 4-1BBL trimer-containing antigen binding molecules comprising these CEA antibodies as well as their use in the treatment of cancer.

### FIELD OF THE INVENTION

The invention relates to new antigen binding molecules that bind to carcinoembryonic antigen (CEA), in particular to new humanized CEA antibodies and to CEA targeting 4-1BBL trimer-containing antigen binding molecules comprising these CEA antibodies as well as their use in the treatment of cancer. The invention further relates to methods of producing these molecules and to methods of using the same.

### BACKGROUND

Carcinoembryonic antigen (CEA), also referred to as carcinoembryonic antigen-related cell adhesion molecule 5 (CEACAM-5 or CD66e), is a glycoprotein having a molecular weight of about 180 kDa. CEA is a member of the immunoglobulin gene superfamily and contains seven domains that are linked to the cell membrane through a glycosylphosphatidyl-inositol (GPI) anchor (Thompson J. A., J Clin Lab Anal. 5:344-366, 1991) The seven domains include a single N-terminal variable domain-like region and three sets of constant-domain like regions (A1B1, A2B2, and A3B3; 92 amino acids for A domains and 86 amino acids for B domains, Hefta L J, et al., Cancer Res. 52:5647-5655, 1992).

The human CEA family contains 29 genes, of which 18 are expressed: 7 belonging to the CEA subgroup and 11 to the pregnancy-specific glycoprotein subgroup. Several CEA subgroup members are thought to possess cell adhesion properties. CEA is thought to have a role in innate immunity. Because of the existence of proteins closely related to CEA, it can be challenging to raise anti-CEA antibodies that are specific for CEA with minimal cross-reactivity to the other closely related proteins.

CEA has long been identified as a tumor-associated antigen (Gold and Freedman, J Exp Med. 1965, 121, 439-462. CEA plays a critical role in cell adhesion, invasion, and metastasis of cancer cells. Endogenous expression of CEA affects expression of various groups of cancer-related genes, especially cell cycle and apoptotic genes, protecting colonic tumor cells from various apoptotic stimuli, such as treatment with 5-fluorouracil (Soeth et al., Clin. Cancer Res. 2001, 7(7), 2022-2030). CEA inhibits a process known as anoikis, whereby cells deprived of their anchorage to the extracellular matrix subsequently undergo apoptosis (Ordoñez et al., Cancer Research 2000, 60(13), 3419-3424). Therefore, CEA expression may be a way by which cancer cells gain a survival benefit and overcome apoptosis-inducing therapies.

High expression of CEA was found in various tumor types (Thompson et al., J. Clin. Lab. Anal. 1991, 5, 344-366). Its high prevalence has been observed in colorectal cancer (CRC), pancreatic cancer, gastric cancer, non-small cell lung cancer (NSCLC), and breast cancer amongst others; low expression was found in small-cell lung cancer and glioblastoma. Tumors of epithelial origin, as well as their metastases, contain CEA as a tumor associated antigen. While the presence of CEA itself does not indicate transformation to a cancerous cell, the distribution of CEA is indicative. In normal tissue, CEA is generally expressed on the apical surface of the cell (Hammarström S., Semin Cancer Biol. 1999, 9(2), 67-81), making it inaccessible to antibody in the blood stream. In contrast to normal tissue, CEA tends to be expressed over the entire surface of cancerous cells. This change of expression pattern makes CEA accessible to antibody binding in cancerous cells. In addition, CEA expression increases in cancerous cells. Furthermore, increased CEA expression promotes increased intercellular adhesions, which may lead to metastasis (Marshall J., Semin. Oncol. 2003, 30 (Suppl. 8):30-36).

CEA is readily cleaved from the cell surface and shed into the blood stream from tumors, either directly or via the lymphatics. Because of this property, the level of serum CEA has been used as a clinical marker for diagnosis of cancers and screening for recurrence of cancers, particularly colorectal cancer. This property also presents one of the challenges for using CEA as a target, since serum CEA binds most of the currently available anti-CEA antibodies, hindering them from reaching their target on the cell surface and limiting potential clinical effects.

Multiple monoclonal antibodies have been raised against CEA for research purposes, as diagnostic tools, and for therapeutic purposes. One is the murine antibody T84.66 (Wagener et al., J Immunol 1983, 130, 2308, Neumaier et al. 1985, J Immunol 135, 3604), which has also been chimerized (WO 1991/01990) and humanized (WO 2005/086875). Another CEA antibody is the mouse monoclonal antibody PR1A3 (Richman et al., Int. J. Cancer 1987, 59, 317-328) which targets the targets the B3 domain and the GPI anchor of the CEA molecule and thus only binds to the membrane-bound CEA and not to soluble CEA. Affinity-matured humanized variants thereof are described in WO 2011/023787. Humanized antibodies derived. from murine antibody A5B7 have been disclosed in WO 92/01059 and WO 2007/071422. However, there is an ongoing need to provide new CEA antibodies with advantageous properties, in particular for the use of targeting therapeutic molecules to tumor cells.

4-1BB (CD137), a member of the TNF receptor superfamily, was first identified as an inducible molecule expressed by activated by T cells (Kwon and Weissman, 1989, Proc Natl Acad Sci USA 86, 1963-1967). Subsequent studies demonstrated that many other immune cells also express 4-1BB, including NK cells, B cells, NKT cells, monocytes, neutrophils, mast cells, dendritic cells (DCs) and cells of non-hematopoietic origin such as endothelial and smooth muscle cells (Vinay and Kwon, 2011, Cell Mol Immunol 8, 281-284). Expression of 4-1BB in different cell types is mostly inducible and driven by various stimulatory signals, such as T-cell receptor (TCR) or B-cell receptor triggering, as well as signaling induced through co-stimulatory molecules or receptors of pro-inflammatory cytokines (Diehl et al., 2002, J Immunol 168, 3755-3762; Zhang et al., 2010, Clin Cancer Res 13, 2758-2767).

4-1BB ligand (4-1BBL or CD137L) was identified in 1993 (Goodwin et al., 1993, Eur J Immunol 23, 2631-2641). It has been shown that expression of 4-1BBL was restricted on professional antigen presenting cells (APC) such as B-cells, DCs and macrophages. Inducible expression of 4-1BBL is characteristic for T-cells, including both αβ and γδ T-cell subsets, and endothelial cells (Shao and Schwarz, 2011, J Leukoc Biol 89, 21-29).

Co-stimulation through the 4-1BB receptor (for example by 4-1BBL ligation) activates multiple signaling cascades within the T cell (both CD4⁺ and CD8⁺ subsets), powerfully augmenting T cell activation (Bartkowiak and Curran, 2015). In combination with TCR triggering, agonistic 4-1BB-specific antibodies enhance proliferation of T-cells, stimulate lymphokine secretion and decrease sensitivity of T-lymphocytes to activation-induced cells death (Snell et al., 2011, Immunol Rev 244, 197-217). This mechanism was further advanced as the first proof of concept in cancer immunotherapy. In a preclinical model administration of an agonistic antibody against 4-1BB in tumor bearing mice led to potent anti-tumor effect (Melero et al., 1997, Nat Med 3, 682-685). Later, accumulating evidence indicated that 4-1BB usually exhibits its potency as an anti-tumor agent only when administered in combination with other immunomodulatory compounds, chemotherapeutic reagents, tumor-specific vaccination or radiotherapy (Bartkowiak and Curran, 2015, Front Oncol 5, 117).

Signaling of the TNFR-superfamily needs cross-linking of the trimerized ligands to engage with the receptors, so does the 4-1BB agonistic antibodies which require wild type Fc-binding (Li and Ravetch, 2011, Science 333, 1030-1034). However, systemic administration of 4-1BB-specific agonistic antibodies with the functionally active Fc domain resulted in influx of CD8⁺ T-cells associated with liver toxicity (Dubrot et al., 2010, Cancer Immunol Immunother 59, 1223-1233) that is diminished or significantly ameliorated in the absence of functional Fc-receptors in mice. In the clinic, an Fc-competent 4-1BB agonistic Ab (BMS-663513) (NCT00612664) caused a grade 4 hepatitis leading to termination of the trial (Simeone and Ascierto, 2012, J Immunotoxicol 9, 241-247). Therefore, there is a need for effective and safer 4-1BB agonists.

New antigen binding molecules that combine a moiety capable of forming a costimulatory 4-1BBL trimer with an antigen binding domain capable of binding to tumor-associated targets so that cross-linking only happens in the presence of the tumor-associated target are described for instance in WO 2016/075278. However, there is still need to optimize the tumor targeting by introducing new CEA antigen binding domains with advantageous properties.

### SUMMARY OF THE INVENTION

The invention relates to new antigen binding molecules that bind to carcinoembryonic antigen (CEA), in particular to new humanized CEA antibodies and to CEA targeting 4-1BBL trimer-containing antigen binding molecules comprising these CEA antibodies as well as their use in the treatment of cancer. The invention further relates to methods of producing these molecules and to methods of using the same.

The CEA targeting 4-1BBL trimer-containing antigen binding molecules have an increased activity on CEA-expressing tumor sites, comprise the natural human 4-1BB ligand and should thus impose less safety issues compared to conventional 4-1BB agonistic antibodies or more artificial fusion proteins. The new antigen binding molecules combine an anti-CEA antigen binding domain with a moiety that is capable of forming a costimulatory 4-1BBL trimer and that is sufficiently stable to be pharmaceutically useful. Surprisingly, antigen binding molecules of the invention provide a trimeric and thus biologically active human 4-1BB ligand, although one of the trimerizing 4-1BBL ectodomains is located on another polypeptide than the other two 4-1BBL ectodomains of the molecule.

In one aspect, the invention provides a 4-1BBL trimer-containing antigen binding molecule comprising

an antigen binding domain capable of specific binding to CEA,
a first and a second polypeptide that are linked to each other by a disulfide bond,
wherein the antigen binding molecule is characterized in that the first polypeptide comprises two ectodomains of 4-1BBL or a fragment thereof that are connected to each other by a peptide linker and in that the second polypeptide comprises one ectodomain of 4-1BBL or a fragment thereof, and
an Fc domain composed of a first and a second subunit capable of stable association,
wherein the antigen binding domain capable of specific binding to CEA comprises
(a) a variable heavy chain domain (VH) comprising (i) CDR-H1 comprising the amino acid sequence of SEQ ID NO:17, (ii) CDR-H2 comprising the amino acid sequence of SEQ ID NO:18, and (iii) CDR-H3 comprising the amino acid sequence of SEQ ID NO:19, and a variable light chain domain (VL) comprising (iv) CDR-L1 comprising the amino acid sequence of SEQ ID NO:20, (v) CDR-L2 comprising the amino acid sequence of SEQ ID NO:21, and (vi) CDR-L3 comprising the amino acid sequence of SEQ ID NO:22, or
(b) a VH domain comprising (i) CDR-H1 comprising the amino acid sequence of SEQ ID NO:25, (ii) CDR-H2 comprising the amino acid sequence of SEQ ID NO:26, and (iii) CDR-H3 comprising the amino acid sequence of SEQ ID NO:27, and a VL domain comprising (iv) CDR-L1 comprising the amino acid sequence of SEQ ID NO:28, (v) CDR-L2 comprising the amino acid sequence of SEQ ID NO:29, and (vi) CDR-L3 comprising the amino acid sequence of SEQ ID NO:30, or
(c) a VH domain comprising (i) CDR-H1 comprising the amino acid sequence of SEQ ID NO:65, (ii) CDR-H2 comprising the amino acid sequence of SEQ ID NO:66 or SEQ ID NO:67, and (iii) CDR-H3 comprising the amino acid sequence of SEQ ID NO:68, and a VL domain comprising (iv) CDR-L1 comprising the amino acid sequence of SEQ ID NO:69 or SEQ ID NO:70 or SEQ ID NO:313, (v) CDR-L2 comprising the amino acid sequence of SEQ ID NO:71 or SEQ ID NO:72 or SEQ ID NO:73, and (vi) CDR-L3 comprising the amino acid sequence of SEQ ID NO:74.

In another aspect, provided is a 4-1BBL trimer-containing antigen binding molecule as defined herein before, wherein the ectodomain of 4-1BBL or a fragment thereof comprises the amino acid sequence selected from the group consisting of SEQ ID NO:87, SEQ ID NO:88, SEQ ID NO:89, SEQ ID NO:90, SEQ ID NO:91, SEQ ID NO:92, SEQ ID NO:93 and SEQ ID NO:94, particularly the amino acid sequence of SEQ ID NO:91.

In a further aspect, provided is a 4-1BBL trimer-containing antigen binding molecule as described herein, comprising

an antigen binding domain capable of specific binding to CEA,
a first and a second polypeptide that are linked to each other by a disulfide bond,
wherein the antigen binding molecule is characterized in that the first polypeptide comprises the amino acid sequence selected from the group consisting of SEQ ID NO:95, SEQ ID NO:96, SEQ ID NO:97 and SEQ ID NO:98 and in that the second polypeptide comprises the amino acid sequence selected from the group consisting of SEQ ID NO:87, SEQ ID NO:91, SEQ ID NO:89 and SEQ ID NO:94, and
an Fc domain composed of a first and a second subunit capable of stable association, wherein the antigen binding domain capable of specific binding to CEA comprises
(a) a variable heavy chain domain (VH) comprising (i) CDR-H1 comprising the amino acid sequence of SEQ ID NO:17, (ii) CDR-H2 comprising the amino acid sequence of SEQ ID NO:18, and (iii) CDR-H3 comprising the amino acid sequence of SEQ ID NO:19, and a variable light chain domain (VL) comprising (iv) CDR-L1 comprising the amino acid sequence of SEQ ID NO:20, (v) CDR-L2 comprising the amino acid sequence of SEQ ID NO:21, and (vi) CDR-L3 comprising the amino acid sequence of SEQ ID NO:22, or
(b) a VH domain comprising (i) CDR-H1 comprising the amino acid sequence of SEQ ID NO:25, (ii) CDR-H2 comprising the amino acid sequence of SEQ ID NO:26, and (iii) CDR-H3 comprising the amino acid sequence of SEQ ID NO:27, and a VL domain comprising (iv) CDR-L1 comprising the amino acid sequence of SEQ ID NO:28, (v) CDR-L2 comprising the amino acid sequence of SEQ ID NO:29, and (vi) CDR-L3 comprising the amino acid sequence of SEQ ID NO:30, or
(c) a VH domain comprising (i) CDR-H1 comprising the amino acid sequence of SEQ ID NO:65, (ii) CDR-H2 comprising the amino acid sequence of SEQ ID NO:66 or SEQ ID NO:67, and (iii) CDR-H3 comprising the amino acid sequence of SEQ ID NO:68, and a VL domain comprising (iv) CDR-L1 comprising the amino acid sequence of SEQ ID NO:69 or SEQ ID NO:70 or SEQ ID NO:313, (v) CDR-L2 comprising the amino acid sequence of SEQ ID NO:71 or SEQ ID NO:72 or SEQ ID NO:73, and (vi) CDR-L3 comprising the amino acid sequence of SEQ ID NO:74.

In one aspect, the Fc domain is an IgG, particularly an IgG1 Fc domain or an IgG4 Fc domain. More particularly, the Fc domain is an IgG1 Fc domain. In a particular aspect, the Fc domain comprises a modification promoting the association of the first and second subunit of the Fc domain. In a particular aspect, the invention provides a 4-1BBL trimer-containing antigen binding molecule, wherein the Fc domain comprises knob-into-hole modifications promoting association of the first and the second subunit of the Fc domain. In a specific aspect, the invention provides a 4-1BBL trimer-containing antigen binding molecule, wherein the first subunit of the Fc domain comprises the amino acid substitutions S354C and T366W (numbering according to Kabat EU index) and the second subunit of the Fc domain comprises the amino acid substitutions Y349C, T366S, L368A and Y407V (numbering according to Kabat EU index).

In another aspect, the invention is concerned with a 4-1BBL trimer-containing antigen binding molecule as defined herein before, comprising (c) an Fc domain composed of a first and a second subunit capable of stable association, wherein the Fc domain comprises one or more amino acid substitution that reduces binding to an Fc receptor, in particular towards Fcγ receptor. In particular, the Fc domain comprises amino acid substitutions at positions 234 and 235 (EU numbering according to Kabat) and/or 329 (EU numbering according to Kabat) of the IgG heavy chains. Particularly, provided is a 4-1BBL trimer-containing antigen binding molecule, wherein the Fc domain is an IgG1 Fc domain comprising the amino acid substitutions the amino acid substitutions L234A, L235A and P329G (numbering according to Kabat EU index).

In one aspect, the 4-1BBL trimer-containing antigen binding molecule is one, wherein wherein the antigen binding domain capable of specific binding to CEA is a Fab molecule capable of specific binding to CEA. In another aspect, the antigen binding domain capable of specific binding to CEA is a cross-over Fab molecule or a scFV molecule capable of specific binding to CEA.

In one aspect, provided is a 4-1BBL trimer-containing antigen binding molecule, wherein the antigen binding domain capable of specific binding to CEA comprises (a) a variable heavy chain domain (VH) comprising (i) CDR-H1 comprising the amino acid sequence of SEQ ID NO:17, (ii) CDR-H2 comprising the amino acid sequence of SEQ ID NO:18, and (iii) CDR-H3 comprising the amino acid sequence of SEQ ID NO:19, and a variable light chain domain (VL) comprising (iv) CDR-L1 comprising the amino acid sequence of SEQ ID NO:20, (v) CDR-L2 comprising the amino acid sequence of SEQ ID NO:21, and (vi) CDR-L3 comprising the amino acid sequence of SEQ ID NO:22, or (b) a VH domain comprising (i) CDR-H1 comprising the amino acid sequence of SEQ ID NO:25, (ii) CDR-H2 comprising the amino acid sequence of SEQ ID NO:26, and (iii) CDR-H3 comprising the amino acid sequence of SEQ ID NO:27, and a VL domain comprising (iv) CDR-L1 comprising the amino acid sequence of SEQ ID NO:28, (v) CDR-L2 comprising the amino acid sequence of SEQ ID NO:29, and (vi) CDR-L3 comprising the amino acid sequence of SEQ ID NO:30.

In one aspect, the antigen binding domain capable of specific binding to CEA comprises a variable heavy chain domain (VH) comprising (i) CDR-H1 comprising the amino acid sequence of SEQ ID NO:17, (ii) CDR-H2 comprising the amino acid sequence of SEQ ID NO:18, and (iii) CDR-H3 comprising the amino acid sequence of SEQ ID NO:19, and a variable light chain domain (VL) comprising (iv) CDR-L1 comprising the amino acid sequence of SEQ ID NO:20, (v) CDR-L2 comprising the amino acid sequence of SEQ ID NO:21, and (vi) CDR-L3 comprising the amino acid sequence of SEQ ID NO:22.

In another aspect, the antigen binding domain capable of specific binding to CEA comprises a VH domain comprising (i) CDR-H1 comprising the amino acid sequence of SEQ ID NO:25, (ii) CDR-H2 comprising the amino acid sequence of SEQ ID NO:26, and (iii) CDR-H3 comprising the amino acid sequence of SEQ ID NO:27, and a VL domain comprising (iv) CDR-L1 comprising the amino acid sequence of SEQ ID NO:28, (v) CDR-L2 comprising the amino acid sequence of SEQ ID NO:29, and (vi) CDR-L3 comprising the amino acid sequence of SEQ ID NO:30.

In a particular aspect, provided is a 4-1BBL trimer-containing antigen binding molecule, wherein the antigen binding domain capable of specific binding to CEA comprises

(a) a VH domain comprising an amino acid sequence of SEQ ID NO:23 and a VL domain comprising an amino acid sequence of SEQ ID NO:24, or
(b) a VH domain comprising an amino acid sequence of SEQ ID NO:31 and a VL domain comprising an amino acid sequence of SEQ ID NO:32, or
(c) a VH domain comprising an amino acid sequence of SEQ ID NO:33 and a VL domain comprising an amino acid sequence of SEQ ID NO:34, or
(d) a VH domain comprising an amino acid sequence of SEQ ID NO:35 and a VL domain comprising an amino acid sequence of SEQ ID NO:36, or
(e) a VH domain comprising an amino acid sequence of SEQ ID NO:37 and a VL domain comprising an amino acid sequence of SEQ ID NO:38, or
(f) a VH domain comprising an amino acid sequence of SEQ ID NO:39 and a VL domain comprising an amino acid sequence of SEQ ID NO:40, or
(g) a VH domain comprising an amino acid sequence of SEQ ID NO:41 and a VL domain comprising an amino acid sequence of SEQ ID NO:42, or
(h) a VH domain comprising an amino acid sequence of SEQ ID NO:43 and a VL domain comprising an amino acid sequence of SEQ ID NO:44, or
(i) a VH domain comprising an amino acid sequence of SEQ ID NO:45 and a VL domain comprising an amino acid sequence of SEQ ID NO:46, or
(j) a VH domain comprising an amino acid sequence of SEQ ID NO:47 and a VL domain comprising an amino acid sequence of SEQ ID NO:48, or
(k) a VH domain comprising an amino acid sequence of SEQ ID NO:49 and a VL domain comprising an amino acid sequence of SEQ ID NO:50.
(l) a VH domain comprising an amino acid sequence of SEQ ID NO:51 and a VL domain comprising an amino acid sequence of SEQ ID NO:52, or
(m) a VH domain comprising an amino acid sequence of SEQ ID NO:53 and a VL domain comprising an amino acid sequence of SEQ ID NO:54.

In a further aspect, the invention provides a 4-1BBL trimer-containing antigen binding molecule as described herein, wherein the antigen binding domain capable of specific binding to CEA comprises a VH domain comprising (i) CDR-H1 comprising the amino acid sequence of SEQ ID NO:65, (ii) CDR-H2 comprising the amino acid sequence of SEQ ID NO:66 or SEQ ID NO:67, and (iii) CDR-H3 comprising the amino acid sequence of SEQ ID NO:68, and a VL domain comprising (iv) CDR-L1 comprising the amino acid sequence of SEQ ID NO:69 or SEQ ID NO:70 or SEQ ID NO:313, (v) CDR-L2 comprising the amino acid sequence of SEQ ID NO:71 or SEQ ID NO:72 or SEQ ID NO:73, and (vi) CDR-L3 comprising the amino acid sequence of SEQ ID NO:74. In one aspect, the antigen binding domain capable of specific binding to CEA comprises a heavy chain variable region (VH) comprising the amino acid sequence of SEQ ID NO:75, SEQ ID NO:76, SEQ ID NO:77, SEQ ID NO:78, SEQ ID NO:79 or SEQ ID NO:80, and a light chain variable region (VL) comprising the amino acid sequence of SEQ ID NO:81, SEQ ID NO:82, SEQ ID NO:83, SEQ ID NO:84, SEQ ID NO:85 or SEQ ID NO:86.

In a particular aspect, provided is a 4-1BBL trimer-containing antigen binding molecule, wherein the antigen binding domain capable of specific binding to CEA comprises

(a) a VH domain comprising an amino acid sequence of SEQ ID NO:75 and a VL domain comprising an amino acid sequence of SEQ ID NO:85, or
(b) a VH domain comprising an amino acid sequence of SEQ ID NO:79 and a VL domain comprising an amino acid sequence of SEQ ID NO:85, or
(c) a VH domain comprising an amino acid sequence of SEQ ID NO:76 and a VL domain comprising an amino acid sequence of SEQ ID NO:85, or
(d) a VH domain comprising an amino acid sequence of SEQ ID NO:80 and a VL domain comprising an amino acid sequence of SEQ ID NO:84, or
(e) a VH domain comprising an amino acid sequence of SEQ ID NO:79 and a VL domain comprising an amino acid sequence of SEQ ID NO:84, or
(f) a VH domain comprising an amino acid sequence of SEQ ID NO:77 and a VL domain comprising an amino acid sequence of SEQ ID NO:84, or
(g) a VH domain comprising an amino acid sequence of SEQ ID NO:75 and a VL domain comprising an amino acid sequence of SEQ ID NO:84.

In another aspect, provided is a 4-1BBL trimer-containing antigen binding molecule, wherein the first peptide comprising two ectodomains of 4-1BBL or a fragment thereof connected to each other by a first peptide linker is fused at its C-terminus by a second peptide linker to a CL domain that is part of a heavy chain, and the second peptide comprising one ectodomain of said 4-1BBL or a fragment thereof is fused at its C-terminus by a third peptide linker to a CH1 domain that is part of a light chain. In another aspect, provided is a 4-1BBL trimer-containing antigen binding molecule, wherein the first peptide comprising two ectodomains of 4-1BBL or a fragment thereof connected to each other by a first peptide linker is fused at its C-terminus by a second peptide linker to a CH domain that is part of a heavy chain, and the second peptide comprising one ectodomain of said 4-1BBL or a fragment thereof is fused at its C-terminus by a third peptide linker to a CL domain that is part of a light chain.

In a further aspect, there is provided a 4-1BBL trimer-containing antigen binding molecule as described herein, wherein the antigen binding molecule comprises

(i) a first heavy chain and a first light chain, both comprising a Fab molecule capable of specific binding to CEA,
(ii) a second heavy chain comprising the amino acid sequence selected from the group consisting of SEQ ID NO:99, SEQ ID NO:101, SEQ ID NO:103 and SEQ ID NO:105, and
(iii) a second light chain comprising the amino acid sequence selected from the group consisting of SEQ ID NO:100, SEQ ID NO:102, SEQ ID NO:104 and SEQ ID NO:106.

Particularly, provided is a 4-1BBL trimer-containing antigen binding molecule as described herein, wherein the antigen binding molecule comprises

(a) a first heavy chain comprising the amino acid sequence of SEQ ID NO:99, a first light chain comprising the amino acid sequence of SEQ ID NO:100, a second heavy chain comprising the amino acid sequence of SEQ ID NO:238 and a second light chain comprising the amino acid sequence of SEQ ID NO:239, or
(b) a first heavy chain comprising the amino acid sequence of SEQ ID NO:99, a first light chain comprising the amino acid sequence of SEQ ID NO:100, a second heavy chain comprising the amino acid sequence of SEQ ID NO:240 and a second light chain comprising the amino acid sequence of SEQ ID NO:241, or
(c) a first heavy chain comprising the amino acid sequence of SEQ ID NO:99, a first light chain comprising the amino acid sequence of SEQ ID NO:100, a second heavy chain comprising the amino acid sequence of SEQ ID NO:242 and a second light chain comprising the amino acid sequence of SEQ ID NO:243, or
(d) a first heavy chain comprising the amino acid sequence of SEQ ID NO:99, a first light chain comprising the amino acid sequence of SEQ ID NO:100, a second heavy chain comprising the amino acid sequence of SEQ ID NO:244 and a second light chain comprising the amino acid sequence of SEQ ID NO:245, or
(e) a first heavy chain comprising the amino acid sequence of SEQ ID NO:99, a first light chain comprising the amino acid sequence of SEQ ID NO:100, a second heavy chain comprising the amino acid sequence of SEQ ID NO:246 and a second light chain comprising the amino acid sequence of SEQ ID NO:247, or
(f) a first heavy chain comprising the amino acid sequence of SEQ ID NO:99, a first light chain comprising the amino acid sequence of SEQ ID NO:100, a second heavy chain comprising the amino acid sequence of SEQ ID NO:248 and a second light chain comprising the amino acid sequence of SEQ ID NO:249, or
(g) a first heavy chain comprising the amino acid sequence of SEQ ID NO:99, a first light chain comprising the amino acid sequence of SEQ ID NO:100, a second heavy chain comprising the amino acid sequence of SEQ ID NO:250 and a second light chain comprising the amino acid sequence of SEQ ID NO:251, or
(h) a first heavy chain comprising the amino acid sequence of SEQ ID NO:99, a first light chain comprising the amino acid sequence of SEQ ID NO:100, a second heavy chain comprising the amino acid sequence of SEQ ID NO:252 and a second light chain comprising the amino acid sequence of SEQ ID NO:253, or
(i) a first heavy chain comprising the amino acid sequence of SEQ ID NO:99, a first light chain comprising the amino acid sequence of SEQ ID NO:100, a second heavy chain comprising the amino acid sequence of SEQ ID NO:254 and a second light chain comprising the amino acid sequence of SEQ ID NO:255, or
(j) a first heavy chain comprising the amino acid sequence of SEQ ID NO:99, a first light chain comprising the amino acid sequence of SEQ ID NO:100, a second heavy chain comprising the amino acid sequence of SEQ ID NO:256 and a second light chain comprising the amino acid sequence of SEQ ID NO:257, or
(k) a first heavy chain comprising the amino acid sequence of SEQ ID NO:99, a first light chain comprising the amino acid sequence of SEQ ID NO:100, a second heavy chain comprising the amino acid sequence of SEQ ID NO:258 and a second light chain comprising the amino acid sequence of SEQ ID NO:259, or
(l) a first heavy chain comprising the amino acid sequence of SEQ ID NO:99, a first light chain comprising the amino acid sequence of SEQ ID NO:100, a second heavy chain comprising the amino acid sequence of SEQ ID NO:260 and a second light chain comprising the amino acid sequence of SEQ ID NO:261, or
(m) a first heavy chain comprising the amino acid sequence of SEQ ID NO:49, a first light chain comprising the amino acid sequence of SEQ ID NO:50, a second heavy chain comprising the amino acid sequence of SEQ ID NO:262 and a second light chain comprising the amino acid sequence of SEQ ID NO:263.

In another particular aspect, provided is a 4-1BBL trimer-containing antigen binding molecule, wherein the antigen binding molecule comprises

(a) a first heavy chain comprising the amino acid sequence of SEQ ID NO:99, a first light chain comprising the amino acid sequence of SEQ ID NO:100, a second heavy chain comprising the amino acid sequence of SEQ ID NO:266 and a second light chain comprising the amino acid sequence of SEQ ID NO:267, or
(b) a first heavy chain comprising the amino acid sequence of SEQ ID NO:99, a first light chain comprising the amino acid sequence of SEQ ID NO:100, a second heavy chain comprising the amino acid sequence of SEQ ID NO:268 and a second light chain comprising the amino acid sequence of SEQ ID NO:267, or
(c) a first heavy chain comprising the amino acid sequence of SEQ ID NO:99, a first light chain comprising the amino acid sequence of SEQ ID NO:100, a second heavy chain comprising the amino acid sequence of SEQ ID NO:269 and a second light chain comprising the amino acid sequence of SEQ ID NO:267, or
(d) a first heavy chain comprising the amino acid sequence of SEQ ID NO:99, a first light chain comprising the amino acid sequence of SEQ ID NO:100, a second heavy chain comprising the amino acid sequence of SEQ ID NO:270 and a second light chain comprising the amino acid sequence of SEQ ID NO:271, or
(e) a first heavy chain comprising the amino acid sequence of SEQ ID NO:99, a first light chain comprising the amino acid sequence of SEQ ID NO:100, a second heavy chain comprising the amino acid sequence of SEQ ID NO:272 and a second light chain comprising the amino acid sequence of SEQ ID NO:271, or
(f) a first heavy chain comprising the amino acid sequence of SEQ ID NO:99, a first light chain comprising the amino acid sequence of SEQ ID NO:100, a second heavy chain comprising the amino acid sequence of SEQ ID NO:273 and a second light chain comprising the amino acid sequence of SEQ ID NO:271, or
(g) a first heavy chain comprising the amino acid sequence of SEQ ID NO:99, a first light chain comprising the amino acid sequence of SEQ ID NO:100, a second heavy chain comprising the amino acid sequence of SEQ ID NO:274 and a second light chain comprising the amino acid sequence of SEQ ID NO:271.

In another aspect, the invention provides a novel humanized antibody that binds to carcinoembryonic antigen (CEA) (hu CEACAM5), comprising

(a) a variable heavy chain domain (VH) comprising (i) CDR-H1 comprising the amino acid sequence of SEQ ID NO:17, (ii) CDR-H2 comprising the amino acid sequence of SEQ ID NO:18, and (iii) CDR-H3 comprising the amino acid sequence of SEQ ID NO:19, and a variable light chain domain (VL) comprising (iv) CDR-L1 comprising the amino acid sequence of SEQ ID NO:20, (v) CDR-L2 comprising the amino acid sequence of SEQ ID NO:21, and (vi) CDR-L3 comprising the amino acid sequence of SEQ ID NO:22, or (b) a VH domain comprising (i) CDR-H1 comprising the amino acid sequence of SEQ ID NO:25, (ii) CDR-H2 comprising the amino acid sequence of SEQ ID NO:26, and (iii) CDR-H3 comprising the amino acid sequence of SEQ ID NO:27, and a VL domain comprising (iv) CDR-L1 comprising the amino acid sequence of SEQ ID NO:28, (v) CDR-L2 comprising the amino acid sequence of SEQ ID NO:29, and (vi) CDR-L3 comprising the amino acid sequence of SEQ ID NO:30.

In one aspect, the invention provides a novel humanized antibody that binds to the A2 domain of carcinoembryonic antigen (CEA) (hu CEACAM5), i.e. to the domain comprising the amino acid sequence of SEQ ID NO:311. Thus, the invention provides a novel humanized antibody that binds to the A2 domain of carcinoembryonic antigen (CEA) (hu CEACAM5), comprising (a) a variable heavy chain domain (VH) comprising (i) CDR-H1 comprising the amino acid sequence of SEQ ID NO:17, (ii) CDR-H2 comprising the amino acid sequence of SEQ ID NO:18, and (iii) CDR-H3 comprising the amino acid sequence of SEQ ID NO:19, and a variable light chain domain (VL) comprising (iv) CDR-L1 comprising the amino acid sequence of SEQ ID NO:20, (v) CDR-L2 comprising the amino acid sequence of SEQ ID NO:21, and (vi) CDR-L3 comprising the amino acid sequence of SEQ ID NO:22, or

(b) a VH domain comprising (i) CDR-H1 comprising the amino acid sequence of SEQ ID NO:25, (ii) CDR-H2 comprising the amino acid sequence of SEQ ID NO:26, and (iii) CDR-H3 comprising the amino acid sequence of SEQ ID NO:27, and a VL domain comprising (iv) CDR-L1 comprising the amino acid sequence of SEQ ID NO:28, (v) CDR-L2 comprising the amino acid sequence of SEQ ID NO:29, and (vi) CDR-L3 comprising the amino acid sequence of SEQ ID NO:30.

In one aspect, the humanized antibody that binds to the A2 domain of carcinoembryonic antigen (CEA) (huCEACAM5), comprises

(a) a VH domain comprising an amino acid sequence of SEQ ID NO:23 and a VL domain comprising an amino acid sequence of SEQ ID NO:24, or
(b) a VH domain comprising an amino acid sequence of SEQ ID NO:31 and a VL domain comprising an amino acid sequence of SEQ ID NO:32, or
(c) a VH domain comprising an amino acid sequence of SEQ ID NO:33 and a VL domain comprising an amino acid sequence of SEQ ID NO:34, or
(d) a VH domain comprising an amino acid sequence of SEQ ID NO:35 and a VL domain comprising an amino acid sequence of SEQ ID NO:36, or
(e) a VH domain comprising an amino acid sequence of SEQ ID NO:37 and a VL domain comprising an amino acid sequence of SEQ ID NO:38, or
(f) a VH domain comprising an amino acid sequence of SEQ ID NO:39 and a VL domain comprising an amino acid sequence of SEQ ID NO:40, or
(g) a VH domain comprising an amino acid sequence of SEQ ID NO:41 and a VL domain comprising an amino acid sequence of SEQ ID NO:42, or
(h) a VH domain comprising an amino acid sequence of SEQ ID NO:43 and a VL domain comprising an amino acid sequence of SEQ ID NO:44, or
(i) a VH domain comprising an amino acid sequence of SEQ ID NO:45 and a VL domain comprising an amino acid sequence of SEQ ID NO:46, or
(j) a VH domain comprising an amino acid sequence of SEQ ID NO:47 and a VL domain comprising an amino acid sequence of SEQ ID NO:48, or
(k) a VH domain comprising an amino acid sequence of SEQ ID NO:49 and a VL domain comprising an amino acid sequence of SEQ ID NO:50.
(l) a VH domain comprising an amino acid sequence of SEQ ID NO:51 and a VL domain comprising an amino acid sequence of SEQ ID NO:52, or
(m) a VH domain comprising an amino acid sequence of SEQ ID NO:53 and a VL domain comprising an amino acid sequence of SEQ ID NO:54.

In another aspect, provided is a novel humanized antibody that binds to carcinoembryonic antigen (CEA), comprising a VH domain comprising (i) CDR-H1 comprising the amino acid sequence of SEQ ID NO:65, (ii) CDR-H2 comprising the amino acid sequence of SEQ ID NO:66 or SEQ ID NO:67, and (iii) CDR-H3 comprising the amino acid sequence of SEQ ID NO:68, and a VL domain comprising (iv) CDR-L1 comprising the amino acid sequence of SEQ ID NO:69 or SEQ ID NO:70 or SEQ ID NO:313, (v) CDR-L2 comprising the amino acid sequence of SEQ ID NO:71 or SEQ ID NO:72 or SEQ ID NO:73, and (vi) CDR-L3 comprising the amino acid sequence of SEQ ID NO:74. In one aspect, the invention provides a humanized antibody that binds to the A1 domain of carcinoembryonic antigen (CEA) (hu CEACAM5), i.e. to the domain comprising the amino acid sequence of SEQ ID NO:312. Thus, the invention provides a humanized antibody that binds to the A1 domain of carcinoembryonic antigen (CEA) (hu CEACAM5), comprising a VH domain comprising (i) CDR-H1 comprising the amino acid sequence of SEQ ID NO:65, (ii) CDR-H2 comprising the amino acid sequence of SEQ ID NO:66 or SEQ ID NO:67, and (iii) CDR-H3 comprising the amino acid sequence of SEQ ID NO:68, and a VL domain comprising (iv) CDR-L1 comprising the amino acid sequence of SEQ ID NO:69 or SEQ ID NO:70 or SEQ ID NO:313, (v) CDR-L2 comprising the amino acid sequence of SEQ ID NO:71 or SEQ ID NO:72 or SEQ ID NO:73, and (vi) CDR-L3 comprising the amino acid sequence of SEQ ID NO:74.

Particularly, the humanized antibody that binds to the A1 domain of carcinoembryonic antigen (CEA) (hu CEACAM5), comprises a heavy chain variable region (VH) comprising the amino acid sequence of SEQ ID NO:75, SEQ ID NO:76, SEQ ID NO:77, SEQ ID NO:78, SEQ ID NO:79 or SEQ ID NO:80, and a light chain variable region (VL) comprising the amino acid sequence of SEQ ID NO:81, SEQ ID NO:82, SEQ ID NO:83, SEQ ID NO:84, SEQ ID NO:85 or SEQ ID NO:86. In one particular aspect, the antigen binding domain capable of specific binding to CEA comprises

(a) a VH domain comprising an amino acid sequence of SEQ ID NO:75 and a VL domain comprising an amino acid sequence of SEQ ID NO:85, or
(b) a VH domain comprising an amino acid sequence of SEQ ID NO:79 and a VL domain comprising an amino acid sequence of SEQ ID NO:85, or
(c) a VH domain comprising an amino acid sequence of SEQ ID NO:76 and a VL domain comprising an amino acid sequence of SEQ ID NO:85, or
(d) a VH domain comprising an amino acid sequence of SEQ ID NO:80 and a VL domain comprising an amino acid sequence of SEQ ID NO:84, or
(e) a VH domain comprising an amino acid sequence of SEQ ID NO:79 and a VL domain comprising an amino acid sequence of SEQ ID NO:84, or
(f) a VH domain comprising an amino acid sequence of SEQ ID NO:77 and a VL domain comprising an amino acid sequence of SEQ ID NO:84, or
(g) a VH domain comprising an amino acid sequence of SEQ ID NO:75 and a VL domain comprising an amino acid sequence of SEQ ID NO:84.

In a further aspect, the humanized antibody that binds to the A2 or to the A1 domain of carcinoembryonic antigen (CEA), respectively, is an antibody fragment, in particular a Fab molecule, that specifically binds to CEA. In one aspect, the antibody is a full length IgG1 antibody.

According to another aspect of the invention, there is provided isolated nucleic acid encoding a 4-1BBL trimer-containing antigen binding molecule or an antibody as defined herein before. The invention further provides a vector, particularly an expression vector, comprising the isolated nucleic acid molecule of the invention and a host cell comprising the isolated nucleic acid or the vector of the invention. In some embodiments the host cell is an eukaryotic cell, particularly a mammalian cell.

In another aspect, provided is a method for producing the 4-1BBL trimer-containing antigen binding molecule of the invention, comprising culturing the host cell of the invention under conditions suitable for expression of the 4-1BBL trimer-containing antigen binding molecule, and isolating the 4-1BBL trimer-containing antigen binding molecule. The invention also encompasses a 4-1BBL trimer-containing antigen binding molecule produced by the method of the invention.

The invention further provides a pharmaceutical composition comprising the 4-1BBL trimer-containing antigen binding molecule of the invention or an antibody of the invention and at least one pharmaceutically acceptable excipient. In another aspect, a pharmaceutical composition is provided comprising the 4-1BBL trimer-containing antigen binding molecule of the invention and at least one pharmaceutically acceptable excipient, further comprising an additional therapeutic agent, e.g. a chemotherapeutic agent and/or other agents for use in cancer immunotherapy. In a further aspect, provided is a pharmaceutical composition further comprising a T-cell activating anti-CD3 bispecific antibody, in particular an anti-CEA anti-CD3 bispecific antibody.

Also encompassed by the invention is the 4-1BBL trimer-containing antigen binding molecule of the invention, or the antibody or the pharmaceutical composition of the invention, for use as a medicament. In one aspect is provided the 4-1BBL trimer-containing antigen binding molecule of the invention, or the pharmaceutical composition of the invention, for use in the treatment of a disease in an individual in need thereof. In a specific aspect, provided is the 4-1BBL trimer-containing antigen binding molecule of the invention, or the antibody or the pharmaceutical composition of the invention, for use in the treatment of cancer. In another aspect, provided is the 4-1BBL trimer-containing antigen binding molecule of the invention, or the pharmaceutical composition of the invention, for use in up-regulating or prolonging cytotoxic T cell activity. In another aspect, provided is the 4-1BBL trimer-containing antigen binding molecule of the invention, or the pharmaceutical composition of the invention, for use in the treatment of cancer, wherein the 4-1BBL trimer-containing antigen binding molecule is used in combination with another therapeutic agent, in particular a T-cell activating anti-CD3 bispecific antibody. In one aspect, the T-cell activating anti-CD3 bispecific antibody is administered concurrently with, prior to, or subsequently to the 4-1BBL trimer-containing antigen binding molecule.

Also provided is the use of the 4-1BBL trimer-containing antigen binding molecule of the invention or the antibody of the invention for the manufacture of a medicament for the treatment of a disease in an individual in need thereof, in particular for the manufacture of a medicament for the treatment of cancer, as well as a method of treating a disease in an individual, comprising administering to said individual a therapeutically effective amount of a composition comprising the 4-1BBL trimer-containing antigen binding molecule as disclosed herein in a pharmaceutically acceptable form. In a specific aspect, the disease is cancer. Further provided is the use of the 4-1BBL trimer-containing antigen binding molecule of the invention for the manufacture of a medicament for the treatment of cancer, wherein the 4-1BBL trimer-containing antigen binding molecule is used in combination with a T-cell activating anti-CD3 bispecific antibody, in particular an anti-CEA/anti-CD3 antibody. Furthermore, provided is a method for treating an individual having cancer comprising administering to the subject an effective amount of the 4-1BBL trimer-containing antigen binding molecule of the invention, or a pharmaceutical composition thereof, and an effective amount of a T-cell activating anti-CD3 bispecific antibody, in particular an anti-Her2/anti-CD3 antibody. Also provided is a method of up-regulating or prolonging cytotoxic T cell activity in an individual having cancer, comprising administering to the individual an effective amount of the 4-1BBL trimer-containing antigen binding molecule of the invention, or the pharmaceutical composition of the invention. In any of the above embodiments the individual is preferably a mammal, particularly a human.

### BRIEF DESCRIPTION OF THE DRAWINGS

FIGS. 1A and 1B show the components for the assembly of the monovalent CEA-targeting split trimeric 4-1BB ligand Fc fusion antigen binding molecules. FIG. 1A shows the dimeric 4-1BB ligand that is fused at the C-terminus to a human IgG1-CL domain with mutations E123R and Q124K (charged variant) and FIG. 1B shows the monomeric 4-1BB ligand fused at its C-terminus to a human IgG1-CH1 domain with mutations K147E and K213E (charged variant). FIG. 1C illustrates schematically the structure of the monovalent CEA-targeting split trimeric 4-1BB ligand Fc (kih) P329G LALA fusion antigen binding molecule comprising CH-CL cross with charged residues. The thick black point stands for the knob-into-hole modification. * symbolizes amino acid modifications in the CH1 and CL domain (so-called charged variant).

FIG. 2 shows the binding of humanized A5B7 huIgG1 P329G LALA variants to MKN-45 as compared to the binding of the parental murine A5B7 antibody. Antibodies were detected with a fluorescently labeled secondary antibody and fluorescence was measured by flow cytometry.

FIGS. 3A to 3C are schematic illustrations of the recombinant proteins displaying different domains of the CEACAM5 protein that were used as antigens in the phage display campaign. FIG. 3A shows construct NABA-avi-His consisting of the 4 Ig-like domains N, A1, B and A2. FIG. 3B shows the construct N(A2B2)A-avi-His and FIG. 3C illustrates the construct NA(B2)A-avi-His.

FIGS. 4A and 4B show the VH and VL sequences, respectively, of humanized CEA antibody A5H1EL1D wherein the randomized positions are marked with X.

Schematic drawings of the phage vectors of the affinity maturation libraries are shown in FIG. 5A (CDRH1/H2 affinity maturation library), FIG. 5B (CDRL1/H2 affinity maturation library) and FIG. 5C (CDRH3/CDRL3 amplification library).

FIGS. 6A and 6B show an alignment of the VH amino acid sequences (FIG. 6A) and VL amino acid sequences (FIG. 6B) of the affinity-matured, humanized CEA(A5H1EL1D) antibody variants.

FIGS. 7A and 7B show an alignment of the VH amino acid sequences (FIG. 7A) and VL amino acid sequences (FIG. 7B) of the humanized 1MFE23 antibody variants.

FIGS. 8A, 8B and 8C show the binding of humanized MFE23 huIgG1 P329G LALA variants to MKN-45 as compared to the binding of the parental murine MFE23 antibody. Antibodies were detected with a fluorescently labeled secondary antibody and fluorescence was measured by flow cytometry. The graph was split into three graphs displaying low binding, intermediate binding and similar binding to the parental MFE23 clone.

FIGS. 9A to 9H relate to simultaneous binding of CEA-targeting trimeric split 4-1BBL molecules to hu4-1BB and huN(A2-B2)A or hu(NA1)BA. FIG. 9A shows the setup of the assay. FIG. 9B shows the simultaneous binding of CEA(A5B7)-4-1BBL to huN(A2-B2)A and hu4-1BB-Fc(kih). FIG. 9C shows simultaneous binding of CEA(A5H1EL1D)-4-1BBL to huN(A2-B2)A and hu4-1BB-Fc(kih). FIG. 9D shows simultaneous binding of CEA(MFE23)-4-1BBL to hu(NA1)BA and hu4-1BB-Fc(kih). FIG. 9E shows simultaneous binding of CEA(MFE23-L28-H24)-4-1BBL to hu(NA1)BA and hu4-1BB-Fc(kih). FIG. 9F shows simultaneous binding of CEA(MFE23-L28-H28)-4-1BBL to hu(NA1)BA and hu4-1BB-Fc(kih). FIG. 9G shows simultaneous binding of CEA(P001.177)-4-1BBL to huN(A2-B2)A and hu4-1BB-Fc(kih). FIG. 9H shows simultaneous binding of CEA(P005.102)-4-1BBL to huN(A2-B2)A and hu4-1BB-Fc(kih). Duplicates or triplicates are shown.

The cell surface CEACAM5 expression level of different CEACAM5 expressing clones used for the binding assays is shown in FIG. 10. Chinese hamster ovary cell line called CHO-k1 (ATCC CRL-9618) was transfected with cynomolgus monkey CEACAM5 (CHO-k1-cyno CEACAM5 clone 8) or human CEACAM5 (CHO-k1-huCEACAM5 clone 11, clone 12, clone 13, clone 17). The expression levels were determined using titrated APC-labeled anti-CD66 specific detection antibody (clone CD66AB.1.1) by flow cytometry. Shown is the median of fluorescence intensity versus the concentration of the detection antibody, whereby the median of fluorescence intensity correlates positively with the amount of bound detection antibody and therefore with the expression level of CEACAM5 molecules on the cell surface. CHO-k1-cynoCEACAM5 clone 8 and CHO-k1-huCEACAM5 clone 11 display a similar cell surface CEACAM5 expression, whereas CHO-k1-huCEACAM5 clone 12, 13 and 17 show a high cell surface CEACAM5 expression level.

In FIGS. 11A to 11E the binding to cynomolgus monkey CEACAM5 or human CEACAM5-expressing CHO-k1 cells is shown. The concentration of different bispecific CEA-4-1BBL molecules containing as CEA-binder either MFE23 (parental) or humanized MFE23 (huMFE23-L28-H24 or huMFE23-L28-H28) or T84.66-LCHA (reference) or control molecules is blotted against the median of fluorescence intensity of the PE-conjugated secondary detection antibody. All values are baseline corrected by subtracting the baseline values of the blank control (e.g. no primary only secondary detection antibody). All CEACAM5 antigen binding domain-containing constructs bind efficiently to human CEACAM5-expressing cells (FIGS. 11B, 11C, 11D and 11E). In contrast, none of the CEA-binders binds detectable to cynomolgus monkey CEACAM5 (FIG. 11A).

Also in FIGS. 12A to 12E the binding to cynomolgus monkey CEACAM5 or human CEACAM5-expressing CHO-k1 cells is shown. The concentration of different bispecific CEA-4-1BBL molecules containing as CEA-binder either A5B7 (parental) or humanized A5B7 (A5H1EL1D or MEDI-565) or T84.66-LCHA (reference) or control molecules is blotted against the median of fluorescence intensity of the PE-conjugated secondary detection antibody. All values are baseline corrected by subtracting the baseline values of the blank control (e.g. no primary only secondary detection antibody). All CEACAM5 antigen binding domain-containing constructs bind efficiently to human CEACAM5-expressing cells (FIGS. 12B, 12C, 12D and 12E) as well as to cynomolgus monkey CEACAM5 (FIG. 12A), e.g. the shown binders are cynomolgus monkey/human cross-reactive.

FIG. 13A to 13C show the binding to cynomolgus monkey CEACAM5 or human CEACAM5-expressing CHO-k1 cells. The concentration of different bispecific CEA-4-1BBL molecules containing as CEA-binder either A5B7 (parental) or humanized A5B7 (A5H1EL1D or MEDI-565) or affinity maturated A5H1EL1D (aff. mat. A5H1EL1D P001.177 and aff. mat. A5H1EL1D P005.102) or T84.66-LCHA (reference) or control molecules is blotted against the median of fluorescence intensity of the PE-conjugated secondary detection antibody. All values are baseline corrected by subtracting the baseline values of the blank control (e.g. no primary only secondary detection antibody). All CEACAM5 antigen binding domain-containing constructs bind efficiently to human CEACAM5-expressing cells (FIGS. 13B and 13C) as well as to cynomolgus monkey CEACAM5 (FIG. 13A), e.g. the shown binders are cynomolgus monkey/human cross-reactive, whereas the binder A5H1EL1D shows a limited affinity. The affinity maturation increased the affinity of A5H1EL1D to human CEACAM5 and in case of affinity maturated A5H1EL1D P005.102 also to cynomolgus monkey CEACAM5.

FIGS. 14A to 14D show the NFκB-mediated luciferase expression activity in 4-1BB expressing reporter cell line Jurkat-hu4-1BB-NFκB-luc2. To test the functionality of bispecific CEA-4-1BBL binding molecules versus controls, molecules were incubated with the reporter cell line Jurkat-hu4-1BB-NFκB-luc2 in the absence or presence of cynomolgus monkey or human CEACAM5 expressing CHO-k1 cell lines in a 1:5 ratio for 5 h. The concentration of CEA-4-1BBL molecules or its controls are blotted against the units of released light (RLU) measured after 5 h of incubation and addition of Luciferase detection solution. All values are baseline corrected by subtracting the baseline values of the blank control (e.g. no antibodies added). In correlation to the binding assay molecules containing the anti-human CEACAM5 specific clone MFE23 (parental) or the humanized versions (huMFE23-L28-H24, huMFE23-L28-H28, sm9b) or the reference clone T84.66-LCHA, molecules bind and are crosslinked via human CEACAM5 and therefore induce 4-1BB-mediated activation of the reporter cell line Jurkat-hu4-1BB-NFκB-luc2 (FIGS. 14C and 14D). In contrast, in the absence of human CEACAM5 (FIG. 14A) or in the presence of cynomolgus CEACAM5 no activation is induced (FIG. 14B).

Also FIGS. 15A to 15D show the NFκB-mediated luciferase expression activity in 4-1BB expressing reporter cell line Jurkat-hu4-1BB-NFκB-luc2. To test the functionality of bispecific CEA-4-1BBL binding molecules versus controls, molecules were incubated with the reporter cell line Jurkat-hu4-1BB-NFκB-luc2 in the absence or presence of cynomolgus monkey or human CEACAM5 expressing CHO-k1 cell lines in a 1:5 ratio for 5 h. The concentration of CEA-4-1BBL molecules or its controls are blotted against the units of released light (RLU) measured after 5 h of incubation and addition of Luciferase detection solution. All values are baseline corrected by subtracting the baseline values of the blank control (e.g. no antibodies added). In correlation to the binding assay molecules containing the anti-human CEACAM5 specific clone A5B7 (parental) or the humanized versions (A5H1EL1D or MEDI-565) or the affinity maturated versions (P005.102 and P001.177) or the reference clone T84.66-LCHA, molecules bind and are cross-linked via human CEACAM5 and therefore induce 4-1BB-mediated activation of the reporter cell line Jurkat-hu4-1BB-NFkB-luc2 (FIGS. 15C and 15D). In contrast, in the absence of human CEACAM5 (FIG. 15A) no molecule induces activation. In the presence of cynomolgus CEACAM5, only molecules containing A5B7 derived clones induce activity whereas CEA(T84.77-LCHA)-4-1BBL does not (FIG. 15B).

### DETAILED DESCRIPTION OF THE INVENTION

### Definitions

Unless defined otherwise, technical and scientific terms used herein have the same meaning as generally used in the art to which this invention belongs. For purposes of interpreting this specification, the following definitions will apply and whenever appropriate, terms used in the singular will also include the plural and vice versa.

As used herein, the term “antigen binding molecule” refers in its broadest sense to a molecule that specifically binds an antigenic determinant. Examples of antigen binding molecules are antibodies, antibody fragments and scaffold antigen binding proteins.

The term “antigen binding domain” refers to the part of an antigen binding molecule that comprises the area which specifically binds to and is complementary to part or all of an antigen. Where an antigen is large, an antigen binding molecule may only bind to a particular part of the antigen, which part is termed an epitope. An antigen binding domain may be provided by, for example, one or more variable domains (also called variable regions). Preferably, an antigen binding domain comprises an antibody light chain variable region (VL) and an antibody heavy chain variable region (VH).

As used herein, the term “antigen binding domain capable of specific binding to CEA” or “moiety capable of specific binding to CEA” refers to a polypeptide molecule that specifically binds to CEA. In one aspect, the antigen binding domain is able to activate or inhibit signaling through CEA. In a particular aspect, the antigen binding domain is able to direct the entity to which it is attached (e.g. the 4-1BBL trimer) to a target site, for example to a specific type of tumor cell bearing CEA on its surface. Antigen binding domains capable of specific binding to CEA include antibodies and fragments thereof as further defined herein. In relation to an antibody or fragment thereof, the term “moiety capable of specific binding to a target cell antigen” refers to the part of the molecule that comprises the area which specifically binds to and is complementary to part or all of an antigen. A moiety capable of specific antigen binding may be provided, for example, by one or more antibody variable domains (also called antibody variable regions). Particularly, a moiety capable of specific antigen binding comprises an antibody light chain variable region (VL) and an antibody heavy chain variable region (VH). By “specifically binds” is meant that the binding is selective for the antigen and can be discriminated from unwanted or nonspecific interactions.

The term “antibody” herein is used in the broadest sense and encompasses various antibody structures, including but not limited to monoclonal antibodies, polyclonal antibodies, monospecific and multispecific antibodies (e.g., bispecific antibodies), and antibody fragments so long as they exhibit the desired antigen-binding activity.

The term “monoclonal antibody” as used herein refers to an antibody obtained from a population of substantially homogeneous antibodies, i.e., the individual antibodies comprising the population are identical and/or bind the same epitope, except for possible variant antibodies, e.g. containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen.

The term “monospecific” antibody as used herein denotes an antibody that has one or more binding sites each of which bind to the same epitope of the same antigen. The term “bispecific” means that the antigen binding molecule is able to specifically bind to at least two distinct antigenic determinants. Typically, a bispecific antigen binding molecule comprises two antigen binding sites, each of which is specific for a different antigenic determinant. In certain embodiments the bispecific antigen binding molecule is capable of simultaneously binding two antigenic determinants, particularly two antigenic determinants expressed on two distinct cells. The term “multispecific” means that the antigen binding molecule is able to specifically bind to at least two distinct antigenic determinants. A multispecific antigen binding molecule can be, for example, a bispecific antigen binding molecule.

The term “valent” as used within the current application denotes the presence of a specified number of binding sites in an antigen binding molecule. As such, the terms “monovalent”, “bivalent”, “tetravalent”, and “hexavalent” denote the presence of one binding site, two binding sites, four binding sites, and six binding sites, respectively, in an antigen binding molecule.

The terms “full length antibody”, “intact antibody”, and “whole antibody” are used herein interchangeably to refer to an antibody having a structure substantially similar to a native antibody structure. “Native antibodies” refer to naturally occurring immunoglobulin molecules with varying structures. For example, native IgG-class antibodies are heterotetrameric glycoproteins of about 150,000 daltons, composed of two light chains and two heavy chains that are disulfide-bonded. From N- to C-terminus, each heavy chain has a variable region (VH), also called a variable heavy domain or a heavy chain variable domain, followed by three constant domains (CH1, CH2, and CH3), also called a heavy chain constant region. Similarly, from N- to C-terminus, each light chain has a variable region (VL), also called a variable light domain or a light chain variable domain, followed by a light chain constant domain (CL), also called a light chain constant region. The heavy chain of an antibody may be assigned to one of five types, called α (IgA), δ (IgD), ε (IgE), γ (IgG), or μ (IgM), some of which may be further divided into subtypes, e.g. γ1 (IgG1), γ2 (IgG2), γ3 (IgG3), γ4 (IgG4), α1 (IgA1) and α2 (IgA2). The light chain of an antibody may be assigned to one of two types, called kappa (κ) and lambda (λ), based on the amino acid sequence of its constant domain.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies, triabodies, tetrabodies, cross-Fab molecules; linear antibodies; single-chain antibody molecules (e.g. scFv); and single domain antibodies. For a review of certain antibody fragments, see Hudson et al., Nat Med 9, 129-134 (2003). For a review of scFv fragments, see e.g. Pluckthun, in The Pharmacology of Monoclonal Antibodies, vol. 113, Rosenburg and Moore eds., Springer-Verlag, New York, pp. 269-315 (1994); see also WO 93/16185; and U.S. Pat. Nos. 5,571,894 and 5,587,458. For discussion of Fab and F(ab′)2 fragments comprising salvage receptor binding epitope residues and having increased in vivo half-life, see U.S. Pat. No. 5,869,046. Diabodies are antibody fragments with two antigen-binding sites that may be bivalent or bispecific, see, for example, EP 404,097; WO 1993/01161; Hudson et al., Nat Med 9, 129-134 (2003); and Hollinger et al., Proc Natl Acad Sci USA 90, 6444-6448 (1993). Triabodies and tetrabodies are also described in Hudson et al., Nat Med 9, 129-134 (2003). Single-domain antibodies are antibody fragments comprising all or a portion of the heavy chain variable domain or all or a portion of the light chain variable domain of an antibody. In certain embodiments, a single-domain antibody is a human single-domain antibody (Domantis, Inc., Waltham, Mass.; see e.g. U.S. Pat. No. 6,248,516 B1). Antibody fragments can be made by various techniques, including but not limited to proteolytic digestion of an intact antibody as well as production by recombinant host cells (e.g. E. coli or phage), as described herein.

Papain digestion of intact antibodies produces two identical antigen-binding fragments, called “Fab” fragments containing each the heavy- and light-chain variable domains and also the constant domain of the light chain and the first constant domain (CH1) of the heavy chain. As used herein, Thus, the term “Fab fragment” or “Fab molecule” refers to an antibody fragment comprising a light chain fragment comprising a VL domain and a constant domain of a light chain (CL), and a VH domain and a first constant domain (CH1) of a heavy chain. Fab′ fragments differ from Fab fragments by the addition of a few residues at the carboxy terminus of the heavy chain CH1 domain including one or more cysteins from the antibody hinge region. Fab′-SH are Fab′ fragments in which the cysteine residue(s) of the constant domains bear a free thiol group. Pepsin treatment yields an F(ab′)₂ fragment that has two antigen-combining sites (two Fab fragments) and a part of the Fc region.

The term “cross-Fab molecule” or “cross-Fab fragment” or “xFab fragment” or “crossover Fab fragment” refers to a Fab molecule, wherein either the variable regions or the constant regions of the heavy and light chain are exchanged. Two different chain compositions of a crossover Fab molecule are possible and comprised in the bispecific antibodies of the invention: On the one hand, the variable regions of the Fab heavy and light chain are exchanged, i.e. the crossover Fab molecule comprises a peptide chain composed of the light chain variable region (VL) and the heavy chain constant region (CH1), and a peptide chain composed of the heavy chain variable region (VH) and the light chain constant region (CL). This crossover Fab molecule is also referred to as CrossFab_{(VLVH)}. On the other hand, when the constant regions of the Fab heavy and light chain are exchanged, the crossover Fab molecule comprises a peptide chain composed of the heavy chain variable region (VH) and the light chain constant region (CL), and a peptide chain composed of the light chain variable region (VL) and the heavy chain constant region (CH1). This crossover Fab molecule is also referred to as CrossFab_{(CLCH1)}.

A “single chain Fab fragment” or “scFab” is a polypeptide consisting of an antibody heavy chain variable domain (VH), an antibody constant domain 1 (CH1), an antibody light chain variable domain (VL), an antibody light chain constant domain (CL) and a linker, wherein said antibody domains and said linker have one of the following orders in N-terminal to C-terminal direction: a) VH-CH1-linker-VL-CL, b) VL-CL-linker-VH-CH1, c) VH-CL-linker-VL-CH1 or d) VL-CH1-linker-VH-CL; and wherein said linker is a polypeptide of at least 30 amino acids, preferably between 32 and 50 amino acids. Said single chain Fab fragments are stabilized via the natural disulfide bond between the CL domain and the CH1 domain. In addition, these single chain Fab molecules might be further stabilized by generation of interchain disulfide bonds via insertion of cysteine residues (e.g. position 44 in the variable heavy chain and position 100 in the variable light chain according to Kabat numbering).

A “crossover single chain Fab fragment” or “x-scFab” is a is a polypeptide consisting of an antibody heavy chain variable domain (VH), an antibody constant domain 1 (CH1), an antibody light chain variable domain (VL), an antibody light chain constant domain (CL) and a linker, wherein said antibody domains and said linker have one of the following orders in N-terminal to C-terminal direction: a) VH-CL-linker-VL-CH1 and b) VL-CH1-linker-VH-CL; wherein VH and VL form together an antigen-binding site which binds specifically to an antigen and wherein said linker is a polypeptide of at least 30 amino acids. In addition, these x-scFab molecules might be further stabilized by generation of interchain disulfide bonds via insertion of cysteine residues (e.g. position 44 in the variable heavy chain and position 100 in the variable light chain according to Kabat numbering).

A “single-chain variable fragment (scFv)” is a fusion protein of the variable regions of the heavy (V_{H}) and light chains (V_{L}) of an antibody, connected with a short linker peptide of ten to about 25 amino acids. The linker is usually rich in glycine for flexibility, as well as serine or threonine for solubility, and can either connect the N-terminus of the VH with the C-terminus of the VL, or vice versa. This protein retains the specificity of the original antibody, despite removal of the constant regions and the introduction of the linker. scFv antibodies are, e.g. described in Houston, J. S., Methods in Enzymol. 203 (1991) 46-96). In addition, antibody fragments comprise single chain polypeptides having the characteristics of a VH domain, namely being able to assemble together with a VL domain, or of a VL domain, namely being able to assemble together with a VH domain to a functional antigen binding site and thereby providing the antigen binding property of full length antibodies.

“Scaffold antigen binding proteins” are known in the art, for example, fibronectin and designed ankyrin repeat proteins (DARPins) have been used as alternative scaffolds for antigen-binding domains, see, e.g., Gebauer and Skerra, Engineered protein scaffolds as next-generation antibody therapeutics. Curr Opin Chem Biol 13:245-255 (2009) and Stumpp et al., Darpins: A new generation of protein therapeutics. Drug Discovery Today 13: 695-701 (2008). In one aspect of the invention, a scaffold antigen binding protein is selected from the group consisting of CTLA-4 (Evibody), Lipocalins (Anticalin), a Protein A-derived molecule such as Z-domain of Protein A (Affibody), an A-domain (Avimer/Maxibody), a serum transferrin (trans-body); a designed ankyrin repeat protein (DARPin), a variable domain of antibody light chain or heavy chain (single-domain antibody, sdAb), a variable domain of antibody heavy chain (nanobody, aVH), V_{NAR} fragments, a fibronectin (AdNectin), a C-type lectin domain (Tetranectin); a variable domain of a new antigen receptor beta-lactamase (V_{NAR} fragments), a human gamma-crystallin or ubiquitin (Affilin molecules); a kunitz type domain of human protease inhibitors, microbodies such as the proteins from the knottin family, peptide aptamers and fibronectin (adnectin). CTLA-4 (Cytotoxic T Lymphocyte-associated Antigen 4) is a CD28-family receptor expressed on mainly CD4⁺ T-cells. Its extracellular domain has a variable domain-like Ig fold. Loops corresponding to CDRs of antibodies can be substituted with heterologous sequence to confer different binding properties. CTLA-4 molecules engineered to have different binding specificities are also known as Evibodies (e.g. U.S. Pat. No. 7,166,697B1). Evibodies are around the same size as the isolated variable region of an antibody (e.g. a domain antibody). For further details see Journal of Immunological Methods 248 (1-2), 31-45 (2001). Lipocalins are a family of extracellular proteins which transport small hydrophobic molecules such as steroids, bilins, retinoids and lipids. They have a rigid beta-sheet secondary structure with a number of loops at the open end of the conical structure which can be engineered to bind to different target antigens. Anticalins are between 160-180 amino acids in size, and are derived from lipocalins. For further details see Biochim Biophys Acta 1482: 337-350 (2000), U.S. Pat. No. 7,250,297B1 and US20070224633. An affibody is a scaffold derived from Protein A of Staphylococcus aureus which can be engineered to bind to antigen. The domain consists of a three-helical bundle of approximately 58 amino acids. Libraries have been generated by randomization of surface residues. For further details see Protein Eng. Des. Sel. 2004, 17, 455-462 and EP 1641818A1. Avimers are multidomain proteins derived from the A-domain scaffold family. The native domains of approximately 35 amino acids adopt a defined disulfide bonded structure. Diversity is generated by shuffling of the natural variation exhibited by the family of A-domains. For further details see Nature Biotechnology 23(12), 1556-1561 (2005) and Expert Opinion on Investigational Drugs 16(6), 909-917 (June 2007). A transferrin is a monomeric serum transport glycoprotein. Transferrins can be engineered to bind different target antigens by insertion of peptide sequences in a permissive surface loop. Examples of engineered transferrin scaffolds include the Trans-body. For further details see J. Biol. Chem 274, 24066-24073 (1999). Designed Ankyrin Repeat Proteins (DARPins) are derived from Ankyrin which is a family of proteins that mediate attachment of integral membrane proteins to the cytoskeleton. A single ankyrin repeat is a 33 residue motif consisting of two alpha-helices and a beta-turn. They can be engineered to bind different target antigens by randomizing residues in the first alpha-helix and a beta-turn of each repeat. Their binding interface can be increased by increasing the number of modules (a method of affinity maturation). For further details see J. Mol. Biol. 332, 489-503 (2003), PNAS 100(4), 1700-1705 (2003) and J. Mol. Biol. 369, 1015-1028 (2007) and US20040132028A1. A single-domain antibody is an antibody fragment consisting of a single monomeric variable antibody domain. The first single domains were derived from the variable domain of the antibody heavy chain from camelids (nanobodies or V_{H}H fragments). Furthermore, the term single-domain antibody includes an autonomous human heavy chain variable domain (aVH) or V_{NAR} fragments derived from sharks. Fibronectin is a scaffold which can be engineered to bind to antigen. Adnectins consists of a backbone of the natural amino acid sequence of the 10th domain of the 15 repeating units of human fibronectin type III (FN3). Three loops at one end of the .beta.-sandwich can be engineered to enable an Adnectin to specifically recognize a therapeutic target of interest. For further details see Protein Eng. Des. Sel. 18, 435-444 (2005), US20080139791, WO2005056764 and U.S. Pat. No. 6,818,418B1. Peptide aptamers are combinatorial recognition molecules that consist of a constant scaffold protein, typically thioredoxin (TrxA) which contains a constrained variable peptide loop inserted at the active site. For further details see Expert Opin. Biol. Ther. 5, 783-797 (2005). Microbodies are derived from naturally occurring microproteins of 25-50 amino acids in length which contain 3-4 cysteine bridges—examples of microproteins include KalataBl and conotoxin and knottins. The microproteins have a loop which can beengineered to include upto 25 amino acids without affecting the overall fold of the microprotein. For further details of engineered knottin domains, see WO2008098796.

Lipocalins are a family of extracellular proteins which transport small hydrophobic molecules such as steroids, bilins, retinoids and lipids. They have a rigid beta-sheet secondary structure with a number of loops at the open end of the conical structure which can be engineered to bind to different target antigens. Anticalins are between 160-180 amino acids in size, and are derived from lipocalins. For further details see Biochim Biophys Acta 1482: 337-350 (2000), Biodrugs 19(5), 279-288 (2005), U.S. Pat. No. 7,250,297B1 and US20070224633.

An “antigen binding molecule that binds to the same epitope” as a reference molecule refers to an antigen binding molecule that blocks binding of the reference molecule to its antigen in a competition assay by 50% or more, and conversely, the reference molecule blocks binding of the antigen binding molecule to its antigen in a competition assay by 50% or more.

As used herein, the term “antigenic determinant” is synonymous with “antigen” and “epitope,” and refers to a site (e.g. a contiguous stretch of amino acids or a conformational configuration made up of different regions of non-contiguous amino acids) on a polypeptide macromolecule to which an antigen binding moiety binds, forming an antigen binding moiety-antigen complex. Useful antigenic determinants can be found, for example, on the surfaces of tumor cells, on the surfaces of virus-infected cells, on the surfaces of other diseased cells, on the surface of immune cells, free in blood serum, and/or in the extracellular matrix (ECM). The proteins useful as antigens herein can be any native form the proteins from any vertebrate source, including mammals such as primates (e.g. humans) and rodents (e.g. mice and rats), unless otherwise indicated. In a particular embodiment the antigen is a human protein. Where reference is made to a specific protein herein, the term encompasses the “full-length”, unprocessed protein as well as any form of the protein that results from processing in the cell. The term also encompasses naturally occurring variants of the protein, e.g. splice variants or allelic variants.

The term “Carcinoembroynic antigen (CEA)”, also known as Carcinoembryonic antigen-related cell adhesion molecule 5 (CEACAM5), refers to any native CEA from any vertebrate source, including mammals such as primates (e.g. humans) non-human primates (e.g. cynomolgus monkeys) and rodents (e.g. mice and rats), unless otherwise indicated. The amino acid sequence of human CEA is shown in UniProt accession no. P06731 (version 151, SEQ ID NO:108). CEA has long been identified as a tumor-associated antigen (Gold and Freedman, J Exp Med., 121:439-462, 1965; Bernstein N. L., J Clin Oncol., 20:2197-2207, 2002). Originally classified as a protein expressed only in fetal tissue, CEA has now been identified in several normal adult tissues. These tissues are primarily epithelial in origin, including cells of the gastrointestinal, respiratory, and urogential tracts, and cells of colon, cervix, sweat glands, and prostate (Nap et al., Tumour Biol., 9(2-3):145-53, 1988; Nap et al., Cancer Res., 52(8):2329-23339, 1992). Tumors of epithelial origin, as well as their metastases, contain CEA as a tumor associated antigen. While the presence of CEA itself does not indicate transformation to a cancerous cell, the distribution of CEA is indicative. In normal tissue, CEA is generally expressed on the apical surface of the cell (Hammarström S., Semin Cancer Biol. 9(2):67-81 (1999)), making it inaccessible to antibody in the blood stream. In contrast to normal tissue, CEA tends to be expressed over the entire surface of cancerous cells (Hammarström S., Semin Cancer Biol. 9(2):67-81 (1999)). This change of expression pattern makes CEA accessible to antibody binding in cancerous cells. In addition, CEA expression increases in cancerous cells. Furthermore, increased CEA expression promotes increased intercellular adhesions, which may lead to metastasis (Marshall J., Semin Oncol., 30 (a Suppl. 8):30-6, 2003). The prevalence of CEA expression in various tumor entities is generally very high. In concordance with published data, own analyses performed in tissue samples confirmed its high prevalence, with approximately 95% in colorectal carcinoma (CRC), 90% in pancreatic cancer, 80% in gastric cancer, 60% in non-small cell lung cancer (NSCLC, where it is co-expressed with HER3), and 40% in breast cancer; low expression was found in small cell lung cancer and glioblastoma.

CEA is readily cleaved from the cell surface and shed into the blood stream from tumors, either directly or via the lymphatics. Because of this property, the level of serum CEA has been used as a clinical marker for diagnosis of cancers and screening for recurrence of cancers, particularly colorectal cancer (Goldenberg D M., The International Journal of Biological Markers, 7:183-188, 1992; Chau I., et al., J Clin Oncol., 22:1420-1429, 2004; Flamini et al., Clin Cancer Res; 12(23):6985-6988, 2006).

The term “anti-CEA antigen binding molecule” or “antigen binding molecule capable of specific binding to CEA” refers to an antigen binding molecule that is capable of binding to CEA with sufficient affinity such that the antigen binding molecule is useful as a diagnostic and/or therapeutic agent in targeting CEA. The antigen binding molecule includes but is not limited to, antibodies, Fab molecules, crossover Fab molecules, single chain Fab molecules, Fv molecules, scFv molecules, single domain antibodies, and VH and scaffold antigen binding protein. In one aspect, the extent of binding of an anti-CEA antigen binding molecule to an unrelated, non-CEA protein is less than about 10% of the binding of the antigen binding molecule to CEA as measured, e.g., by surface plasmon resonance (SPR). In particular, an antigen binding molecule that is capable of specific binding to CEA has a dissociation constant (K_{d}) of ≤1 μM, ≤500 nM, ≤200 nM, ≤100 nM, ≤10 nM, ≤1 nM, ≤0.1 nM, ≤0.01 nM, or ≤0.001 nM (e.g. 10⁻⁸M or less, e.g. from 10⁻⁸ M to 10⁻¹³M, e.g., from 10⁻⁹M to 10⁻¹³M). In certain aspects, an anti-CEA antigen binding molecule binds to CEA from different species. In certain aspects, the anti-CEA antigen binding molecule binds to human and cynomolgus CEA.

The term “epitope” denotes the site on an antigen, either proteinaceous or non-proteinaceous, to which an anti-CEA antibody binds. Epitopes can be formed from contiguous amino acid stretches (linear epitope) or comprise non-contiguous amino acids (conformational epitope), e.g., coming in spatial proximity due to the folding of the antigen, i.e. by the tertiary folding of a proteinaceous antigen. Linear epitopes are typically still bound by an antibody after exposure of the proteinaceous antigen to denaturing agents, whereas conformational epitopes are typically destroyed upon treatment with denaturing agents. An epitope comprises at least 3, at least 4, at least 5, at least 6, at least 7, or 8-10 amino acids in a unique spatial conformation.

By “specific binding” is meant that the binding is selective for the antigen and can be discriminated from unwanted or non-specific interactions. The ability of an antigen binding molecule to bind to a specific antigen can be measured either through an enzyme-linked immunosorbent assay (ELISA) or other techniques familiar to one of skill in the art, e.g. Surface Plasmon Resonance (SPR) technique (analyzed on a BIAcore instrument) (Liljeblad et al., Glyco J 17, 323-329 (2000)), and traditional binding assays (Heeley, Endocr Res 28, 217-229 (2002)). In one embodiment, the extent of binding of an antigen binding molecule to an unrelated protein is less than about 10% of the binding of the antigen binding molecule to the antigen as measured, e.g. by SPR. In certain embodiments, a molecule that binds to the antigen has a dissociation constant (Kd) of ≤1 μM, ≤100 nM, ≤10 nM, ≤1 nM, ≤0.1 nM, ≤0.01 nM, or ≤0.001 nM (e.g. 10⁻⁸M or less, e.g. from 10⁻⁸ M to 10⁻¹³M, e.g. from 10⁻⁹M to 10⁻¹³M).

“Affinity” or “binding affinity” refers to the strength of the sum total of non-covalent interactions between a single binding site of a molecule (e.g. an antibody) and its binding partner (e.g. an antigen). Unless indicated otherwise, as used herein, “binding affinity” refers to intrinsic binding affinity which reflects a 1:1 interaction between members of a binding pair (e.g. antibody and antigen). The affinity of a molecule X for its partner Y can generally be represented by the dissociation constant (Kd), which is the ratio of dissociation and association rate constants (k_{off} and kₒₙ, respectively). Thus, equivalent affinities may comprise different rate constants, as long as the ratio of the rate constants remains the same. Affinity can be measured by common methods known in the art, including those described herein. A particular method for measuring affinity is Surface Plasmon Resonance (SPR).

As used herein, the term “affinity matured” in the context of antigen binding molecules (e.g., antibodies) refers to an antigen binding molecule that is derived from a reference antigen binding molecule, e.g., by mutation, binds to the same antigen, preferably binds to the same epitope, as the reference antibody; and has a higher affinity for the antigen than that of the reference antigen binding molecule. Affinity maturation generally involves modification of one or more amino acid residues in one or more CDRs of the antigen binding molecule. Typically, the affinity matured antigen binding molecule binds to the same epitope as the initial reference antigen binding molecule.

A “target cell antigen” as used herein refers to an antigenic determinant presented on the surface of a target cell, for example a T-cell or B-cell, a cell in a tumor such as a cancer cell or a cell of the tumor stroma. In certain aspects, the target cell antigen is an antigen on the surface of cancer cell. In one aspect, the target cell antigen is CEA.

The term “variable domain” or “variable region” refers to the domain of an antibody heavy or light chain that is involved in binding the antigen binding molecule to antigen. The variable domains of the heavy chain and light chain (VH and VL, respectively) of a native antibody generally have similar structures, with each domain comprising four conserved framework regions (FRs) and three hypervariable regions (HVRs). See, e.g., Kindt et al., Kuby Immunology, 6th ed., W.H. Freeman and Co., page 91 (2007). A single VH or VL domain may be sufficient to confer antigen-binding specificity.

The term “hypervariable region” or “HVR” as used herein refers to each of the regions of an antigen binding variable domain which are hypervariable in sequence and which determine antigen binding specificity, for example “complementarity determining regions” (“CDRs”). Generally, antigen binding domains comprise six CDRs: three in the VH (CDR-H1, CDR-H2, CDR-H3), and three in the VL (CDR-L1, CDR-L2, CDR-L3). Exemplary CDRs herein include:

(a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));

(b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, Md. (1991)); and

(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262: 732-745 (1996)).

Unless otherwise indicated, the CDRs are determined according to Kabat et al., supra. One of skill in the art will understand that the CDR designations can also be determined according to Chothia, supra, McCallum, supra, or any other scientifically accepted nomenclature. Kabat et al. also defined a numbering system for variable region sequences that is applicable to any antibody. One of ordinary skill in the art can unambiguously assign this system of “Kabat numbering” to any variable region sequence, without reliance on any experimental data beyond the sequence itself. As used herein, “Kabat numbering” refers to the numbering system set forth by Kabat et al., U.S. Dept. of Health and Human Services, “Sequence of Proteins of Immunological Interest” (1983). Unless otherwise specified, references to the numbering of specific amino acid residue positions in an antibody variable region are according to the Kabat numbering system.

As used herein, the term “affinity matured” in the context of antigen binding molecules (e.g., antibodies) refers to an antigen binding molecule that is derived from a reference antigen binding molecule, e.g., by mutation, binds to the same antigen, preferably binds to the same epitope, as the reference antibody; and has a higher affinity for the antigen than that of the reference antigen binding molecule. Affinity maturation generally involves modification of one or more amino acid residues in one or more CDRs of the antigen binding molecule. Typically, the affinity matured antigen binding molecule binds to the same epitope as the initial reference antigen binding molecule.

“Framework” or “FR” refers to variable domain residues other than complementary determining regions (CDRs). The FR of a variable domain generally consists of four FR domains: FR1, FR2, FR3, and FR4. Accordingly, the CDR and FR sequences generally appear in the following sequence in VH (or VL): FR1-CDR-H1(CDR-L1)-FR2-CDR-H2(CDR-L2)-FR3-CDR-H3(CDR-L3)-FR4.

An “acceptor human framework” for the purposes herein is a framework comprising the amino acid sequence of a light chain variable domain (VL) framework or a heavy chain variable domain (VH) framework derived from a human immunoglobulin framework or a human consensus framework, as defined below. An acceptor human framework “derived from” a human immunoglobulin framework or a human consensus framework may comprise the same amino acid sequence thereof, or it may contain amino acid sequence changes. In some embodiments, the number of amino acid changes are 10 or less, 9 or less, 8 or less, 7 or less, 6 or less, 5 or less, 4 or less, 3 or less, or 2 or less. In some embodiments, the VL acceptor human framework is identical in sequence to the VL human immunoglobulin framework sequence or human consensus framework sequence.

The term “chimeric” antibody refers to an antibody in which a portion of the heavy and/or light chain is derived from a particular source or species, while the remainder of the heavy and/or light chain is derived from a different source or species.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g. IgG₁, IgG₂, IgG₃, IgG₄, IgA₁, and IgA₂. The heavy chain constant domains that correspond to the different classes of immunoglobulins are called a, α, δ, ε, γ, and μ respectively.

The terms “constant region derived from human origin” or “human constant region” denote a constant heavy chain region of a human antibody of the subclass IgG1, IgG2, IgG3, or IgG4 and/or a constant light chain kappa or lambda region. Such constant regions are well known in the state of the art and e.g. described by Kabat, E. A., et al., Sequences of Proteins of Immunological Interest, 5th ed., Public Health Service, National Institutes of Health, Bethesda, Md. (1991) (see also e.g. Johnson, G., and Wu, T. T., Nucleic Acids Res. 28 (2000) 214-218; Kabat, E. A., et al., Proc. Natl. Acad. Sci. USA 72 (1975) 2785-2788). Unless otherwise specified herein, numbering of amino acid residues in the constant region is according to the EU numbering system, also called the EU index of Kabat, as described in Kabat, E. A. et al., Sequences of Proteins of Immunological Interest, 5th ed., Public Health Service, National Institutes of Health, Bethesda, Md. (1991), NIH Publication 91-3242.

The term “CH1 domain” denotes the part of an antibody heavy chain polypeptide that extends approximately from EU position 118 to EU position 215 (EU numbering system according to Kabat). In one aspect, a CH1 domain has the amino acid sequence of ASTKGPSVFP LAPSSKSTSG GTAALGCLVK DYFPEPVTVS WNSGALTSGV HTFPAVLQSS GLYSLSSVVT VPSSSLGTQT YICNVNHKPS NTKVDKKV (SEQ ID NO: 301). Usually, a segment having the amino acid sequence of EPKSC (SEQ ID NO:302) is following to link the CH1 domain to the hinge region.

The term “hinge region” denotes the part of an antibody heavy chain polypeptide that joins in a wild-type antibody heavy chain the CH1 domain and the CH2 domain, e. g. from about position 216 to about position 230 according to the EU number system of Kabat, or from about position 226 to about position 230 according to the EU number system of Kabat. The hinge regions of other IgG subclasses can be determined by aligning with the hinge-region cysteine residues of the IgG1 subclass sequence. The hinge region is normally a dimeric molecule consisting of two polypeptides with identical amino acid sequence. The hinge region generally comprises up to 25 amino acid residues and is flexible allowing the associated target binding sites to move independently. The hinge region can be subdivided into three domains: the upper, the middle, and the lower hinge domain (see e.g. Roux, et al., J. Immunol. 161 (1998) 4083).

In one aspect, the hinge region has the amino acid sequence DKTHTCPXCP (SEQ ID NO: 303), wherein X is either S or P. In one aspect, the hinge region has the amino acid sequence HTCPXCP (SEQ ID NO: 304), wherein X is either S or P. In one aspect, the hinge region has the amino acid sequence CPXCP (SEQ ID NO:305), wherein X is either S or P.

The term “Fc domain” or “Fc region” herein is used to define a C-terminal region of an immunoglobulin heavy chain that contains at least a portion of the constant region. The term includes native sequence Fc domains and variant Fc domains. In one aspect, a human IgG heavy chain Fc region extends from Cys226, or from Pro230, to the carboxyl-terminus of the heavy chain. However, antibodies produced by host cells may undergo post-translational cleavage of one or more, particularly one or two, amino acids from the C-terminus of the heavy chain. Therefore, an antibody produced by a host cell by expression of a specific nucleic acid molecule encoding a full-length heavy chain may include the full-length heavy chain, or it may include a cleaved variant of the full-length heavy chain. This may be the case where the final two C-terminal amino acids of the heavy chain are glycine (G446) and lysine (K447, numbering according to EU index). Therefore, the C-terminal lysine (Lys447), or the C-terminal glycine (Gly446) and lysine (Lys447), of the Fc region may or may not be present. Amino acid sequences of heavy chains including an Fc region are denoted herein without C-terminal glycine-lysine dipeptide if not indicated otherwise. In one aspect, a heavy chain including an Fc region as specified herein, comprised in an antigen binding molecule according to the invention, comprises an additional C-terminal glycine-lysine dipeptide (G446 and K447, numbering according to EU index). In one aspect, a heavy chain including an Fc region as specified herein, comprised in an antigen binding molecule according to the invention, comprises an additional C-terminal glycine residue (G446, numbering according to EU index). Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, Md., 1991.

An IgG Fc region comprises an IgG CH2 and an IgG CH3 domain. The “CH2 domain” of a human IgG Fc region usually extends from an amino acid residue at about position 231 to an amino acid residue at about position 340. In one aspect, a carbohydrate chain is attached to the CH2 domain. The “CH2 domain” of a human IgG Fc region usually extends from an amino acid residue at about EU position 231 to an amino acid residue at about EU position 340 (EU numbering system according to Kabat). In one aspect, a CH2 domain has the amino acid sequence of APELLGGPSV FL FPPKPKDT LMI SRTPEVT CVWDVSHEDP EVKFNWYVDG VEVHNAKTKP REEQESTYRW SVLTVLHQDW LNGKEYKCKV SNKALPAPIE KTISKAK (SEQ ID NO: 299). The CH2 domain is unique in that it is not closely paired with another domain. Rather, two N-linked branched carbohydrate chains are interposed between the two CH2 domains of an intact native Fc-region. It has been speculated that the carbohydrate may provide a substitute for the domain-domain pairing and help stabilize the CH2 domain. Burton, Mol. Immunol. 22 (1985) 161-206. In one embodiment, a carbohydrate chain is attached to the CH2 domain. The CH2 domain herein may be a native sequence CH2 domain or variant CH2 domain.

The “CH3 domain” comprises the stretch of residues C-terminal to a CH2 domain in an Fc region (i.e. from an amino acid residue at about position 341 to an amino acid residue at about position 445 of an IgG, EU numbering system according to Kabat). In one aspect, the CH3 domain has the amino acid sequence of GQPREPQVYT LPPSRDELTK NQVSLTCLVK GFYPSDIAVE WESNGQPENN YKTTPPVLDS DGSFFLYSKL TVDKSRWQQG NVFSCSVMHE ALHNHYTQKS LSLSP (SEQ ID NO: 300). The CH3 region herein may be a native sequence CH3 domain or a variant CH3 domain (e.g. a CH3 domain with an introduced “protuberance” (“knob”) in one chain thereof and a corresponding introduced “cavity” (“hole”) in the other chain thereof; see U.S. Pat. No. 5,821,333, expressly incorporated herein by reference). Such variant CH3 domains may be used to promote heterodimerization of two non-identical antibody heavy chains as herein described.

The “knob-into-hole” technology is described e.g. in U.S. Pat. Nos. 5,731,168; 7,695,936; Ridgway et al., Prot Eng 9, 617-621 (1996) and Carter, J Immunol Meth 248, 7-15 (2001). Generally, the method involves introducing a protuberance (“knob”) at the interface of a first polypeptide and a corresponding cavity (“hole”) in the interface of a second polypeptide, such that the protuberance can be positioned in the cavity so as to promote heterodimer formation and hinder homodimer formation. Protuberances are constructed by replacing small amino acid side chains from the interface of the first polypeptide with larger side chains (e.g. tyrosine or tryptophan). Compensatory cavities of identical or similar size to the protuberances are created in the interface of the second polypeptide by replacing large amino acid side chains with smaller ones (e.g. alanine or threonine). The protuberance and cavity can be made by altering the nucleic acid encoding the polypeptides, e.g. by site-specific mutagenesis, or by peptide synthesis. In a specific embodiment a knob modification comprises the amino acid substitution T366W in one of the two subunits of the Fc domain, and the hole modification comprises the amino acid substitutions T366S, L368A and Y407V in the other one of the two subunits of the Fc domain. In a further specific embodiment, the subunit of the Fc domain comprising the knob modification additionally comprises the amino acid substitution S354C, and the subunit of the Fc domain comprising the hole modification additionally comprises the amino acid substitution Y349C. Introduction of these two cysteine residues results in the formation of a disulfide bridge between the two subunits of the Fc region, thus further stabilizing the dimer (Carter, J Immunol Methods 248, 7-15 (2001)). The numbering is according to EU index of Kabat et al, Sequences of Proteins of Immunological Interest, 5th Ed. Public Health Service, National Institutes of Health, Bethesda, Md., 1991.

A “region equivalent to the Fc region of an immunoglobulin” is intended to include naturally occurring allelic variants of the Fc region of an immunoglobulin as well as variants having alterations which produce substitutions, additions, or deletions but which do not decrease substantially the ability of the immunoglobulin to mediate effector functions (such as antibody-dependent cellular cytotoxicity). For example, one or more amino acids can be deleted from the N-terminus or C-terminus of the Fc region of an immunoglobulin without substantial loss of biological function. Such variants can be selected according to general rules known in the art so as to have minimal effect on activity (see, e.g., Bowie, J. U. et al., Science 247:1306-10 (1990)).

The term “wild-type Fc domain” denotes an amino acid sequence identical to the amino acid sequence of an Fc domain found in nature. Wild-type human Fc domains include a native human IgG1 Fc-region (non-A and A allotypes), native human IgG2 Fc-region, native human IgG3 Fc-region, and native human IgG4 Fc-region as well as naturally occurring variants thereof. Wild-type Fc-domains are comprised in SEQ ID NO: 306 (IgG1, caucasian allotype), SEQ ID NO: 307 (IgG1, afroamerican allotype), SEQ ID NO: 308 (IgG2), SEQ ID NO:309 (IgG3) and SEQ ID NO:310 (IgG4).

The term “variant (human) Fc domain” denotes an amino acid sequence which differs from that of a “wild-type” (human) Fc domain amino acid sequence by virtue of at least one “amino acid mutation”. In one aspect, the variant Fc-region has at least one amino acid mutation compared to a native Fc-region, e.g. from about one to about ten amino acid mutations, and in one aspect from about one to about five amino acid mutations in a native Fc-region. In one aspect, the (variant) Fc-region has at least about 95% homology with a wild-type Fc-region.

A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization. Other forms of “humanized antibodies” encompassed by the present invention are those in which the constant region has been additionally modified or changed from that of the original antibody to generate the properties according to the invention, especially in regard to C1q binding and/or Fc receptor (FcR) binding.

The term “effector functions” refers to those biological activities attributable to the Fc region of an antibody, which vary with the antibody isotype. Examples of antibody effector functions include: C1q binding and complement dependent cytotoxicity (CDC), Fc receptor binding, antibody-dependent cell-mediated cytotoxicity (ADCC), antibody-dependent cellular phagocytosis (ADCP), cytokine secretion, immune complex-mediated antigen uptake by antigen presenting cells, down regulation of cell surface receptors (e.g. B cell receptor), and B cell activation.

An “activating Fc receptor” is an Fc receptor that following engagement by an Fc region of an antibody elicits signaling events that stimulate the receptor-bearing cell to perform effector functions. Activating Fc receptors include FcγRIIIa (CD16a), FcγRI (CD64), FcγRIIa (CD32), and FcαRI (CD89). A particular activating Fc receptor is human FcγRIIIa (see UniProt accession no. P08637, version 141).

Antibody-dependent cell-mediated cytotoxicity (ADCC) is an immune mechanism leading to the lysis of antibody-coated target cells by immune effector cells. The target cells are cells to which antibodies or derivatives thereof comprising an Fc region specifically bind, generally via the protein part that is N-terminal to the Fc region. As used herein, the term “reduced ADCC” is defined as either a reduction in the number of target cells that are lysed in a given time, at a given concentration of antibody in the medium surrounding the target cells, by the mechanism of ADCC defined above, and/or an increase in the concentration of antibody in the medium surrounding the target cells, required to achieve the lysis of a given number of target cells in a given time, by the mechanism of ADCC. The reduction in ADCC is relative to the ADCC mediated by the same antibody produced by the same type of host cells, using the same standard production, purification, formulation and storage methods (which are known to those skilled in the art), but that has not been engineered. For example, the reduction in ADCC mediated by an antibody comprising in its Fc domain an amino acid substitution that reduces ADCC, is relative to the ADCC mediated by the same antibody without this amino acid substitution in the Fc domain. Suitable assays to measure ADCC are well known in the art (see e.g. PCT publication no. WO 2006/082515 or PCT publication no. WO 2012/130831)

The term “TNF ligand family member” or “TNF family ligand” refers to a proinflammatory cytokine. Cytokines in general, and in particular the members of the TNF ligand family, play a crucial role in the stimulation and coordination of the immune system. At present, nineteen cytokines have been identified as members of the TNF (tumour necrosis factor) ligand superfamily on the basis of sequence, functional, and structural similarities. All these ligands are type II transmembrane proteins with a C-terminal extracellular domain (ectodomain), N-terminal intracellular domain and a single transmembrane domain. The C-terminal extracellular domain, known as TNF homology domain (THD), has 20-30% amino acid identity between the superfamily members and is responsible for binding to the receptor. The TNF ectodomain is also responsible for the TNF ligands to form trimeric complexes that are recognized by their specific receptors. Members of the TNF ligand family are selected from the group consisting of Lymphotoxin a (also known as LTA or TNFSF1), TNF (also known as TNFSF2), LTβ (also known as TNFSF3), OX40L (also known as TNFSF4), CD40L (also known as CD154 or TNFSF5), FasL (also known as CD95L, CD178 or TNFSF6), CD27L (also known as CD70 or TNFSF7), CD30L (also known as CD153 or TNFSF8), 4-1BBL (also known as TNFSF9), TRAIL (also known as APO2L, CD253 or TNFSF10), RANKL (also known as CD254 or TNFSF11), TWEAK (also known as TNFSF12), APRIL (also known as CD256 or TNFSF13), BAFF (also known as CD257 or TNFSF13B), LIGHT (also known as CD258 or TNFSF14), TL1A (also known as VEGI or TNFSF15), GITRL (also known as TNFSF18), EDA-A1 (also known as ectodysplasin A1) and EDA-A2 (also known as ectodysplasin A2). The term refers to any native TNF family ligand from any vertebrate source, including mammals such as primates (e.g. humans), non-human primates (e.g. cynomolgus monkeys) and rodents (e.g. mice and rats), unless otherwise indicated. The term “costimulatory TNF ligand family member” or “costimulatory TNF family ligand” refers to a subgroup of TNF ligand family members, which are able to costimulate proliferation and cytokine production of T-cells. These TNF family ligands can costimulate TCR signals upon interaction with their corresponding TNF receptors and the interaction with their receptors leads to recruitment of TNFR-associated factors (TRAF), which initiate signalling cascades that result in T-cell activation. Costimulatory TNF family ligands are selected from the group consisting of 4-1BBL, OX40L, GITRL, CD70, CD30L and LIGHT, more particularly the costimulatory TNF ligand family member is 4-1BBL.

As described herein before, 4-1BBL is a type II transmembrane protein and one member of the TNF ligand family. Complete or full length 4-1BBL having the amino acid sequence of SEQ ID NO:297 has been described to form trimers on the surface of cells. The formation of trimers is enabled by specific motives of the ectodomain of 4-1BBL. Said motives are designated herein as “trimerization region”. The amino acids 50-254 of the human 4-1BBL sequence (SEQ ID NO:298) form the extracellular domain of 4-1BBL, but even fragments thereof are able to form the trimers. In specific embodiments of the invention, the term “ectodomain of 4-1BBL or a fragment thereof” refers to a polypeptide having an amino acid sequence selected from SEQ ID NO:90 (amino acids 52-254 of human 4-1BBL), SEQ ID NO:87 (amino acids 71-254 of human 4-1BBL), SEQ ID NO:89 (amino acids 80-254 of human 4-1BBL) and SEQ ID NO:88 (amino acids 85-254 of human 4-1BBL) or a polypeptide having an amino acid sequence selected from SEQ ID NO:91 (amino acids 71-248 of human 4-1BBL), SEQ ID NO:94 (amino acids 52-248 of human 4-1BBL), SEQ ID NO:93 (amino acids 80-248 of human 4-1BBL) and SEQ ID NO:92 (amino acids 85-248 of human 4-1BBL), but also other fragments of the ectodomain capable of trimerization are included herein.

An “ectodomain” is the domain of a membrane protein that extends into the extracellular space (i.e. the space outside the target cell). Ectodomains are usually the parts of proteins that initiate contact with surfaces, which leads to signal transduction. The ectodomain of TNF ligand family member as defined herein thus refers to the part of the TNF ligand protein that extends into the extracellular space (the extracellular domain), but also includes shorter parts or fragments thereof that are responsible for the trimerization and for the binding to the corresponding TNF receptor. The term “ectodomain of a TNF ligand family member or a fragment thereof” thus refers to the extracellular domain of the TNF ligand family member that forms the extracellular domain or to parts thereof that are still able to bind to the receptor (receptor binding domain).

The term “peptide linker” refers to a peptide comprising one or more amino acids, typically about 2 to 20 amino acids. Peptide linkers are known in the art or are described herein. Suitable, non-immunogenic linker peptides are, for example, (G₄S)ₙ, (SG₄)ₙ or G₄(SG₄)ₙ peptide linkers, wherein “n” is generally a number between 1 and 10, typically between 1 and 4, in particular 2, i.e. the peptides selected from the group consisting of GGGGS (SEQ ID NO:112), GGGGSGGGGS (SEQ ID NO:113), SGGGGSGGGG (SEQ ID NO:114), (G₄S)₃ or GGGGSGGGGSGGGGS (SEQ ID NO:117), GGGGSGGGGSGGGG or G4(SG4)₂ (SEQ ID NO:115), and (G₄S)₄ or GGGGSGGGGSGGGGSGGGGS (SEQ ID NO:118), but also include the sequences GSPGSSSSGS (SEQ ID NO:116), GSGSGSGS (SEQ ID NO:119), GSGSGNGS (SEQ ID NO:120), GGSGSGSG (SEQ ID NO:121), GGSGSG (SEQ ID NO:122), GGSG (SEQ ID NO:123), GGSGNGSG (SEQ ID NO:124), GGNGSGSG (SEQ ID NO:125) and GGNGSG (SEQ ID NO:126). Peptide linkers of particular interest are (G4S)₁ or GGGGS (SEQ ID NO:112), (G₄S)₂ or GGGGSGGGGS (SEQ ID NO:113) and (G₄S)₃ (SEQ ID NO:117).

The term “amino acid” as used within this application denotes the group of naturally occurring carboxy α-amino acids comprising alanine (three letter code: ala, one letter code: A), arginine (arg, R), asparagine (asn, N), aspartic acid (asp, D), cysteine (cys, C), glutamine (gln, Q), glutamic acid (glu, E), glycine (gly, G), histidine (his, H), isoleucine (ile, I), leucine (leu, L), lysine (lys, K), methionine (met, M), phenylalanine (phe, F), proline (pro, P), serine (ser, S), threonine (thr, T), tryptophan (trp, W), tyrosine (tyr, Y), and valine (val, V).

The terms “full length antibody”, “intact antibody”, and “whole antibody” are used herein interchangeably to refer to an antibody having a structure substantially similar to a native antibody structure or having heavy chains that contain an Fc region as defined herein.

A “fusion polypeptide” or “fusion protein” as used herein refers to a single chain polypeptide composed of an antibody fragment and a peptide that is not derived from an antibody. In one aspect, a fusion polypeptide is composed of one or two ectodomains of 4-1BBL or a fragment thereof fused to a part of antigen binding domain or Fc part. The fusion may occur by directly linking the N or C-terminal amino acid of the antigen binding moiety via a peptide linker to the C- or N-terminal amino acid of the ectodomain of said 4-1BBL or fragment thereof.

By “fused” or “connected” is meant that the components (e.g. a polypeptide and an ectodomain of said TNF ligand family member) are linked by peptide bonds, either directly or via one or more peptide linkers.

“Percent (%) amino acid sequence identity” with respect to a reference polypeptide (protein) sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN. SAWI or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary. In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

In certain embodiments, amino acid sequence variants of the TNF ligand trimer-containing antigen binding molecules provided herein are contemplated. For example, it may be desirable to improve the binding affinity and/or other biological properties of the TNF ligand trimer-containing antigen binding molecules. Amino acid sequence variants of the TNF ligand trimer-containing antigen binding molecules may be prepared by introducing appropriate modifications into the nucleotide sequence encoding the molecules, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding. Sites of interest for substitutional mutagenesis include the HVRs and Framework (FRs). Conservative substitutions are provided in Table A under the heading “Preferred Substitutions” and further described below in reference to amino acid side chain classes (1) to (6). Amino acid substitutions may be introduced into the molecule of interest and the products screened for a desired activity, e.g., retained/improved antigen binding, decreased immunogenicity, or improved ADCC or CDC.

TABLE A

| Original | Exemplary | Preferred |
|---|---|---|
| Residue | Substitutions | Substitutions |
| Ala (A) | Val; Leu; ILe | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

The term “amino acid sequence variants” includes substantial variants wherein there are amino acid substitutions in one or more hypervariable region residues of a parent antigen binding molecule (e.g. a humanized or human antibody). Generally, the resulting variant(s) selected for further study will have modifications (e.g., improvements) in certain biological properties (e.g., increased affinity, reduced immunogenicity) relative to the parent antigen binding molecule and/or will have substantially retained certain biological properties of the parent antigen binding molecule. An exemplary substitutional variant is an affinity matured antibody, which may be conveniently generated, e.g., using phage display-based affinity maturation techniques such as those described herein. Briefly, one or more CDR residues are mutated and the variant antigen binding molecules displayed on phage and screened for a particular biological activity (e.g. binding affinity). In certain embodiments, substitutions, insertions, or deletions may occur within one or more CDRs so long as such alterations do not substantially reduce the ability of the antigen binding molecule to bind antigen. For example, conservative alterations (e.g., conservative substitutions as provided herein) that do not substantially reduce binding affinity may be made in CDRs. A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as Arg, Asp, His, Lys, and Glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antigen binding molecule complex to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include a 4-1BBL trimer-containing antigen binding molecule with an N-terminal methionyl residue. Other insertional variants of the molecule include the fusion to the N- or C-terminus to a polypeptide which increases the serum half-life of the 4-1BBL trimer-containing antigen binding molecule.

In certain aspect, the CEA antibodies or 4-1BBL trimer-containing antigen binding molecules provided herein are altered to increase or decrease the extent to which the antibody is glycosylated. Glycosylation variants of the molecules may be conveniently obtained by altering the amino acid sequence such that one or more glycosylation sites is created or removed. Where the 4-1BBL trimer-containing antigen binding molecule comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in 4-1BBL ligand trimer-containing antigen binding molecule may be made in order to create variants with certain improved properties. In one aspect, variants of 4-1BBL trimer-containing antigen binding molecules are provided having a carbohydrate structure that lacks fucose attached (directly or indirectly) to an Fc region. Such fucosylation variants may have improved ADCC function, see e.g. US Patent Publication Nos. US 2003/0157108 (Presta, L.) or US 2004/0093621 (Kyowa Hakko Kogyo Co., Ltd). Further variants of the 4-1BBL trimer-containing antigen binding molecules of the invention include those with bisected oligosaccharides, e.g., in which a biantennary oligosaccharide attached to the Fc region is bisected by GlcNAc. Such variants may have reduced fucosylation and/or improved ADCC function, see for example WO 2003/011878 (Jean-Mairet et al.); U.S. Pat. No. 6,602,684 (Umana et al.); and US 2005/0123546 (Umana et al.). Variants with at least one galactose residue in the oligosaccharide attached to the Fc region are also provided. Such antibody variants may have improved CDC function and are described, e.g., in WO 1997/30087 (Patel et al.); WO 1998/58964 (Raju, S.); and WO 1999/22764 (Raju, S.).

In certain embodiments, it may be desirable to create cysteine engineered variants of the CEA antibody or 4-1BBL trimer-containing antigen binding molecule of the invention, e.g., “thioMAbs,” in which one or more residues of the molecule are substituted with cysteine residues. In particular embodiments, the substituted residues occur at accessible sites of the molecule. By substituting those residues with cysteine, reactive thiol groups are thereby positioned at accessible sites of the antibody and may be used to conjugate the antibody to other moieties, such as drug moieties or linker-drug moieties, to create an immunoconjugate. In certain embodiments, any one or more of the following residues may be substituted with cysteine: V205 (Kabat numbering) of the light chain; A118 (EU numbering) of the heavy chain; and 5400 (EU numbering) of the heavy chain Fc region. Cysteine engineered antigen binding molecules may be generated as described, e.g., in U.S. Pat. No. 7,521,541.

In certain aspects, the CEA antibodies or 4-1BBL trimer-containing antigen binding molecules provided herein may be further modified to contain additional non-proteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer is attached, they can be the same or different molecules. In general, the number and/or type of polymers used for derivatization can be determined based on considerations including, but not limited to, the particular properties or functions of the antibody to be improved, whether the bispecific antibody derivative will be used in a therapy under defined conditions, etc. In another aspect, conjugates of an antibody and non-proteinaceous moiety that may be selectively heated by exposure to radiation are provided. In one embodiment, the non-proteinaceous moiety is a carbon nanotube (Kam, N. W. et al., Proc. Natl. Acad. Sci. USA 102 (2005) 11600-11605). The radiation may be of any wavelength, and includes, but is not limited to, wavelengths that do not harm ordinary cells, but which heat the non-proteinaceous moiety to a temperature at which cells proximal to the antibody-non-proteinaceous moiety are killed.

In another aspect, immunoconjugates of the CEA antibodies or the 4-1 BBL trimer-containing antigen binding molecules provided herein may be obtained. An “immunoconjugate” is an antibody conjugated to one or more heterologous molecule(s), including but not limited to a cytotoxic agent.

The term “nucleic acid molecule” or “polynucleotide” includes any compound and/or substance that comprises a polymer of nucleotides. Each nucleotide is composed of a base, specifically a purine- or pyrimidine base (i.e. cytosine (C), guanine (G), adenine (A), thymine (T) or uracil (U)), a sugar (i.e. deoxyribose or ribose), and a phosphate group. Often, the nucleic acid molecule is described by the sequence of bases, whereby said bases represent the primary structure (linear structure) of a nucleic acid molecule. The sequence of bases is typically represented from 5′ to 3′. Herein, the term nucleic acid molecule encompasses deoxyribonucleic acid (DNA) including e.g., complementary DNA (cDNA) and genomic DNA, ribonucleic acid (RNA), in particular messenger RNA (mRNA), synthetic forms of DNA or RNA, and mixed polymers comprising two or more of these molecules. The nucleic acid molecule may be linear or circular. In addition, the term nucleic acid molecule includes both, sense and antisense strands, as well as single stranded and double stranded forms. Moreover, the herein described nucleic acid molecule can contain naturally occurring or non-naturally occurring nucleotides. Examples of non-naturally occurring nucleotides include modified nucleotide bases with derivatized sugars or phosphate backbone linkages or chemically modified residues. Nucleic acid molecules also encompass DNA and RNA molecules which are suitable as a vector for direct expression of an antibody of the invention in vitro and/or in vivo, e.g., in a host or patient. Such DNA (e.g., cDNA) or RNA (e.g., mRNA) vectors, can be unmodified or modified. For example, mRNA can be chemically modified to enhance the stability of the RNA vector and/or expression of the encoded molecule so that mRNA can be injected into a subject to generate the antibody in vivo (see e.g., Stadler et al, Nature Medicine 2017, published online 12 Jun. 2017, doi:10.1038/nm.4356 or EP 2 101 823 B1).

By “isolated” nucleic acid molecule or polynucleotide is intended a nucleic acid molecule, DNA or RNA, which has been removed from its native environment. For example, a recombinant polynucleotide encoding a polypeptide contained in a vector is considered isolated for the purposes of the present invention. Further examples of an isolated polynucleotide include recombinant polynucleotides maintained in heterologous host cells or purified (partially or substantially) polynucleotides in solution. An isolated polynucleotide includes a polynucleotide molecule contained in cells that ordinarily contain the polynucleotide molecule, but the polynucleotide molecule is present extrachromosomally or at a chromosomal location that is different from its natural chromosomal location. Isolated RNA molecules include in vivo or in vitro RNA transcripts of the present invention, as well as positive and negative strand forms, and double-stranded forms. Isolated polynucleotides or nucleic acids according to the present invention further include such molecules produced synthetically. In addition, a polynucleotide or a nucleic acid may be or may include a regulatory element such as a promoter, ribosome binding site, or a transcription terminator.

By a nucleic acid or polynucleotide having a nucleotide sequence at least, for example, 95% “identical” to a reference nucleotide sequence of the present invention, it is intended that the nucleotide sequence of the polynucleotide is identical to the reference sequence except that the polynucleotide sequence may include up to five point mutations per each 100 nucleotides of the reference nucleotide sequence. In other words, to obtain a polynucleotide having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. These alterations of the reference sequence may occur at the 5′ or 3′ terminal positions of the reference nucleotide sequence or anywhere between those terminal positions, interspersed either individually among residues in the reference sequence or in one or more contiguous groups within the reference sequence. As a practical matter, whether any particular polynucleotide sequence is at least 80%, 85%, 90%, 95%, 96%, 97%, 98% or 99% identical to a nucleotide sequence of the present invention can be determined conventionally using known computer programs, such as the ones discussed above for polypeptides (e.g. ALIGN-2).

The term “expression cassette” refers to a polynucleotide generated recombinantly or synthetically, with a series of specified nucleic acid elements that permit transcription of a particular nucleic acid in a target cell. The recombinant expression cassette can be incorporated into a plasmid, chromosome, mitochondrial DNA, plastid DNA, virus, or nucleic acid fragment. Typically, the recombinant expression cassette portion of an expression vector includes, among other sequences, a nucleic acid sequence to be transcribed and a promoter. In certain embodiments, the expression cassette of the invention comprises polynucleotide sequences that encode bispecific antigen binding molecules of the invention or fragments thereof.

The term “vector” or “expression vector” is synonymous with “expression construct” and refers to a DNA molecule that is used to introduce and direct the expression of a specific gene to which it is operably associated in a target cell. The term includes the vector as a self-replicating nucleic acid structure as well as the vector incorporated into the genome of a host cell into which it has been introduced. The expression vector of the present invention comprises an expression cassette. Expression vectors allow transcription of large amounts of stable mRNA. Once the expression vector is inside the target cell, the ribonucleic acid molecule or protein that is encoded by the gene is produced by the cellular transcription and/or translation machinery. In one embodiment, the expression vector of the invention comprises an expression cassette that comprises polynucleotide sequences that encode bispecific antigen binding molecules of the invention or fragments thereof.

The terms “host cell”, “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein. A host cell is any type of cellular system that can be used to generate the bispecific antigen binding molecules of the present invention. Host cells include cultured cells, e.g. mammalian cultured cells, such as CHO cells, BHK cells, NS0 cells, SP2/0 cells, YO myeloma cells, P3X63 mouse myeloma cells, PER cells, PER.C6 cells or hybridoma cells, yeast cells, insect cells, and plant cells, to name only a few, but also cells comprised within a transgenic animal, transgenic plant or cultured plant or animal tissue.

An “effective amount” of an agent refers to the amount that is necessary to result in a physiological change in the cell or tissue to which it is administered.

A “therapeutically effective amount” of an agent, e.g. a pharmaceutical composition, refers to an amount effective, at dosages and for periods of time necessary, to achieve the desired therapeutic or prophylactic result. A therapeutically effective amount of an agent for example eliminates, decreases, delays, minimizes or prevents adverse effects of a disease.

An “individual” or “subject” is a mammal. Mammals include, but are not limited to, domesticated animals (e.g. cows, sheep, cats, dogs, and horses), primates (e.g. humans and non-human primates such as monkeys), rabbits, and rodents (e.g. mice and rats). Particularly, the individual or subject is a human.

The term “pharmaceutical composition” refers to a preparation which is in such form as to permit the biological activity of an active ingredient contained therein to be effective, and which contains no additional components which are unacceptably toxic to a subject to which the formulation would be administered.

A “pharmaceutically acceptable excipient” refers to an ingredient in a pharmaceutical composition, other than an active ingredient, which is nontoxic to a subject. A pharmaceutically acceptable excipient includes, but is not limited to, a buffer, a stabilizer, or a preservative.

The term “package insert” is used to refer to instructions customarily included in commercial packages of therapeutic products, that contain information about the indications, usage, dosage, administration, combination therapy, contraindications and/or warnings concerning the use of such therapeutic products.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, the molecules of the invention are used to delay development of a disease or to slow the progression of a disease.

The term “cancer” as used herein refers to proliferative diseases, such as lymphomas, carcinoma, lymphoma, blastoma, sarcoma, leukemia, lymphocytic leukemias, lung cancer, non-small cell lung (NSCL) cancer, bronchioloalviolar cell lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, gastric cancer, colorectal cancer (CRC), pancreatic cancer, breast cancer, triple-negative breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, cancer of the bladder, cancer of the kidney or ureter, renal cell carcinoma, carcinoma of the renal pelvis, mesothelioma, hepatocellular cancer, biliary cancer, neoplasms of the central nervous system (CNS), spinal axis tumors, brain stem glioma, glioblastoma multiforme, astrocytomas, schwanomas, ependymonas, medulloblastomas, meningiomas, squamous cell carcinomas, pituitary adenoma and Ewings sarcoma, melanoma, multiple myeloma, B-cell cancer (lymphoma), chronic lymphocytic leukemia (CLL), acute lymphoblastic leukemia (ALL), hairy cell leukemia, chronic myeloblastic leukemia, including refractory versions of any of the above cancers, or a combination of one or more of the above cancers.

The term “metastatic cancer” means the state of cancer where the cancer cells are transmitted from the original site to one or more sites elsewhere in the body, by the blood vessels or lymphatics, to form one or more secondary tumors in one or more organs besides the breast.

An “advanced” cancer is one which has spread outside the site or organ of origin, either by local invasion or metastasis. Accordingly, the term “advanced” cancer includes both locally advanced and metastatic disease.

A “recurrent” cancer is one which has regrown, either at the initial site or at a distant site, after a response to initial therapy, such as surgery. A “locally recurrent” cancer is cancer that returns after treatment in the same place as a previously treated cancer. An “operable” or “resectable” cancer is cancer which is confined to the primary organ and suitable for surgery (resection). A “non-resectable” or “unresectable” cancer is not able to be removed (resected) by surgery.

Antigen Binding Molecules Binding to CEA According to the Invention

The invention provides novel antigen binding molecules binding to carcinoembryonic antigen (CEA) with particularly advantageous properties such as the epitope they bind to, cynomolgus monkey/human cross-reactivity, producibility, stability, binding affinity, biological activity, targeting efficiency, reduced toxicity and the ability to be combined with other CEA-targeted antigen binding molecules wherein the CEA antigen binding domain binds to a different epitope.

In a first aspect, the invention provides new humanized antibodies that bind to the A2 domain of carcinoembryonic antigen (CEACAM5). Thus, in one aspect, new humanized antibodies are provided that bind to the domain comprising the amino acid sequence of SEQ ID NO:311. In one aspect, provided are antibodies that compete for binding with an antibody that comprises a variable heavy chain domain (VH) comprising (i) CDR-H1 comprising the amino acid sequence of SEQ ID NO:1, (ii) CDR-H2 comprising the amino acid sequence of SEQ ID NO:2, and (iii) CDR-H3 comprising the amino acid sequence of SEQ ID NO:3, and a variable light chain domain (VL) comprising (iv) CDR-L1 comprising the amino acid sequence of SEQ ID NO:4, (v) CDR-L2 comprising the amino acid sequence of SEQ ID NO:5, and (vi) CDR-L3 comprising the amino acid sequence of SEQ ID NO:6. In particular, these antibodies compete for binding with an antibody comprising a VH domain comprising the amino acid sequence of SEQ ID NO:7 and a VL domain comprising the amino acid sequence of SEQ ID NO:8. Provided are thus antibodies capable of specific binding to CEA that do not compete with an antibody comprising the amino acid sequence of comprising a VH domain comprising the amino acid sequence of SEQ ID NO:63 and a VL domain comprising the amino acid sequence of SEQ ID NO:64.

In one aspect, provided is a humanized antibody capable of specific binding to CEA, wherein the antibody comprises a variable heavy chain domain (VH) comprising (i) CDR-H1 comprising the amino acid sequence of SEQ ID NO:17, (ii) CDR-H2 comprising the amino acid sequence of SEQ ID NO:18, and (iii) CDR-H3 comprising the amino acid sequence of SEQ ID NO:19, and a variable light chain domain (VL) comprising (iv) CDR-L1 comprising the amino acid sequence of SEQ ID NO:20, (v) CDR-L2 comprising the amino acid sequence of SEQ ID NO:21, and (vi) CDR-L3 comprising the amino acid sequence of SEQ ID NO:22 and wherein the framework of the VH domain is based on a human acceptor framework comprising the amino acid sequence of SEQ ID NO:127 (IGHV3-23-02) and wherein the framework of VL domain is based on a human acceptor framework comprising the amino acid sequence of SEQ ID NO:139 (IGKV3-11). In a particular aspect, an antibody capable of specific binding to CEA comprises a VH domain comprising an amino acid sequence of SEQ ID NO:23 and a VL domain comprising an amino acid sequence of SEQ ID NO:24. Thus, in one aspect a humanized antibody is provided that competes for binding with an antibody that comprises a variable heavy chain domain (VH) comprising (i) CDR-H1 comprising the amino acid sequence of SEQ ID NO:1, (ii) CDR-H2 comprising the amino acid sequence of SEQ ID NO:2, and (iii) CDR-H3 comprising the amino acid sequence of SEQ ID NO:3, and a variable light chain domain (VL) comprising (iv) CDR-L1 comprising the amino acid sequence of SEQ ID NO:4, (v) CDR-L2 comprising the amino acid sequence of SEQ ID NO:5, and (vi) CDR-L3 comprising the amino acid sequence of SEQ ID NO:6, wherein the antibody comprises a VH domain comprising an amino acid sequence of SEQ ID NO:23 and a VL domain comprising an amino acid sequence of SEQ ID NO:24.

In a further aspect, a humanized and affinity-matured antibody is provided. In one aspect, said humanized and affinity-matured antibody comprises a VH domain comprising (i) CDR-H1 comprising the amino acid sequence of SEQ ID NO:25, (ii) CDR-H2 comprising the amino acid sequence of SEQ ID NO:26, and (iii) CDR-H3 comprising the amino acid sequence of SEQ ID NO:27, and a VL domain comprising (iv) CDR-L1 comprising the amino acid sequence of SEQ ID NO:28, (v) CDR-L2 comprising the amino acid sequence of SEQ ID NO:29, and (vi) CDR-L3 comprising the amino acid sequence of SEQ ID NO:30.

In a particular aspect, the humanized and affinity-matured antibody capable of specific binding to CEA comprises

(b) a VH domain comprising an amino acid sequence of SEQ ID NO:31 and a VL domain comprising an amino acid sequence of SEQ ID NO:32, or
(c) a VH domain comprising an amino acid sequence of SEQ ID NO:33 and a VL domain comprising an amino acid sequence of SEQ ID NO:34, or
(d) a VH domain comprising an amino acid sequence of SEQ ID NO:35 and a VL domain comprising an amino acid sequence of SEQ ID NO:36, or
(e) a VH domain comprising an amino acid sequence of SEQ ID NO:37 and a VL domain comprising an amino acid sequence of SEQ ID NO:38, or
(f) a VH domain comprising an amino acid sequence of SEQ ID NO:39 and a VL domain comprising an amino acid sequence of SEQ ID NO:40, or
(g) a VH domain comprising an amino acid sequence of SEQ ID NO:41 and a VL domain comprising an amino acid sequence of SEQ ID NO:42, or
(h) a VH domain comprising an amino acid sequence of SEQ ID NO:43 and a VL domain comprising an amino acid sequence of SEQ ID NO:44, or
(i) a VH domain comprising an amino acid sequence of SEQ ID NO:45 and a VL domain comprising an amino acid sequence of SEQ ID NO:46, or
(j) a VH domain comprising an amino acid sequence of SEQ ID NO:47 and a VL domain comprising an amino acid sequence of SEQ ID NO:48, or
(k) a VH domain comprising an amino acid sequence of SEQ ID NO:49 and a VL domain comprising an amino acid sequence of SEQ ID NO:50.
(l) a VH domain comprising an amino acid sequence of SEQ ID NO:51 and a VL domain comprising an amino acid sequence of SEQ ID NO:52, or
(m) a VH domain comprising an amino acid sequence of SEQ ID NO:53 and a VL domain comprising an amino acid sequence of SEQ ID NO:54.

In one aspect, the humanized and affinity-matured antibody capable of specific binding to CEA comprises a VH domain comprising an amino acid sequence of SEQ ID NO:39 and a VL domain comprising an amino acid sequence of SEQ ID NO:40, or a VH domain comprising an amino acid sequence of SEQ ID NO:41 and a VL domain comprising an amino acid sequence of SEQ ID NO:42.

In a second aspect, the invention provides new humanized antibodies that bind to the A1 domain of carcinoembryonic antigen (CEA). Thus, in one aspect, new humanized antibodies are provided that bind to a domain comprising the amino acid sequence of SEQ ID NO:312. In one aspect, provided are antibodies that compete for binding with an antibody that comprises a variable heavy chain domain (VH) comprising (i) CDR-H1 comprising the amino acid sequence of SEQ ID NO:55, (ii) CDR-H2 comprising the amino acid sequence of SEQ ID NO:56, and (iii) CDR-H3 comprising the amino acid sequence of SEQ ID NO:57, and a variable light chain domain (VL) comprising (iv) CDR-L1 comprising the amino acid sequence of SEQ ID NO:58, (v) CDR-L2 comprising the amino acid sequence of SEQ ID NO:59, and (vi) CDR-L3 comprising the amino acid sequence of SEQ ID NO:60. In particular, these antibodies compete for binding with an antibody comprising a VH domain comprising the amino acid sequence of SEQ ID NO:61 and a VL domain comprising the amino acid sequence of SEQ ID NO.62. Provided are thus antibodies capable of specific binding to CEA that do not compete with an antibody comprising the amino acid sequence of comprising a VH domain comprising the amino acid sequence of SEQ ID NO:63 and a VL domain comprising the amino acid sequence of SEQ ID NO:64.

In one aspect, provided is a humanized antibody capable of specific binding to CEA, wherein the antibody comprises a variable heavy chain domain (VH) comprising (i) CDR-H1 comprising the amino acid sequence of SEQ ID NO:65, (ii) CDR-H2 comprising the amino acid sequence of SEQ ID NO:66 or SEQ ID NO:67, and (iii) CDR-H3 comprising the amino acid sequence of SEQ ID NO:68, and a VL domain comprising (iv) CDR-L1 comprising the amino acid sequence of SEQ ID NO:69 or SEQ ID NO:70 or SEQ ID NO:313, (v) CDR-L2 comprising the amino acid sequence of SEQ ID NO:71 or SEQ ID NO:72 or SEQ ID NO:73, and (vi) CDR-L3 comprising the amino acid sequence of SEQ ID NO:74. In particular, the framework of the VH domain is based on a human acceptor framework comprising the amino acid sequence of SEQ ID NO:232 (IGHV1-2-02) and the framework of VL domain is based on a human acceptor framework comprising the amino acid sequence of SEQ ID NO:235 (IGKV1-39-01), In one aspect, provided is a humanized antibody capable of specific binding to CEA, wherein the antigen binding domain capable of specific binding to CEA comprises a heavy chain variable region (VH) comprising the amino acid sequence of SEQ ID NO:75, SEQ ID NO:76, SEQ ID NO:77, SEQ ID NO:78, SEQ ID NO:79 or SEQ ID NO:80, and a light chain variable region (VL) comprising the amino acid sequence of SEQ ID NO:81, SEQ ID NO:82, SEQ ID NO:83, SEQ ID NO:84, SEQ ID NO:85 or SEQ ID NO:86.

In a particular aspect, there is provided a humanized antibody capable of specific binding to CEA, wherein the antigen binding domain capable of specific binding to CEA comprises

(a) a VH domain comprising an amino acid sequence of SEQ ID NO:75 and a VL domain comprising an amino acid sequence of SEQ ID NO:85, or
(b) a VH domain comprising an amino acid sequence of SEQ ID NO:79 and a VL domain comprising an amino acid sequence of SEQ ID NO:85, or
(c) a VH domain comprising an amino acid sequence of SEQ ID NO:76 and a VL domain comprising an amino acid sequence of SEQ ID NO:85, or
(d) a VH domain comprising an amino acid sequence of SEQ ID NO:80 and a VL domain comprising an amino acid sequence of SEQ ID NO:84, or
(e) a VH domain comprising an amino acid sequence of SEQ ID NO:79 and a VL domain comprising an amino acid sequence of SEQ ID NO:84, or
(f) a VH domain comprising an amino acid sequence of SEQ ID NO:77 and a VL domain comprising an amino acid sequence of SEQ ID NO:84, or
(g) a VH domain comprising an amino acid sequence of SEQ ID NO:75 and a VL domain comprising an amino acid sequence of SEQ ID NO:84.

In one aspect, provided is a humanized antibody capable of specific binding to CEA, wherein the antibody comprises a variable heavy chain domain (VH) comprising (i) CDR-H1 comprising the amino acid sequence of SEQ ID NO:65, (ii) CDR-H2 comprising the amino acid sequence of SEQ ID NO:66, and (iii) CDR-H3 comprising the amino acid sequence of SEQ ID NO:68, and a VL domain comprising (iv) CDR-L1 comprising the amino acid sequence of SEQ ID NO:313, (v) CDR-L2 comprising the amino acid sequence of SEQ ID NO:71, and (vi) CDR-L3 comprising the amino acid sequence of SEQ ID NO:74. In one particular aspect, provided is a humanized antibody capable of specific binding to CEA comprising variable heavy chain domain (VH) comprising an amino acid sequence of SEQ ID NO:75 and a variable light chain domain (VL) comprising an amino acid sequence of SEQ ID NO:85.

4-1BBL Trimer-Containing Antigen Binding Molecules of the Invention

The invention provides novel 4-1BBL trimer-containing antigen binding molecules with particularly advantageous properties such as binding to a different epitope, producibility, stability, binding affinity, biological activity, targeting efficiency, and reduced toxicity. The 4-1BBL trimer-containing antigen binding molecules of the invention are particularly useful for (co-)stimulation of cytotoxic T cells, e.g. in combination with a T-cell activating agent such as a T cell bispecific antibody (TCB). The antibodies of the invention can also efficiently activate other 4-1BB-expressing immune cells without the need for simultaneous stimulation through an activating Fc receptor such as FcγRIIIa (CD16a). Through avoiding the need for Fc receptor binding and activation for their function, the 4-1BBL trimer-containing antigen binding molecules of the present invention may enable efficient immune cell activation, with a smaller risk of systemic side effects than an 4-1BB antibody requiring Fc receptor binding and activation for its function. Through their binding to CEA, the 4-1BBL trimer-containing antigen binding molecules achieve tumor-specific immune cell activation and the risk of of systemic side effects is reduced.

In a first aspect, the invention provides a 4-1BBL trimer-containing antigen binding molecule comprising

an antigen binding domain capable of specific binding to CEA,
a first and a second polypeptide that are linked to each other by a disulfide bond,
wherein the antigen binding molecule is characterized in that the first polypeptide comprises two ectodomains of 4-1BBL or a fragment thereof that are connected to each other by a peptide linker and in that the second polypeptide comprises one ectodomain of 4-1BBL or a fragment thereof, and
an Fc domain composed of a first and a second subunit capable of stable association,

wherein the antigen binding domain capable of specific binding to CEA comprises

(a) a variable heavy chain domain (VH) comprising (i) CDR-H1 comprising the amino acid sequence of SEQ ID NO:17, (ii) CDR-H2 comprising the amino acid sequence of SEQ ID NO:18, and (iii) CDR-H3 comprising the amino acid sequence of SEQ ID NO:19, and a variable light chain domain (VL) comprising (iv) CDR-L1 comprising the amino acid sequence of SEQ ID NO:20, (v) CDR-L2 comprising the amino acid sequence of SEQ ID NO:21, and (vi) CDR-L3 comprising the amino acid sequence of SEQ ID NO:22, or
(b) a VH domain comprising (i) CDR-H1 comprising the amino acid sequence of SEQ ID NO:25, (ii) CDR-H2 comprising the amino acid sequence of SEQ ID NO:26, and (iii) CDR-H3 comprising the amino acid sequence of SEQ ID NO:27, and a VL domain comprising (iv) CDR-L1 comprising the amino acid sequence of SEQ ID NO:28, (v) CDR-L2 comprising the amino acid sequence of SEQ ID NO:29, and (vi) CDR-L3 comprising the amino acid sequence of SEQ ID NO:30, or
(c) a VH domain comprising (i) CDR-H1 comprising the amino acid sequence of SEQ ID NO:65, (ii) CDR-H2 comprising the amino acid sequence of SEQ ID NO:66 or SEQ ID NO:67, and (iii) CDR-H3 comprising the amino acid sequence of SEQ ID NO:68, and a VL domain comprising (iv) CDR-L1 comprising the amino acid sequence of SEQ ID NO:69 or SEQ ID NO:70 or SEQ ID NO:313, (v) CDR-L2 comprising the amino acid sequence of SEQ ID NO:71 or SEQ ID NO:72 or SEQ ID NO:73, and (vi) CDR-L3 comprising the amino acid sequence of SEQ ID NO:74.

In another aspect, provided is a 4-1BBL trimer-containing antigen binding molecule as defined herein before, wherein the ectodomain of 4-1BBL or a fragment thereof comprises the amino acid sequence selected from the group consisting of SEQ ID NO:87, SEQ ID NO:88, SEQ ID NO:89, SEQ ID NO:90, SEQ ID NO:91, SEQ ID NO:92, SEQ ID NO:93 and SEQ ID NO:94, particularly the amino acid sequence of SEQ ID NO:91.

In a further aspect, provided is a 4-1BBL trimer-containing antigen binding molecule as described herein, comprising

an antigen binding domain capable of specific binding to CEA,
a first and a second polypeptide that are linked to each other by a disulfide bond,
wherein the antigen binding molecule is characterized in that the first polypeptide comprises the amino acid sequence selected from the group consisting of SEQ ID NO:95, SEQ ID NO:96, SEQ ID NO:97 and SEQ ID NO:98 and in that the second polypeptide comprises the amino acid sequence selected from the group consisting of SEQ ID NO:87, SEQ ID NO:91, SEQ ID NO:89 and SEQ ID NO:94, and
an Fc domain composed of a first and a second subunit capable of stable association, wherein the antigen binding domain capable of specific binding to CEA comprises
(a) a variable heavy chain domain (VH) comprising (i) CDR-H1 comprising the amino acid sequence of SEQ ID NO:17, (ii) CDR-H2 comprising the amino acid sequence of SEQ ID NO:18, and (iii) CDR-H3 comprising the amino acid sequence of SEQ ID NO:19, and a variable light chain domain (VL) comprising (iv) CDR-L1 comprising the amino acid sequence of SEQ ID NO:20, (v) CDR-L2 comprising the amino acid sequence of SEQ ID NO:21, and (vi) CDR-L3 comprising the amino acid sequence of SEQ ID NO:22, or
(b) a VH domain comprising (i) CDR-H1 comprising the amino acid sequence of SEQ ID NO:25, (ii) CDR-H2 comprising the amino acid sequence of SEQ ID NO:26, and (iii) CDR-H3 comprising the amino acid sequence of SEQ ID NO:27, and a VL domain comprising (iv) CDR-L1 comprising the amino acid sequence of SEQ ID NO:28, (v) CDR-L2 comprising the amino acid sequence of SEQ ID NO:29, and (vi) CDR-L3 comprising the amino acid sequence of SEQ ID NO:30, or
(c) a VH domain comprising (i) CDR-H1 comprising the amino acid sequence of SEQ ID NO:65, (ii) CDR-H2 comprising the amino acid sequence of SEQ ID NO:66 or SEQ ID NO:67, and (iii) CDR-H3 comprising the amino acid sequence of SEQ ID NO:68, and a VL domain comprising (iv) CDR-L1 comprising the amino acid sequence of SEQ ID NO:69 or SEQ ID NO:70 or SEQ ID NO:313, (v) CDR-L2 comprising the amino acid sequence of SEQ ID NO:71 or SEQ ID NO:72 or SEQ ID NO:73, and (vi) CDR-L3 comprising the amino acid sequence of SEQ ID NO:74.

In one aspect, the 4-1BBL trimer-containing antigen binding molecule is one, wherein wherein the antigen binding domain capable of specific binding to CEA is a Fab molecule capable of specific binding to CEA. In another aspect, the antigen binding domain capable of specific binding to CEA is a cross-over Fab molecule or a scFV molecule capable of specific binding to CEA.

In one aspect, provided is a 4-1BBL trimer-containing antigen binding molecule, wherein the antigen binding domain capable of specific binding to CEA comprises

(a) a variable heavy chain domain (VH) comprising (i) CDR-H1 comprising the amino acid sequence of SEQ ID NO:17, (ii) CDR-H2 comprising the amino acid sequence of SEQ ID NO:18, and (iii) CDR-H3 comprising the amino acid sequence of SEQ ID NO:19, and a variable light chain domain (VL) comprising (iv) CDR-L1 comprising the amino acid sequence of SEQ ID NO:20, (v) CDR-L2 comprising the amino acid sequence of SEQ ID NO:21, and (vi) CDR-L3 comprising the amino acid sequence of SEQ ID NO:22, or
(b) a VH domain comprising (i) CDR-H1 comprising the amino acid sequence of SEQ ID NO:25, (ii) CDR-H2 comprising the amino acid sequence of SEQ ID NO:26, and (iii) CDR-H3 comprising the amino acid sequence of SEQ ID NO:27, and a VL domain comprising (iv) CDR-L1 comprising the amino acid sequence of SEQ ID NO:28, (v) CDR-L2 comprising the amino acid sequence of SEQ ID NO:29, and (vi) CDR-L3 comprising the amino acid sequence of SEQ ID NO:30.

In one aspect, the antigen binding domain capable of specific binding to CEA comprises a variable heavy chain domain (VH) comprising (i) CDR-H1 comprising the amino acid sequence of SEQ ID NO:17, (ii) CDR-H2 comprising the amino acid sequence of SEQ ID NO:18, and (iii) CDR-H3 comprising the amino acid sequence of SEQ ID NO:19, and a variable light chain domain (VL) comprising (iv) CDR-L1 comprising the amino acid sequence of SEQ ID NO:20, (v) CDR-L2 comprising the amino acid sequence of SEQ ID NO:21, and (vi) CDR-L3 comprising the amino acid sequence of SEQ ID NO:22.

In another aspect, the antigen binding domain capable of specific binding to CEA comprises a VH domain comprising (i) CDR-H1 comprising the amino acid sequence of SEQ ID NO:25, (ii) CDR-H2 comprising the amino acid sequence of SEQ ID NO:26, and (iii) CDR-H3 comprising the amino acid sequence of SEQ ID NO:27, and a VL domain comprising (iv) CDR-L1 comprising the amino acid sequence of SEQ ID NO:28, (v) CDR-L2 comprising the amino acid sequence of SEQ ID NO:29, and (vi) CDR-L3 comprising the amino acid sequence of SEQ ID NO:30.

In a particular aspect, provided is a 4-1BBL trimer-containing antigen binding molecule, wherein the antigen binding domain capable of specific binding to CEA comprises

(a) a VH domain comprising an amino acid sequence of SEQ ID NO:23 and a VL domain comprising an amino acid sequence of SEQ ID NO:24, or
(b) a VH domain comprising an amino acid sequence of SEQ ID NO:31 and a VL domain comprising an amino acid sequence of SEQ ID NO:32, or
(c) a VH domain comprising an amino acid sequence of SEQ ID NO:33 and a VL domain comprising an amino acid sequence of SEQ ID NO:34, or
(d) a VH domain comprising an amino acid sequence of SEQ ID NO:35 and a VL domain comprising an amino acid sequence of SEQ ID NO:36, or
(e) a VH domain comprising an amino acid sequence of SEQ ID NO:37 and a VL domain comprising an amino acid sequence of SEQ ID NO:38, or
(f) a VH domain comprising an amino acid sequence of SEQ ID NO:39 and a VL domain comprising an amino acid sequence of SEQ ID NO:40, or
(g) a VH domain comprising an amino acid sequence of SEQ ID NO:41 and a VL domain comprising an amino acid sequence of SEQ ID NO:42, or
(h) a VH domain comprising an amino acid sequence of SEQ ID NO:43 and a VL domain comprising an amino acid sequence of SEQ ID NO:44, or
(i) a VH domain comprising an amino acid sequence of SEQ ID NO:45 and a VL domain comprising an amino acid sequence of SEQ ID NO:46, or
(j) a VH domain comprising an amino acid sequence of SEQ ID NO:47 and a VL domain comprising an amino acid sequence of SEQ ID NO:48, or
(k) a VH domain comprising an amino acid sequence of SEQ ID NO:49 and a VL domain comprising an amino acid sequence of SEQ ID NO:50.
(l) a VH domain comprising an amino acid sequence of SEQ ID NO:51 and a VL domain comprising an amino acid sequence of SEQ ID NO:52, or
(m) a VH domain comprising an amino acid sequence of SEQ ID NO:53 and a VL domain comprising an amino acid sequence of SEQ ID NO:54.

In a further aspect, the invention provides a 4-1BBL trimer-containing antigen binding molecule as described herein, wherein the antigen binding domain capable of specific binding to CEA comprises a VH domain comprising (i) CDR-H1 comprising the amino acid sequence of SEQ ID NO:65, (ii) CDR-H2 comprising the amino acid sequence of SEQ ID NO:66 or SEQ ID NO:67, and (iii) CDR-H3 comprising the amino acid sequence of SEQ ID NO:68, and a VL domain comprising (iv) CDR-L1 comprising the amino acid sequence of SEQ ID NO:69 or SEQ ID NO:70 or SEQ ID NO:313, (v) CDR-L2 comprising the amino acid sequence of SEQ ID NO:71 or SEQ ID NO:72 or SEQ ID NO:73, and (vi) CDR-L3 comprising the amino acid sequence of SEQ ID NO:74. In one aspect, the antigen binding domain capable of specific binding to CEA comprises a heavy chain variable region (VH) comprising the amino acid sequence of SEQ ID NO:75, SEQ ID NO:76, SEQ ID NO:77, SEQ ID NO:78, SEQ ID NO:79 or SEQ ID NO:80, and a light chain variable region (VL) comprising the amino acid sequence of SEQ ID NO:81, SEQ ID NO:82, SEQ ID NO:83, SEQ ID NO:84, SEQ ID NO:85 or SEQ ID NO:86.

In a particular aspect, provided is a 4-1BBL trimer-containing antigen binding molecule, wherein the antigen binding domain capable of specific binding to CEA comprises

(a) a VH domain comprising an amino acid sequence of SEQ ID NO:75 and a VL domain comprising an amino acid sequence of SEQ ID NO:85, or
(b) a VH domain comprising an amino acid sequence of SEQ ID NO:79 and a VL domain comprising an amino acid sequence of SEQ ID NO:85, or
(c) a VH domain comprising an amino acid sequence of SEQ ID NO:76 and a VL domain comprising an amino acid sequence of SEQ ID NO:85, or
(d) a VH domain comprising an amino acid sequence of SEQ ID NO:80 and a VL domain comprising an amino acid sequence of SEQ ID NO:84, or
(e) a VH domain comprising an amino acid sequence of SEQ ID NO:79 and a VL domain comprising an amino acid sequence of SEQ ID NO:84, or
(f) a VH domain comprising an amino acid sequence of SEQ ID NO:77 and a VL domain comprising an amino acid sequence of SEQ ID NO:84, or
(g) a VH domain comprising an amino acid sequence of SEQ ID NO:75 and a VL domain comprising an amino acid sequence of SEQ ID NO:84.

In another aspect, provided is a 4-1BBL trimer-containing antigen binding molecule, wherein the first peptide comprising two ectodomains of 4-1BBL or a fragment thereof connected to each other by a first peptide linker is fused at its C-terminus by a second peptide linker to a CL domain that is part of a heavy chain, and the second peptide comprising one ectodomain of said 4-1BBL or a fragment thereof is fused at its C-terminus by a third peptide linker to a CH1 domain that is part of a light chain. In another aspect, provided is a 4-1BBL trimer-containing antigen binding molecule, wherein the first peptide comprising two ectodomains of 4-1BBL or a fragment thereof connected to each other by a first peptide linker is fused at its C-terminus by a second peptide linker to a CH domain that is part of a heavy chain, and the second peptide comprising one ectodomain of said 4-1BBL or a fragment thereof is fused at its C-terminus by a third peptide linker to a CL domain that is part of a light chain.

In a further aspect, there is provided a 4-1BBL trimer-containing antigen binding molecule as described herein, wherein the antigen binding molecule comprises

(i) a first heavy chain and a first light chain, both comprising a Fab molecule capable of specific binding to CEA,
(ii) a second heavy chain comprising the amino acid sequence selected from the group consisting of SEQ ID NO:99, SEQ ID NO:101, SEQ ID NO:103 and SEQ ID NO:105, and
(iii) a second light chain comprising the amino acid sequence selected from the group consisting of SEQ ID NO:100, SEQ ID NO:102, SEQ ID NO:104 and SEQ ID NO:106.

Particularly, provided is a 4-1BBL trimer-containing antigen binding molecule as described herein, wherein the antigen binding molecule comprises

(a) a first heavy chain comprising the amino acid sequence of SEQ ID NO:99, a first light chain comprising the amino acid sequence of SEQ ID NO:100, a second heavy chain comprising the amino acid sequence of SEQ ID NO:238 and a second light chain comprising the amino acid sequence of SEQ ID NO:239, or
(b) a first heavy chain comprising the amino acid sequence of SEQ ID NO:99, a first light chain comprising the amino acid sequence of SEQ ID NO:100, a second heavy chain comprising the amino acid sequence of SEQ ID NO:240 and a second light chain comprising the amino acid sequence of SEQ ID NO:241, or
(c) a first heavy chain comprising the amino acid sequence of SEQ ID NO:99, a first light chain comprising the amino acid sequence of SEQ ID NO:100, a second heavy chain comprising the amino acid sequence of SEQ ID NO:242 and a second light chain comprising the amino acid sequence of SEQ ID NO:243, or
(d) a first heavy chain comprising the amino acid sequence of SEQ ID NO:99, a first light chain comprising the amino acid sequence of SEQ ID NO:100, a second heavy chain comprising the amino acid sequence of SEQ ID NO:244 and a second light chain comprising the amino acid sequence of SEQ ID NO:245, or
(e) a first heavy chain comprising the amino acid sequence of SEQ ID NO:99, a first light chain comprising the amino acid sequence of SEQ ID NO:100, a second heavy chain comprising the amino acid sequence of SEQ ID NO:246 and a second light chain comprising the amino acid sequence of SEQ ID NO:247, or
(f) a first heavy chain comprising the amino acid sequence of SEQ ID NO:99, a first light chain comprising the amino acid sequence of SEQ ID NO:100, a second heavy chain comprising the amino acid sequence of SEQ ID NO:248 and a second light chain comprising the amino acid sequence of SEQ ID NO:249, or
(g) a first heavy chain comprising the amino acid sequence of SEQ ID NO:99, a first light chain comprising the amino acid sequence of SEQ ID NO:100, a second heavy chain comprising the amino acid sequence of SEQ ID NO:250 and a second light chain comprising the amino acid sequence of SEQ ID NO:251, or
(h) a first heavy chain comprising the amino acid sequence of SEQ ID NO:99, a first light chain comprising the amino acid sequence of SEQ ID NO:100, a second heavy chain comprising the amino acid sequence of SEQ ID NO:252 and a second light chain comprising the amino acid sequence of SEQ ID NO:253, or
(i) a first heavy chain comprising the amino acid sequence of SEQ ID NO:99, a first light chain comprising the amino acid sequence of SEQ ID NO:100, a second heavy chain comprising the amino acid sequence of SEQ ID NO:254 and a second light chain comprising the amino acid sequence of SEQ ID NO:255, or
(j) a first heavy chain comprising the amino acid sequence of SEQ ID NO:99, a first light chain comprising the amino acid sequence of SEQ ID NO:100, a second heavy chain comprising the amino acid sequence of SEQ ID NO:256 and a second light chain comprising the amino acid sequence of SEQ ID NO:257, or
(k) a first heavy chain comprising the amino acid sequence of SEQ ID NO:99, a first light chain comprising the amino acid sequence of SEQ ID NO:100, a second heavy chain comprising the amino acid sequence of SEQ ID NO:258 and a second light chain comprising the amino acid sequence of SEQ ID NO:259, or
(l) a first heavy chain comprising the amino acid sequence of SEQ ID NO:99, a first light chain comprising the amino acid sequence of SEQ ID NO:100, a second heavy chain comprising the amino acid sequence of SEQ ID NO:260 and a second light chain comprising the amino acid sequence of SEQ ID NO:261, or
(m) a first heavy chain comprising the amino acid sequence of SEQ ID NO:49, a first light chain comprising the amino acid sequence of SEQ ID NO:50, a second heavy chain comprising the amino acid sequence of SEQ ID NO:262 and a second light chain comprising the amino acid sequence of SEQ ID NO:263.

In another particular aspect, provided is a 4-1BBL trimer-containing antigen binding molecule, wherein the antigen binding molecule comprises

(a) a first heavy chain comprising the amino acid sequence of SEQ ID NO:99, a first light chain comprising the amino acid sequence of SEQ ID NO:100, a second heavy chain comprising the amino acid sequence of SEQ ID NO:266 and a second light chain comprising the amino acid sequence of SEQ ID NO:267, or
(b) a first heavy chain comprising the amino acid sequence of SEQ ID NO:99, a first light chain comprising the amino acid sequence of SEQ ID NO:100, a second heavy chain comprising the amino acid sequence of SEQ ID NO:268 and a second light chain comprising the amino acid sequence of SEQ ID NO:267, or
(c) a first heavy chain comprising the amino acid sequence of SEQ ID NO:99, a first light chain comprising the amino acid sequence of SEQ ID NO:100, a second heavy chain comprising the amino acid sequence of SEQ ID NO:269 and a second light chain comprising the amino acid sequence of SEQ ID NO:267, or
(d) a first heavy chain comprising the amino acid sequence of SEQ ID NO:99, a first light chain comprising the amino acid sequence of SEQ ID NO:100, a second heavy chain comprising the amino acid sequence of SEQ ID NO:270 and a second light chain comprising the amino acid sequence of SEQ ID NO:271, or
(e) a first heavy chain comprising the amino acid sequence of SEQ ID NO:99, a first light chain comprising the amino acid sequence of SEQ ID NO:100, a second heavy chain comprising the amino acid sequence of SEQ ID NO:272 and a second light chain comprising the amino acid sequence of SEQ ID NO:271, or
(f) a first heavy chain comprising the amino acid sequence of SEQ ID NO:99, a first light chain comprising the amino acid sequence of SEQ ID NO:100, a second heavy chain comprising the amino acid sequence of SEQ ID NO:273 and a second light chain comprising the amino acid sequence of SEQ ID NO:271, or
(g) a first heavy chain comprising the amino acid sequence of SEQ ID NO:99, a first light chain comprising the amino acid sequence of SEQ ID NO:100, a second heavy chain comprising the amino acid sequence of SEQ ID NO:274 and a second light chain comprising the amino acid sequence of SEQ ID NO:271.

The 4-1BBL trimer-containing antigen binding molecule as defined herein before comprises an Fc domain composed of a first and a second subunit capable of stable association. In one aspect, the 4-1BBL trimer-containing antigen binding molecule of the invention comprises (a) a Fab molecule capable of specific binding to CEA, wherein the Fab heavy chain is fused at the C-terminus to the N-terminus of a CH2 domain in the Fc domain and (c) an Fc domain composed of a first and a second subunit capable of stable association.

In particular, the Fc domain is an IgG, particularly an IgG1 Fc domain or an IgG4 Fc domain. More particularly, the Fc domain is an IgG1 Fc domain.

Fc Domain Modifications Reducing Fc Receptor Binding and/or Effector Function

The Fc domain of the 4-1BBL trimer-containing antigen binding molecules of the invention consists of a pair of polypeptide chains comprising heavy chain domains of an immunoglobulin molecule. For example, the Fc domain of an immunoglobulin G (IgG) molecule is a dimer, each subunit of which comprises the CH2 and CH3 IgG heavy chain constant domains. The two subunits of the Fc domain are capable of stable association with each other.

The Fc domain confers favorable pharmacokinetic properties to the antigen binding molecules of the invention, including a long serum half-life which contributes to good accumulation in the target tissue and a favorable tissue-blood distribution ratio. At the same time it may, however, lead to undesirable targeting of the bispecific antibodies of the invention to cells expressing Fc receptors rather than to the preferred antigen-bearing cells. Accordingly, in particular aspects, the Fc domain of the 4-1BBL trimer-containing antigen binding molecule of the invention exhibits reduced binding affinity to an Fc receptor and/or reduced effector function, as compared to a native IgG1 Fc domain. In one aspect, the Fc does not substantially bind to an Fc receptor and/or does not induce effector function. In a particular aspect the Fc receptor is an Fcγ receptor. In one aspect, the Fc receptor is a human Fc receptor. In a specific aspect, the Fc receptor is an activating human Fcγ receptor, more specifically human FcγRIIIa, FcγRI or FcγRIIa, most specifically human FcγRIIIa. In one aspect, the Fc domain does not induce effector function. The reduced effector function can include, but is not limited to, one or more of the following: reduced complement dependent cytotoxicity (CDC), reduced antibody-dependent cell-mediated cytotoxicity (ADCC), reduced antibody-dependent cellular phagocytosis (ADCP), reduced cytokine secretion, reduced immune complex-mediated antigen uptake by antigen-presenting cells, reduced binding to NK cells, reduced binding to macrophages, reduced binding to monocytes, reduced binding to polymorphonuclear cells, reduced direct signaling inducing apoptosis, reduced dendritic cell maturation, or reduced T cell priming.

In certain aspects, one or more amino acid modifications may be introduced into the Fc region of a 4-1BBL trimer-containing antigen binding molecule provided herein, thereby generating an Fc region variant. The Fc region variant may comprise a human Fc region sequence (e.g., a human IgG1, IgG2, IgG3 or IgG4 Fc region) comprising an amino acid modification (e.g. a substitution) at one or more amino acid positions.

In a particular aspect, the invention provides a 4-1BBL trimer-containing antigen binding molecule comprising

(a) an antigen binding domain capable of specific binding to CEA,
(b) a first and a second polypeptide that are linked to each other by a disulfide bond, wherein the antigen binding molecule is characterized in that the first polypeptide comprises two ectodomains of 4-1BBL or a fragment thereof that are connected to each other by a peptide linker and in that the second polypeptide comprises one ectodomain of 4-1BBL or a fragment thereof, and
(c) an Fc domain composed of a first and a second subunit capable of stable association, wherein the Fc domain comprises one or more amino acid substitution that reduces binding to an Fc receptor, in particular towards Fcγ receptor,
wherein the antigen binding domain capable of specific binding to CEA comprises
(a) a variable heavy chain domain (VH) comprising (i) CDR-H1 comprising the amino acid sequence of SEQ ID NO:17, (ii) CDR-H2 comprising the amino acid sequence of SEQ ID NO:18, and (iii) CDR-H3 comprising the amino acid sequence of SEQ ID NO:19, and a variable light chain domain (VL) comprising (iv) CDR-L1 comprising the amino acid sequence of SEQ ID NO:20, (v) CDR-L2 comprising the amino acid sequence of SEQ ID NO:21, and (vi) CDR-L3 comprising the amino acid sequence of SEQ ID NO:22, or
(b) a VH domain comprising (i) CDR-H1 comprising the amino acid sequence of SEQ ID NO:25, (ii) CDR-H2 comprising the amino acid sequence of SEQ ID NO:26, and (iii) CDR-H3 comprising the amino acid sequence of SEQ ID NO:27, and a VL domain comprising (iv) CDR-L1 comprising the amino acid sequence of SEQ ID NO:28, (v) CDR-L2 comprising the amino acid sequence of SEQ ID NO:29, and (vi) CDR-L3 comprising the amino acid sequence of SEQ ID NO:30, or
(c) a VH domain comprising (i) CDR-H1 comprising the amino acid sequence of SEQ ID NO:65, (ii) CDR-H2 comprising the amino acid sequence of SEQ ID NO:66 or SEQ ID NO:67, and (iii) CDR-H3 comprising the amino acid sequence of SEQ ID NO:68, and a VL domain comprising (iv) CDR-L1 comprising the amino acid sequence of SEQ ID NO:69 or SEQ ID NO:70 or SEQ ID NO:313, (v) CDR-L2 comprising the amino acid sequence of SEQ ID NO:71 or SEQ ID NO:72 or SEQ ID NO:73, and (vi) CDR-L3 comprising the amino acid sequence of SEQ ID NO:74.

In one aspect, the Fc domain of the 4-1BBL trimer-containing antigen binding molecule of the invention comprises one or more amino acid mutation that reduces the binding affinity of the Fc domain to an Fc receptor and/or effector function. Typically, the same one or more amino acid mutation is present in each of the two subunits of the Fc domain. In particular, the Fc domain comprises an amino acid substitution at a position of E233, L234, L235, N297, P331 and P329 (EU numbering). In particular, the Fc domain comprises amino acid substitutions at positions 234 and 235 (EU numbering) and/or 329 (EU numbering) of the IgG heavy chains. More particularly, provided is a trimeric TNF family ligand-containing antigen binding molecule according to the invention which comprises an Fc domain with the amino acid substitutions L234A, L235A and P329G (“P329G LALA”, EU numbering) in the IgG heavy chains. The amino acid substitutions L234A and L235A refer to the so-called LALA mutation. The “P329G LALA” combination of amino acid substitutions almost completely abolishes Fcγ receptor binding of a human IgG1 Fc domain and is described in International Patent Appl. Publ. No. WO 2012/130831 A1 which also describes methods of preparing such mutant Fc domains and methods for determining its properties such as Fc receptor binding or effector functions. “EU numbering” refers to the numbering according to EU index of Kabat et al, Sequences of Proteins of Immunological Interest, 5th Ed. Public Health Service, National Institutes of Health, Bethesda, Md., 1991.

Fc domains with reduced Fc receptor binding and/or effector function also include those with substitution of one or more of Fc domain residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

In another aspect, the Fc domain is an IgG4 Fc domain. IgG4 antibodies exhibit reduced binding affinity to Fc receptors and reduced effector functions as compared to IgG1 antibodies. In a more specific aspect, the Fc domain is an IgG4 Fc domain comprising an amino acid substitution at position 5228 (Kabat numbering), particularly the amino acid substitution S228P. In a more specific aspect, the Fc domain is an IgG4 Fc domain comprising amino acid substitutions L235E and S228P and P329G (EU numbering). Such IgG4 Fc domain mutants and their Fcγ receptor binding properties are also described in WO 2012/130831.

Mutant Fc domains can be prepared by amino acid deletion, substitution, insertion or modification using genetic or chemical methods well known in the art. Genetic methods may include site-specific mutagenesis of the encoding DNA sequence, PCR, gene synthesis, and the like. The correct nucleotide changes can be verified for example by sequencing.

Binding to Fc receptors can be easily determined e.g. by ELISA, or by Surface Plasmon Resonance (SPR) using standard instrumentation such as a BIAcore instrument (GE Healthcare), and Fc receptors such as may be obtained by recombinant expression. A suitable such binding assay is described herein. Alternatively, binding affinity of Fc domains or cell activating bispecific antigen binding molecules comprising an Fc domain for Fc receptors may be evaluated using cell lines known to express particular Fc receptors, such as human NK cells expressing FcγIIIa receptor.

Effector function of an Fc domain, or bispecific antibodies of the invention comprising an Fc domain, can be measured by methods known in the art. A suitable assay for measuring ADCC is described herein. Other examples of in vitro assays to assess ADCC activity of a molecule of interest are described in U.S. Pat. No. 5,500,362; Hellstrom et al. Proc Natl Acad Sci USA 83, 7059-7063 (1986) and Hellstrom et al., Proc Natl Acad Sci USA 82, 1499-1502 (1985); U.S. Pat. No. 5,821,337; Bruggemann et al., J Exp Med 166, 1351-1361 (1987). Alternatively, non-radioactive assays methods may be employed (see, for example, ACTI™ non-radioactive cytotoxicity assay for flow cytometry (CellTechnology, Inc. Mountain View, Calif.); and CytoTox 96® non-radioactive cytotoxicity assay (Promega, Madison, Wis.)). Useful effector cells for such assays include peripheral blood mononuclear cells (PBMC) and Natural Killer (NK) cells. Alternatively, or additionally, ADCC activity of the molecule of interest may be assessed in vivo, e.g. in a animal model such as that disclosed in Clynes et al., Proc Natl Acad Sci USA 95, 652-656 (1998).

In some embodiments, binding of the Fc domain to a complement component, specifically to C1q, is reduced. Accordingly, in some embodiments wherein the Fc domain is engineered to have reduced effector function, said reduced effector function includes reduced CDC. C1q binding assays may be carried out to determine whether the bispecific antibodies of the invention is able to bind C1q and hence has CDC activity. See e.g., C1q and C3c binding ELISA in WO 2006/029879 and WO 2005/100402. To assess complement activation, a CDC assay may be performed (see, for example, Gazzano-Santoro et al., J Immunol Methods 202, 163 (1996); Cragg et al., Blood 101, 1045-1052 (2003); and Cragg and Glennie, Blood 103, 2738-2743 (2004)).

In a particular aspect, the Fc domain comprises a modification promoting the association of the first and second subunit of the Fc domain.

Fc Domain Modifications Promoting Heterodimerization

In one aspect, the 4-1BBL trimer-containing antigen binding molecules of the invention comprise (a) an antigen binding domain capable of specific binding to CEA,

(b) a first and a second polypeptide that are linked to each other by a disulfide bond, wherein the antigen binding molecule is characterized in that the first polypeptide comprises two ectodomains of 4-1BBL or a fragment thereof that are connected to each other by a peptide linker and in that the second polypeptide comprises one ectodomain of 4-1BBL or a fragment thereof, and (c) an Fc domain composed of a first and a second subunit capable of stable association. Thus, they comprise different moieties, fused to one or the other of the two subunits of the Fc domain that are typically comprised in two non-identical polypeptide chains (“heavy chains”). Recombinant co-expression of these polypeptides and subsequent dimerization leads to several possible combinations of the two polypeptides. To improve the yield and purity of the 4-1BBL trimer-containing antigen binding molecules in recombinant production, it will thus be advantageous to introduce in the Fc domain of the 4-1BBL trimer-containing antigen binding molecules of the invention a modification promoting the association of the desired polypeptides.

Accordingly, the Fc domain of the 4-1BBL trimer-containing antigen binding molecules of the invention comprises a modification promoting the association of the first and the second subunit of the Fc domain. The site of most extensive protein-protein interaction between the two subunits of a human IgG Fc domain is in the CH3 domain of the Fc domain. Thus, said modification is particularly in the CH3 domain of the Fc domain.

In a specific aspect, said modification is a so-called “knob-into-hole” modification, comprising a “knob” modification in one of the two subunits of the Fc domain and a “hole” modification in the other one of the two subunits of the Fc domain. Thus, in a particular aspect, the invention relates to a 4-1BBL trimer-containing antigen binding molecule as described herein before which comprises an IgG molecule, wherein the Fc part of the first heavy chain comprises a first dimerization module and the Fc part of the second heavy chain comprises a second dimerization module allowing a heterodimerization of the two heavy chains of the IgG molecule and the first dimerization module comprises knobs and the second dimerization module comprises holes according to the knob-into-hole technology.

The knob-into-hole technology is described e.g. in U.S. Pat. Nos. 5,731,168; 7,695,936; Ridgway et al., Prot Eng 9, 617-621 (1996) and Carter, J Immunol Meth 248, 7-15 (2001). Generally, the method involves introducing a protuberance (“knob”) at the interface of a first polypeptide and a corresponding cavity (“hole”) in the interface of a second polypeptide, such that the protuberance can be positioned in the cavity so as to promote heterodimer formation and hinder homodimer formation. Protuberances are constructed by replacing small amino acid side chains from the interface of the first polypeptide with larger side chains (e.g. tyrosine or tryptophan). Compensatory cavities of identical or similar size to the protuberances are created in the interface of the second polypeptide by replacing large amino acid side chains with smaller ones (e.g. alanine or threonine).

Accordingly, in a particular aspect, in the CH3 domain of the first subunit of the Fc domain of the 4-1BBL trimer-containing antigen binding molecules of the invention an amino acid residue is replaced with an amino acid residue having a larger side chain volume, thereby generating a protuberance within the CH3 domain of the first subunit which is positionable in a cavity within the CH3 domain of the second subunit, and in the CH3 domain of the second subunit of the Fc domain an amino acid residue is replaced with an amino acid residue having a smaller side chain volume, thereby generating a cavity within the CH3 domain of the second subunit within which the protuberance within the CH3 domain of the first subunit is positionable.

The protuberance and cavity can be made by altering the nucleic acid encoding the polypeptides, e.g. by site-specific mutagenesis, or by peptide synthesis.

In a specific aspect, in the CH3 domain of the first subunit of the Fc domain the threonine residue at position 366 is replaced with a tryptophan residue (T366W), and in the CH3 domain of the second subunit of the Fc domain the tyrosine residue at position 407 is replaced with a valine residue (Y407V). More particularly, in the second subunit of the Fc domain additionally the threonine residue at position 366 is replaced with a serine residue (T366S) and the leucine residue at position 368 is replaced with an alanine residue (L368A). More particularly, in the first subunit of the Fc domain additionally the serine residue at position 354 is replaced with a cysteine residue (S354C), and in the second subunit of the Fc domain additionally the tyrosine residue at position 349 is replaced by a cysteine residue (Y349C). The introduction of these two cysteine residues results in the formation of a disulfide bridge between the two subunits of the Fc domain. The disulfide bridge further stabilizes the dimer (Carter, J Immunol Methods 248, 7-15 (2001)).

In an alternative aspect, a modification promoting association of the first and the second subunit of the Fc domain comprises a modification mediating electrostatic steering effects, e.g. as described in PCT publication WO 2009/089004. Generally, this method involves replacement of one or more amino acid residues at the interface of the two Fc domain subunits by charged amino acid residues so that homodimer formation becomes electrostatically unfavorable but heterodimerization electrostatically favorable.

In a particular aspect, the invention provides a 4-1BBL trimer-containing antigen binding molecule, wherein the first subunit of the Fc domain comprises the amino acid substitutions S354C and T366W (numbering according to Kabat EU index) and the second subunit of the Fc domain comprises the amino acid substitutions Y349C, T366S, L368A and Y407V (numbering according to Kabat EU index).

Modifications in the CH1/CL Domains

To further improve correct pairing, the 4-1BBL trimer-containing antigen binding molecules can contain different charged amino acid substitutions (so-called “charged residues”). These modifications are introduced in the crossed or non-crossed CH1 and CL domains. In a particular aspect, the invention relates to a 4-1BBL trimer-containing antigen binding molecule, wherein in one of CL domains the amino acid at position 123 (EU numbering) has been replaced by arginine (R) and the amino acid at position 124 (EU numbering) has been substituted by lysine (K) and wherein in one of the CH1 domains the the amino acids at position 147 (EU numbering) and at position 213 (EU numbering) have been substituted by glutamic acid (E).

More particularly, the invention relates to a 4-1BBL trimer-containing antigen binding molecule, wherein in the CL domain adjacent to the TNF ligand family member the amino acid at position 123 (EU numbering) has been replaced by arginine (R) and the amino acid at position 124 (EU numbering) has been substituted by lysine (K), and wherein in the CH1 domain adjacent to the TNF ligand family member the amino acids at position 147 (EU numbering) and at position 213 (EU numbering) have been substituted by glutamic acid (E).

Thus, in a particular aspect, provided is a 4-1BBL trimer-containing antigen binding molecule comprising

(a) an antigen binding domain capable of specific binding to CEA,
(b) a first polypeptide containing a CL domain comprising the amino acid mutations E123R and Q124K and a second polypeptide containing a CH1 domain comprising the amino acid mutations K147E and K213E, wherein the second polypeptide is linked to the first polypeptide by a disulfide bond between the CH1 and CL domain,
and wherein the antigen binding molecule is characterized in that the first polypeptide comprises two ectodomains of 4-1BBL or a fragment thereof that are connected to each other and to the CL domain by a peptide linker and in that the second polypeptide comprises one 4-1BBL or a fragment thereof connected via a peptide linker to the CH1 domain of said polypeptide; and
(c) an Fc domain composed of a first and a second subunit capable of stable association.

In one aspect, the invention provides a 4-1BBL trimer-containing antigen binding molecule, wherein in the CL domain adjacent to the TNF ligand family member the amino acid at position 123 (EU numbering) has been replaced by arginine (R) and the amino acid at position 124 (EU numbering) has been substituted by lysine (K), and wherein in the CH1 domain adjacent to the TNF ligand family member the amino acids at position 147 (EU numbering) and at position 213 (EU numbering) have been substituted by glutamic acid (E). These modifications lead to so-called charged residues with advantageous properties that avoid undesired effects such as for example mispairing.

In particular, the CL domain comprises the amino acid mutations E123R and Q124K and the CH1 domain comprises the amino acid mutations K147E and K213E.

Polynucleotides

The invention further provides isolated nucleic acid molecules encoding a 4-1BBL trimer-containing antigen binding molecule or an antibody as described herein or a fragment thereof.

The isolated polynucleotides encoding 4-1BBL trimer-containing antigen binding molecules of the invention may be expressed as a single polynucleotide that encodes the entire antigen binding molecule or as multiple (e.g., two or more) polynucleotides that are co-expressed. Polypeptides encoded by polynucleotides that are co-expressed may associate through, e.g., disulfide bonds or other means to form a functional antigen binding molecule. For example, the light chain portion of an immunoglobulin may be encoded by a separate polynucleotide from the heavy chain portion of the immunoglobulin. When co-expressed, the heavy chain polypeptides will associate with the light chain polypeptides to form the immunoglobulin.

In some aspects, the isolated nucleic acid molecule encodes the entire 4-1BBL trimer-containing antigen binding molecule according to the invention as described herein. In particular, the isolated polynucleotide encodes a polypeptide comprised in the 4-1BBL trimer-containing antigen binding molecule according to the invention as described herein.

In one aspect, the present invention is directed to isolated nucleic acid molecules encoding a 4-1BBL trimer-containing antigen binding molecule, wherein the nucleic acid molecule comprises (a) a sequence that encodes an antigen binding domain capable of specific binding to a CEA, (b) a sequence that encodes a polypeptide comprising two ectodomains of 4-1BBL or a fragment thereof that are connected to each other by a peptide linker and (c) a sequence that encodes a polypeptide comprising one ectodomain of said 4-1BBL or a fragment thereof.

In another aspect, provided is isolated nucleic acid encoding a 4-1BB ligand trimer-containing antigen binding molecule, wherein the polynucleotide comprises (a) a sequence that encodes a moiety capable of specific binding to CEA, (b) a sequence that encodes a polypeptide comprising two ectodomains of 4-1BBL or two fragments thereof that are connected to each other by a peptide linker and (c) a sequence that encodes a polypeptide comprising one ectodomain of 4-1BBL or a fragment thereof.

In another aspect, provided is isolated nucleic acid encoding an antibody capable of specific binding to CEA as described herein.

In certain aspects, the polynucleotide or nucleic acid is DNA. In other embodiments, a polynucleotide of the present invention is RNA, for example, in the form of messenger RNA (mRNA). RNA of the present invention may be single stranded or double stranded.

Recombinant Methods

4-1BBL trimer-containing antigen binding molecules of the invention may be obtained, for example, by solid-state peptide synthesis (e.g. Merrifield solid phase synthesis) or recombinant production. For recombinant production one or more polynucleotide encoding the 4-1BBL trimer-containing antigen binding molecule or polypeptide fragments thereof, e.g., as described above, is isolated and inserted into one or more vectors for further cloning and/or expression in a host cell. Such polynucleotide may be readily isolated and sequenced using conventional procedures. In one aspect of the invention, a vector, preferably an expression vector, comprising one or more of the polynucleotides of the invention is provided. Methods which are well known to those skilled in the art can be used to construct expression vectors containing the coding sequence of the 4-1BBL trimer-containing antigen binding molecule (fragment) along with appropriate transcriptional/translational control signals. These methods include in vitro recombinant DNA techniques, synthetic techniques and in vivo recombination/genetic recombination. See, for example, the techniques described in Maniatis et al., MOLECULAR CLONING: A LABORATORY MANUAL, Cold Spring Harbor Laboratory, N.Y. (1989); and Ausubel et al., CURRENT PROTOCOLS IN MOLECULAR BIOLOGY, Greene Publishing Associates and Wiley Interscience, N.Y. (1989). The expression vector can be part of a plasmid, virus, or may be a nucleic acid fragment. The expression vector includes an expression cassette into which the polynucleotide encoding the 4-1BBL trimer-containing antigen binding molecule or polypeptide fragments thereof (i.e. the coding region) is cloned in operable association with a promoter and/or other transcription or translation control elements. As used herein, a “coding region” is a portion of nucleic acid which consists of codons translated into amino acids. Although a “stop codon” (TAG, TGA, or TAA) is not translated into an amino acid, it may be considered to be part of a coding region, if present, but any flanking sequences, for example promoters, ribosome binding sites, transcriptional terminators, introns, 5′ and 3′ untranslated regions, and the like, are not part of a coding region. Two or more coding regions can be present in a single polynucleotide construct, e.g. on a single vector, or in separate polynucleotide constructs, e.g. on separate (different) vectors. Furthermore, any vector may contain a single coding region, or may comprise two or more coding regions, e.g. a vector of the present invention may encode one or more polypeptides, which are post- or co-translationally separated into the final proteins via proteolytic cleavage. In addition, a vector, polynucleotide, or nucleic acid of the invention may encode heterologous coding regions, either fused or unfused to a polynucleotide encoding the 4-1BBL trimer-containing antigen binding molecule of the invention or polypeptide fragments thereof, or variants or derivatives thereof. Heterologous coding regions include without limitation specialized elements or motifs, such as a secretory signal peptide or a heterologous functional domain. An operable association is when a coding region for a gene product, e.g. a polypeptide, is associated with one or more regulatory sequences in such a way as to place expression of the gene product under the influence or control of the regulatory sequence(s). Two DNA fragments (such as a polypeptide coding region and a promoter associated therewith) are “operably associated” if induction of promoter function results in the transcription of mRNA encoding the desired gene product and if the nature of the linkage between the two DNA fragments does not interfere with the ability of the expression regulatory sequences to direct the expression of the gene product or interfere with the ability of the DNA template to be transcribed. Thus, a promoter region would be operably associated with a nucleic acid encoding a polypeptide if the promoter was capable of effecting transcription of that nucleic acid. The promoter may be a cell-specific promoter that directs substantial transcription of the DNA only in predetermined cells. Other transcription control elements, besides a promoter, for example enhancers, operators, repressors, and transcription termination signals, can be operably associated with the polynucleotide to direct cell-specific transcription.

Suitable promoters and other transcription control regions are disclosed herein. A variety of transcription control regions are known to those skilled in the art. These include, without limitation, transcription control regions, which function in vertebrate cells, such as, but not limited to, promoter and enhancer segments from cytomegaloviruses (e.g. the immediate early promoter, in conjunction with intron-A), simian virus 40 (e.g. the early promoter), and retroviruses (such as, e.g. Rous sarcoma virus). Other transcription control regions include those derived from vertebrate genes such as actin, heat shock protein, bovine growth hormone and rabbit 5.-globin, as well as other sequences capable of controlling gene expression in eukaryotic cells. Additional suitable transcription control regions include tissue-specific promoters and enhancers as well as inducible promoters (e.g. promoters inducible tetracyclins). Similarly, a variety of translation control elements are known to those of ordinary skill in the art. These include, but are not limited to ribosome binding sites, translation initiation and termination codons, and elements derived from viral systems (particularly an internal ribosome entry site, or IRES, also referred to as a CITE sequence). The expression cassette may also include other features such as an origin of replication, and/or chromosome integration elements such as retroviral long terminal repeats (LTRs), or adeno-associated viral (AAV) inverted terminal repeats (ITRs).

Polynucleotide and nucleic acid coding regions of the present invention may be associated with additional coding regions which encode secretory or signal peptides, which direct the secretion of a polypeptide encoded by a polynucleotide of the present invention. For example, if secretion of the 4-1BBL trimer-containing antigen binding molecule or polypeptide fragments thereof is desired, DNA encoding a signal sequence may be placed upstream of the nucleic acid encoding a 4-1BBL trimer-containing antigen binding molecule of the invention or polypeptide fragments thereof. According to the signal hypothesis, proteins secreted by mammalian cells have a signal peptide or secretory leader sequence which is cleaved from the mature protein once export of the growing protein chain across the rough endoplasmic reticulum has been initiated. Those of ordinary skill in the art are aware that polypeptides secreted by vertebrate cells generally have a signal peptide fused to the N-terminus of the polypeptide, which is cleaved from the translated polypeptide to produce a secreted or “mature” form of the polypeptide. In certain embodiments, the native signal peptide, e.g. an immunoglobulin heavy chain or light chain signal peptide is used, or a functional derivative of that sequence that retains the ability to direct the secretion of the polypeptide that is operably associated with it. Alternatively, a heterologous mammalian signal peptide, or a functional derivative thereof, may be used. For example, the wild-type leader sequence may be substituted with the leader sequence of human tissue plasminogen activator (TPA) or mouse β-glucuronidase.

DNA encoding a short protein sequence that could be used to facilitate later purification (e.g. a histidine tag) or assist in labeling the fusion protein may be included within or at the ends of the polynucleotide encoding a 4-1BBL trimer-containing antigen binding molecule of the invention or polypeptide fragments thereof.

In a further aspect of the invention, a host cell comprising one or more polynucleotides of the invention is provided. In certain embodiments a host cell comprising one or more vectors of the invention is provided. The polynucleotides and vectors may incorporate any of the features, singly or in combination, described herein in relation to polynucleotides and vectors, respectively. In one aspect, a host cell comprises (e.g. has been transformed or transfected with) a vector comprising a polynucleotide that encodes (part of) a 4-1BBL trimer-containing antigen binding molecule of the invention of the invention. As used herein, the term “host cell” refers to any kind of cellular system which can be engineered to generate the fusion proteins of the invention or fragments thereof. Host cells suitable for replicating and for supporting expression of antigen binding molecules are well known in the art. Such cells may be transfected or transduced as appropriate with the particular expression vector and large quantities of vector containing cells can be grown for seeding large scale fermenters to obtain sufficient quantities of the antigen binding molecule for clinical applications. Suitable host cells include prokaryotic microorganisms, such as E. coli, or various eukaryotic cells, such as Chinese hamster ovary cells (CHO), insect cells, or the like. For example, polypeptides may be produced in bacteria in particular when glycosylation is not needed. After expression, the polypeptide may be isolated from the bacterial cell paste in a soluble fraction and can be further purified. In addition to prokaryotes, eukaryotic microbes such as filamentous fungi or yeast are suitable cloning or expression hosts for polypeptide-encoding vectors, including fungi and yeast strains whose glycosylation pathways have been “humanized”, resulting in the production of a polypeptide with a partially or fully human glycosylation pattern. See Gerngross, Nat Biotech 22, 1409-1414 (2004), and Li et al., Nat Biotech 24, 210-215 (2006).

Suitable host cells for the expression of (glycosylated) polypeptides are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells. Plant cell cultures can also be utilized as hosts. See e.g. U.S. Pat. Nos. 5,959,177, 6,040,498, 6,420,548, 7,125,978, and 6,417,429 (describing PLANTIBODIES™ technology for producing antibodies in transgenic plants). Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293T cells as described, e.g., in Graham et al., J Gen Virol 36, 59 (1977)), baby hamster kidney cells (BHK), mouse sertoli cells (TM4 cells as described, e.g., in Mather, Biol Reprod 23, 243-251 (1980)), monkey kidney cells (CV1), African green monkey kidney cells (VERO-76), human cervical carcinoma cells (HELA), canine kidney cells (MDCK), buffalo rat liver cells (BRL 3A), human lung cells (W138), human liver cells (Hep G2), mouse mammary tumor cells (MMT 060562), TRI cells (as described, e.g., in Mather et al., Annals N.Y. Acad Sci 383, 44-68 (1982)), MRC 5 cells, and FS4 cells. Other useful mammalian host cell lines include Chinese hamster ovary (CHO) cells, including dhfr-CHO cells (Urlaub et al., Proc Natl Acad Sci USA 77, 4216 (1980)); and myeloma cell lines such as YO, NS0, P3X63 and Sp2/0. For a review of certain mammalian host cell lines suitable for protein production, see, e.g., Yazaki and Wu, Methods in Molecular Biology, Vol. 248 (B. K. C. Lo, ed., Humana Press, Totowa, N.J.), pp. 255-268 (2003). Host cells include cultured cells, e.g., mammalian cultured cells, yeast cells, insect cells, bacterial cells and plant cells, to name only a few, but also cells comprised within a transgenic animal, transgenic plant or cultured plant or animal tissue. In one embodiment, the host cell is a eukaryotic cell, preferably a mammalian cell, such as a Chinese Hamster Ovary (CHO) cell, a human embryonic kidney (HEK) cell or a lymphoid cell (e.g., YO, NS0, Sp20 cell). Standard technologies are known in the art to express foreign genes in these systems. Cells expressing a polypeptide comprising either the heavy or the light chain of an immunoglobulin, may be engineered so as to also express the other of the immunoglobulin chains such that the expressed product is an immunoglobulin that has both a heavy and a light chain.

In one aspect, a method of producing a 4-1BBL trimer-containing antigen binding molecule of the invention or polypeptide fragments thereof is provided, wherein the method comprises culturing a host cell comprising polynucleotides encoding the 4-1BBL trimer-containing antigen binding molecule of the invention or polypeptide fragments thereof, as provided herein, under conditions suitable for expression of the 4-1BBL trimer-containing antigen binding molecule of the invention or polypeptide fragments thereof, and recovering the 4-1BBL trimer-containing antigen binding molecule of the invention or polypeptide fragments thereof from the host cell (or host cell culture medium).

In the 4-1BBL trimer-containing antigen binding molecule of the invention, the components (at least one moiety capable of specific binding to a target cell antigen, one polypeptide comprising two ectodomains of a TNF ligand family member or fragments thereof and a polypeptide comprising one ectodomain of said 4-1BBL family member or a fragment thereof) are not genetically fused to each other. The polypeptides are designed such that its components (two ectodomains of a TNF ligand family member or fragments thereof and other components such as CH or CL) are fused to each other directly or through a linker sequence. The composition and length of the linker may be determined in accordance with methods well known in the art and may be tested for efficacy. Examples of linker sequences between different components of the antigen binding molecules of the invention are found in the sequences provided herein. Additional sequences may also be included to incorporate a cleavage site to separate the individual components of the fusion protein if desired, for example an endopeptidase recognition sequence.

In certain embodiments the moieties capable of specific binding to a target cell antigen (e.g. Fab fragments) forming part of the antigen binding molecule comprise at least an immunoglobulin variable region capable of binding to an antigen. Variable regions can form part of and be derived from naturally or non-naturally occurring antibodies and fragments thereof. Methods to produce polyclonal antibodies and monoclonal antibodies are well known in the art (see e.g. Harlow and Lane, “Antibodies, a laboratory manual”, Cold Spring Harbor Laboratory, 1988). Non-naturally occurring antibodies can be constructed using solid phase-peptide synthesis, can be produced recombinantly (e.g. as described in U.S. Pat. No. 4,186,567) or can be obtained, for example, by screening combinatorial libraries comprising variable heavy chains and variable light chains (see e.g. U.S. Pat. No. 5,969,108 to McCafferty).

Any animal species of immunoglobulin can be used in the invention. Non-limiting immunoglobulins useful in the present invention can be of murine, primate, or human origin. If the fusion protein is intended for human use, a chimeric form of immunoglobulin may be used wherein the constant regions of the immunoglobulin are from a human. A humanized or fully human form of the immunoglobulin can also be prepared in accordance with methods well known in the art (see e. g. U.S. Pat. No. 5,565,332 to Winter). Humanization may be achieved by various methods including, but not limited to (a) grafting the non-human (e.g., donor antibody) CDRs onto human (e.g. recipient antibody) framework and constant regions with or without retention of critical framework residues (e.g. those that are important for retaining good antigen binding affinity or antibody functions), (b) grafting only the non-human specificity-determining regions (SDRs or a-CDRs; the residues critical for the antibody-antigen interaction) onto human framework and constant regions, or (c) transplanting the entire non-human variable domains, but “cloaking” them with a human-like section by replacement of surface residues. Humanized antibodies and methods of making them are reviewed, e.g., in Almagro and Fransson, Front Biosci 13, 1619-1633 (2008), and are further described, e.g., in Riechmann et al., Nature 332, 323-329 (1988); Queen et al., Proc Natl Acad Sci USA 86, 10029-10033 (1989); U.S. Pat. Nos. 5,821,337, 7,527,791, 6,982,321, and 7,087,409; Jones et al., Nature 321, 522-525 (1986); Morrison et al., Proc Natl Acad Sci 81, 6851-6855 (1984); Morrison and Oi, Adv Immunol 44, 65-92 (1988); Verhoeyen et al., Science 239, 1534-1536 (1988); Padlan, Molec Immun 31(3), 169-217 (1994); Kashmiri et al., Methods 36, 25-34 (2005) (describing SDR (a-CDR) grafting); Padlan, Mol Immunol 28, 489-498 (1991) (describing “resurfacing”); Dall'Acqua et al., Methods 36, 43-60 (2005) (describing “FR shuffling”); and Osbourn et al., Methods 36, 61-68 (2005) and Klimka et al., Br J Cancer 83, 252-260 (2000) (describing the “guided selection” approach to FR shuffling). Particular immunoglobulins according to the invention are human immunoglobulins. Human antibodies and human variable regions can be produced using various techniques known in the art. Human antibodies are described generally in van Dijk and van de Winkel, Curr Opin Pharmacol 5, 368-74 (2001) and Lonberg, Curr Opin Immunol 20, 450-459 (2008). Human variable regions can form part of and be derived from human monoclonal antibodies made by the hybridoma method (see e.g. Monoclonal Antibody Production Techniques and Applications, pp. 51-63 (Marcel Dekker, Inc., New York, 1987)). Human antibodies and human variable regions may also be prepared by administering an immunogen to a transgenic animal that has been modified to produce intact human antibodies or intact antibodies with human variable regions in response to antigenic challenge (see e.g. Lonberg, Nat Biotech 23, 1117-1125 (2005). Human antibodies and human variable regions may also be generated by isolating Fv clone variable region sequences selected from human-derived phage display libraries (see e.g., Hoogenboom et al. in Methods in Molecular Biology 178, 1-37 (O'Brien et al., ed., Human Press, Totowa, N.J., 2001); and McCafferty et al., Nature 348, 552-554; Clackson et al., Nature 352, 624-628 (1991)). Phage typically display antibody fragments, either as single-chain Fv (scFv) fragments or as Fab fragments.

In certain aspects, the moieties capable of specific binding to a target cell antigen (e.g. Fab fragments) comprised in the antigen binding molecules of the present invention are engineered to have enhanced binding affinity according to, for example, the methods disclosed in PCT publication WO 2012/020006 (see Examples relating to affinity maturation) or U.S. Pat. Appl. Publ. No. 2004/0132066. The ability of the antigen binding molecules of the invention to bind to a specific antigenic determinant can be measured either through an enzyme-linked immunosorbent assay (ELISA) or other techniques familiar to one of skill in the art, e.g. surface plasmon resonance technique (Liljeblad, et al., Glyco J 17, 323-329 (2000)), and traditional binding assays (Heeley, Endocr Res 28, 217-229 (2002)). Competition assays may be used to identify an antigen binding molecule that competes with a reference antibody for binding to a particular antigen. In certain embodiments, such a competing antigen binding molecule binds to the same epitope (e.g. a linear or a conformational epitope) that is bound by the reference antigen binding molecule. Detailed exemplary methods for mapping an epitope to which an antigen binding molecule binds are provided in Morris (1996) “Epitope Mapping Protocols”, in Methods in Molecular Biology vol. 66 (Humana Press, Totowa, N.J.). In an exemplary competition assay, immobilized antigen is incubated in a solution comprising a first labeled antigen binding molecule that binds to the antigen and a second unlabeled antigen binding molecule that is being tested for its ability to compete with the first antigen binding molecule for binding to the antigen. The second antigen binding molecule may be present in a hybridoma supernatant. As a control, immobilized antigen is incubated in a solution comprising the first labeled antigen binding molecule but not the second unlabeled antigen binding molecule. After incubation under conditions permissive for binding of the first antibody to the antigen, excess unbound antibody is removed, and the amount of label associated with immobilized antigen is measured. If the amount of label associated with immobilized antigen is substantially reduced in the test sample relative to the control sample, then that indicates that the second antigen binding molecule is competing with the first antigen binding molecule for binding to the antigen. See Harlow and Lane (1988) Antibodies: A Laboratory Manual ch. 14 (Cold Spring Harbor Laboratory, Cold Spring Harbor, N.Y.).

4-1BBL trimer-containing antigen binding molecules of the invention prepared as described herein may be purified by art-known techniques such as high performance liquid chromatography, ion exchange chromatography, gel electrophoresis, affinity chromatography, size exclusion chromatography, and the like. The actual conditions used to purify a particular protein will depend, in part, on factors such as net charge, hydrophobicity, hydrophilicity etc., and will be apparent to those having skill in the art. For affinity chromatography purification an antibody, ligand, receptor or antigen can be used to which the 4-1BBL trimer-containing antigen binding molecule binds. For example, for affinity chromatography purification of fusion proteins of the invention, a matrix with protein A or protein G may be used. Sequential Protein A or G affinity chromatography and size exclusion chromatography can be used to isolate an antigen binding molecule essentially as described in the Examples. The purity of the 4-1BBL trimer-containing antigen binding molecule or fragments thereof can be determined by any of a variety of well-known analytical methods including gel electrophoresis, high pressure liquid chromatography, and the like. For example, the 4-1BBL trimer-containing antigen binding molecules expressed as described in the Examples were shown to be intact and properly assembled as demonstrated by reducing and non-reducing SDS-PAGE.

Assays

The antigen binding molecules provided herein may be identified, screened for, or characterized for their physical/chemical properties and/or biological activities by various assays known in the art. Biological activity may include, e.g., the ability to enhance the activation and/or proliferation of different immune cells especially T-cells. E.g. they enhance secretion of immunomodulating cytokines. Other immunomodulating cytokines which are or can be enhanced are e.g IL2, Granzyme B etc. Biological activity may also include, cynomolgus binding crossreactivity, as well as binding to different cell types. Antigen binding molecules having such biological activity in vivo and/or in vitro are also provided.

1. Affinity Assays

The affinity of the 4-1BBL trimer-containing antigen binding molecule provided herein for 4-1BB (CD137) can be determined in accordance with the methods set forth in the Examples by surface plasmon resonance (SPR), using standard instrumentation such as a BIAcore instrument (GE Healthcare), and receptors or target proteins such as may be obtained by recombinant expression. The affinity of the 4-1BBL trimer-containing antigen binding molecule for CEA or the affinity of the antibody capable of specific binding to CEA can also be determined by surface plasmon resonance (SPR), using standard instrumentation such as a BIAcore instrument (GE Healthcare), and receptors or target proteins such as may be obtained by recombinant expression. A specific illustrative and exemplary embodiment for measuring binding affinity is described in Example 1.1.5 or in Example 2.2. According to one aspect, K_{D} is measured by surface plasmon resonance using a BIACORE® T100 machine (GE Healthcare) at 25° C.

2. Binding Assays and Other Assays

Binding of the 4-1BBL trimer-containing antigen binding molecule provided herein to the corresponding receptor expressing cells may be evaluated using cell lines expressing the particular receptor or target antigen, for example by flow cytometry (FACS). In one aspect, fresh peripheral blood mononuclear cells (PBMCs) expressing 4-1BB can be used in the binding assay. These cells are used directly after isolation (naïve PMBCs) or after stimulation (activated PMBCs). In another aspect, activated mouse splenocytes (expressing 4-1BB) can be used to demonstrate the binding of the 4-1BBL trimer-containing antigen binding molecule of the invention to 4-1BB expressing cells.

In a further aspect, cell lines expressing CEA were used to demonstrate the binding of the antigen binding molecules to this target cell antigen. Binding assays to CEACAM5 are described in more detail in Example 3.1.

In another aspect, competition assays may be used to identify an antigen binding molecule that competes with a specific antibody or antigen binding molecule for binding to CEA or 4-1BB, respectively. In certain embodiments, such a competing antigen binding molecule binds to the same epitope (e.g., a linear or a conformational epitope) that is bound by a specific anti-CEA antibody or a specific 4-1BB antibody. Detailed exemplary methods for mapping an epitope to which an antibody binds are provided in Morris (1996) “Epitope Mapping Protocols,” in Methods in Molecular Biology vol. 66 (Humana Press, Totowa, N.J.).

3. Activity Assays

In one aspect, assays are provided for identifying 4-1BBL trimer-containing antigen binding molecules that bind to CEA and to 4-1BB having biological activity. Biological activity may include, e.g., agonistic signalling through 4-1BB on cancer cells expressing CEA. 4-1BBL trimer-containing antigen binding molecules identified by the assays as having such biological activity in vitro are also provided.

In certain aspects, a 4-1BBL trimer-containing antigen binding molecule of the invention is tested for such biological activity. Assays for detecting the biological activity of the molecules of the invention are described in Example 3.2. Furthermore, assays for detecting cell lysis (e.g. by measurement of LDH release), induced apoptosis kinetics (e.g. by measurement of Caspase 3/7 activity) or apoptosis (e.g. using the TUNEL assay) are well known in the art. In addition, the biological activity of such complexes can be assessed by evaluating their effects on survival, proliferation and lymphokine secretion of various lymphocyte subsets such as NK cells, NKT-cells or γδ T-cells or assessing their capacity to modulate phenotype and function of antigen presenting cells such as dendritic cells, monocytes/macrophages or B-cells.

Pharmaceutical Compositions, Formulations and Routes of Administration

In a further aspect, the invention provides pharmaceutical compositions comprising any of the 4-1BBL trimer-containing antigen binding molecules provided herein, e.g., for use in any of the below therapeutic methods. In one embodiment, a pharmaceutical composition comprises any of the 4-1BBL trimer-containing antigen binding molecules provided herein and at least one pharmaceutically acceptable excipient. In another embodiment, a pharmaceutical composition comprises any of the 4-1BBL trimer-containing antigen binding molecules provided herein and at least one additional therapeutic agent, e.g., as described below. In a further aspect, provided are also pharmaceutical compositions comprising any of the antibodies capable of specific binding to CEA.

Pharmaceutical compositions of the present invention comprise a therapeutically effective amount of one or more 4-1BBL trimer-containing antigen binding molecules or CEA antibodies dissolved or dispersed in a pharmaceutically acceptable excipient. The phrases “pharmaceutical or pharmacologically acceptable” refers to molecular entities and compositions that are generally non-toxic to recipients at the dosages and concentrations employed, i.e. do not produce an adverse, allergic or other untoward reaction when administered to an animal, such as, for example, a human, as appropriate. The preparation of a pharmaceutical composition that contains at least one 4-1BBL trimer-containing antigen binding molecule or antibody capable of specific binding to CEA and optionally an additional active ingredient will be known to those of skill in the art in light of the present disclosure, as exemplified by Remington's Pharmaceutical Sciences, 18th Ed. Mack Printing Company, 1990, incorporated herein by reference. In particular, the compositions are lyophilized formulations or aqueous solutions. As used herein, “pharmaceutically acceptable excipient” includes any and all solvents, buffers, dispersion media, coatings, surfactants, antioxidants, preservatives (e.g. antibacterial agents, antifungal agents), isotonic agents, salts, stabilizers and combinations thereof, as would be known to one of ordinary skill in the art.

Parenteral compositions include those designed for administration by injection, e.g. subcutaneous, intradermal, intralesional, intravenous, intraarterial intramuscular, intrathecal or intraperitoneal injection. For injection, the 4-1BBL trimer-containing antigen binding molecules of the invention may be formulated in aqueous solutions, preferably in physiologically compatible buffers such as Hanks' solution, Ringer's solution, or physiological saline buffer. The solution may contain formulatory agents such as suspending, stabilizing and/or dispersing agents. Alternatively, the fusion proteins may be in powder form for constitution with a suitable vehicle, e.g., sterile pyrogen-free water, before use. Sterile injectable solutions are prepared by incorporating the fusion proteins of the invention in the required amount in the appropriate solvent with various of the other ingredients enumerated below, as required. Sterility may be readily accomplished, e.g., by filtration through sterile filtration membranes. Generally, dispersions are prepared by incorporating the various sterilized active ingredients into a sterile vehicle which contains the basic dispersion medium and/or the other ingredients. In the case of sterile powders for the preparation of sterile injectable solutions, suspensions or emulsion, the preferred methods of preparation are vacuum-drying or freeze-drying techniques which yield a powder of the active ingredient plus any additional desired ingredient from a previously sterile-filtered liquid medium thereof. The liquid medium should be suitably buffered if necessary and the liquid diluent first rendered isotonic prior to injection with sufficient saline or glucose. The composition must be stable under the conditions of manufacture and storage, and preserved against the contaminating action of microorganisms, such as bacteria and fungi. It will be appreciated that endotoxin contamination should be kept minimally at a safe level, for example, less that 0.5 ng/mg protein. Suitable pharmaceutically acceptable excipients include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Aqueous injection suspensions may contain compounds which increase the viscosity of the suspension, such as sodium carboxymethyl cellulose, sorbitol, dextran, or the like. Optionally, the suspension may also contain suitable stabilizers or agents which increase the solubility of the compounds to allow for the preparation of highly concentrated solutions. Additionally, suspensions of the active compounds may be prepared as appropriate oily injection suspensions. Suitable lipophilic solvents or vehicles include fatty oils such as sesame oil, or synthetic fatty acid esters, such as ethyl cleats or triglycerides, or liposomes.

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences (18th Ed. Mack Printing Company, 1990). Sustained-release preparations may be prepared. Suitable examples of sustained-release preparations include semipermeable matrices of solid hydrophobic polymers containing the polypeptide, which matrices are in the form of shaped articles, e.g. films, or microcapsules. In particular embodiments, prolonged absorption of an injectable composition can be brought about by the use in the compositions of agents delaying absorption, such as, for example, aluminum monostearate, gelatin or combinations thereof.

Exemplary pharmaceutically acceptable excipients herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

Exemplary lyophilized antibody formulations are described in U.S. Pat. No. 6,267,958. Aqueous antibody formulations include those described in U.S. Pat. No. 6,171,586 and WO2006/044908, the latter formulations including a histidine-acetate buffer.

In addition to the compositions described previously, the fusion proteins may also be formulated as a depot preparation. Such long acting formulations may be administered by implantation (for example subcutaneously or intramuscularly) or by intramuscular injection. Thus, for example, the fusion proteins may be formulated with suitable polymeric or hydrophobic materials (for example as an emulsion in an acceptable oil) or ion exchange resins, or as sparingly soluble derivatives, for example, as a sparingly soluble salt.

Pharmaceutical compositions comprising the fusion proteins of the invention may be manufactured by means of conventional mixing, dissolving, emulsifying, encapsulating, entrapping or lyophilizing processes. Pharmaceutical compositions may be formulated in conventional manner using one or more physiologically acceptable carriers, diluents, excipients or auxiliaries which facilitate processing of the proteins into preparations that can be used pharmaceutically. Proper formulation is dependent upon the route of administration chosen.

The 4-1BBL trimer-containing antigen binding molecules or antibodies capable of specific binding to CEA may be formulated into a composition in a free acid or base, neutral or salt form. Pharmaceutically acceptable salts are salts that substantially retain the biological activity of the free acid or base. These include the acid addition salts, e.g. those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine. Pharmaceutical salts tend to be more soluble in aqueous and other protic solvents than are the corresponding free base forms.

The composition herein may also contain more than one active ingredients as necessary for the particular indication being treated, preferably those with complementary activities that do not adversely affect each other. Such active ingredients are suitably present in combination in amounts that are effective for the purpose intended.

In one aspect, the pharmaceutical compositions may comprise any of the 4-1BBL trimer-containing antigen binding molecules provided herein and at least one additional therapeutic agent. In one aspect, the pharmaceutical compositions may comprise any of the 4-1BBL trimer-containing antigen binding molecules provided herein and a T-cell activating anti-CD3 bispecific antibody.

The formulations to be used for in vivo administration are generally sterile. Sterility may be readily accomplished, e.g., by filtration through sterile filtration membranes.

Therapeutic Methods and Compositions

Any of the 4-1BBL trimer-containing antigen binding molecules or antibodies capable of specific binding to CEA provided herein may be used in therapeutic methods.

For use in therapeutic methods, 4-1BBL trimer-containing antigen binding molecules or CEA antibodies of the invention can be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners.

In one aspect, 4-1BBL trimer-containing antigen binding molecules or antibodies capable of specific binding to CEA of the invention for use as a medicament are provided. In further aspects, 4-1BBL trimer-containing antigen binding molecules or CEA antibodies of the invention for use in treating a disease, in particular for use in the treatment of cancer, are provided. In certain aspects, 4-1BBL trimer-containing antigen binding molecules of the invention for use in a method of treatment are provided. In one aspect, the invention provides a 4-1BBL trimer-containing antigen binding molecule as described herein for use in the treatment of a disease in an individual in need thereof. In certain aspects, the invention provides a 4-1BBL trimer-containing antigen binding molecule for use in a method of treating an individual having a disease comprising administering to the individual a therapeutically effective amount of the antigen binding molecule. In certain aspects, the disease to be treated is CEA-positive cancer. Examples of CEA-positive cancers include colon cancer, pancreatic cancer, gastric cancer, non-small-cell lung cancer (NSCLC), breast cancer, ovarian cancer, bladder cancer, esophageal cancer, cervix, carcinoma or endom adenocarcinoma, salivary gland, endometrial cancer and head & neck small cell cancer. In one aspect, the CEA-positive cancer is selected from the group consisting of colon adenocarcinoma, pancreas adenocarcinoma, gastric adenocarcinoma, non-small cell lung cancer (NSCLC), breast cancer, Cervix carcinoma and Esophageal adenocarcinoma. In particular, the CEA-positive cancer is colon cancer or non-small-cell lung cancer (NSCLC). Thus, a 4-1BBL trimer-containing antigen binding molecule as described herein for use in the treatment of these cancers is provided. The subject, patient, or “individual” in need of treatment is typically a mammal, more specifically a human.

In another aspect, provided is a 4-1BBL trimer-containing antigen binding molecule as described herein for use in the treatment of infectious diseases, in particular for the treatment of viral infections. In a further aspect, provided is a 4-1BBL trimer-containing antigen binding molecule as described herein for use in the treatment of autoimmune diseases such as for example Lupus disease.

In a further aspect, the invention relates to the use of a 4-1BBL trimer-containing antigen binding molecule in the manufacture or preparation of a medicament for the treatment of a disease in an individual in need thereof. In one aspect, the medicament is for use in a method of treating a disease comprising administering to an individual having the disease a therapeutically effective amount of the medicament. In certain embodiments the disease to be treated is a proliferative disorder, particularly cancer. Thus, in one aspect, the invention relates to the use of a 4-1BBL trimer-containing antigen binding molecule of the invention in the manufacture or preparation of a medicament for the treatment of cancer, in particular CEA-positive cancers. Examples of CEA-positive cancers include colon cancer, pancreatic cancer, gastric cancer, non-small-cell lung cancer (NSCLC), breast cancer, ovarian cancer, bladder cancer, esophageal cancer, cervix, carcinoma or endom adenocarcinoma, salivary gland, endometrial cancer and head & neck small cell cancer. In one aspect, the CEA-positive cancer is selected from the group consisting of colon adenocarcinoma, pancreas adenocarcinoma, gastric adenocarcinoma, non-small cell lung cancer (NSCLC), breast cancer, Cervix carcinoma and Esophageal adenocarcinoma. In particular, the CEA-positive cancer is colon cancer or non-small-cell lung cancer (NSCLC). A skilled artisan may recognize that in some cases the 4-1BBL trimer-containing antigen binding molecule may not provide a cure but may only provide partial benefit. In some aspects, a physiological change having some benefit is also considered therapeutically beneficial. Thus, in some aspects, an amount of 4-1BBL trimer-containing antigen binding molecule that provides a physiological change is considered an “effective amount” or a “therapeutically effective amount”.

In a further aspect, the invention provides a method for treating a disease in an individual, comprising administering to said individual a therapeutically effective amount of a 4-1BBL trimer-containing antigen binding molecule of the invention. In one aspect a composition is administered to said individual, comprising a fusion protein of the invention in a pharmaceutically acceptable form. In certain aspects, the disease to be treated is a proliferative disorder. In a particular aspect, the disease is cancer. In certain aspects, the method further comprises administering to the individual a therapeutically effective amount of at least one additional therapeutic agent, e.g. an anti-cancer agent if the disease to be treated is cancer. An “individual” according to any of the above embodiments may be a mammal, preferably a human.

For the prevention or treatment of disease, the appropriate dosage of a 4-1BBL trimer-containing antigen binding molecule of the invention (when used alone or in combination with one or more other additional therapeutic agents) will depend on the type of disease to be treated, the route of administration, the body weight of the patient, the type of antigen binding molecule, the severity and course of the disease, whether the fusion protein is administered for preventive or therapeutic purposes, previous or concurrent therapeutic interventions, the patient's clinical history and response to the fusion protein, and the discretion of the attending physician. The practitioner responsible for administration will, in any event, determine the concentration of active ingredient(s) in a composition and appropriate dose(s) for the individual subject. Various dosing schedules including but not limited to single or multiple administrations over various time-points, bolus administration, and pulse infusion are contemplated herein.

The 4-1BBL trimer-containing antigen binding molecule is suitably administered to the patient at one time or over a series of treatments. Depending on the type and severity of the disease, about 1 μg/kg to 15 mg/kg (e.g. 0.1 mg/kg-10 mg/kg) of 4-1BBL trimer-containing antigen binding molecule can be an initial candidate dosage for administration to the patient, whether, for example, by one or more separate administrations, or by continuous infusion. One typical daily dosage might range from about 1 μg/kg to 100 mg/kg or more, depending on the factors mentioned above. For repeated administrations over several days or longer, depending on the condition, the treatment would generally be sustained until a desired suppression of disease symptoms occurs. One exemplary dosage of the fusion protein would be in the range from about 0.005 mg/kg to about 10 mg/kg. In other examples, a dose may also comprise from about 1 μg/kg body weight, about 5 μg/kg body weight, about 10 μg/kg body weight, about 50 μg/kg body weight, about 100 μg/kg body weight, about 200 μg/kg body weight, about 350 μg/kg body weight, about 500 μg/kg body weight, about 1 mg/kg body weight, about 5 mg/kg body weight, about 10 mg/kg body weight, about 50 mg/kg body weight, about 100 mg/kg body weight, about 200 mg/kg body weight, about 350 mg/kg body weight, about 500 mg/kg body weight, to about 1000 mg/kg body weight or more per administration, and any range derivable therein. In examples of a derivable range from the numbers listed herein, a range of about 5 mg/kg body weight to about 100 mg/kg body weight, about 5 μg/kg body weight to about 500 mg/kg body weight etc., can be administered, based on the numbers described above. Thus, one or more doses of about 0.5 mg/kg, 2.0 mg/kg, 5.0 mg/kg or 10 mg/kg (or any combination thereof) may be administered to the patient. Such doses may be administered intermittently, e.g. every week or every three weeks (e.g. such that the patient receives from about two to about twenty, or e.g. about six doses of the fusion protein). An initial higher loading dose, followed by one or more lower doses may be administered. However, other dosage regimens may be useful. The progress of this therapy is easily monitored by conventional techniques and assays.

The 4-1BBL trimer-containing antigen binding molecules of the invention will generally be used in an amount effective to achieve the intended purpose. For use to treat or prevent a disease condition, the 4-1BBL trimer-containing antigen binding molecules of the invention, or pharmaceutical compositions thereof, are administered or applied in a therapeutically effective amount. Determination of a therapeutically effective amount is well within the capabilities of those skilled in the art, especially in light of the detailed disclosure provided herein.

For systemic administration, a therapeutically effective dose can be estimated initially from in vitro assays, such as cell culture assays. A dose can then be formulated in animal models to achieve a circulating concentration range that includes the IC50 as determined in cell culture. Such information can be used to more accurately determine useful doses in humans.

Initial dosages can also be estimated from in vivo data, e.g., animal models, using techniques that are well known in the art. One having ordinary skill in the art could readily optimize administration to humans based on animal data.

Dosage amount and interval may be adjusted individually to provide plasma levels of the 4-1BBL trimer-containing antigen binding molecules which are sufficient to maintain therapeutic effect. Usual patient dosages for administration by injection range from about 0.1 to 50 mg/kg/day, typically from about 0.5 to 1 mg/kg/day. Therapeutically effective plasma levels may be achieved by administering multiple doses each day. Levels in plasma may be measured, for example, by HPLC.

In cases of local administration or selective uptake, the effective local concentration of the 4-1BBL trimer-containing antigen binding molecule may not be related to plasma concentration. One skilled in the art will be able to optimize therapeutically effective local dosages without undue experimentation.

A therapeutically effective dose of the 4-1BBL trimer-containing antigen binding molecules described herein will generally provide therapeutic benefit without causing substantial toxicity. Toxicity and therapeutic efficacy of a fusion protein can be determined by standard pharmaceutical procedures in cell culture or experimental animals. Cell culture assays and animal studies can be used to determine the LD₅₀ (the dose lethal to 50% of a population) and the ED₅₀ (the dose therapeutically effective in 50% of a population). The dose ratio between toxic and therapeutic effects is the therapeutic index, which can be expressed as the ratio LD₅₀/ED₅₀. 4-1BBL trimer-containing antigen binding molecules that exhibit large therapeutic indices are preferred. In one embodiment, the 4-1BBL trimer-containing antigen binding molecule according to the present invention exhibits a high therapeutic index. The data obtained from cell culture assays and animal studies can be used in formulating a range of dosages suitable for use in humans. The dosage lies preferably within a range of circulating concentrations that include the ED₅₀ with little or no toxicity. The dosage may vary within this range depending upon a variety of factors, e.g., the dosage form employed, the route of administration utilized, the condition of the subject, and the like. The exact formulation, route of administration and dosage can be chosen by the individual physician in view of the patient's condition (see, e.g., Fingl et al., 1975, in: The Pharmacological Basis of Therapeutics, Ch. 1, p. 1, incorporated herein by reference in its entirety).

The attending physician for patients treated with fusion proteins of the invention would know how and when to terminate, interrupt, or adjust administration due to toxicity, organ dysfunction, and the like. Conversely, the attending physician would also know to adjust treatment to higher levels if the clinical response were not adequate (precluding toxicity). The magnitude of an administered dose in the management of the disorder of interest will vary with the severity of the condition to be treated, with the route of administration, and the like. The severity of the condition may, for example, be evaluated, in part, by standard prognostic evaluation methods. Further, the dose and perhaps dose frequency will also vary according to the age, body weight, and response of the individual patient.

Other Agents and Treatments

The 4-1BBL trimer-containing antigen binding molecules of the invention may be administered in combination with one or more other agents in therapy. For instance, a fusion protein of the invention may be co-administered with at least one additional therapeutic agent. The term “therapeutic agent” encompasses any agent that can be administered for treating a symptom or disease in an individual in need of such treatment. Such additional therapeutic agent may comprise any active ingredients suitable for the particular indication being treated, preferably those with complementary activities that do not adversely affect each other. In certain embodiments, an additional therapeutic agent is another anti-cancer agent.

Such other agents are suitably present in combination in amounts that are effective for the purpose intended. The effective amount of such other agents depends on the amount of 4-1BBL trimer-containing antigen binding molecule used, the type of disorder or treatment, and other factors discussed above. The 4-1BBL trimer-containing antigen binding molecules are generally used in the same dosages and with administration routes as described herein, or about from 1 to 99% of the dosages described herein, or in any dosage and by any route that is empirically/clinically determined to be appropriate.

Such combination therapies noted above encompass combined administration (where two or more therapeutic agents are included in the same or separate compositions), and separate administration, in which case, administration of the 4-1BBL trimer-containing antigen binding molecule of the invention can occur prior to, simultaneously, and/or following, administration of the additional therapeutic agent and/or adjuvant.

Thus, in one aspect a 4-1BBL trimer-containing antigen binding molecule as described herein for use in the treatment of cancer, in particular CEA positive cancer is provided, wherein the 4-1BBL trimer-containing antigen binding molecule is used in combination with a T-cell activating anti-CD3 bispecific antibody, in particular anti-CEA/anti-CD3 bispecific antibody.

In one aspect, the anti-CEA/anti-CD3 antibody comprises a first antigen binding domain that binds to CD3, and a second antigen binding domain that binds to CEA. In a particular aspect the second binding domain binding to CEA binds to a different epitope on CEA than the 4-1BBL trimer-containing antigen binding molecule.

In one aspect, the anti-CEA/anti-CD3 bispecific antibody as used herein comprises a first antigen binding domain comprising a heavy chain variable region (V_{H}CD3) comprising CDR-H1 sequence of SEQ ID NO:275, CDR-H2 sequence of SEQ ID NO:276, and CDR-H3 sequence of SEQ ID NO:277; and/or a light chain variable region (V_{L}CD3) comprising CDR-L1 sequence of SEQ ID NO:278, CDR-L2 sequence of SEQ ID NO:279, and CDR-L3 sequence of SEQ ID NO:280. More particularly, the anti-CEA/anti-CD3 bispecific antibody comprises a first antigen binding domain comprising a heavy chain variable region (V_{H}CD3) that is at least 90%, 95%, 96%, 97%, 98%, or 99% identical to the amino acid sequence of SEQ ID NO:281 and/or a light chain variable region (V_{L}CD3) that is at least 90%, 95%, 96%, 97%, 98%, or 99% identical to the amino acid sequence of SEQ ID NO:282. In a further aspect, the anti-CEA/anti-CD3 bispecific antibody comprises a heavy chain variable region (V_{H}CD3) comprising the amino acid sequence of SEQ ID NO:281 and/or a light chain variable region (V_{L}CD3) comprising the amino acid sequence of SEQ ID NO:282.

In another aspect, the anti-CEA/anti-CD3 bispecific antibody comprises a second antigen binding domain comprising

(a) a heavy chain variable region (V_{H}CEA) comprising CDR-H1 sequence of SEQ ID NO:283, CDR-H2 sequence of SEQ ID NO:284, and CDR-H3 sequence of SEQ ID NO:285, and/or a light chain variable region (V_{L}CEA) comprising CDR-L1 sequence of SEQ ID NO:286, CDR-L2 sequence of SEQ ID NO:287, and CDR-L3 sequence of SEQ ID NO:288, or
(b) a heavy chain variable region (V_{H}CEA) comprising CDR-H1 sequence of SEQ ID NO:291, CDR-H2 sequence of SEQ ID NO:292, and CDR-H3 sequence of SEQ ID NO:293, and/or a light chain variable region (V_{L}CEA) comprising CDR-L1 sequence of SEQ ID NO:294, CDR-L2 sequence of SEQ ID NO:295, and CDR-L3 sequence of SEQ ID NO:296.

In one aspect, the anti-CEA/anti-CD3 bispecific comprises a second antigen binding domain comprising a heavy chain variable region (V_{H}CEA) that is at least 90%, 95%, 96%, 97%, 98%, or 99% identical to the amino acid sequence of SEQ ID NO:289 and/or a light chain variable region (V_{L}CEA) that is at least 90%, 95%, 96%, 97%, 98%, or 99% identical to the amino acid sequence of SEQ ID NO:290. In a further aspect, the anti-CEA/anti-CD3 bispecific comprises a second antigen binding domain comprising a heavy chain variable region (V_{H}CEA) comprising the amino acid sequence of SEQ ID NO:63 and/or a light chain variable region (V_{L}CEA) comprising the amino acid sequence of SEQ ID NO:64.

In one aspect, the anti-CEA/anti-CD3 bispecific antibody comprises a first antigen binding domain comprising a heavy chain variable region (V_{H}CD3) comprising the amino acid sequence of SEQ ID NO:281 and/or a light chain variable region (V_{L}CD3) comprising the amino acid sequence of SEQ ID NO:282 and a second antigen binding domain comprising a heavy chain variable region (V_{H}CEA) comprising the amino acid sequence of SEQ ID NO:289 and/or a light chain variable region (V_{L}CEA) comprising the amino acid sequence of SEQ ID NO:290.

In a further aspect, the 4-1BBL trimer-containing antigen binding molecule is used in combination with a T-cell activating anti-CD3 bispecific antibody and the T-cell activating anti-CD3 bispecific antibody is administered concurrently with, prior to, or subsequently to the 4-1BBL trimer-containing antigen binding molecule.

In a further aspect, provided is the use of the 4-1BBL trimer-containing antigen binding molecule for the manufacture of a medicament for the treatment of cancer, wherein the 4-1BBL trimer-containing antigen binding molecule is used in combination with a T-cell activating anti-CD3 bispecific antibody, in particular an anti-CEA/anti-CD3 bispecific antibody. In certain aspects, the disease to be treated is CEA-positive cancers. Examples of CEA-positive cancers include colon cancer, pancreatic cancer, gastric cancer, non-small-cell lung cancer (NSCLC), breast cancer, ovarian cancer, bladder cancer, esophageal cancer, cervix carcinoma or endom adenocarcinoma, salivary gland, endometrial cancer and head & neck small cell cancer. In one aspect, the CEA-positive cancer is selected from the group consisting of colon adenocarcinoma, pancreas adenocarcinoma, gastric adenocarcinoma, non-small cell lung cancer (NSCLC), breast cancer, Cervix carcinoma and Esophageal adenocarcinoma. In particular, the CEA-positive cancer is colon cancer or non-small-cell lung cancer (NSCLC).

In a further aspect, the invention provides a method for treating cancer in an individual, comprising administering to said individual a therapeutically effective amount of a 4-1BBL trimer-containing antigen binding molecule of the invention and an effective amount a T-cell activating anti-CD3 bispecific antibody, in particular an anti-CEA/anti-CD3 bispecific antibody as defined above. In certain aspects, the method is for CEA-positive cancers. Examples of CEA-positive cancers include breast cancer, ovarian cancer, gastric cancer, bladder cancer, salivary gland, endometrial cancer, pancreatic cancer and non-small-cell lung cancer (NSCLC). In one aspect, the method is for treating CEA-positive metastatic breast cancer.

In a further aspect, the 4-1BBL trimer-containing antigen binding molecule of the invention is used in combination with an agent blocking PD-L1/PD-1 interaction and the agent blocking PD-L1/PD-1 interaction is administered concurrently with, prior to, or subsequently to the 4-1BBL trimer-containing antigen binding molecule. In this aspect, an agent blocking PD-L1/PD-1 interaction is a PD-L1 binding antagonist or a PD-1 binding antagonist. In particular, the agent blocking PD-L1/PD-1 interaction is an anti-PD-L1 antibody or an anti-PD-1 antibody. In a particular aspect, the agent blocking PD-L1/PD-1 interaction is an anti-PD-L1 antibody. In a specific aspect, the anti-PD-L1 antibody is selected from the group consisting of atezolizumab (MPDL3280A, RG7446), durvalumab (MEDI4736), avelumab (MSB0010718C) and MDX-1105. More particularly, the anti-PD-L1 antibody is atezolizumab. In another aspect, the agent blocking PD-L1/PD-1 interaction is an anti-PD-1 antibody. In a specific aspect, the anti-PD-1 antibody is selected from the group consisting of MDX 1106 (nivolumab), MK-3475 (pembrolizumab), CT-011 (pidilizumab), MEDI-0680 (AMP-514), PDR001, REGN2810, and BGB-108, in particular from pembrolizumab and nivolumab.

Articles of Manufacture

In another aspect of the invention, an article of manufacture containing materials useful for the treatment, prevention and/or diagnosis of the disorders described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper that is pierceable by a hypodermic injection needle). At least one active agent in the composition is a 4-1BBL trimer-containing antigen binding molecule of the invention.

The label or package insert indicates that the composition is used for treating the condition of choice. Moreover, the article of manufacture may comprise (a) a first container with a composition contained therein, wherein the composition comprises a 4-1BBL trimer-containing antigen binding molecule of the invention; and (b) a second container with a composition contained therein, wherein the composition comprises a further cytotoxic or otherwise therapeutic agent. The article of manufacture in this embodiment of the invention may further comprise a package insert indicating that the compositions can be used to treat a particular condition.

Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

TABLE B
(Sequences):

| SEQ | | |
|---|---|---|
| ID | | |
| NO: | Name | Sequence |
| 1 | CEA (A5B7)-CDR-H1 | DYYMN |
| 2 | CEA (A5B7)-CDR-H2 | FIGNKANGYTTEYSASVKG |
| 3 | CEA (A5B7)-CDR-H3 | DRGLRFYFDY |
| 4 | CEA (A5B7)-CDR-L1 | RASSSVTYIH |
| 5 | CEA (A5B7)-CDR-L2 | ATSNLAS |
| 6 | CEA (A5B7)-CDR-L3 | QHWSSKPPT |
| 7 | CEA (A5B7) VH | EVKLVESGGGLVQPGGSLRLSCATSGFTFTDYYM |
| | (parental) | NWVRQPPGKALEWLGFIGNKANGYTTEYSASVKG |
| | | RFTISRDKSQSILYLQMNTLRAEDSATYYCTRDR |
| | | GLRFYFDYWGQGTTLTVSS |
| 8 | CEA (A5B7) VL | QTVLSQSPAILSASPGEKVTMTCRASSSVTYIHW |
| | (parental) | YQQKPGSSPKSWIYATSNLASGVPARFSGSGSGT |
| | | SYSLTISRVEAEDAATYYCQHWSSKPPTFGGGTK |
| | | LEIK |
| 9 | CEA (MED1-565)-CDR-H1 | SYWMH |
| 10 | CEA (MEDI-565)-CDR-H2 | FIRNKANGGTTEYAAS |
| 11 | CEA (MEDI-565)-CDR-H3 | DRGLRFYFDY |
| 12 | CEA (MEDI-565)-CDR-L1 | TLRRGINVGAYSIY |
| 13 | CEA (MEDI-565)-CDR-L2 | YKSDSDKQQGS |
| 14 | CEA (MEDI-565)-CDR-L3 | MIWHSGASAV |
| 15 | CEA (MEDI-565) VH | EVQLVESGGGLVQPGRSLRLSCAASGFTVSSYWM |
| | | HWVRQAPGKGLEWVGFIRNKANGGTTEYAASVKG |
| | | RFTISRDDSKNTLYLQMNSLRAEDTAVYYCARDR |
| | | GLRFYFDYWGQGTTVTVSS |
| 16 | CEA (MEDI-565) VL | QAVLTQPASLSASPGASASLTCTLRRGINVGAYS |
| | | IYWYQQKPGSPPQYLLRYKSDSDKQQGSGVSSRF |
| | | SASKDASANAGILLISGLQSEDEADYYCMIWHSG |
| | | ASAVFGGGTKLTVL |
| 17 | CEA (A5H1EL1D)-CDR-H1 | DYYMN |
| 18 | CEA (A5H1EL1D)-CDR-H2 | FIGNKANAYTTEYSASVKG |
| 19 | CEA (A5H1EL1D)-CDR-H3 | DRGLRFYFDY |
| 20 | CEA (A5H1EL1D)-CDR-L1 | RASSSVTYIH |
| 21 | CEA (A5H1EL1D)-CDR-L2 | ATSNLAS |
| 22 | CEA (A5H1EL1D)-CDR-L3 | QHWSSKPPT |
| 23 | CEA (A5H1EL1D) | EVQLLESGGGLVQPGGSLRLSCAASGFTFTDYYM |
| | VH (3-23A5- | NWVRQAPGKGLEWLGFIGNKANAYTTEYSASVKG |
| | 1E) | RFTISRDKSKNTLYLQMNSLRAEDTATYYCTRDR |
| | | GLRFYFDYWGQGTTVTVSS |
| 24 | CEA (A5H1EL1D) | EIVLTQSPATLSLSPGERATLSCRASSSVTYIHW |
| | VL (A5-L1D) | YQQKPGQAPRSWIYATSNLASGIPARFSGSGSGT |
| | | DFTLTISSLEPEDFAVYYCQHWSSKPPTFGQGTK |
| | | LEIK |
| 25 | CEA (A5H1EL1D | GFX₁FX₂DYX₃MN, wherein |
| | aff. mat.) CDR- | X₁ is T or Y, |
| | H1 consensus | X₂ is T or S, and |
| | | X₃ is Y or A or E |
| 26 | CEA (A5H1EL1D | X₄IX₅NKANAYTTEYSASVKG, wherein |
| | aff. mat.)CDR- | X₅ is F or V, |
| | H2 consensus | X₆ is G or S |
| 27 | CEA (A5H1EL1D | DRGX₆RFX₇FDY, wherein |
| | aff. niat.)CDR- | X₆ is L or I, |
| | H3 consensus | X₇ is Y or G or Q or S |
| 28 | CEA (A5H1EL1D | X₈ASSSVTYIH, wherein |
| | aff. mat.) CDR- | X₈ is R or H |
| | L1 consensus | |
| 29 | CEA (A5H1EL1D | ATSNLAS |
| | aff. mat.) CDR- | |
| | L2 consensus | |
| 30 | CEA (A5H1EL1D | QHWSSX₉X₁₀PT, wherein |
| | aff. mat.) CDR- | X₉ is K or V or Q or I, |
| | L3 consensus | X₁₀ is P or S |
| 31 | CEA (P006.038) VH | EVQLLESGGGLVQPGGSLRLSCAASGFTFTD |
| | | YYMNWVRQAPGKGLEWLGFIGNKANAYTTEY |
| | | SASVKGRFTISRDKSKNTLYLQMNSLRAEDT |
| | | ATYYCTRDRGIRFGFDYWGQGTTVTVSS |
| 32 | CEA (P006.038) VL | EIVLTQSPATLSLSPGERATLSCRASSSVTY |
| | | IHWYQQKPGQAPRSWIYATSNLASGIPARFS |
| | | GSGSGTDFTLTISSLEPEDFAVYYCQHWSSV |
| | | PPTFGQGTKLEIK |
| 33 | CEA (P005.097) VH | EVQLLESGGGLVQPGGSLRLSCAASGFTFTD |
| | | YYMNWVRQAPGKGLEWLGFIGNKANAYTTEY |
| | | SASVKGRFTISRDKSKNTLYLQMNSLRAEDT |
| | | ATYYCTRDRGLRFSFDYWGQGTTVTVSS |
| 34 | CEA (P005.097) VL | EIVLTQSPATLSLSPGERATLSCRASSSVTY |
| | | IHWYQQKPGQAPRSWIYATSNLASGIPARFS |
| | | GSGSGTDFTLTISSLEPEDFAVYYCQHWSSQ |
| | | PPTFGQGTKLEIK |
| 35 | CEA (P005.103) VH | EVQLLESGGGLVQPGGSLRLSCAASGFTFTD |
| | | YYMNWVRQAPGKGLEWLGFIGNKANAYTTEY |
| | | SASVKGRFTISRDKSKNTLYLQMNSLRAEDT |
| | | ATYYCTRDRGIRFYFDYWGQGTTVTVSS |
| 36 | CEA (P005.103) VL | EIVLTQSPATLSLSPGERATLSCRASSSVTY |
| | | IHWYQQKPGQAPRSWIYATSNLASGIPARFS |
| | | GSGSGTDFTLTISSLEPEDFAVYYCQHWSSI |
| | | SPTFGQGTKLEIK |
| 37 | CEA (P002.139) VH | EVQLLESGGGLVQPGGSLRLSCAASGFYFTD |
| | | YAMNWVRQAPGKGLEWLGVISNKANAYTTEY |
| | | SASVKGRFTISRDKSKNTLYLQMNSLRAEDT |
| | | ATYYCTRDRGLRFYFDYWGQGTTVTVSS |
| 38 | CEA (P002.139) VL | EIVLTQSPATLSLSPGERATLSCHASSSVTY |
| | | IHWYQQKPGQAPRSWIYATSNLASGIPARFS |
| | | GSGSGTDFTLTISSLEPEDFAVYYCQHWSSK |
| | | PPTFGQGTKLEIK |
| 39 | CEA (P001.177) VH | EVQLLESGGGLVQPGGSLRLSCAASGFYFTD |
| | | YYMNWVRQAPGKGLEWLGFISNKANAYTTEY |
| | | SASVKGRFTISRDKSKNTLYLQMNSLRAEDT |
| | | ATYYCTRDRGLRFYFDYWGQGTTVTVSS |
| 40 | CEA (P001.177) VL | EIVLTQSPATLSLSPGERATLSCRASSSVTY |
| | | IHWYQQKPGQAPRSWIYATSNLASGIPARFS |
| | | GSGSGTDFTLTISSLEPEDFAVYYCQHWSSK |
| | | PPTFGQGTKLEIK |
| 41 | CEA (P005.102) VH | EVQLLESGGGLVQPGGSLRLSCAASGFTFTD |
| | | YYMNWVRQAPGKGLEWLGFIGNKANAYTTEY |
| | | SASVKGRFTISRDKSKNTLYLQMNSLRAEDT |
| | | ATYYCTRDRGIRFQFDYWGQGTTVTVSS |
| 42 | CEA (P005.102) VL | EIVLTQSPATLSLSPGERATLSCRASSSVTY |
| | | IHWYQQKPGQAPRSWIYATSNLASGIPARFS |
| | | GSGSGTDFTLTISSLEPEDFAVYYCQHWSSK |
| | | SPTFGQGTKLEIK |
| 43 | CEA (P005.102 | EVQLLESGGGLVQPGGSLRLSCAASGFYFTD |
| | combo1) VH | YYMNWVRQAPGKGLEWLGVISNKANAYTTEY |
| | | SASVKGRFTISRDKSKNTLYLQMNSLRAEDT |
| | | ATYYCTRDRGIRFQFDYWGQGTTVTVSS |
| 44 | CEA (P005.102 | EIVLTQSPATLSLSPGERATLSCRASSSVTY |
| | combo1) VL | IHWYQQKPGQAPRSWIYATSNLASGIPARFS |
| | | GSGSGTDFTLTISSLEPEDFAVYYCQHWSSK |
| | | SPTFGQGTKLEIK |
| 62 | CEA (MFE23) VL | ENVLTQSPAIMSASPGEKVTITCSASSSVSYMHW |
| | | FQQKPGTSPKLWIYSTSNLASGVPARFSGSGSGT |
| | | SYSLTISRMEAEDAATYYCQQRSSYPLTFGAGTK |
| | | LELK |
| 63 | CEA (T84.66-LCHA) VH | QVQLVQSGAEVKKPGSSVKVSCKASGFNIKDTYM |
| | | HWVRQAPGQGLEWMGRIDPANGNSKYVPKFQGRV |
| | | TITADTSTSTAYMELSSLRSEDTAVYYCAPFGYY |
| | | VSDYAMAYWGQGTLVTVSS |
| 64 | CEA (T84.66-LCHA) VL | EIVLTQSPATLSLSPGERATLSCRAGESVDIFGV |
| | | GFLHWYQQKPGQAPRLLIYRASNRATGIPARFSG |
| | | SGSGTDFTLTISSLEPEDFAVYYCQQTNEDPYTF |
| | | GQGTKLEIK |
| 65 | CEA (MFE-H24 to | DSYMH |
| | H29)-CDR-H1 | |
| 66 | CEA (MFE-H24, | WIDPENGDTEYAPKFQG |
| | H25, H27, H28, | |
| | H29)-CDR-H2 | |
| 67 | CEA (MFE-H26)- | WIDPENGGTNYAQKFQG |
| | CDR-H2 | |
| 68 | CEA (MFE-H24 to | GTPTGPYYFDY |
| | H29)-CDR-H3 | |
| 69 | CEA (MFE-L24, | RASSSVSYMH |
| | L25)-CDR-L1 | |
| 70 | CEA (MFE-H26)- | RASQSISSYM |
| | CDR-L1 | |
| 71 | CEA (MFE-L24, | STSNLAS |
| | L25, L27, L28)- | |
| | CDR-L2 | |
| 72 | CEA (MFE-L26)- | YTSNLAS |
| | CDR-L2 | |
| 73 | CEA (MFE-L29)- | STSSLQS |
| | CDR-L2 | |
| 74 | CEA (MFE-L24, | QQRSSYPLT |
| | L25, L27, L26, | |
| | L28, L29)- | |
| | CDR-L3 | |
| 75 | MFE-H24 | QVQLVQSGAEVKKPGASVKVSCKASGFNIKDSYMHWVR |
| | | QAPGQGLEWMGWIDPENGDTEYAPKFQGRVTMTTDTSI |
| | | STAYMELSRLRSDDTAVYYCNEGTPTGPYYFDYWGQGT |
| | | LVTVSS |
| 76 | MFE-H25 | QVQLVQSGAEVKKPGASVKVSCKASGYTFKDSYMHWVR |
| | | QAPGQGLEWMGWIDPENGDTEYAPKFQGRVTMTTDTSI |
| | | STAYMELSRLRSDDTAVYYCNEGTPTGPYYFDYWGQGT |
| | | LVTVSS |
| 77 | MFE-H26 | QVQLVQSGAEVKKPGASVKVSCKASGFNIKDSYMHWVR |
| | | QAPGQGLEWMGWIDPENGGTNYAQKFQGRVTMTTDTSI |
| | | STAYMELSRLRSDDTAVYYCNEGTPTGPYYFDYWGQGT |
| | | LVTVSS |
| 78 | MFE-H27 | QVQLVQSGAEVKKPGASVKVSCKASGFNIKDSYMHWVR |
| | | QAPGQGLEWMGWIDPENGDTEYAPKFQGRVTMTTDTSI |
| | | STAYMELSRLRSDDTAVYYCARGTPTGPYYFDYWGQGT |
| | | LVTVSS |
| 79 | MFE-H28 | QVQLVQSGAEVKKPGASVKVSCKASGFNIKDSYMHWVR |
| | | QAPGQGLEWMGWIDPENGDTEYAPKFQGRVTMTRDTSI |
| | | STAYMELSRLRSDDTAVYYCNEGTPTGPYYFDYWGQGT |
| | | LVTVSS |
| 80 | MFE-H29 | QVQLVQSGAEVKKPGSSVKVSCKASGFNIKDSYMHWVR |
| | | QAPGQGLEWMGWIDPENGDTEYAPKFQGRVTITTDEST |
| | | STAYMELSSLRSEDTAVYYCNEGTPTGPYYFDYWGQGT |
| | | LVTVSS |
| 81 | MFE-L24 | DIQMTQSPSSLSASVGDRVTITCRASSSVSYMHWYQQK |
| | | PGKAPKLLIYSTSNLASGVPSRFSGSGSGTDFTLTISS |
| | | LQPEDFATYYCQQRSSYPLTFGGGTKLEIK |
| 82 | MFE-L25 | EIQMTQSPSSLSASVGDRVTITCRASSSVSYMHWYQQK |
| | | PGKAPKLLIYSTSNLASGVPSRFSGSGSGTDFTLTISS |
| | | LQPEDFATYYCQQRSSYPLTFGGGTKLEIK |
| 83 | MFE-L26 | EIQMTQSPSSLSASVGDRVTITCRASQSISSYMHWYQQ |
| | | KPGKAPKLLIYSTSNLASGVPSRFSGSGSGTDFTLTIS |
| | | SLQPEDFATYYCQQRSSYPLTFGGGTKLEIK |
| 84 | MFE-L27 | EIQMTQSPSSLSASVGDRVTITCRASSSVPYMHWYQQK |
| | | PGKAPKLLIYSTSNLASGVPSRFSGSGSGTDFTLTISS |
| | | VQPEDFATYYCQQRSSYPLTFGGGTKLEIK |
| 85 | MFE-L28 | EIQMTQSPSSLSASVGDRVTITCRASSSVPYMHWLQQK |
| | | PGKAPKLLIYSTSNLASGVPSRFSGSGSGTDFTLTISS |
| | | VQPEDFATYYCQQRSSYPLTFGGGTKLEIK |
| 86 | MFE-L29 | EIQMTQSPSSLSASVGDRVTITCRASSSVPYMHWLQQK |
| | | PGKAPKLLIYSTSSLQSGVPSRFSGSGSGTDFTLTISS |
| | | VQPEDFATYYCQQRSSYPLTFGGGTKLEIK |
| 87 | Human (hu) 4-1BBL | REGPELSPDDPAGLLDLRQGMFAQLVAQNVLLIDGPLS |
| | (71-254) | WYSDPGLAGVSLTGGLSYKEDTKELVVAKAGVYYVFFQ |
| | | LELRRVVAGEGSGSVSLALHLQPLRSAAGAAALALTVD |
| | | LPPASSEARNSAFGFQGRLLHLSAGQRLGVHLHTEARA |
| | | RHAWQLTQGATVLGLFRVTPEIPAGLPSPRSE |
| 88 | hu 4-1BBL (85-254) | LDLRQGMFAQLVAQNVLLIDGPLSWYSDPGLAGVSLTG |
| | | GLSYKEDTKELVVAKAGVYYVFFQLELRRVVAGEGSGS |
| | | VSLALHLQPLRSAAGAAALALTVDLPPASSEARNSAFG |
| | | FQGRLLHLSAGQRLGVHLHTEARARHAWQLTQGATVLG |
| | | LFRVTPEIPAGLPSPRSE |
| 89 | hu 4-1BBL (80-254) | DPAGLLDLRQGMFAQLVAQNVLLIDGPLSWYSDPGLAG |
| | | VSLTGGLSYKEDTKELVVAKAGVYYVFFQLELRRVVAG |
| | | EGSGSVSLALHLQPLRSAAGAAALALTVDLPPASSEAR |
| | | NSAFGFQGRLLHLSAGQRLGVHLHTEARARHAWQLTQG |
| | | ATVLGLFRVTPEIPAGLPSPRSE |
| 90 | hu 4-1BBL (52-254) | PWAVSGARASPGSAASPRLREGPELSPDDPAGLLDLRQ |
| | | GMFAQLVAQNVLLIDGPLSWYSDPGLAGVSLTGGLSYK |
| | | EDTKELVVAKAGVYYVFFQLELRRVVAGEGSGSVSLAL |
| | | HLQPLRSAAGAAALALTVDLPPASSEARNSAFGFQGRL |
| | | LHLSAGQRLGVHLHTEARARHAWQLTQGATVLGLFRVT |
| | | PEIPAGLPSPRSE |
| 91 | Human (hu) 4-1BBL | REGPELSPDDPAGLLDLRQGMFAQLVAQNVLLIDGPLS |
| | (71-248) | WYSDPGLAGVSLTGGLSYKEDTKELVVAKAGVYYVFFQ |
| | | LELRRVVAGEGSGSVSLALHLQPLRSAAGAAALALTVD |
| | | LPPASSEARNSAFGFQGRLLHLSAGQRLGVHLHTEARA |
| | | RHAWQLTQGATVLGLFRVTPEIPAGL |
| 92 | hu 4-1BBL (85-248) | LDLRQGMFAQLVAQNVLLIDGPLSWYSDPGLAGVSLTG |
| | | GLSYKEDTKELVVAKAGVYYVFFQLELRRVVAGEGSGS |
| | | VSLALHLQPLRSAAGAAALALTVDLPPASSEARNSAFG |
| | | FQGRLLHLSAGQRLGVHLHTEARARHAWQLTQGATVLG |
| | | LFRVTPEIPAGL |
| 93 | hu 4-1BBL (80-248) | DPAGLLDLRQGMFAQLVAQNVLLIDGPLSWYSDPGLAG |
| | | VSLTGGLSYKEDTKELVVAKAGVYYVFFQLELRRVVAG |
| | | EGSGSVSLALHLQPLRSAAGAAALALTVDLPPASSEAR |
| | | NSAFGFQGRLLHLSAGQRLGVHLHTEARARHAWQLTQG |
| | | ATVLGLFRVTPEIPAGL |
| 94 | hu 4-1BBL (52-248) | PWAVSGARASPGSAASPRLREGPELSPDDPAGLLDLRQ |
| | | GMFAQLVAQNVLLIDGPLSWYSDPGLAGVSLTGGLSYK |
| | | EDTKELVVAKAGVYYVFFQLELRRVVAGEGSGSVSLAL |
| | | HLQPLRSAAGAAALALTVDLPPASSEARNSAFGFQGRL |
| | | LHLSAGQRLGVHLHTEARARHAWQLTQGATVLGLFRVT |
| | | PEIPAGL |
| 95 | dimeric hu 4-1BBL | REGPELSPDDPAGLLDLRQGMFAQLVAQNVLLIDGPLS |
| | (71-254) connected | WYSDPGLAGVSLTGGLSYKEDTKELVVAKAGVYYVFFQ |
| | by (G4S)₂ linker | LELRRVVAGEGSGSVSLALHLQPLRSAAGAAALALTVD |
| | | LPPASSEARNSAFGFQGRLLHLSAGQRLGVHLHTEARA |
| | | RHAWQLTQGATVLGLFRVTPEIPAGLPSPRSEGGGGSG |
| | | GGGSREGPELSPDDPAGLLDLRQGMFAQLVAQNVLLID |
| | | GPLSWYSDPGLAGVSLTGGLSYKEDTKELVVAKAGVYY |
| | | VFFQLELRRVVAGEGSGSVSLALHLQPLRSAAGAAALA |
| | | LTVDLPPASSEARNSAFGFQGRLLHLSAGQRLGVHLHT |
| | | EARARHAWQLTQGATVLGLFRVTPElPAGLPSPRSE |
| 96 | dimeric hu 4-1BBL | REGPELSPDDPAGLLDLRQGMFAQLVAQNVLLIDGPLS |
| | (71-248) | WYSDPGLAGVSLTGGLSYKEDTKELVVAKAGVYYVFFQ |
| | connected by | LELRRVVAGEGSGSVSLALHLQPLRSAAGAAALALTVD |
| | (G4S)₂ linker | LPPASSEARNSAFGFQGRLLHLSAGQRLGVHLHTEARA |
| | | RHAWQLTQGATVLGLFRVTPEIPAGLGGGGSGGGGSRE |
| | | GPELSPDDPAGLLDLRQGMFAQLVAQNVLLIDGPLSWY |
| | | SDPGLAGVSLTGGLSYKEDTKELVVAKAGVYYVFFQLE |
| | | LRRVVAGEGSGSVSLALHLQPLRSAAGAAALALTVDLP |
| | | PASSEARNSAFGFQGRLLHLSAGQRLGVHLHTEARARH |
| | | AWQLTQGATVLGLFRVTPEIPAGL |
| 97 | dimeric hu 4-1BBL | DPAGLLDLRQGMFAQLVAQNVLLIDGPLSWYSDPGLAG |
| | (80-254) | VSLTGGLSYKEDTKELVVAKAGVYYVFFQLELRRVVAG |
| | connected by | EGSGSVSLALHLQPLRSAAGAAALALTVDLPPASSEAR |
| | (G4S)₂ linker | NSAFGFQGRLLHLSAGQRLGVHLHTEARARHAWQLTQG |
| | | ATVLGLFRVTPEIPAGLPSPRSEGGGGSGGGGSDPAGL |
| | | LDLRQGMFAQLVAQNVLLIDGPLSWYSDPGLAGVSLTG |
| | | GLSYKEDTKELVVAKAGVYYVFFQLELRRVVAGEGSGS |
| | | VSLALHLQPLRSAAGAAALALTVDLPPASSEARNSAFG |
| | | FQGRLLHLSAGQRLGVHLHTEARARHAWQLTQGATVLG |
| | | LFRVTPEIPAGLPSPRSE |
| 98 | dimeric hu | PWAVSGARASPGSAASPRLREGPELSPDDPAGLLDLRQ |
| | 4-1BBL (52-254) | GMFAQLVAQNVLLIDGPLSWYSDPGLAGVSLTGGLSYK |
| | connected by | EDTKELVVAKAGVYYVFFQLELRRVVAGEGSGSVSLAL |
| | (G4S)₂ linker | HLQPLRSAAGAAALALTVDLPPASSEARNSAFGFQGRL |
| | | LHLSAGQRLGVHLHTEARARHAWQLTQGATVLGLFRVT |
| | | PEIPAGLPSPRSEGGGGSGGGGSPWAVSGARASPGSAA |
| | | SPRLREGPELSPDDPAGLLDLRQGMFAQLVAQNVLLID |
| | | GPLSWYSDPGLAGVSLTGGLSYKEDTKELVVAKAGVYY |
| | | VFFQLELRRVVAGEGSGSVSLALHLQPLRSAAGAAALA |
| | | LTVDLPPASSEARNSAFGFQGRLLHLSAGQRLGVHLHT |
| | | EARARHAWQLTQGATVLGLFRVTPEIPAGLPSPRSE |
| 99 | Dimeric 4-1BB | See Table 20 |
| | ligand (71-248)- | |
| | CL* Fc knob | |
| | chain | |
| 100 | Monomeric 4-1BB | See Table 20 |
| | ligand (71-248)- | |
| | CH1* | |
| 101 | Dimeric 4-1BB | REGPELSPDDPAGLLDLRQGMFAQLVAQNVLLIDGPLS |
| | ligand (71-248) | WYSDPGLAGVSLTGGLSYKEDTKELVVAKAGVYYVFFQ |
| | CL Fc knob chain | LELRRVVAGEGSGSVSLALHLQPLRSAAGAAALALTVD |
| | | LPPASSEARNSAFGFQGRLLHLSAGQRLGVHLHTEARA |
| | | RHAWQLTQGATVLGLFRVTPEIPAGLGGGGSGGGGSRE |
| | | GPELSPDDPAGLLDLRQGMFAQLVAQNVLLIDGPLSWY |
| | | SDPGLAGVSLTGGLSYKEDTKELVVAKAGVYYVFFQLE |
| | | LRRVVAGEGSGSVSLALHLQPLRSAAGAAALALTVDLP |
| | | PASSEARNSAFGFQGRLLHLSAGQRLGVHLHTEARARH |
| | | AWQLTQGATVLGLFRVTPEIPAGLGGGGSGGGGSRTVA |
| | | APSVFIFPPSDEQLKSGTASVVCLLNNFYPREAKVQWK |
| | | VDNALQSGNSQESVTEQDSKDSTYSLSSTLTLSKADYE |
| | | KHKVYACEVTHQGLSSPVTKSFNRGECDKTHTCPPCPA |
| | | PEAAGGPSVFLFPPKPKDTLMISRTPEVTCVVVDVSHE |
| | | DPEVKFNWYVDGVEVHNAKTKPREEQYNSTYRVVSVLT |
| | | VLHQDWLNGKEYKCKVSNKALGAPIEKTISKAKGQPRE |
| | | PQVYTLPPCRDELTKNQVSLWCLVKGFYPSDIAVEWES |
| | | NGQPENNYKTTPPVLDSDGSFFLYSKLTVDKSRWQQGN |
| | | VFSCSVMHEALHNHYTQKSLSLSP |
| 102 | Monomeric 4-1BB | REGPELSPDDPAGLLDLRQGMFAQLVAQNVLLIDGPLS |
| | ligand (71-248)- | WYSDPGLAGVSLTGGLSYKEDTKELVVAKAGVYYVFFQ |
| | CH1 | LELRRVVAGEGSGSVSLALHLQPLRSAAGAAALALTVD |
| | | LPPASSEARNSAFGFQGRLLHLSAGQRLGVHLHTEARA |
| | | RHAWQLTQGATVLGLFRVTPEIPAGLGGGGSGGGGSAS |
| | | TKGPSVFPLAPSSKSTSGGTAALGCLVKDYFPEPVTVS |
| | | WNSGALTSGVHTFPAVLQSSGLYSLSSVVTVPSSSLGT |
| | | QTYICNVNHKPSNTKVDKKVEPKSC |
| 103 | Dimeric 4-1BB | REGPELSPDDPAGLLDLRQGMFAQLVAQNVLLIDGPLS |
| | ligand (71-254) | WYSDPGLAGVSLTGGLSYKEDTKELVVAKAGVYYVFFQ |
| | CL* Fc knob chain | LELRRVVAGEGSGSVSLALHLQPLRSAAGAAALALTVD |
| | | LPPASSEARNSAFGFQGRLLHLSAGQRLGVHLHTEARA |
| | | RHAWQLTQGATVLGLFRVTPEIPAGLPSPRSEGGGGSG |
| | | GGGSREGPELSPDDPAGLLDLRQGMFAQLVAQNVLLID |
| | | GPLSWYSDPGLAGVSLTGGLSYKEDTKELVVAKAGVYY |
| | | VFFQLELRRVVAGEGSGSVSLALHLQPLRSAAGAAALA |
| | | LTVDLPPASSEARNSAFGFQGRLLHLSAGQRLGVHLHT |
| | | EARARHAWQLTQGATVLGLFRVTPEIPAGLPSPRSEGG |
| | | GGSGGGGSRTVAAPSVFIFPPSDRKLKSGTASVVCLLN |
| | | NFYPREAKVQWKVDNALQSGNSQESVTEQDSKDSTYSL |
| | | SSTLTLSKADYEKHKVYACEVTHQGLSSPVTKSFNRGE |
| | | CDKTHTCPPCPAPEAAGGPSVFLFPPKPKDTLMISRTP |
| | | EVTCVVVDVSHEDPEVKFNWYVDGVEVHNAKTKPREEQ |
| | | YNSTYRVVSVLTVLHQDWLNGKEYKCKVSNKALGAPIE |
| | | KTISKAKGQPREPQVYTLPPCRDELTKNQVSLWCLVKG |
| | | FYPSDIAVEWESNGQPENNYKTTPPVLDSDGSFFLYSK |
| | | LTVDKSRWQQGNVFSCSVMHEALHNHYTQKSLSLSP |
| 104 | Monomeric 4-1BB | REGPELSPDDPAGLLDLRQGMFAQLVAQNVLLIDGPLS |
| | ligand (71-254)- | WYSDPGLAGVSLTGGLSYKE0TKELVVAKAGVYYVFFQ |
| | CH1* | LELRRVVAGEGSGSVSLALHLQPLRSAAGAAALALTVD |
| | | LPPASSEARNSAFGFQGRLLHLSAGQRLGVHLHTEARA |
| | | RHAWQLTQGATVLGLFRVTPEIPAGLPSPRSEGGGGSG |
| | | GGGSASTKGPSVFPLAPSSKSTSGGTAALGCLVEDYFP |
| | | EPVTVSWNSGALTSGVHTFPAVLQSSGLYSLSSVVTVP |
| | | SSSLGTQTYICNVNHKPSNTKVDEKVEPKSC |
| 105 | Dimeric 4-1BB ligand | REGPELSPDDPAGLLDLRQGMFAQLVAQNVLLIDGPLS |
| | (71-254)- | WYSDPGLAGVSLTGGLSYKEDTKELVVAKAGVYYVFFQ |
| | CL Fc knob chain | LELRRVVAGEGSGSVSLALHLQPLRSAAGAAALALTVD |
| | | LPPASSEARNSAFGFQGRLLHLSAGQRLGVHLHTEARA |
| | | RHAWQLTQGATVLGLFRVTPEIPAGLPSPRSEGGGGSG |
| | | GGGSREGPELSPDDPAGLLDLRQGMFAQLVAQNVLLID |
| | | GPLSWYSDPGLAGVSLTGGLSYKEDTKELVVAKAGVYY |
| | | VFFQLELRRVVAGEGSGSVSLALHLQPLRSAAGAAALA |
| | | LTVDLPPASSEARNSAFGFQGRLLHLSAGQRLGVHLHT |
| | | EARARHAWQLTQGATVLGLFRVTPElPAGLPSPRSEGG |
| | | GGSGGGGSRTVAAPSVFIFPPSDEQLKSGTASVVCLLN |
| | | NFYPREAKVQWKVDNALQSGNSQESVTEQDSKDSTYSL |
| | | SSTLTLSKADYEKHKVYACEVTHQGLSSPVTKSFNRGE |
| | | CDKTHTCPPCPAPEAAGGPSVFLFPPKPKDTLMISRTP |
| | | EVTCVVVDVSHEDPEVKFNWYVDGVEVHNAKTKPREEQ |
| | | YNSTYRVVSVLTVLHQDWLNGKEYKCKVSNKALGAPIE |
| | | KTISKAKGQPREPQVYTLPPCRDELTKNQVSLWCLVKG |
| | | FYPSDIAVEWESNGQPENNYKTTPPVLDSDGSFFLYSK |
| | | LTVDKSRWQQGNVFSCSVMHEALHNHYTQKSLSLSP |
| 106 | Monomeric 4-1BB | REGPELSPDDPAGLLDLRQGMFAQLVAQNVLLIDGPLS |
| | ligand (71-254)- | WYSDPGLAGVSLTGGLSYKEDTKELVVAKAGVYYVFFQ |
| | CH1 | LELRRVVAGEGSGSVSLALHLQPLRSAAGAAALALTVD |
| | | LPPASSEARNSAFGFQGRLLHLSAGQRLGVHLHTEARA |
| | | RHAWQLTQGATVLGLFRVTPEIPAGLPSPRSEGGGGSG |
| | | GGGSASTKGPSVFPLAPSSKSTSGGTAALGCLVKDYFP |
| | | EPVTVSWNSGALTSGVHTFPAVLQSSGLYSLSSVVTVP |
| | | SSSLGTQTYICNVNHKPSNTKVDKKVEPKSC |
| 107 | Fc hole chain | ASTKGPSVFPLAPSSKSTSGGTAALGCLVKDYFPEPVT |
| | | VSWNSGALTSGVHTFPAVLQSSGLYSLSSVVTVPSSSL |
| | | GTQTYICNVNHKPSNTKVDKKVEPKSCDKTHTCPPCPA |
| | | PEAAGGPSVFLFPPKPKDTLMISRTPEVTCVVVDVSHE |
| | | DPEVKFNWYVDGVEVHNAKTKPREEQYNSTYRVVSVLT |
| | | VLHQDWLNGKEYKCKVSNKALGAPIEKTISKAKGQPRE |
| | | PQVCTLPPSRDELTKNQVSLSCAVKGFYPSDIAVEWES |
| | | NGQPENNYKTTPPVLDSDGSFFLVSKLTVDKSRWQQGN |
| | | VFSCSVMHEALHNHYTQKSLSLSP |
| 108 | human CEA | UniProt no. P06731 |
| 109 | human 4-1BB | UniProt no. Q07011 |
| 110 | murine 4-1BB | UniProt no. P20334 |
| 111 | cynomolgus 4-1BB | Uniprot no. F6W5G6 |
| 112 | Peptide linker (G4S) | GGGGS |
| 113 | Peptide linker (G4S)₂ | GGGGSGGGGS |
| 114 | Peptide linker (SG4)₂ | SGGGGSGGGG |
| 115 | Peptide linker G4(SG4)₂ | GGGGSGGGGSGGGG |
| 116 | peptide linker | GSPGSSSSGS |
| 117 | (G4S)₃ peptide linker | GGGGSGGGGSGGGGS₃ |
| 118 | (G4S)₄ peptide linker | GGGGSGGGGSGGGGSGGGGS |
| 119 | peptide linker X | GSGSGSGS |
| 120 | peptide linker X1 | GSGSGNGS |
| 121 | peptide linker X2 | GGSGSGSG |
| 122 | peptide linker X3 | GGSGSG |
| 123 | peptide linker X4 | GGSG |
| 124 | peptide linker X5 | GGSGNGSG |
| 125 | peptide linker X6 | GGNGSGSG |
| 126 | peptide linker X7 | GGNGSG |
| 127 | IGHV3-23-02 | EVQLLESGGGLVQPGGSLRLSCAASGFTFSSYAMSWVR |
| | | QAPGKGLEWVSAISGSGGSTYYGDSVKGRFTISRDNSK |
| | | NTLYLQMNSLRAEDTAVYYCAK |
| 128 | IGHV3-15*01 | EVQLVESGGGLVKPGGSLRLSCAASGFTFSNAWM |
| | | SWVRQAPGKGLEWVGRIKSKTDGGTTDYAAPVKG |
| | | RFTISRDDSKNTLYLQMNSLKTEDTAVYYCTT |
| 129 | 3-23A5-1 | EVQLLESGGGLVQPGGSLRLSCAASGFTFTDYYMNWVR |
| | | QAPGKGLEWVGFIGNKANGYTTEYSASVKGRFTISRDN |
| | | SKNTLYLQMNSLRAEDTAVYYCARDRGLRFYFDYWGQG |
| | | TTVTVSS |
| 130 | 3-23A5-2 | EVQLLESGGGLVQPGGSLRLSCAASGFTFTDYYMNWVR |
| | | QAPGKGLEWVGFIGNKANGYTTYYGDSVKGRFTISRDN |
| | | SKNTLYLQMNSLRAEDTAVYYCARDRGLRFYFDYWGQG |
| | | TTVTVSS |
| 131 | 3-23A5-3 | EVQLLESGGGLVQPGGSLRLSCAASGFTFTDYYMNWVR |
| | | QAPGKGLEWVGFIGNKGYTTEYSASVKGRFTISRDNSK |
| | | NTLYLQMNSLRAEDTAVYYCARDRGLRFYFDYWGQGTT |
| | | VTVSS |
| 132 | 3-23A5-4 | EVQLLESGGGLVQPGGSLRLSCAASGFTFTDYYMSWVR |
| | | QAPGKGLEWVGFIGNKANGYTTEYSASVKGRFTISRDN |
| | | SKNTLYLQMNSLRAEDTAVYYCARDRGLRFYFDYWGQG |
| | | TTVTVSS |
| 133 | 3-23A5-1A | EVQLLESGGGLVQPGGSLRLSCAASGFTFTDYYMNWVR |
| | (all_backmutations) | QAPGKGLEWLGFIGNKANGYTTEYSASVKGRFTISRDK |
| | | SKNTLYLQMNSLRAEDTATYYCTRDRGLRFYFDYWGQG |
| | | TTVTVSS |
| 134 | 3-23A5-1C (A93T) | EVQLLESGGGLVQPGGSLRLSCAASGFTFTDYYMNWVR |
| | | QAPGKGLEWVGFIGNKANGYTTEYSASVKGRFTISRDN |
| | | SKNTLYLQMNSLRAEDTAVYYCTRDRGLRFYFDYWGQG |
| | | TTVTVSS |
| 135 | 3-23A5-1D (K.73) | EVQLLESGGGLVQPGGSLRLSCAASGFTFTDYYMNWVR |
| | | QAPGKGLEWVGFIGNKANGYTTEYSASVKGRFTISRDK |
| | | SKNTLYLQMNSLRAEDTAVYYCARDRGLRFYFDYWGQG |
| | | TTVTVSS |
| 136 | 3-15A5-1 | EVQLVESGGGLVKPGGSLRLSCAASGFTFTDYYM |
| | | NWVRQAPGKGLEWVG FIGNKANGYTT EY SASVKG |
| | | RFTISRDDSKNTLYLQMNSLKTEDTAVYYCTRDR |
| | | GLRFY FDYWGQGTTVTVSS |
| 137 | 3-15A5-2 | EVQLVESGGGLVKPGGSLRLSCAASGFTFTDYYMNWVR |
| | | QAPGKGLEWVGFIGNKANGYTTEYAAPVKGRFTISRDD |
| | | SKNTLYLQMNSLKTEDTAVYYCTRDRGLRFYFDYWGQG |
| | | TTVTVSS |
| 138 | 3-15A5-3 | EVQLVESGGGLVKPGGSLRLSCAASGFTFTDYYMNWVR |
| | | QAPGKGLEWVGFIGNKANGGTTDYAAPVKGRFTISRDD |
| | | SKNTLYLQMNSLKTEDTAVYYCTRDRGLRFYFDYWGQG |
| | | TTVTVSS |
| 139 | IGKV3-11 | EIVLTQSPATLSLSPGERATLSCRASQSVSSYLAWYQQ |
| | | KPGQAPRLLIYDASNRATGIPARFSGSGSGTDFTLTIS |
| | | SLEPEDFAVYYCQQRSNWP |
| 140 | A5-L1 | EIVLTQSPATLSLSPGERATLSCRASSSVTYIHWYQQK |
| | | PGQAPRLLIYATSNLASGIPARFSGSGSGTDFTLTISS |
| | | LEPEDFAVYYCQHWSSKPPTFGQGTKLEIK |
| 141 | A5-L2 | EIVLTQSPATLSLSPGERATLSCRASQSVSSYIHWYQQ |
| | | KPGQAPRLLIYATSNLASGIPARFSGSGSGTDFTLTIS |
| | | SLEPEDFAVYYCQHWSSKPPTFGQGTKLEIK |
| 142 | A5-L3 | EIVLTQSPATLSLSPGERATLSCRASSSVTYIHWYQQK |
| | | PGQAPRLLIYDASNRATGIPARFSGSGSGTDFTLTISS |
| | | LEPEDFAVYYCQHWSSKPPTFGQGTKLEIK |
| 143 | A5-L4 | EIVLTQSPATLSLSPGERATLSCRASSSVTYIHWYQQK |
| | | PGQAPRLLIYATSNLASGIPARFSGSGSGTDFTLTISS |
| | | LE P EDFAVYYCQQWSS KP PTFGQGTKLEIK |
| 144 | A5-L1A | QTVLTQSPATLSLSPGERATLSCRASSSVTYIHWYQQK |
| | (all_backmutations) | PGSSPKSWIYATSNLASGIPARFSGSGSGTDYTLTISS |
| | | LEPEDFAVYYCQHWSSKPPTFGQGTKLEIK |
| 145 | A5-L1B (Q1T2) | QTVLTQSPATLSLSPGERATLSCRASSSVTYIHWYQQK |
| | | PGQAPRLLIYATSNLASGIPARFSGSGSGTDFTLTISS |
| | | LEPEDFAVYYCQHWSSKPPTFGQGTKLEIK |
| 146 | A5-L1C (FR2) | EIVLTQSPATLSLSPGERATLSCRASSSVTYIHWYQQK |
| | | PGSSPKSWIYATSNLASGIPARFSGSGSGTDFTLTISS |
| | | LEPEDFAVYYCQHWSSKPPTFGQGTKLEIK |
| 147 | NABA-avi-His | See Table 6 |
| 148 | N(A2B2)A-avi-His | See Table 6 |
| 149 | NA(B2)A-avi-His | See Table 6 |
| 150 | A5H1EL1D_H1_rev_TN | See Table 7 |
| 151 | A5H1EL1D_H2_for_TN | See Table 7 |
| 152 | LMB3 long | See Table 7 |
| 153 | HCDR3-rev-constant | See Table 7 |
| 154 | A5H1EL1D_L1_rev_TN | See Table 8 |
| 155 | A5H1ELID_L2_for_TN | See Table 8 |
| 156 | A5H1EL1D | See Table 9 |
| | L3_for_TN | |
| 157 | A5H1EL1D | See Table 9 |
| | H3_rev_TN | |
| 158 | LCDR3-rev-constant | See Table 9 |
| 159 | HCDR3 amplification | See Table 9 |
| 160 | CEA (P006.038)- | See Table 13 |
| | CDR-H1 | |
| 161 | CEA (P006.038)- | See Table 13 |
| | CDR-H2 | |
| 162 | CEA (P006.038)- | See Table 13 |
| | CDR-H3 | |
| 163 | CEA (P006.038)- | See Table 14 |
| | CDR-L1 | |
| 164 | CEA (P006.038)- | See Table 14 |
| | CDR-L2 | |
| 165 | CEA (P006.038)- | See Table 14 |
| | CDR-L3 | |
| 166 | CEA (P005.097)- | See Table 13 |
| | CDR-H1 | |
| 167 | CEA (P005.097)- | See Table 13 |
| | CDR-H2 | |
| 168 | CEA (P005.097)- | See Table 13 |
| | CDR-H3 | |
| 169 | CEA (P005.097)- | See Table 14 |
| | CDR-L1 | |
| 170 | CEA (P005.097)- | See Table 14 |
| | CDR-L2 | |
| 171 | CEA (P005.097)- | See Table 14 |
| | CDR-L3 | |
| 172 | CEA (P005.103)- | See Table 13 |
| | CDR-H1 | |
| 173 | CEA (P005.103)- | See Table 13 |
| | CDR-H2 | |
| 174 | CEA (P005.103)- | See Table 13 |
| | CDR-H3 | |
| 175 | CEA (P005.103)- | See Table 14 |
| | CDR-L1 | |
| 176 | CEA (P005.103)- | See Table 14 |
| | CDR-L2 | |
| 177 | CEA (P005.103)- | See Table 14 |
| | CDR-L3 | |
| 178 | CEA (P002.139)- | See Table 13 |
| | CDR-H1 | |
| 179 | CEA (P002.139)- | See Table 13 |
| | CDR-H2 | |
| 180 | CEA (P002.139)- | See Table 13 |
| | CDR-H3 | |
| 181 | CEA (P002.139)- | See Table 14 |
| | CDR-L1 | |
| 182 | CEA (P002.139)- | See Table 14 |
| | CDR-L2 | |
| 183 | CEA (P002.139)- | See Table 14 |
| | CDR-L3 | |
| 184 | CEA (P001.177)- | See Table 13 |
| | CDR-H1 | |
| 185 | CEA (P001.177)- | See Table 13 |
| | CDR-H2 | |
| 186 | CEA (P001.177)- | See Table 13 |
| | CDR-H3 | |
| 187 | CEA (P001.177)- | See Table 14 |
| | CDR-L1 | |
| 188 | CEA (P001.177)- | See Table 14 |
| | CDR-L2 | |
| 189 | CEA (P001.177)- | See Table 14 |
| | CDR-L3 | |
| 190 | CEA (P005.102)- | See Table 13 |
| | CDR-H1 | |
| 191 | CEA (P005.102)- | See Table 13 |
| | CDR-H2 | |
| 192 | CEA (P005.102)- | See Table 13 |
| | CDR-H3 | |
| 193 | CEA (P005.102)- | See Table 14 |
| | CDR-L1 | |
| 194 | CEA (P005.102)- | See Table 14 |
| | CDR-L2 | |
| 195 | CEA (P005.102)- | See Table 14 |
| | CDR-L3 | |
| 196 | CEA (P005.102- | See Table 13 |
| | combo1)- | |
| | CDR-H1 | |
| 197 | CEA (P005.102- | See Table 13 |
| | combo1)- | |
| | CDR-H2 | |
| 198 | CEA (P005.102- | See Table 13 |
| | combo1)- | |
| | CDR-H3 | |
| 199 | CEA (P005.102- | See Table 14 |
| | combo1)- | |
| | CDR-L1 | |
| 200 | CEA (P005.102- | See Table 14 |
| | combo1)- | |
| | CDR-L2 | |
| 201 | CEA (P005.102- | See Table 14 |
| | combo1)- | |
| | CDR-L3 | |
| 202 | CEA (P005.102- | See Table 13 |
| | combo2)-CDR-H1 | |
| 203 | CEA (P005.102- | See Table 13 |
| | combo2)-CDR-H2 | |
| 204 | CEA (P005.102- | See Table 13 |
| | combo2)-CDR-H3 | |
| 205 | CEA (P005.102- | See Table 14 |
| | combo2)-CDR-L1 | |
| 206 | CEA (P005.102- | See Table 14 |
| | combo2)-CDR-L2 | |
| 207 | CEA (P005.102- | See Table 14 |
| | combo2)-CDR-L3 | |
| 208 | CEA (P005.103- | See Table 13 |
| | combo1)-CDR-H1 | |
| 209 | CEA (P005.103- | See Table 13 |
| | combo1)-CDR-H2 | |
| 210 | CEA (P005.103- | See Table 13 |
| | combo1)-CDR-H3 | |
| 211 | CEA (P005.103- | See Table 14 |
| | combo1)-CDR-L1 | |
| 212 | CEA (P005.103- | See Table 14 |
| | combo1)-CDR-L2 | |
| 213 | CEA (P005.103- | See Table 14 |
| | combo1)-CDR-L3 | |
| 214 | CEA (P005.103- | See Table 13 |
| | combo2)-CDR-H1 | |
| 215 | CEA (P005.103- | See Table 13 |
| | combo2)-CDR-H2 | |
| 216 | CEA (P005 103- | See Table 13 |
| | combo2)-CDR-H3 | |
| 217 | CEA (P005.103- | See Table 14 |
| | combo2)-CDR-L1 | |
| 218 | CEA (P005.103- | See Table 14 |
| | combo2)-CDR-L2 | |
| 219 | CEA (P005.103- | See Table 14 |
| | combo2)-CDR-L3 | |
| 220 | CEA (P006.038- | See Table 13 |
| | combo1)-CDR-H1 | |
| 221 | CEA (P006.038- | See Table 13 |
| | combo1)-CDR-H2 | |
| 222 | CEA (P006.038- | See Table 13 |
| | combo1)-CDR-H3 | |
| 223 | CEA (P006.038- | See Table 14 |
| | combo1)-CDR-L1 | |
| 224 | CEA (P006.038- | See Table 14 |
| | combo1)-CDR-L2 | |
| 225 | CEA (P006.038- | See Table 14 |
| | combo1)-CDR-L3 | |
| 226 | CEA (P006.038- | See Table 13 |
| | combo2)-CDR-H1 | |
| 227 | CEA (P006.038- | See Table 13 |
| | combo2)-CDR-H2 | |
| 228 | CEA (P006.038- | See Table 13 |
| | combo2)-CDR-H3 | |
| 229 | CEA (P006.038- | See Table 14 |
| | combo2)-CDR-L1 | |
| 230 | CEA (P006.038- | See Table 14 |
| | combo2)-CDR-L2 | |
| 231 | CEA (P006.038- | See Table 14 |
| | combo2)-CDR-L3 | |
| 232 | IGHV1-2-02 human | QLVQSGAEVKKPGASVKVSCKASGYTFTGYYMHWVRQA |
| | acceptor | PGQGLEWMGWINPNSGGTNYAQKFQGRVTMTRDTSIST |
| | sequence | AYMELSRLRSDDTAVYYCAR |
| 233 | IGHV1-69-01 | QVQLVQSGAEVKKPGSSVKVSCKASGGTFSSYAISWVR |
| | human acceptor | QAPGQGLEWMGGIIPIFGTANYAQKFQGRVTITADEST |
| | sequence | STAYMELSSLRSEDTAVYYCAR |
| 234 | IGHV1-69-05 | QVQLVQSGAEVKKPGSSVKVSCKASGGTFSSYAISWVR |
| | human acceptor | QAPGQGLEWMGGIIPIFGTANYAQKFQGRVTITTDEST |
| | sequence | STAYMELSSLRSEDTAVYYCAR |
| 235 | IGKV1-39-01 | DIQMTQSPSSLSASVGDRVTITCRASQSISSYLNWYQQ |
| | | KPGKAPKLLIYAASSLQSGVPSRFSGSGSGTDFTLTIS |
| | | SLQPEDFATYYCQQSYSTP |
| 236 | anti-CEA(A5B7) | See Table 20 |
| | Fc hole chain | |
| 237 | anti-CEA(A5B7) | See Table 20 |
| | light chain | |
| 238 | anti-CEA(A5H1EL1D) | See Table 21 |
| | Fc hole | |
| | chain | |
| 239 | anti-CEA(A5H1EL1D) | See Table 21 |
| | light chain | |
| 240 | anti-CEA(P006.038) | See Table 22 |
| | Fc hole chain | |
| 241 | anti-CEA(P006.038) | See Table 22 |
| | light chain | |
| 242 | anti-CEA(P005.097) | See Table 23 |
| | Fc hole chain | |
| 243 | anti-CEA(P005.097) | See Table 23 |
| | light chain | |
| 244 | anti-CEA(P005.103) | See Table 24 |
| | Fc hole chain | |
| 245 | anti-CEA(P005.103) | See Table 24 |
| | light chain | |
| 246 | anti-CEA(P002.139) | See Table 25 |
| | Fc hole chain | |
| 247 | anti-CEA(P002.139) | See Table 25 |
| | light chain | |
| 248 | anti-CEA(P001.177) | See Table 26 |
| | Fc hole chain | |
| 249 | anti-CEA(P001.177) | See Table 26 |
| | light chain | |
| 250 | anti-CEA(P005.102) | See Table 27 |
| | Fc hole chain | |
| 251 | anti-CEA(P005.102) | See Table 27 |
| | light chain | |
| 252 | anti-CEA(P005.103- | See Table 28 |
| | combo1) Fc | |
| | hole chain | |
| 253 | anti-CEA(P005.103- | See Table 28 |
| | combo1) light | |
| | chain | |
| 254 | anti-CEA(P005.103- | See Table 29 |
| | combo2) Fc | |
| | hole chain | |
| 255 | anti-CEA(P005.103- | See Table 29 |
| | combo2) light | |
| | chain | |
| 256 | anti-CEA(P005.102- | See Table 30 |
| | combo1) Fc | |
| | hole chain | |
| 257 | anti-CEA(P005.102- | See Table 30 |
| | combo1) light | |
| | chain | |
| 258 | anti-CEA(P005.102- | See Table 31 |
| | combo2) Fc | |
| | hole chain | |
| 259 | anti-CEA(P005.102- | See Table 31 |
| | combo2) light | |
| | chain | |
| 260 | anti-CEA(P006.038- | See Table 32 |
| | combo1) Fc | |
| | hole chain | |
| 261 | anti-CEA(P006.038- | See Table 32 |
| | combo1) light | |
| | chain | |
| 262 | anti-CEA(P006.038- | See Table 33 |
| | combo2) Fc | |
| | hole chain | |
| 263 | anti-CEA(P006.038- | See Table 33 |
| | combo2) light | |
| | chain | |
| 264 | anti-CEA(MFE23) | See Table 34 |
| | Fc hole chain | |
| 265 | anti-CEA(MFE23) | See Table 34 |
| | light chain | |
| 266 | anti-CEA(huMFE23- | See Table 35 |
| | L28-H24) Fc | |
| | hole chain | |
| 267 | anti-CEA(huMFE23- | See Table 35 |
| | L28-H24) | |
| | light chain | |
| 268 | anti-CEA(huMFE23- | See Table 36 |
| | L28-H28) Fc | |
| | hole chain | |
| 269 | anti-CEA(huMFE23- | See Table 37 |
| | L28-H25) Fc | |
| | hole chain | |
| 270 | anti-CEA(huMFE23- | See Table 38 |
| | L27-H29) Fc | |
| | hole chain | |
| 271 | anti-CEA(huMFE23- | See Table 38 |
| | L27-H29) | |
| | light chain | |
| 272 | anti-CEA(huMFE23- | See Table 39 |
| | L27-H28) Fc | |
| | hole chain | |
| 273 | anti-CEA(huMFE23- | See Table 40 |
| | L27-H26) Fc | |
| | hole chain | |
| 274 | anti-CEA(huMFE23- | See Table 41 |
| | L27-H24) Fc | |
| | hole chain | |
| 275 | CDR-H1, CD3 | TYAMN |
| 276 | CDR-H2, CD3 | RIRSKYNNYATYYADSVKG |
| 277 | CDR-H3, CD3 | HGNFGNSYVSWFAY |
| 278 | CDR-L1, CD3 | GSSTGAVTTSNYAN |
| 279 | CDR-L2, CD3 | GTNKRAP |
| 280 | CDR-L3, CD3 | ALWYSNLWV |
| 281 | heavy chain variable | EVQLLESGGGLVQPGGSLRLSCAASGFTFSTYAM |
| | domain VH, | NWVRQAPGKGLEWVSRIRSKYNNYATYYADSVKG |
| | CD3 | RFTISRDDSKNTLYLQMNSLRAEDTAVYYCVRHG |
| | | NFGNSYVSWFAYWGQGTLVTVSS |
| 282 | light chain variable | QAVVTQEPSLTVSPGGTVTLTCGSSTGAVTTSNY |
| | domain VL, | ANWVQEKPGQAFRGLIGGTNKRAPGTPARFSGSL |
| | CD3 | LGGKAALTLSGAQPEDEAEYYCALWYSNLWVFGG |
| | | GTKLTVL |
| 283 | CEA (_{CH1A1A 98/99})- | EFGMN |
| | CDR-H1 | |
| 284 | CEA (_{CH1A1A 98/99})- | WINTKTGEATYVEEFKG |
| | CDR-H2 | |
| 285 | CEA (_{CH1A1A 98/99})- | WDFAYYVEAMDY |
| | CDR-H3 | |
| 286 | CEA (2F1)-CDR-L1 | KASAAVGTYVA |
| 287 | CEA (2F1)-CDR-L2 | SASYRKR |
| 288 | CEA (2F1)-CDR-L3 | HQYYTYPLFT |
| 289 | VH (CEA chiaia98/99) | QVQLVQSGAEVKKPGASVKVSCKASGYTFTEFGMNWVR |
| | | QAPGQGLEWMGWINTKTGEATYVEEFKGRVTFTTDTST |
| | | STAYMELRSLRSDDTAVYYCARWDFAYYVEAMDYWGQG |
| | | TTVTVSS |
| 290 | VL (CEA_{2F1}) | DIQMTQSPSSLSASVGDRVTITCKASAAVGTYVAWYQQ |
| | | KPGKAPKLLIYSASYRKRGVPSRFSGSGSGTDFTLTIS |
| | | SLQPEDFATYYCHQYYTYPLFTFGQGTKLEIK |
| 291 | CEA (T84.66-LCHA)-CDR-H1 | DTYMH |
| 292 | CEA (T84.66-LCHA)-CDR-H2 | RIDPANGNSKYVPKFQG |
| 293 | CEA (T84.66-LCHA)-CDR-H3 | FGYYVSDYAMAY |
| 294 | CEA (T84.66-LCHA)-CDR-L1 | RAGESVDIFGVGFLH |
| 295 | CEA (T84.66-LCHA)-CDR-L2 | RASNRAT |
| 296 | CEA (T84.66-LCHA)-CDR-L3 | QQTNEDPYT |
| 297 | Human 4-1BBL | UniProt no. P41273 |
| 298 | hu 4-1BBL (50-254) | ACPWAVSGARASPGSAASPRLREGPELSPDDPAGLLDL |
| | | RQGMFAQLVAQNVLLIDGPLSWYSDPGLAGVSLTGGLS |
| | | YKEDTKELVVAKAGVYYVFFQLELRRVVAGEGSGSVSL |
| | | ALHLQPLRSAAGAAALALTVDLPPASSEARNSAFGFQG |
| | | RLLHLSAGQRLGVHLHTEARARHAWQLTQGATVLGLFR |
| | | VTPEIPAGL |
| 299 | IgG CH2 domain | APELLGGPSV FLFPPKPKDT LMISRTPEVT |
| | | CVWDVSHEDP EVKFNWYVDG VEVHNAKTKP |
| | | REEQESTYRW SVLTVLHQDW LNGKEYKCKV |
| | | SNKALPAPIE KTISKAK |
| 300 | IgG CH3 domain | GQPREPQVYT LPPSRDELTK NQVSLTCLVK |
| | | GFYPSDIAVE WESNGQPENN YKTTPPVLDS |
| | | DGSFFLYSKL TVDKSRWQQG NVFSCSVMHE |
| | | ALHNHYTQKS LSLSP |
| 301 | IgG CH1 domain | ASTKGPSVFP LAPSSKSTSG GTAALGCLVK |
| | | DYFPEPVTVS WNSGALTSGV HTFPAVLQSS |
| | | GLYSLSSVVT VPSSSLGTQT YICNVNHKPS |
| | | NTKVDKKV |
| 302 | Linker to hinge region | EPKSC |
| 303 | Hinge full | DKTHTCPXCP with X being S or P |
| 304 | Hinge middle | HTCPXCP with X being S or P |
| 305 | Hinge short | CPXCP with X being S or P |
| 306 | IgG1, Caucasian | ASTKGPSVFPLAPSSKSTSGGTAALGCLVKDYFPEPVT |
| | allotype | VSWNSGALTSGVHTFPAVLQSSGLYSLSSVVTVPSSSL |
| | | GTQTYICNVNHKPSNTKVDKKVEPKSCDKTHTCPPCPA |
| | | PELLGGPSVFLFPPKPKDTLMISRTPEVTCVVVDVSHE |
| | | DPEVKFNWYVDGVEVHNAKTKPREEQYNSTYRVVSVLT |
| | | VLHQDWLNGKEYKCKVSNKALPAPIEKTISKAKGQPRE |
| | | PQVYTLPPSRDELTKNQVSLTCLVKGFYPSDIAVEWES |
| | | NGQPENNYKTTPPVLDSDGSFFLYSKLTVDKSRWQQGN |
| | | VFSCSVMHEALHNHYTQKSLSLSPGK |
| 307 | IgG1, afroamerican | ASTKGPSVFPLAPSSKSTSGGTAALGCLVKDYFPEPVT |
| | allotype | VSWNSGALTSGVHTFPAVLQSSGLYSLSSVVTVPSSSL |
| | | GTQTYICNVNHKPSNTKVDKKVEPKSCDKTHTCPPCPA |
| | | PELLGGPSVFLFPPKPKDTLMISRTPEVTCVVVDVSHE |
| | | DPEVKFNWYVDGVEVHNAKTKPREEQYNSTYRVVSVLT |
| | | VLHQDWLNGKEYKCKVSNKALPAPIEKTISKAKGQPRE |
| | | PQVYTLPPSREEMTKNQVSLTCLVKGFYPSDIAVEWES |
| | | NGQPENNYKTTPPVLDSDGSFFLYSKLTVDKSRWQQGN |
| | | VFSCSVMHEALHNHYTQKSLSLSPGK |
| 308 | IgG2 | ASTKGPSVFPLAPCSRSTSESTAALGCLVKDYFPEPVT |
| | | VSWNSGALTSGVHTFPAVLQSSGLYSLSSVVTVPSSNF |
| | | GTQTYTCNVDHKPSNTKVDKTVERKCCVECPPCPAPPV |
| | | AGPSVFLFPPKPKDTLMISRTPEVTCVVVDVSHEDPEV |
| | | QFNWYVDGVEVHNAKTKPREEQFNSTFRVVSVLTVVHQ |
| | | DWLNGKEYKCKVSNKGLPAPIEKTISKTKGQPREPQVY |
| | | TLPPSREEMTKNQVSLTCLVKGFYPSDISVEWESNGQP |
| | | ENNYKTTPPMLDSDGSFFLYSKLTVDKSRWQQGNVFSC |
| | | SVMHEALHNHYTQKSLSLSPGK |
| 309 | IgG3 | ASTKGPSVFPLAPCSRSTSGGTAALGCLVKDYFPEPVT |
| | | VSWNSGALTSGVHTFPAVLQSSGLYSLSSVVTVPSSSL |
| | | GTQTYTCNVNHKPSNTKVDKRVELKTPLGDTTHTCPRC |
| | | PEPKSCDTPPPCPRCPEPKSCDTPPPCPRCPEPKSCDT |
| | | PPPCPRCPAPELLGGPSVFLFPPKPKDTLMISRTPEVT |
| | | CVVVDVSHEDPEVQFKWYVDGVEVHNAKTKPREEQYNS |
| | | TFRVVSVLTVLHQDWLNGKEYKCKVSNKALPAPIEKTI |
| | | SKTKGQPREPQVYTLPPSREEMTKNQVSLTCLVKGFYP |
| | | SDIAVEWESSGQPENNYNTTPPMLDSDGSFFLYSKLTV |
| | | DKSRWQQGNIFSCSVMHEALHNRFTQKSLSLSPGK |
| 310 | IgG4 | ASTKGPSVFPLAPCSRSTSESTAALGCLVKDYFPEPVT |
| | | VSWNSGALTSGVHTFPAVLQSSGLYSLSSVVTVPSSSL |
| | | GTKTYTCNVDHKPSNTKVDKRVESKYGPPCPSCPAPEF |
| | | LGGPSVFLFPPKPKDTLMISRTPEVTCVVVDVSQEDPE |
| | | VQFNWYVDGVEVHNAKTKPREEQFNSTYRVVSVLTVLH |
| | | QDWLNGKEYKCKVSNKGLPSSIEKTISKAKGQPREPQV |
| | | YTLPPSQEEMTKNQVSLTCLVKGFYPSDIAVEWESNGQ |
| | | PENNYKTTPPVLDSDGSFFLYSRLTVDKSRWQEGNVFS |
| | | CSVMHEALHNHYTQKSLSLSLGK |
| 311 | A2 domain of hu | PKPFITSNNSMPVEDEDAVALTCEPEIQNTTYLW |
| | CEACAM5 | WVNNQSLPVSPRLQLSNDMRTLTLLSVTRNDVGF |
| | | YECGIQNKLSVDHSDPVILN |
| 312 | A1 domain of hu | PKPSISSNNSKPVEDKDAVAFTCBPETQDATYLW |
| | CEACAM5 | WVNNQSLPVSPRLQLSNGNRTLTLFNVTRNDTAS |
| | | YKCETQNFVSARRSDSVILN |
| 313 | CEA (MFE-L27, L28, | RASSSVPYMH |
| | L29)-CDR-L1 | |

General information regarding the nucleotide sequences of human immunoglobulins light and heavy chains is given in: Kabat, E. A., et al., Sequences of Proteins of Immunological Interest, 5th ed., Public Health Service, National Institutes of Health, Bethesda, Md. (1991). Amino acids of antibody chains are numbered and referred to according to the EU numbering systems according to Kabat (Kabat, E. A., et al., Sequences of Proteins of Immunological Interest, 5th ed., Public Health Service, National Institutes of Health, Bethesda, Md. (1991)) as defined above.

### EXAMPLES

The following are examples of methods and compositions of the invention. It is understood that various other embodiments may be practiced, given the general description provided above.

Recombinant DNA Techniques

Standard methods were used to manipulate DNA as described in Sambrook et al., Molecular cloning: A laboratory manual; Cold Spring Harbor Laboratory Press, Cold Spring Harbor, N.Y., 1989. The molecular biological reagents were used according to the manufacturer's instructions. General information regarding the nucleotide sequences of human immunoglobulin light and heavy chains is given in: Kabat, E. A. et al., (1991) Sequences of Proteins of Immunological Interest, Fifth Ed., NIH Publication No 91-3242.

DNA Sequencing

DNA sequences were determined by double strand sequencing.

Gene Synthesis

Desired gene segments were either generated by PCR using appropriate templates or were synthesized by Geneart AG (Regensburg, Germany) from synthetic oligonucleotides and PCR products by automated gene synthesis. In cases where no exact gene sequence was available, oligonucleotide primers were designed based on sequences from closest homologues and the genes were isolated by RT-PCR from RNA originating from the appropriate tissue. The gene segments flanked by singular restriction endonuclease cleavage sites were cloned into standard cloning/sequencing vectors. The plasmid DNA was purified from transformed bacteria and concentration determined by UV spectroscopy. The DNA sequence of the subcloned gene fragments was confirmed by DNA sequencing. Gene segments were designed with suitable restriction sites to allow sub-cloning into the respective expression vectors. All constructs were designed with a 5′-end DNA sequence coding for a leader peptide which targets proteins for secretion in eukaryotic cells.

Cell Culture Techniques

Standard cell culture techniques were used as described in Current Protocols in Cell Biology (2000), Bonifacino, J. S., Dasso, M., Harford, J. B., Lippincott-Schwartz, J. and Yamada, K. M. (eds.), John Wiley & Sons, Inc.

Protein Purification

Proteins were purified from filtered cell culture supernatants referring to standard protocols. In brief, antigen binding molecules were applied to a Protein A-affinity chromatography (equilibration buffer: 20 mM sodium citrate, 20 mM sodium phosphate, pH 7.5; elution buffer: 20 mM sodium citrate, pH 3.0). Elution was achieved at pH 3.0 followed by immediate pH neutralization of the sample. Aggregated protein was separated from monomeric antibodies by size exclusion chromatography (Superdex 200, GE Healthcare) in PBS or in 20 mM Histidine, 140 mM NaCl at pH 6.0. Monomeric antigen binding molecule fractions can be pooled, concentrated (if required) using e.g., a MILLIPORE Amicon Ultra (30 MWCO) centrifugal concentrator, frozen and stored at −20° C. or −80° C. Part of the samples can be provided for subsequent protein analytics and analytical characterization e.g. by SDS-PAGE, size exclusion chromatography (SEC) or mass spectrometry.

### Example 1

### Generation and Production of Anti-CEA Antibodies

### 1.1 Generation of Humanized Variants of Anti-CEA Antibody A5B7

### 1.1.1 Methodology

Anti-CEA antibody A5B7 is for example disclosed by M. J. Banfield et al, Proteins 1997, 29(2), 161-171 and its structure can be found as PDB ID:1CLO in the Protein structural database PDB (www.rcsb.org, H. M. Berman et al, The Protein Data Bank, Nucleic Acids Research, 2000, 28, 235-242). This entry includes the heavy and the light chain variable domain sequence. For the identification of a suitable human acceptor framework during the humanization of the anti-CEA binder A5B7, a classical approach was taken by searching for an acceptor framework with high sequence homology, grafting of the CDRs on this framework, and evaluating which back-mutations can be envisaged. More explicitly, each amino acid difference of the identified frameworks to the parental antibody was judged for impact on the structural integrity of the binder, and back mutations towards the parental sequence were introduced whenever appropriate. The structural assessment was based on Fv region homology models of both the parental antibody and its humanized versions created with an in-house antibody structure homology modeling tool implemented using the Biovia Discovery Studio Environment, version 4.5.

### 1.1.2 Choice of Acceptor Framework and Adaptations Thereof

The acceptor framework was chosen as described in Table 1 below:

TABLE 1
Acceptor framework

| | | Choice of human |
|---|---|---|
| | Closest murine | acceptor V-region |
| | V-region germline | germline |
| A5B7 VH | mu-IGHV7-3-02 | IGHV3-23-01 or |
| | | IGHV3-15-01 |
| A5B7 VL | mu-IGKV4-72-01 | IGKV3-11-01 |

Post-CDR3 framework regions were adapted from human J-element germline IGJH6 for the heavy chain, and a sequence similar to the kappa J-element IGKJ2, for the light chain.

Based on structural considerations, back mutations from the human acceptor framework to the amino acid in the parental binder were introduced at positions 93 and 94 of the heavy chain.

### 1.1.3 VII and VL Regions of the Resulting Humanized CEA Antibodies

The resulting VH domains of humanized CEA antibodies can be found in Table 2 below and the resulting VL domains of humanized CEA antibodies are listed in Table 3 below.

TABLE 2
Amino acid sequences of the VH domains of
humanized CEA antibodies, based 011 human
acceptor framework IGHV3-23 or IGHV3-15

| | | Seq |
|---|---|---|
| | | ID |
| Description | Sequence | No |
| A5B7 VH | EVKLVESGGGLVQPG | 7 |
| murine donor | GSLRLSCATSGFTFT | |
| sequence | DYYMNWVRQPPGKAL | |
| | EWLGFIGNKANGYTT | |
| | EYSASVKGRFTISRD | |
| | KSQSILYLQMNTLRA | |
| | EDSATYYCTRDRGLR | |
| | FYFDYWGQGTTLTVS | |
| | S | |
| IGHV3-23-02 | EVQLLESGGGLVQPG | 127 |
| human | GSLRLSCAASGFTFS | |
| acceptor | SYAMSWVRQAPGKGL | |
| sequence | EWVSAISGSGGSTYY | |
| | GDSVKGRFTISRDNS | |
| | KNTLYLQMNSLRAED | |
| | TAVYYCAK | |
| Humanized | | |
| variants | | |
| 3-23A5-1 | EVQLLESGGGLVQPG | 129 |
| | GSLRLSCAASGFTFT | |
| | DYYMNWVRQAPGKGL | |
| | EWVGFIGNKANGYTT | |
| | EYSASVKGRFTISRD | |
| | NSKNTLYLQMNSLRA | |
| | EDTAVYYCARDRGLR | |
| | FYFDYWGQGTTVTVS | |
| | S | |
| 3-23A5-2 | EVQLLESGGGLVQPG | 130 |
| | GSLRLSCAASGFTFT | |
| | DYYMNWVRQAPGKGL | |
| | EWVGFIGNKANGYTT | |
| | YYGDSVKGRFTISRD | |
| | NSKNTLYLQMNSLRA | |
| | EDTAVYYCARDRGLR | |
| | FYFDYWGQGTTVTVS | |
| | S | |
| 3-23A5-3 | EVQLLESGGGLVQPG | 131 |
| | GSLRLSCAASGFTFT | |
| | DYYMNWVRQAPGKGL | |
| | EWVGFIGNKGYTTEY | |
| | SASVKGRFTISRDNS | |
| | KNTLYLQMNSLRAED | |
| | TAVYYCARDRGLRFY | |
| | FDYWGQGTTVTVSS | |
| 3-23A5-4 | EVQLLESGGGLVQPG | 132 |
| | GSLRLSCAASGFTFT | |
| | DYYMSWVRQAPGKGL | |
| | EWVGFIGNKANGYTT | |
| | EYSASVKGRFTISRD | |
| | NSKNTLYLQMNSLRA | |
| | EDTAVYYCARDRGLR | |
| | FYFDYWGQGTTVTVS | |
| | S | |
| 3-23A5-1A | EVQLLESGGGLVQPG | 133 |
| (all_ | GSLRLSCAASGFTFT | |
| backmutations) | DYYMNWVRQAPGKGL | |
| | EWLGFIGNKANGYTT | |
| | EYSASVKGRFTISRD | |
| | KSKNTLYLQMNSLRA | |
| | EDTATYYCTRDRGLR | |
| | FYFDYWGQGTTVTVS | |
| | S | |
| 3-23A5-1C | EVQLLESGGGLVQPG | 134 |
| (A93T) | GSLRLSCAASGFTFT | |
| | DYYMNWVRQAPGKGL | |
| | EWVGFIGNKANGYTT | |
| | EYSASVKGRFT!SRD | |
| | NSKNTLYLQMNSLRA | |
| | EDTAVYYCTR | |
| | DRGLRFYFDYWGQGT | |
| | TVTVSS | |
| 3-23A5-1D | EVQLLESGGGLVQPG | 135 |
| (K73) | GSLRLSCAASGFTFT | |
| | DYYMNWVRQAPGKGL | |
| | EWVGFIGNKANGYTT | |
| | EYSASVKGRFTISRD | |
| | KSKNTLYLQMNSLRA | |
| | EDTAVYYCARDRGLR | |
| | FYFDYWGQGTTVTVS | |
| | S | |
| 3-23A5-1E | EVQLLESGGGLVQPG | 23 |
| (G54A) | GSLRLSCAASGFTFT | |
| | DYYMNWVRQAPGKGL | |
| | EWLGFIGNKANAYTT | |
| | EYSASVKGRFTISRD | |
| | KSKNTLYLQMNSLRA | |
| | EDTATYYCTRDRGLR | |
| | FYFDYWGQGTTVTVS | |
| | S | |
| IGHV3-15*01 | EVQLVESGGGLVKPG | 128 |
| human | GSLRLSCAASGFTFS | |
| acceptor | NAWMSWVRQAPGKGL | |
| sequence | EWVGRIKSKTDGGTT | |
| | DYAAPVKGRFTISRD | |
| | DSKNTLYLQMNSLKT | |
| | EDTAVYYCTT | |
| Humanized | | |
| variants | | |
| 3-15A5-1 | EVQLVESGGGLVKPG | 136 |
| | GSLRLSCAASGFTFT | |
| | DYYMNWVRQAPGKGL | |
| | EWVGFIGNKANGYTT | |
| | EYSASVKGRFTISRD | |
| | DSKNTLYLQMNSLKT | |
| | EDTAVYYCTRDRGLR | |
| | FYFDYWGQGTTVTVS | |
| | S | |
| 3-15A5-2 | EVQLVESGGGLVKPG | 137 |
| | GSLRLSCAASGFTFT | |
| | DYYMNWVRQAPGKGL | |
| | EWVGFIGNKANGYTT | |
| | EYAAPVKGRFTISRD | |
| | DSKNTLYLQMNSLKT | |
| | EDTAVYYCTRDRGLR | |
| | FYFDYWGQGTTVTVS | |
| | S | |
| 3-15A5-3 | EVQLVESGGGLVKPG | 138 |
| | GSLRLSCAASGFTFT | |
| | DYYMNWVRQAPGKGL | |
| | EWVGFIGNKANGGTT | |
| | DYAAPVKGRFTISRD | |
| | DSKNTLYLQMNSLKT | |
| | EDTAVYYCTRDRGLR | |
| | FYFDYWGQGTTVTVS | |
| | S | |

For the heavy chain, the initial variant 3-23A5-1 was found suitable in binding assays (but showed slightly less binding than the parental murine antibody) and was chosen as starting point for further modifications. The variants based on IGHV3-15 showed less binding activity compared to humanized variant 3-23A5-1.

In order to restore the full binding activity of the parental chimeric antibody, variants 3-23A5-1A, 3-23A5-1C and 3-23A5-1D were created. It was also tested for variant 3-23A5-1 whether the length of CDR-H2 could be adapted to the human acceptor sequence, but this construct completely lost binding activity. Since a putative deamidation hotspot was present in CDR-H2 (Asn53-Gly54), we changed that motif to Asn53-Ala54. Another possible hotspot Asn73-Ser74 was backmutated to Lys73-Ser74. Thus, variant 3-23A5-1E was created.

TABLE 3
Amino acid sequences of the VL domains
of humanized CEA antibodies, based
011 human acceptor framework IGKV3-11.

| | | Seq |
|---|---|---|
| | | ID |
| Description | Sequence | No |
| A5B7 VL | QTVLSQSPAILSASP | 8 |
| murine | GEKVTMTCRASSSVT | |
| donor | YIHWYQQKPGSSPKS | |
| sequence | WIYATSNLASGVPAR | |
| | FSGSGSGTSYSLTIS | |
| | RVEAEDAATYYCQHW | |
| | SSKPPTFGGGTKLEI | |
| | K | |
| IGKV3-11 | EIVLTQSPATLSLSP | 139 |
| human | GERATLSCRASQSVS | |
| acceptor | SYLAWYQQKPGQAPR | |
| sequence | LLIYDASNRATGIPA | |
| | RFSGSGSGTDFTLTI | |
| | SSLEPEDFAVYYCQQ | |
| | RSNWP | |
| humanized | | |
| variants | | |
| A5-L1 | EIVLTQSPATLSLSP | 140 |
| | GERATLSCRASSSVT | |
| | YIHWYQQKPGQAPRL | |
| | LIYATSNLASGIPAR | |
| | FSGSGSGTDFTLTIS | |
| | SLEPEDFAVYYCQHW | |
| | SSKPPTFGQGTKLEI | |
| | K | |
| A5-L2 | EIVLTQSPATLSLSP | 141 |
| | GERATLSCRASQSVS | |
| | SYIHWYQQKPGQAPR | |
| | LLIYATSNLASGIPA | |
| | RFSGSGSGTDFTLTI | |
| | SSLEPEDFAVYYCQH | |
| | WSSKPPTFGQGTKLE | |
| | IK | |
| A5-L3 | EIVLTQSPATLSLSP | 142 |
| | GERATLSCRASSSVT | |
| | YIHWYQQKPGQAPRL | |
| | LIYDASNRATGIPAR | |
| | FSGSGSGTDFTLTIS | |
| | SLEPEDFAVYYCQHW | |
| | SSKPPTFGQGTKLEI | |
| | K | |
| A5-L4 | EIVLTQSPATLSLSP | 143 |
| | GERATLSCRASSSVT | |
| | YIHWYQQKPGQAPRL | |
| | LIYATSNLASGIPAR | |
| | FSGSGSGTDFTLTIS | |
| | SLEPEDFAVYYCQQW | |
| | SSKPPTFGQGTKLEI | |
| | K | |
| A5-L1A | QTVLTQSPATLSLSP | 144 |
| (all_ | GERATLSCRASSSVT | |
| backmutations) | YIHWYQQKPGSSPKS | |
| | WIYATSNLASGIPAR | |
| | FSGSGSGTDYTLTIS | |
| | SLEPEDFAVYYCQHW | |
| | SSKPPTFGQGTKLEI | |
| | K | |
| A5-L1B | QTVLTQSPATLSLSP | 145 |
| (Q1T2) | GERATLSCRASSSVT | |
| | YIHWYQQKPGQAPRL | |
| | LIYATSNLASGIPAR | |
| | FSGSGSGTDFTLTIS | |
| | SLEPEDFAVYYCQHW | |
| | SSKPPTFGQGTKLEI | |
| | K | |
| A5-L1C | EIVLTQSPATLSLSP | 146 |
| (FR2) | GERATLSCRASSSVT | |
| | YIHWYQQKPGSSPKS | |
| | WIYATSNLASGIPAR | |
| | FSGSGSGTDFTLTIS | |
| | SLEPEDFAVYYCQHW | |
| | SSKPPTFGQGTKLEI | |
| | K | |
| A5-L1D | EIVLTQSPATLSLSP | 24 |
| (46, 47) | GERATLSCRASSSVT | |
| | YIHWYQQKPGQAPRS | |
| | WIYATSNLASGIPAR | |
| | FSGSGSGTDFTLTIS | |
| | SLEPEDFAVYYCQHW | |
| | SSKPPTFGQGTKLEI | |
| | K | |

The light chain was humanized based on the human IGKV3-11 acceptor framework. In the series A5-L1 to A5-L4, it was learned that variant A5-L1 shows good binding activity (but slightly less than the parental antibody). Partial humanization of CDR-L1 (variant A5-L2; Kabat positions 30 and 31) fully abrogates the binding. Likewise, humanization of CDR-H2 (variant A5-L3; Kabat positions 50 to 56) also fully abrogates the binding. The position 90 (variant A5-L4) shows significant contribution to the binding properties. The Histidine at this position is important for binding. Thus, variant A5-L1 was chosen for further modification.

The series A5-L1A to A5-L1D addressed the question which backmutations are required to restore the full binding potential of the parental chimeric antibody. Variant A5-L1A showed that backmutations at Kabat positions 1, 2, the entire framework 2, and Kabat position 71 do not add any further binding activity. Variants A5-L1B, and A5-L1C addressed subsets of those positions and confirm that they do not alter the binding properties. Variant A5-L1D with back mutations at Kabat positions 46 and 47 showed the best binding activity.

### 1.1.4 Selection of Humanized A5B7 Antibodies

Based on the new humanization variants of VH and VL new CEA antibodies were expressed as huIgG1 antibodies with an effector silent Fc (P329G; L234A, L235A) to abrogate binding to Fcγ receptors according to the method described in WO 2012/130831 A1 and their binding to CEA expressed on MKN45 cells was tested and compared to the respective parental murine A5B7 antibody.

TABLE 4
VH/VL combinations expressed as huIgG1_LALA_PG antibodies

| | A5-L1A | A5-L1B | A5-L1C | A5-L1D |
|---|---|---|---|---|
| 3-23A5-1A | P1AE2164 | P1AE2165 | P1AE2166 | P1AE2167 |
| 3-23A5-1C | — | — | P1AE2176 | P1AE2177 |
| 3-23A5-1D | P1AE2179 | — | P1AE2181 | P1AE2182 |

MKN45 (DSMZ ACC 409) is a human gastric adenocarcinoma cell line expressing CEA. The cells were cultured in advanced RPMI+2% FCS+1% Glutamax. Viability of MKN-45 cells was checked and cells were re-suspended and adjusted to a density of 1 Mio cells/ml. 100 μl of this cell suspension (containing 0.1 Mio cells) were seeded into a 96 well round bottom plate. The plate was centrifuged for 4 min at 400×g and the supernatant was removed. Then 40 μl of the diluted antibodies or FACS buffer were added to the cells and incubated for 30 min at 4° C. After the incubation the cells were washed twice with 150 μl FACS buffer per well. Then 20 μl of the diluted secondary PE anti-human Fc specific secondary antibody (109-116-170, Jackson ImmunoResearch) was added to the cells. The cells were incubated for an additional 30 min at 4° C. To remove unbound antibody, the cells were washed again twice with 150 μl per well FACS buffer. To fix the cells 100 μl of FACS buffer containing 1% PFA were added to the wells. Before measuring the cells were re-suspended in 150 μl FACS buffer. The fluorescence was measured using a BD flow cytometer.

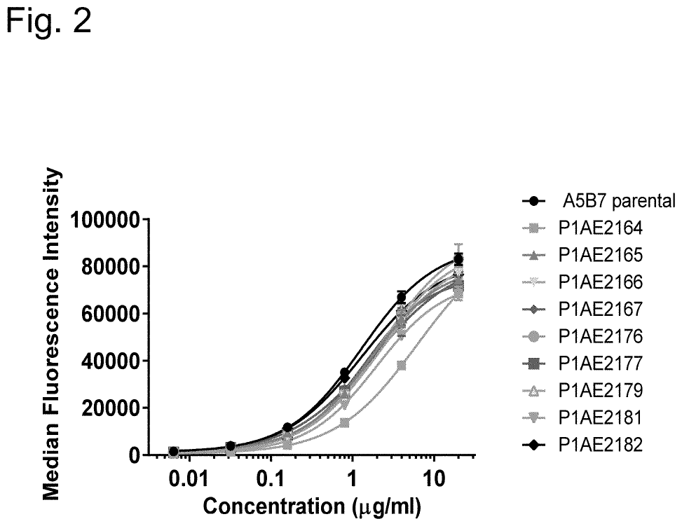

In FIG. 2 binding curves of the humanized A5B7 variants are shown. All tested binders were able to bind to MKN45 cells but binding capacity was slightly reduced compared to the parental A5B7 antibody. The clone P1AE2167 had the best binding of all tested variants and was selected for further development.

### 1.1.5 Determination of Affinities of Fab Fragments of Humanized Variants of Murine CEA-Antibody A5B7 to Human CEA Using Surface Plasmon Resonance (BIACORE)

The affinities of Fab fragments of the humanized variants of murine CEA antibody A5B7 to human CEA were assessed by surface plasmon resonance using a BIACORE T200 instrument. On a CM5 chip, human CEA (hu N(A2-B2)A-avi-His B) was immobilized at a 40 nM concentration by standard amine coupling on flow cell 2 for 30 s to about 100RU. The Fab fragments of the humanized variants of murine CEA antibody A5B7 were subsequently injected as analytes in 3-fold dilutions ranging from 500-0.656 nM for a contact time of 120 s, a dissociation time of 250 or 1000 s and at a flow rate of 30 μl/min. Regeneration at the level of human CEA (hu N(A2-B2)A-avi-His B) was achieved by 2 pulses of 10 mM glycine/HCl pH2.0 for 60 s. Data were double-referenced against the unimmobilized flow cell 1 and a zero concentration of the analyte. The sensorgrams of the analytes were fitted to a simple 1:1 Langmuir interaction model. Affinity constants [K_{D}] for human CEA (A2 domain) are summarized in Table 5 below.

TABLE 5
Affinity constants of Fab fragments representing
different humanized variants of murine CEA
antibody A5B7 to human CEA (A2 domain).

| | | Affinity to human |
|---|---|---|
| | | N(A2-B2)A-avi-His B |
| Tapir ID | Name | [M] |
| P1AE0289 | CEA (A5B7) Fab (parental | 5.59E−10 |
| | murine antibody) | |
| P1AE4135 | Fab derived from P1AE2164 | 1.70E−09 |
| P1AE4136 | Fab derived from P1AE2165 | 1.25E−09 |
| P1AE4137 | Fab derived from P1AE2166 | 1.13E−08 |
| P1AE4138 | Fab derived from P1AE2167 | 1.47E−09 |
| P1AE4139 | Fab derived from P1AE2176 | 7.58E−09 |
| P1AE4140 | Fab derived from P1AE2177 | 7.62E−09 |
| P1AE4141 | Fab derived from P1AE2179 | 1.83E−09 |
| P1AE4142 | Fab derived from P1AE2181 | 2.64E−09 |
| P1AE4143 | Fab derived from P1AE2182 | 2.92E−09 |

The humanized variants of the murine CEA antibody A5B7 are of lower affinities than the parental murine antibody. The Fab fragment P1AE4138, derived from P1AE2167 (heavy chain with VH variant 3-23A5-1A and Ckappa light chain with VL variant A5-L1D) was chosen as final humanized variant. Moreover, a glycine to alanine mutation at Kabat position 54 (G54A) was introduced into the VH domain in order to remove a deamidation site, leading to VL variant 3-23A5-1E. The final humanized antibody (heavy chain with VH variant 3-23A5-1E and Ckappa light chain with VL variant A5-L1D) has been named A5H1EL1D or huA5B7.

### 1.2 Generation of A5H1EL1D-Derived Affinity-Matured Anti-CEA Antibodies

### 1.2.1 Preparation, Purification and Characterization of Antigens for Phage Display Campaign

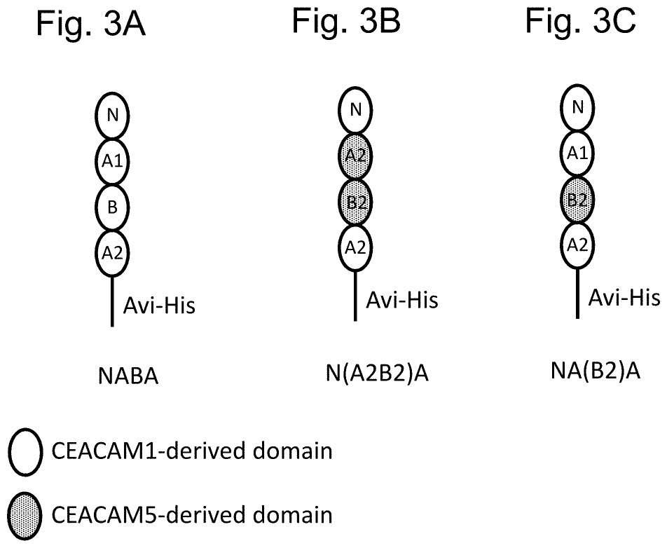

The murine antibody A5B7 and its humanized derivative A5H1EL1D bind to the A2 domain of CEACAM5 (CEA) with an affinity of about 0.8 and about 2.5 nM, respectively. For the generation of affinity-matured variants of A5H1EL1D by phage display, 3 different recombinant soluble antigens were generated. Each protein contained a C-terminal avi tag for site-specific biotinylation and a his-tag for purification: The first protein consisted of the extra-cellular part of CEACAM1 consisting of the 4 Ig-like domains N, A1, B, A2 (NABA-avi-His, SEQ ID NO: 147, Table 6). The second protein was a chimeric protein consisting of 2 CEACAM5 and 2 CEACAM1 Ig domains. Based on the sequence of the four domains of CEACAM1, the DNA encoding the second and third domain of CEACAM1 (A1 and B domains) was replaced by the DNA encoding the A2 and B2 domains of CEACAM5 (N(A2B2)A-avi-His, SEQ ID NO:148, Table 6). The third protein was a chimeric protein consisting of 1 CEACAM5 and 3 CEACAM1 Ig domains. Based on the sequence of the four domains of CEACAM1, the DNA encoding the third domain of CEACAM1 (B domain) was replaced by the DNA encoding the B2 domain of CEACAM5 (NA(B2)A-avi-His, SEQ ID NO:149, Table 6). A schematic description of the three constructs is shown in FIGS. 3A, 3B and 3C.

TABLE 6
Amino acid sequences of used CEA antigens

| | | SEQ ID |
|---|---|---|
| Antigen | Sequence | NO |
| NABA-avi- | QLTTESMPFNVAEGK | 147 |
| His | EVLLLVHNLPQQLFG | |
| | YSWYKGERVDGNRQI | |
| | VGYAIGTQQATPGPA | |
| | NSGRETIYPNASLLI | |
| | QNVTQNDTGFYTLQV | |
| | IKSDLVNEEATGQFH | |
| | VYPELPKPSISSNNS | |
| | NPVEDKDAMAFTCEP | |
| | ETQDTTYLWWINNQS | |
| | LPVSPRLQLSNGNRT | |
| | LTLLSVTRNDTGPYE | |
| | CEIQNPVSANRSDPV | |
| | TLNVTYGPDTPTISP | |
| | SDTYYRPGANLSLSC | |
| | YAASNPPAQYSWLIN | |
| | GTFQQSTQELFIPNI | |
| | TVNNSGSYTCHANNS | |
| | VTGCNRTTVKTIIVT | |
| | ELSPVVAKPQIKASK | |
| | TTVTGDKDSVNLTCS | |
| | TNDTGISIRWFFKNQ | |
| | SLPSSERMKLSQGNI | |
| | TLSINPVKREDAGTY | |
| | WCEVFNPISKNQSDP | |
| | IMLNVNYNALPQENL | |
| | INVDLEVLFQGPGSG | |
| | LNDIFEAQKIEWHEA | |
| | RAHHHHHH | |
| N(A2B2)A- | QLTTESMPFNVAEGK | 148 |
| avi-His | EVLLLVHNLPQQLFG | |
| | YSWYKGERVDGNRQI | |
| | VGYAIGTQQATPGPA | |
| | NSGRETIYPNASLLI | |
| | QNVTQNDTGFYTLQV | |
| | IKSDLVNEEATGQFH | |
| | VYPELPKPFITSNNS | |
| | NPVEDEDAVALTCEP | |
| | EIQNTTYLWWVNNQS | |
| | LPVSPRLQLSNDNRT | |
| | LTLLSVTRNDVGPYE | |
| | CGIQNKLSVDHSDPV | |
| | ILNVLYGPDDPTISP | |
| | SYTYYRPGVNLSLSC | |
| | HAASNPPAQYSWLID | |
| | GNIQQHTQELFISNI | |
| | TEKNSGLYTCQANNS | |
| | ASGHSRTTVKTITVS | |
| | ALSPVVAKPQIKASK | |
| | TTVTGDKDSVNLTCS | |
| | TNDTGISIRWFFKNQ | |
| | SLPSSERMKLSQGNI | |
| | TLSINPVKREDAGTY | |
| | WCEVFNPISKNQSDP | |
| | IMLNVNYNALPQENL | |
| | INVDGSGLNDIFEAQ | |
| | KIEWHEARAHHHHHH | |
| NA(B2)A- | QLTTESMPFNVAEGK | 149 |
| avi-His | EVLLLVHNLPQQLFG | |
| | YSWYKGERVDGNRQI | |
| | VGYAIGTQQATPGPA | |
| | NSGRETIYPNASLLI | |
| | QNVTQNDTGFYTLQV | |
| | IKSDLVNEEATGQFH | |
| | VYPELPKPSISSNNS | |
| | NPVEDKDAMAFTCEP | |
| | ETQDTTYLWWINNQS | |
| | LPVSPRLQLSNGNRT | |
| | LTLLSVTRNDTGPYE | |
| | CEIQNPVSANRSDPV | |
| | TLNVTYGPDDPTISP | |
| | SYTYYRPGVNLSLSC | |
| | HAASNPPAQYSWLID | |
| | GNIQQHTQELFISNI | |
| | TEKNSGLYTCQANNS | |
| | ASGHSRTTVKTITVS | |
| | ALSPVVAKPQIKASK | |
| | TTVTGDKDSVNLTCS | |
| | TNDTGISIRWFFKNQ | |
| | SLPSSERMKLSQGNI | |
| | TLSINPVKREDAGTY | |
| | WCEVFNPISKNQSDP | |
| | IMLNVNYNALPQENL | |
| | INVDGSGLNDIFEAQ | |
| | KIEWHEARAHHHHHH | |

The respective plasmids were transiently transfected into HEK 293 cells, stably expressing the EBV-derived protein EBNA (HEK EBNA). A simultaneously co-transfected plasmid encoding the biotin ligase BirA allowed avi-tag-specific biotinlylation in vivo. Proteins were purified from filtered cell culture supernatants referring to standard protocols using immobilized metal affinity chromatography (IMAC) followed by gel filtration. Monomeric protein fractions were pooled, concentrated (if required), frozen and stored at −80° C. Part of the samples were provided for subsequent protein analytics and analytical characterization e.g. by SDS-PAGE, size exclusion chromatography (SEC) or mass spectrometry.

### 1.2.2 Selection of Affinity Matured A5H1EL1D-Derived Antibodies

Humanization of antibody A5B7 resulted in an about 3 to 4-fold reduction of the affinity to CEA measured by SPR. While the affinity for A5B7 was about 0.8 nM, an affinity of about 2.5 nM was measured for A5H1EL1D. FACS experiments using cell lines with different CEA expression levels confirmed this finding. In order to improve the affinity of the humanized clone A5H1EL1D, 3 different affinity-maturation libraries were made and used for the selection of clones with improved affinities by phage display.

1.2.2.1 Generation of A5H1EL1D Affinity Maturation Libraries

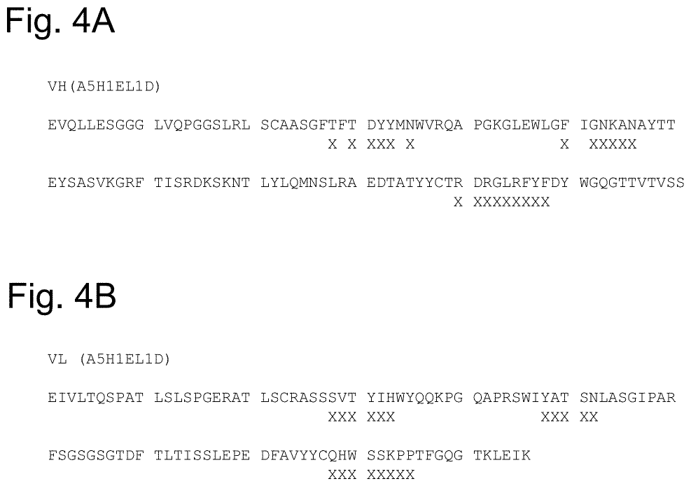

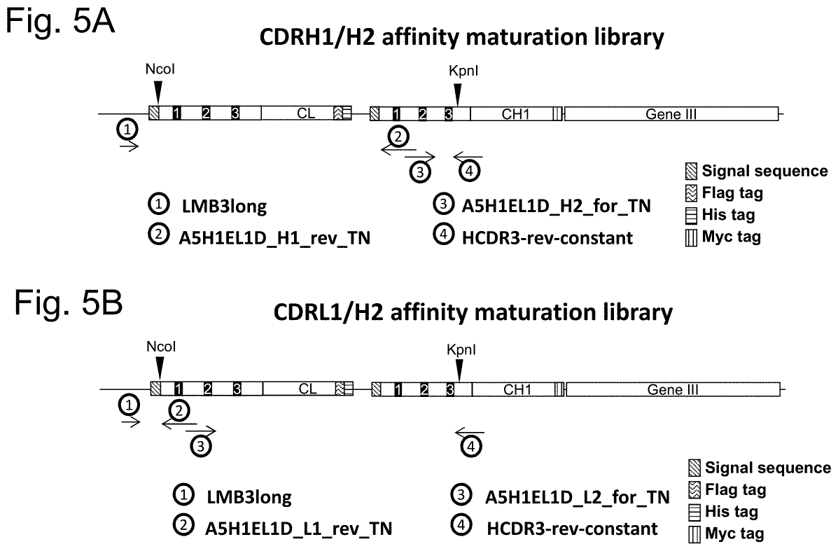

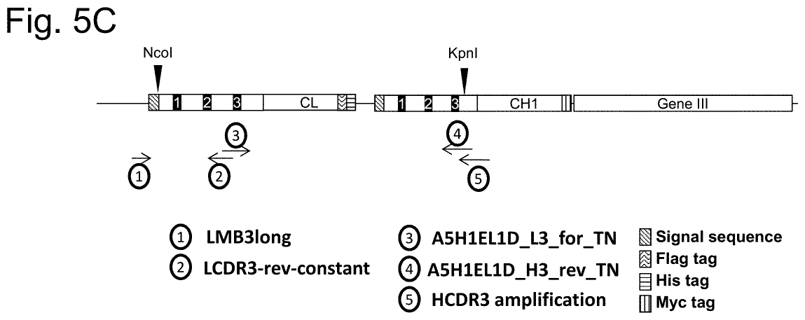

Generation of affinity-matured A5H1EL1D-derived antibodies was carried out by phage display using standard protocols (Silacci et al, 2005). In a first step, DNA sequences encoding the VH and VL of the humanized parental clone A5H1EL1D (amino acid sequences SEQ ID Nos: 23 and 24) were cloned into a phagemid which was then used as a template for randomization. In a next step, three libraries were generated for the selection of favourable clones by phage display. Maturation libraries 1 and 2 were randomized either in CDR1 and CDR2 of the heavy chain or in CDR1 and CDR2 of the light chain. The third maturation library was randomized in the CDR3 regions of both the heavy and the light chain. The randomized positions in the respective CDR regions are shown in FIGS. 4A and 4B. For the generation of the maturation library 1, randomized in CDR1 and 2 of the heavy chain, two fragments were assembled by “splicing by overlapping extension” (SOE) PCR and cloned into the phage vector (FIG. 5A). The following primer combinations were used to generate the library fragments: fragment 1 (LMB3 (SEQ ID NO: 152, Table 7) and A5H1EL1D_H1_rev_TN (SEQ ID NO: 150, Table 7) and fragment 2 (A5H1EL1D_H2_for_TN (SEQ ID NO: 151, Table 7) and HCDR3-rev-constant (SEQ ID NO: 153, Table 22) (Table 7).

TABLE 7
Primers for A5H1EL1D affinity maturation library H1/H2

| | | SEQ ID |
|---|---|---|
| Name | Sequence | NO: |
| A5H1EL1D_ | CA CCA CTC GAG GCC TTT ACC CGG TGC TTG | 150 |
| H1_rev_TN | GCG TAC CCA X17 CAT X16 X15 X14 X13 GAA | |
| | X12 GAA GCC AGA AGC CGC GCA GCT GAG ACG | |
| | X12: 60% T; 5% A/S/G/Y/N/D/E/Q | |
| | X13: 50% T; 20% S; 4.3 A/G/Y/N/D/E/Q | |
| | X14: 50% D; 20% S; 4.3% | |
| | X15: 60% Y; 4% G/V/H/S/E/Q/N/D/R/FG/Y/T/N/A/E/Q | |
| | X16: 50% Y; 20% A; 3.75% G/V/T/H/L/I/R/F | |
| | X17: 50% N; 20% S; 3% D/E/Q/G/Y/V/T/H/A/L | |
| A5H1EL1D_ | CGC CAA GCA CCG GGT AAA GGC CTC GAG TGG | 151 |
| H2_for_TN | CTG GGT X18 ATC X19 X20 X21 X22 X23 GCG | |
| | TAC ACC ACG GAA TAC TCC GCC TCC | |
| | X18: 60% F; 10% A; 6% Y/V/L/I/G | |
| | X19: 50% G; 20% S; 3% A/K/T/V/N/D/E/Q/L/I | |
| | X20: 50% N; 20% G; 3.75% D/E/Q/S/Y/T/H/A | |
| | X21: 60% K; 5% A/T/Y/N/D/E/Q/R | |
| | X22: 60% A; 4% V/G/D/P/H/N/E/Q/L/I | |
| | X23: 60% N; 5% D/E/Q; 4.17% G/T/H/S/A/R | |
| LMB3 long | CAG GAA ACA GCT ATG ACC ATG ATT AC | 152 |
| HCDR3-rev- | AC GGT CAC CGT GGT ACC CTG GCC CCA GTA | 153 |
| constant | GTC GAA ATA GAA GCG CAG ACC AC | |

For the generation of the maturation library 2, randomized in CDR1 and 2 of the light chain, two fragments were assembled by “splicing by overlapping extension” (SOE) PCR and cloned into the phage vector (FIG. 5B). The following primer combinations were used to generate the library fragments: fragment 1 (LMB3 (SEQ ID NO: 152, Table 8) and A5H1EL1D_L1_rev_TN (SEQ ID NO: 154, Table 8) and fragment 2 (A5H1EL1D_L2_for_TN (SEQ ID NO: 155, Table 8) and HCDR3-rev-constant (SEQ ID NO: 153, Table 8).

TABLE 8
Primers for A5H1EL1D affinity maturation library L1/L2

| | | SEQ ID |
|---|---|---|
| Name | Sequence | NO: |
| A5H1EL1D_ | GGA ACG CGG GGC CTG GCC TGG TTT TTG CTG | 154 |
| H1_rev_TN | ATA CCA X06 X05 X04 X03 X02 X01 GCT GGA | |
| | TGC GCG GCA AGA CAG GGT AGC ACG | |
| | x01: 50% S; 20% V; 3.33% T/A/G/N/D/E/Q/Y/H | |
| | X2: 50% V; 20% S; 3.33% T/A/G/N/Q/F/Y/P/H | |
| | X3: 50% T; 20% S; 2.72% | |
| | A/G/Y/V/P/H/N/D/E/Q/R | |
| | X4: 60% Y; 4% F/G/A/V/T/H/S/N/Q/R | |
| | X5: 70% I; 30% L | |
| | X6: 50% H; 20% A; 3.33% R/K/G/S/T/Q/Y/N/V | |
| A5H1EL1D_ | CAG CAA AAA CCA GGC CAG GCC CCG CGT TCC | 155 |
| L2_for_TN | TGG ATC X07 X08 X09 X10 X11 CTC GCT TCT | |
| | GGT ATC CCG GCA CGT TIC TCC GGC | |
| | X7: 60% Y; 10% F; 7.5% H/K/N/S | |
| | X8: 50% A; 20% D; 3.33% V/G/5/T/Y/H/N/E/4 | |
| | X9: 50% T; 20% A; 3.33% 5/G/V/F/H/N/D/E/4 | |
| | X10: 60% S; 4% T/A/G/N/D/E/Q/Y/V/H | |
| | X11: 60% N; 4% D/E/Q/Y/K/T/H/S/A/R | |
| LA1B3 long | CAG GAA ACA GCT ATG ACC ATG ATT AC | 152 |
| HCDR3-rev- | AAC GGT CAC CGT GGT ACC CTG GCC CCA GTA | 153 |
| constant | GTC GAA ATA GAA GCG CAG ACC AC | |

For the generation of the maturation library 3, randomized in CDR3 of the light and heavy chains, two fragments were assembled by “splicing by overlapping extension” (SOE) PCR and cloned into the phage vector (FIG. 5C). The following primer combinations were used to generate the library fragments: fragment 1 (LMB3 (SEQ ID NO:152, Table 9) and LCDR3-rev-constant (SEQ ID NO:158, Table 9) and fragment 2 (A5H1EL1D_L3_for_TN (SEQ ID NO: 156, Table 9) and A5H1EL1D_H3 rev_TN (SEQ ID NO: 157, Table 9).

TABLE 9
Primers for A5H1EL1D affinity maturation library L3/H3

| | | SEQ ID |
|---|---|---|
| Name | Sequence | NO: |
| A5H1EL1D_ | GAG CCT GAA GAT TTT GCC GTA TAC TAT TGT | 156 |
| L3_for_TN | X24 X25 X26 X27 X28 X29 X30 X31 ACT TTC | |
| | GGT CAG GGC ACC AG CTG GAA ATC | |
| | X24: 90% Q; 10% H | |
| | X25: 60% H; 5% R/K/Q/E/Y/F/N/D | |
| | X26: 65% W; 7% F/Y/V/L/I | |
| | X27: 58% S; 4% T/A/G/N/D/E/Q; 2% | |
| | Y/V/P/H/L/I/R | |
| | X28: 58% S; 4% T/A/G/N/D/E/Q; 2% | |
| | Y/V/P/H/L/I/R | |
| | X29: 60% K; 5% R/H; 2.72% | |
| | A/V/T/P/Y/N/D/E/Q/L/I | |
| | X30: 70% P; 5% A/S/T/R/S/L | |
| | X31: 60% P; 5% L/G/R/M; 2.86% A/V/L/I/F/S/R | |
| A5H1EL1D_ | AC GGT CAC CGT GGT ACC CTG GCC CCA GTA | 157 |
| H3_rev_TN | GTC X40 X39 X38 X37 X36 X35 X34 X33 X32 | |
| | AGT ACA GTA GTA GGT GGC GGT GTC TIC TGC | |
| | X32: 60% R; 10% K; 2.72% | |
| | A/V/T/P/Y/N/D/E/Q/L/H | |
| | X33: 60% D; 5% N/E/Q; 2.5% | |
| | G/Y/V/T/H/S/A/L/I/R | |
| | X34: 60% R; 5% K/H; 2.72% | |
| | A/V/T/P/Y/N/D/E/Q/L/I | |
| | X35: 60% G; 5% A/S/T; 2.5% | |
| | Y/V/P/H/N/D/E/Q/L/I | |
| | X36: 60% L; 4% I/V/A/F; 2.4% | |
| | G/Y/T/P/H/S/N/D/E/Q | |
| | X37: 60% R; 5% K/H; 2.72% | |
| | A/V/T/P/Y/N/D/E/Q/L/I | |
| | X38: 65% F; 5% Y/W/A/V/L/I/G | |
| | X39: 60% Y; 5% F/W; 2.14% | |
| | G/A/V/T/P/H/S/N/D/E/Q/L/I/R | |
| | X40: 80% F; 10% I/L | |
| LCDR3-rev- | ACA ATA GTA TAC GGC AAA ATC TTC AGG CTC | 158 |
| constant | | |
| LA4B3 long | CAG GAA ACA GCT ATG ACC ATG ATT AC | 152 |
| HCDR3 | AGA AC GGT CAC CGT GGT ACC CTG GCC CCA | 159 |
| amplification | GTA GTC | |

For the assembly of the fragments of each library, equimolar amounts of each fragment were used and amplified with the respective outer primers. For the assembly of the fragments of the third library, randomized in HCDR3 and LCDR3, primer LMB3 (SEQ ID NO:148, Table 7) was used in combination with the primer “HCDR3 amplification” (SEQ ID NO:155, Table 9). This primer was used in order to extend the C-terminal end of VH with the sequence containing a KpnI site. After assembly of sufficient amounts of full length randomized fragments for all libraries, they were digested with NcoI/KpnI alongside with identically treated acceptor phagemid vector. A 3-fold molar excess of library insert was ligated with 20 μg of phagemid vector. Purified ligations were used for 20 transformations resulting in about 0.7×10⁹ to 2×10⁹ transformants. Phagemid particles displaying the A5H1EL1D affinity maturation libraries were rescued and purified by PEG/NaCl purification to be used for selections.

1.2.2.2 Selection of Affinity Matured A5H1EL1D-Derived Clones

For the selection of affinity-matured clones, phage display selection with all 3 libraries was performed using recombinant soluble antigens. Panning rounds were performed in solution according to the following pattern: 1. Pre-clearing of non-specific phagemid particles by incubation with 200 nM biotinylated NA(B2)A-avi-His and NABA-avi-his for 0.5 h, 2. capture of biotinylated NA(B2)A-avi-His, NABA-avi-his, and bound phagemid particles by addition of 5.4×10⁷ streptavidin-coated magnetic beads for 10 min, 3. Isolation of non-bound phagemid particles from supernatant for further selection, 4. binding of phagemid particles to 20 nM biotinylated N(A2B2)A-avi-His for 0.5 h in a total volume of 1 ml, 5. capture of biotinylated N(A2B2)A-avi-His protein and specifically bound phage particles by addition of 5.4×10⁷ streptavidin-coated magnetic beads for 10 min, 6. washing of beads using 5×1 ml PBS/Tween20 and 5×1 ml PBS, 7. elution of phage particles by addition of 1 ml of 100 mM TEA for 10 min and neutralization by adding 500 μl 1M Tris/HCl pH 7.4, 8. infection of exponentially growing E. coli TG1 bacteria, 9.infection with helperphage VCSM13, and 10. subsequent PEG/NaCl precipitation of phagemid particles to be used in subsequent selection rounds. Selections were carried out over 3 rounds using decreasing antigen concentrations (20×10⁻⁹M, 10×10⁻⁹M, and 2×10⁻⁹M). In round 3, streptavidin beads were washed with 20×1 ml PBS/Tween20 and 5×1 ml PBS.

Specific binders were identified by ELISA as follows: 100 μl of either 10 nM biotinylated N(A2B2)A-avi-His protein or 40 nM biotinylated NA(B2)A-avi-His protein per well were coated on neutravidin plates. Fab-containing bacterial supernatants were added and binding Fabs were detected via their Flag-tags using an anti-Flag/HRP secondary antibody. Clones that were ELISA-positive on recombinant N(A2B2)A-avi-His protein but not on NA(B2)A-avi-His protein were further tested by SPR.

1.2.2.3 Identification of Affinity-Matured A5H1EL1D-Derived Variants by SPR

In order to further characterize the ELISA-positive clones, the off-rate was measured by surface plasmon resonance using a Proteon XPR36 machine and the results were compared with the parental humanized clone A5H1EL1D.

For this experiment, about 2000, 1000, and 500 RU of biotinylated N(A2B2)A-avi-His were immobilized on 3 channels using a Streptavidin-coated NLC chip in vertical orientation. As a control for non-specific binding, 2000 RU of biotinylated NA(B2)A-avi-His protein was immobilized on channel 4. For the off-rate analysis of the identified ELISA-positive clones, injection direction was changed to horizontal orientation. Before injection, each Fab-containing bacterial supernatant was filtered and 3-fold diluted with PBS. The association time was 100 s at 100 μl/minute and dissociation times were either 600 or 1200 s. Bacterial supernatant without Fab fragment was used for referencing. Regeneration was performed with 10 mM glycine pH 1.5 for 35 s at 50 μl/min (vertical orientation).

Dissociation rate constants (koff) were calculated using a simple one-to-one Langmuir binding model in ProteOn Manager v3.1 software by simultaneously fitting the sensorgrams. Clones expressing Fabs with the slowest dissociation rate constants were identified and shortlisted. Shortlisted clones were re-evaluated in an additional SPR experiments under the same conditions. This time, during each injection, 4 affinity-matured clones were directly compared in parallel with the parental clone A5H1EL1D. Bacterial supernatant without Fab fragment was used for referencing. Clones that showed a slower dissociation rate than A5H1EL1D on N(A2B2)A-avi-His and no binding to NA(B2)A-avi-His were selected and the variable domains of the corresponding phagemids were sequenced. The measured dissociation rates of the best clones are shown in Table 10 and the sequences of the respective variable domains are listed in Table 11.

TABLE 10
Kinetic dissociation constants (koff) of selected clones
obtained in screening analysis with bacterial supernatant

| clone | Dissociation constant kd (1/s) |
|---|---|
| A5H1EL1D | 3.10E−04 |
| P006.038 | 7.41E−05 |
| P005.097 | 8.87E−05 |
| P005.103 | 5.37E−05 |
| P002.139 | 6.47E−05 |
| P001.177 | 9.81E−05 |
| P005.102 | 4.24E−05 |

TABLE 11
Amino acid sequences of the parental clone A5H1EL1D and
selected affinity-matured clones

| | | SEQ ID | |
|---|---|---|---|
| Clone | Chain | NO | Sequence |
| A5H1EL1D | VL | 24 | EIVLTQSPATLSLSPGERATLSCRASSSVTYIHWY |
| | | | QQKPGQAPRSWIYATSNLASGIPARFSGSGSGTDF |
| | | | TLTISSLEPEDFAVYYCQHWSSKPPTFGQGTKLEI |
| | | | K |
| | VH | 23 | EVQLLESGGGLVQPGGSLRLSCAASGFTFTDYYMN |
| | | | WVRQAPGKGLEWLGFIGNKANAYTTEYSASVKGRF |
| | | | TISRDKSKNTLYLQMNSLRAEDTATYYCTRDRGLR |
| | | | FYFDYWGQGTTVTVSS |
| P006.038 | VL | 32 | EIVLTQSPATLSLSPGERATLSCRASSSVTYIHWY |
| | | | QQKPGQAPRSWIYATSNLASGIPARFSGSGSGTDF |
| | | | TLTISSLEPEDFAVYYCQHWSSVPPTFGQGTKLEI |
| | | | K |
| | VH | 31 | EVQLLESGGGLVQPGGSLRLSCAASGFTFTDYYMN |
| | | | WVRQAPGKGLEWLGFIGNKANAYTTEYSASVKGRF |
| | | | TISRDKSKNTLYLQMNSLRAEDTATYYCTRDRGIR |
| | | | FGFDYWGQGTTVTVSS |
| P005.097 | VL | 34 | EIVLTQSPATLSLSPGERATLSCRASSSVTYIHWY |
| | | | QQKPGQAPRSWIYATSNLASGIPARFSGSGSGTDF |
| | | | TLTISSLEPEDFAVYYCQHWSSQPPTFGQGTKLEI |
| | | | K |
| | VH | 33 | EVQLLESGGGLVQPGGSLRLSCAASGFTFTDYYMN |
| | | | WVRQAPGKGLEWLGFIGNKANAYTTEYSASVKGRF |
| | | | TISRDKSKNTLYLQMNSLRAEDTATYYCTRDRGLR |
| | | | FSFDYWGQGTTVTVSS |
| P005.103 | VL | 36 | EIVLTQSPATLSLSPGERATLSCRASSSVTYIHWY |
| | | | QQKPGQAPRSWIYATSNLASGIPARFSGSGSGTDF |
| | | | TLTISSLEPEDFAVYYCQHWSSISPTFGQGTKLEI |
| | | | K |
| | VH | 35 | EVQLLESGGGLVQPGGSLRLSCAASGFTFTDYYMN |
| | | | WVRQAPGKGLEWLGFIGNKANAYTTEYSASVKGRF |
| | | | TISRDKSKNTLYLQMNSLRAEDTATYYCTRDRGIR |
| | | | FYFDYWGQGTTVTVSS |
| P002.139 | VL | 38 | EIVLTQSPATLSLSPGERATLSCHASSSVTYIHWY |
| | | | QQKPGQAPRSWIYATSNLASGIPARFSGSGSGTDF |
| | | | TLTISSLEPEDFAVYYCQHWSSKPPTFGQGTKLEI |
| | | | K |
| | VH | 37 | EVQLLESGGGLVQPGGSLRLSCAASGFYFTDYAMN |
| | | | WVRQAPGKGLEWLGVISNKANAYTTEYSASVKGRF |
| | | | TISRDKSKNTLYLQMNSLRAEDTATYYCTRDRGLR |
| | | | FYFDYWGQGTTVTVSS |
| P001.177 | VL | 40 | EIVLTQSPATLSLSPGERATLSCRASSSVTYIHWY |
| | | | QQKPGQAPRSWIYATSNLASGIPARFSGSGSGTDF |
| | | | TLTISSLEPEDFAVYYCQHWSSKPPTFGQGTKLEI |
| | | | K |
| | VH | 39 | EVQLLESGGGLVQPGGSLRLSCAASGFYFTDYYMN |
| | | | WVRQAPGKGLEWLGFISNKANAYTTEYSASVKGRF |
| | | | TISRDKSKNTLYLQMNSLRAEDTATYYCTRDRGLR |
| | | | FYFDYWGQGTTVTVSS |
| P005.102 | VL | 42 | EIVLTQSPATLSLSPGERATLSCRASSSVTYIHWY |
| | | | QQKPGQAPRSWIYATSNLASGIPARFSGSGSGTDF |
| | | | TLTISSLEPEDFAVYYCQHWSSKSPTFGQGTKLEI |
| | | | K |
| | VH | 41 | EVQLLESGGGLVQPGGSLRLSCAASGFTFTDYYMN |
| | | | WVRQAPGKGLEWLGFIGNKANAYTTEYSASVKGRF |
| | | | TISRDKSKNTLYLQMNSLRAEDTATYYCTRDRGIR |
| | | | FQFDYWGQGTTVTVSS |
| P005.102- | VL | 44 | EIVLTQSPATLSLSPGERATLSCRASSSVTYIHWY |
| combo1 | | | QQKPGQAPRSWIYATSNLASGIPARFSGSGSGTDF |
| | | | TLTISSLEPEDFAVYYCQHWSSKSPTFGQGTKLEI |
| | | | K |
| | VH | 43 | EVQLLESGGGLVQPGGSLRLSCASGFYFTDYYMN |
| | | | WVRQAPGKGLEWLGVISNKANAYTTEYSASVKGRF |
| | | | TISRDKSKNTLYLQMNSLRAEDTATYYCTRDRGIR |
| | | | FQFDYWGQGTTVTVSS |
| P005.102- | VL | 46 | EIVLTQSPATLSLSPGERATLSCRASSSVTYIHWY |
| combo2 | | | QQKPGQAPRSWIYATSNLASGIPARFSGSGSGTDF |
| | | | TLTISSLEPEDFAVYYCQHWSSKSPTFGQGTKLEI |
| | | | K |
| | VH | 45 | EVQLLESGGGLVQPGGSLRLSCAASGFYFSDYYMN |
| | | | WVRQAPGKGLEWLGVISNKANAYTTEYSASVKGRF |
| | | | TISRDKSKNTLYLQMNSLRAEDTATYYCTRDRGIR |
| | | | FQFDYWGQGTTVTVSS |
| P005.103- | VL | 48 | EIVLTQSPATLSLSPGERATLSCRASSSVTYIHWY |
| combo1 | | | QQKPGQAPRSWIYATSNLASGIPARFSGSGSGTDF |
| | | | TLTISSLEPEDFAVYYCQHWSSISPTFGQGTKLEI |
| | | | K |
| | VH | 47 | EVQLLESGGGLVQPGGSLRLSCAASGFTFTDYYMN |
| | | | WVRQAPGKGLEWLGFIGNKANAYTTEYSASVKGRF |
| | | | TISRDKSKNTLYLQMNSLRAEDTATYYCTRDRGIR |
| | | | FSFDYWGQGTTVTVSS |
| P005.103- | VL | 50 | EIVLTQSPATLSLSPGERATLSCRASSSVTYIHWY |
| combo2 | | | QQKPGQAPRSWIYATSNLASGIPARFSGSGSGTDF |
| | | | TLTISSLEPEDFAVYYCQHWSSISPTFGQGTKLEI |
| | | | K |
| | VH | 49 | EVQLLESGGGLVQPGGSLRLSCAASGFYFTDYYMN |
| | | | WVRQAPGKGLEWLGVISNKANAYTTEYSASVKGRF |
| | | | TISRDKSKNTLYLQMNSLRAEDTATYYCTRDRGIR |
| | | | FSFDYWGQGTTVTVSS |
| P006.038- | VL | 52 | EIVLTQSPATLSLSPGERATLSCRASSSVTYIHWY |
| combo1 | | | QQKPGQAPRSWIYATSNLASGIPARFSGSGSGTDF |
| | | | TLTISSLEPEDFAVYYCQHWSSVPPTFGQGTKLEI |
| | | | K |
| | VH | 51 | EVQLLESGGGLVQPGGSLRLSCAASGFYFTDYAMN |
| | | | WVRQAPGKGLEWLGVISNKANAYTTEYSASVKGRF |
| | | | TISRDKSKNTLYLQMNSLRAEDTATYYCTRDRGIR |
| | | | FGFDYWGQGTTVTVSS |
| P006.038- | VL | 54 | EIVLTQSPATLSLSPGERATLSCRASSSVTYIHWY |
| combo2 | | | QQKPGQAPRSWIYATSNLASGIPARFSGSGSGTDF |
| | | | TLTISSLEPEDFAVYYCQHWSSVPPTFGQGTKLEI |
| | | | K |
| | VH | 53 | EVQLLESGGGLVQPGGSLRLSCAASGFTFSDYEMN |
| | | | WVRQAPGKGLEWLGFISNKANAYTTEYSASVKGRF |
| | | | TISRDKSKNTLYLQMNSLRAEDTATYYCTRDRGIR |
| | | | FGFDYWGQGTTVTVSS |

1.2.2.4 Fab Purification of Affinity-Matured A5H1EL1D Clones

In order to further characterize the affinity-matured clones, the respective Fab fragments were purified for the exact analysis of the kinetic parameters. For each clone, a 500 ml culture was inoculated with bacteria harboring the corresponding phagemid and induced with 1 mM IPTG at an optical density measured at 600 nm (0D600) of 0.9. Afterwards, the cultures were incubated at 25° C. overnight and harvested by centrifugation. After incubation of the re-suspended pellet for 20 min in 25 ml PPB buffer (30 mM Tris-HCl pH8, 1 mM EDTA, 20% sucrose), bacteria were centrifuged again and the supernatant was harvested. This incubation step was repeated once with 25 ml of a 5 mM MgSO4 solution. The supernatants of both incubation steps were pooled, filtered and loaded on an IMAC column (His gravitrap, GE Healthcare). Subsequently, the column was washed with 40 ml washing buffer (500 mM NaCl, 20 mM Imidazole, 20 mM NaH₂PO₄ pH 7.4). After the elution (500 mM NaCl, 500 mM Imidazole, 20 mM NaH₂PO₄ pH 7.4) the eluate was re-buffered using PD10 columns (GE Healthcare). The yield of purified protein was in the range of 300 to 500 μg/l.

1.2.2.5 SPR Analysis of Purified Affinity-Matured A5H1EL1D Fab Fragments

Affinity (KD) of purified Fab fragments was measured by surface plasmon resonance using a Proteon XPR36 machine using the same setup as described before.

About 2000, 1000, 500, and 250 RU of biotinylated N(A2B2)A-avi-His were immobilized on 4 channels of a Streptavidin-coated NLC chip in vertical orientation. As a control for non-specific binding, 2000 RU of biotinylated NA(B2)A-avi-His protein was immobilized on channel 5. For the determination of the affinity (KD) of the purified clones, injection direction was changed to horizontal orientation. Two-fold dilution series of purified Fab fragments (varying concentration ranges between 100 and 3 nM) were injected simultaneously at 100 μl/min along separate channels 1-5, with association times of 100 s, and dissociation times of 1200 s. Buffer (PBST) was injected along the sixth channel to provide an “in-line” blank for referencing. Regeneration was performed with 10 mM glycine pH 1.5 for 35 s at 50 μl/min (vertical orientation).

Association rate constants (kon) and dissociation rate constants (koff) were calculated using a simple one-to-one Langmuir binding model in ProteOn Manager v3.1 software by simultaneously fitting the association and dissociation sensorgrams. The equilibrium dissociation constant (KD) was calculated as the ratio koff/kon. The kinetic and thermodynamic data are listed in Table 12.

TABLE 12
Determination of kinetic and thermodynamic
parameters of purified Fab-fragments by SPR

| Clone ID | k on (1/Ms) | k off (1/s) | K_{D} (nM) |
|---|---|---|---|
| A5H1EL1D | 1.08E+5 | 2.48E−4 | 2.3 |
| P006.038 | 2.25E+5 | 5.78E−5 | 0.25 |
| P005.097 | 0.94E+5 | 8.54E−5 | 0.91 |
| P005.103 | 1.00E+5 | 4.99E−5 | 0.5 |
| P002.139 | 1.05E+5 | 6.53E−5 | 0.63 |
| P001.177 | 2.67E+5 | 7.85E−4 | 0.29 |
| P005.102 | 1.34E+5 | 3.92E−4 | 0.29 |

1.2.2.6 Combination of CDR Positions of Affinity-Matured Clones

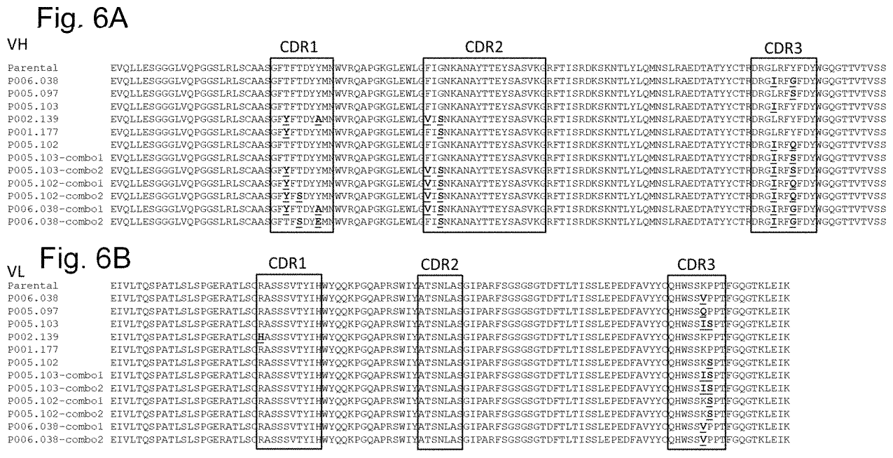

In an attempt to further increase the affinity to CEA, CDR positions of several previously identified affinity-matured binders were combined with each other. This includes not only specific positions within a CDR but also combinations of CDRs from different binders. An alignment of all clones, phage display-derived and combinatorial clones, is shown in FIGS. 6A and 6B. The CDRs of all heavy chains and light chains are listed in Table 13 and 14, respectively. The VH and VL domains are summarized in Table 11.

TABLE 13
CDR sequences of affinity-matured heavy chains

| | SEQ | | SEQ | | SEQ | |
|---|---|---|---|---|---|---|
| | ID | | ID | | ID | |
| Clone | NO | LCDR1 | NO | LCDR2 | NO | LCDR3 |
| A5H1EL1D | 20 | GFTFTDYY | 21 | FIGNKANAYT | 22 | DRGLRFYFDY |
| | | MN | | TEYSASVKG | | |
| P006.038 | 163 | GFTFTDYY | 164 | FIGNKANAYT | 165 | DRGIRFGFDY |
| | | MN | | TEYSASVKG | | |
| P005.097 | 169 | GFTFTDYY | 170 | FIGNKANAYT | 171 | DRGLRFSFDY |
| | | MN | | TEYSASVKG | | |
| P005.103 | 175 | GFTFTDYY | 176 | FIGNKANAYT | 177 | DRGIRFYFDY |
| | | MN | | TEYSASVKG | | |
| P002.139 | 181 | GFYFTDYA | 182 | VISNKANAYT | 183 | DRGLRFYFDY |
| | | MN | | TEYSASVKG | | |
| P001.177 | 187 | GFYFTDYY | 188 | FISNKANAYT | 189 | DRGLRFYFDY |
| | | MN | | TEYSASVKG | | |
| P005.102 | 193 | GFTFTDYY | 194 | FIGNKANAYT | 195 | DRGIRFQFDY |
| | | MN | | TEYSASVKG | | |
| P005.102- | 199 | GFYFTDYY | 200 | VISNKANAYT | 201 | DRGIRFQFDY |
| combo1 | | MN | | TEYSASVKG | | |
| P005.102- | 205 | GFYFSDYY | 206 | VISNKANAYT | 207 | DRGIRFQFDY |
| combo2 | | MN | | TEYSASVKG | | |
| P005.103- | 211 | GFTFTDYY | 212 | FIGNKANAYT | 213 | DRGIRFSFDY |
| combo1 | | MN | | TEYSASVKG | | |
| P005.103- | 217 | GFYFTDYY | 218 | VISNKANAYT | 219 | DRGIRFSFDY |
| combo2 | | MN | | TEYSASVKG | | |
| P006.038- | 223 | GFYFTDYA | 224 | VISNKANAYT | 225 | DRGIRFGFDY |
| combo1 | | MN | | TEYSASVKG | | |
| P006.038- | 229 | GFTFSDYE | 230 | FISNKANAYT | 231 | DRGIRFGFDY |
| combo2 | | MN | | TEYSASVKG | | |

TABLE 14
CDR sequences of affinity-matured light chains

| | SEQ | | SEQ | | SEQ | |
|---|---|---|---|---|---|---|
| | ID | | ID | | ID | |
| Clone | NO | LCDR1 | NO | LCDR2 | NO | LCDR3 |
| A5H1EL1D | 17 | RASSSVTYIH | 18 | ATSNLAS | 19 | QHWSSKPPT |
| P006.038 | 160 | RASSSVTYIH | 161 | ATSNLAS | 162 | QHWSSVPPT |
| P005.097 | 167 | RASSSVTYIH | 168 | ATSNLAS | 169 | QHWSSQPPT |
| P005.103 | 172 | RASSSVTYIH | 173 | ATSNLAS | 174 | QHWSSISPT |
| P002.139 | 178 | HASSSVTYIH | 179 | ATSNLAS | 180 | QHWSSKPPT |
| P001.177 | 184 | RASSSVTYIH | 185 | ATSNLAS | 186 | QHWSSKPPT |
| P005.102 | 190 | RASSSVTYIH | 191 | ATSNLAS | 192 | QHWSSKSPT |
| P005.102- | 196 | RASSSVTYIH | 197 | ATSNLAS | 198 | QHWSSKSPT |
| combo1 | | | | | | |
| P005.102- | 202 | RASSSVTYIH | 203 | ATSNLAS | 204 | QHWSSKSPT |
| combo2 | | | | | | |
| P005.103- | 208 | RASSSVTYIH | 209 | ATSNLAS | 210 | QHWSSISPT |
| combo1 | | | | | | |
| P005.103- | 214 | RASSSVTYIH | 215 | ATSNLAS | 216 | QHWSSISPT |
| combo2 | | | | | | |
| P006.038- | 220 | RASSSVTYIH | 221 | ATSNLAS | 222 | QHWSSVPPT |
| combo1 | | | | | | |
| P006.038- | 226 | RASSSVTYIH | 227 | ATSNLAS | 228 | QHWSSVPPT |
| combo2 | | | | | | |

### 1.3 Generation of Humanized Variants of Anti-CEA Antibody MFE23

### 1.3.1 Methodology

Anti-CEA antibody MFE23 is for example disclosed by M. K. Boehm et al, Biochem. J. 2000, 346, 519-528 and its structure can be found as PDB ID:11QOK in the Protein structural database PDB (www.rcsb.org, H. M. Berman et al, The Protein Data Bank, Nucleic Acids Research, 2000, 28, 235-242). This entry includes the heavy and the light chain variable domain sequence. For the identification of a suitable human acceptor framework during the humanization of the anti-CEA binder 1MFE23, a classical approach was taken by searching for an acceptor framework with high sequence homology, grafting of the CDRs on this framework, and evaluating which back-mutations can be envisaged. More explicitly, each amino acid difference of the identified frameworks to the parental antibody was judged for impact on the structural integrity of the binder, and back mutations towards the parental sequence were introduced whenever appropriate. The structural assessment was based on Fv region homology models of both the parental antibody and its humanized versions created with an in-house antibody structure homology modeling tool implemented using the Biovia Discovery Studio Environment, version 4.5.

In order to increase confidence in the choice of back mutations, we identified the closest murine homologous sequence, from which this antibody might have derived. There we looked for positions that have undergone extensive somatic hypermutation during the maturation of this antibody in the murine B-cell. Those mutations would be of potential importance to be incorporated in the humanized construct.

### 1.3.2 Choice of Acceptor Framework and Adaptations Thereof

The acceptor framework was chosen as described in Table 15 below:

TABLE 15
Acceptor framework

| | | Choice of human |
|---|---|---|
| | Closest murine | acceptor V-region |
| | V-region germline | germline |
| MFE23 VH | m-IGHV14-4-02 | IGHV1-2-02 |
| MFE23 VL | m-IGKV4-57-01 | IGKV1-39-01 |

Post-CDR3 framework regions were adapted from human J-element germline IGHJ4-01 for the heavy chain, and a sequence similar to the kappa J-element IGKJ4-01, for the light chain. Based on structural considerations, back mutations from the human acceptor framework to the amino acid in the parental binder were introduced at Kabat positions 71 and 93 of the heavy chain. Based on considerations that framework mutations in the murine germline, leading to the final matured 1MFE23 sequence, would be of importance, the residues at Kabat position 94 of VH was changed back to the murine sequence.

In order to evaluate further affinity and/or stability improvements on the MFE23 sequence, we incorporated the following mutations in the light chain sequence: Phe26Leu, Ser30Pro, or Tyr, Leu78Val as described by C. P. Graff et al., Protein Engineering, Design & Selection 2004, 17(4), 293-304.

### 1.3.3 VII and VL Domains of the Resulting Humanized CEA Antibodies

The resulting VH domains of humanized CEA antibodies can be found in Table 16 below and the resulting VL domains of humanized CEA antibodies are listed in Table 17 below.

TABLE 16
Amino acid sequences of the VII domains of humanized CEA antibodies,
based on human acceptor framework IGHV1-2-02

| | | Seq ID |
|---|---|---|
| Description | Sequence | No |
| MFE23 VH | QVKLQQSGAELVRSGTSVKLSCTASGFNIKDSYMHWLRQGPEQGLEWIGW | 63 |
| murine donor | IDPENGDTEYAPKFQGKATFTTDTSSNTAYLQLSSLTSEDTAVYYCNEGT | |
| sequence | PTGPYYFDYWGQGTTVTVSS | |
| IGHV1-2-02 | QLVQSGAEVKKPGASVKVSCKASGYTFTGYYMHWVRQAPGQGLEWMGWIN | 232 |
| human | PNSGGTNYAQKFQGRVTMTRDTSISTAYMELSRLRSDDTAVYYCAR | |
| acceptor | | |
| sequence | | |
| IGHV1-69-01 | QVQLVQSGAEVKKPGSSVKVSCKASGGTFSSYAISWVRQAPGQGLEWMGG | 233 |
| human | IIPIFGTANYAQKFQGRVTITADESTSTAYMELSSLRSEDTAVYYCAR | |
| acceptor | | |
| sequence | | |
| IGHV1-69-05 | QVQLVQSGAEVKKPGSSVKVSCKASGGTFSSYAISWVRQAPGQGLEWMGG | 234 |
| human | IIPIFGTANYAQKFQGRVTITTDESTSTAYMELSSLRSEDTAVYYCAR | |
| acceptor | | |
| sequence | | |
| Humanized | | |
| variants | | |
| MFE-H24 | QVQLVQSGAEVKKPGASVKVSCKASGFNIKDSYMHWVRQAPGQGLEWMGW | 75 |
| | IDPENGDTEYAPKFQGRVTMTTDTSISTAYMELSRLRSDDTAVYYCNEGT | |
| | PTGPYYFDYWGQGTLVTVSS | |
| MFE-H25 | QVQLVQSGAEVKKPGASVKVSCKASGYTFKDSYMHWVRQAPGQGLEWMGW | 76 |
| | IDPENGDTEYAPKFQGRVTMTTDTSISTAYMELSRLRSDDTAVYYCNEGT | |
| | PTGPYYFDYWGQGTLVTVSS | |
| MFE-H26 | QVQLVQSGAEVKKPGASVKVSCKASGFNIKDSYMHWVRQAPGQGLEWMGW | 77 |
| | IDPENGGTNYAQKFQGRVTMTTDTSISTAYMELSRLRSDDTAVYYCNEGT | |
| | PTGPYYFDYWGQGTLVTVSS | |
| MFE-H27 | QVQLVQSGAEVKKPGASVKVSCKASGFNIKDSYMHWVRQAPGQGLEWMGW | 78 |
| | IDPENGDTEYAPKFQGRVTMTTDTSISTAYMELSRLRSDDTAVYYCARGT | |
| | PTGPYYFDYWGQGTLVTVSS | |
| MFE-H28 | QVQLVQSGAEVKKPGASVKVSCKASGFNIKDSYMHWVRQAPGQGLEWMGW | 79 |
| | IDPENGDTEYAPKFQGRVTMTRDTSISTAYMELSRLRSDDTAVYYCNEGT | |
| | PTGPYYFDYWGQGTLVTVSS | |
| MFE-H29 | QVQLVQSGAEVKKPGSSVKVSCKASGFNIKDSYMHWVRQAPGQGLEWMGW | 80 |
| | IDPENGDTEYAPKFQGRVTITTDESTSTAYMELSSLRSEDTAVYYCNEGT | |
| | PTGPYYFDYWGQGTLVTVSS | |

TABLE 17
Amino acid sequences of the VL domains of humanized CEA
antibodies, based on human acceptor framework IGKV1-39-01

| | | Seq ID |
|---|---|---|
| Description | Sequence | No |
| MFE23 VL | ENVLTQSPAIMSASPGEKVTITCSASSSVSYMHWFQQKPGTSPKLWIYST | 64 |
| murine donor | SNLASGVPARFSGSGSGTSYSLTISRMEAEDAATYYCQQRSSYPLTFGAG | |
| sequence | TKLELK | |
| IGKV1-39-01 | DIQMTQSPSSLSASVGDRVTITCRASQSISSYLNWYQQKPGKAPKLLIYA | 235 |
| human | ASSLQSGVPSRFSGSGSGTDFTLTISSLQPEDFATYYCQQSYSTP | |
| acceptor | | |
| sequence | | |
| Humanized | | |
| variants | | |
| MFE-L24 | DIQMTQSPSSLSASVGDRVTITCRASSSVSYMHWYQQKPGKAPKLLIYST | 81 |
| | SNLASGVPSRFSGSGSGTDFTLTISSLQPEDFATYYCQQRSSYPLTFGGG | |
| | TKLEIK | |
| MFE-L25 | EIQMTQSPSSLSASVGDRVTITCRASSSVSYMHWYQQKPGKAPKLLIYST | 82 |
| | SNLASGVPSRFSGSGSGTDFTLTISSLQPEDFATYYCQQRSSYPLTFGGG | |
| | TKLEIK | |
| MFE-L26 | EIQMTQSPSSLSASVGDRVTITCRASQSISSYMHWYQQKPGKAPKLLIYS | 83 |
| | TSNLASGVPSRFSGSGSGTDFTLTISSLQPEDFATYYCQQRSSYPLTFGG | |
| | GTKLEIK | |
| MFE-L27 | EIQMTQSPSSLSASVGDRVTITCRASSSVPYMHWYQQKPGKAPKLLIYST | 84 |
| | SNLASGVPSRFSGSGSGTDFTLTISSVQPEDFATYYCQQRSSYPLTFGGG | |
| | TKLEIK | |
| MFE-L28 | EIQMTQSPSSLSASVGDRVTITCRASSSVPYMHWLQQKPGKAPKLLIYST | 85 |
| | SNLASGVPSRFSGSGSGTDFTLTISSVQPEDFATYYCQQRSSYPLTFGGG | |
| | TKLEIK | |
| MFE-L29 | EIQMTQSPSSLSASVGDRVTITCRASSSVPYMHWLQQKPGKAPKLLIYST | 86 |
| | SSLQSGVPSRFSGSGSGTDFTLTISSVQPEDFATYYCQQRSSYPLTFGGG | |
| | TKLEIK | |

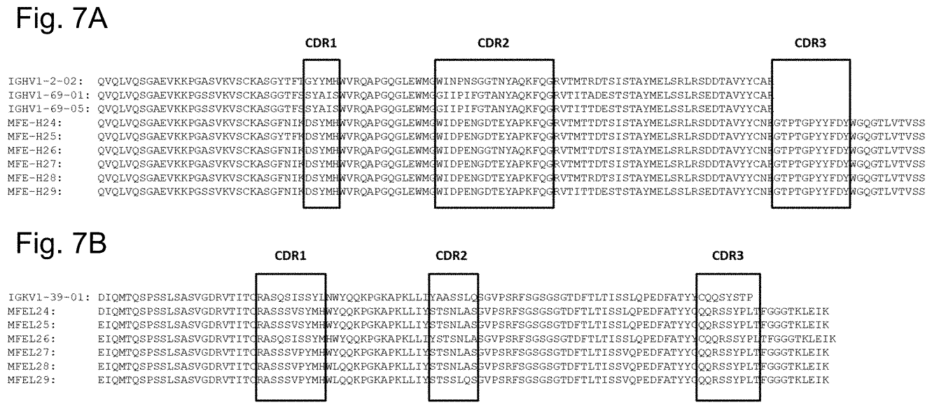

FIG. 7 shows an alignment of the sequences as listed in Table 16 and 17, respectively.

The variable region of six heavy and six light chain DNA sequences, encoding the humanized CEA binder, were subcloned in frame with either the constant heavy chain or the constant light chain of human IgG1 containing P239G, L234A and L235A mutations to abrogate binding to Fcγ receptors (WO 2012/130831 A1). The antibodies were produced as described below. The resulting 36 variants (Table 18) were tested for binding on MKN45 cells; and 7 variants were selected for further development.

TABLE 18
Nomenclature for VH/VL combinations expressed
as huIgG1_LALA_PG antibodies

| | MFE-L24 | MFE-L25 | MFE-L26 | MFE-L27 | MFE-L28 | MFE-L29 |
|---|---|---|---|---|---|---|
| MFE-H24 | P1AE3125 | P1AE3119 | P1AE3113 | P1AE3107 | P1AE3101 | P1AE3095 |
| MFE-H25 | P1AE3124 | P1AE3118 | P1AE3112 | P1AE3106 | P1AE3100 | P1AE3094 |
| MFE-H26 | P1AE3123 | P1AE3117 | P1AE3111 | P1AE3105 | P1AE3099 | P1AE3093 |
| MFE-H27 | P1AE3122 | P1AE3116 | P1AE3110 | P1AE3104 | P1AE3098 | P1AE3092 |
| MFE-H28 | P1AE3121 | P1AE3115 | P1AE3109 | P1AE3103 | P1AE3097 | P1AE3091 |
| MFE-H29 | P1AE3120 | P1AE3114 | P1AE3108 | P1AE3102 | P1AE3096 | P1AE3090 |

### 1.3.4 Selection of Humanized MFE23 Antibodies

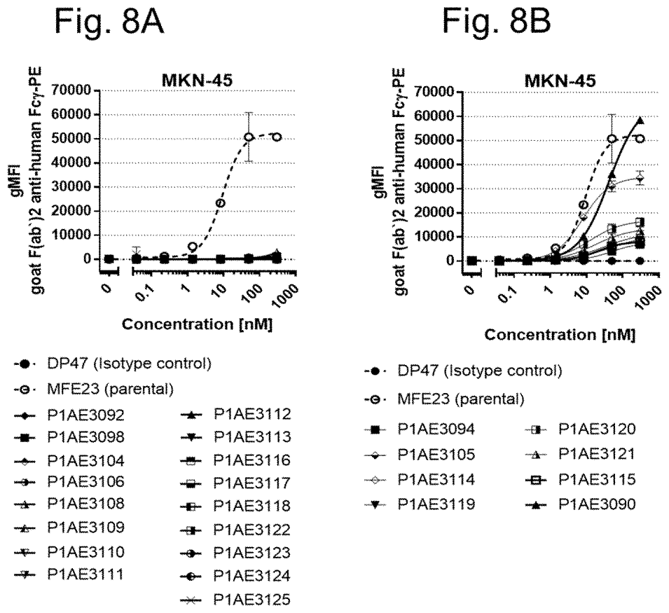

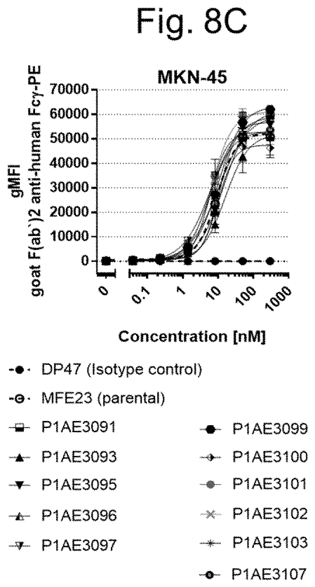

Binding of the 36 humanized MFE23 huIgG1 P329G LALA variants to CEA expressed on MKN45 cells was compared to the respective parental murine MFE23 huIgG1 P329G LALA antibody. Seventeen clones lost their binding capacity to human CEACAM5 expressing MKN45 cells (FIG. 8A). Eight clones showed reduced binding if compared to the parental clone MFE23 (FIG. 8B). Eleven clones showed similar binding if compared to the parental clone 1MFE23 (FIG. 8C). The fitting EC₅₀ values and area under the curve values (AUC) of these binding curves are displayed in Table 19.

TABLE 19
EC₅₀ values and area under the curve (AUC) of binding
curves of different humanized MFE23 huIgG1 P329G
LALA antibodies displayed in FIGS. 8A, 8B and 8C

| binding to MKN45 | EC₅₀ [nM] | AUC |
|---|---|---|
| P1AD5108-002 DP47 huIgG1 PG LALA | n.d. | 420 |
| (Isotype control) | | |
| P1AE0096-001 MFE23 huIgG1 PG LALA | 9.36 | 83489 |
| (parental) | | |
| P1AE3104 | n.d. | 414 |
| P1AE3122 | n.d. | 422 |
| P1AE3116 | n.d. | 443 |
| P1AE3092 | n.d. | 482 |
| P1AE3110 | n.d. | 547 |
| P1AE3112 | n.d. | 551 |
| P1AE3118 | n.d. | 559 |
| P1AE3111 | n.d. | 578 |
| P1AE3109 | n.d. | 695 |
| P1AE3113 | n.d. | 757 |
| P1AE3124 | n.d. | 847 |
| P1AE3123 | n.d. | 1090 |
| P1AE3106 | n.d. | 1126 |
| P1AE3117 | n.d. | 1181 |
| P1AE3098 | n.d. | 1191 |
| P1AE3108 | n.d. | 2086 |
| P1AE3125 | n.d. | 3145 |
| P1AE3094 | 47.57 | 6908 |
| P1AE3115 | 20.68 | 10177 |
| P1AE3119 | 41.90 | 10327 |
| P1AE3114 | 30.60 | 12769 |
| P1AE3121 | 17.13 | 17464 |
| P1AE3120 | 12.37 | 24181 |
| P1AE3105 | 7.92 | 55868 |
| P1AE3090 | 42.00 | 61809 |
| P1AE3093 | 16.14 | 68082 |
| P1AE3100 | 9.89 | 74137 |
| P1AE3091 | 14.60 | 83061 |
| P1AE3095 | 11.12 | 84917 |
| P1AE3096 | 10.46 | 87775 |
| P1AE3107 | 5.83 | 91203 |
| P1AE3102 | 5.41 | 91481 |
| P1AE3103 | 5.88 | 92448 |
| P1AE3099 | 10.21 | 95311 |
| P1AE3097 | 6.45 | 98656 |
| P1AE3101 | 6.58 | 103966 |

### Example 2

### Generation and Production of CEA-Targeting 4-1BB Agonistic Antigen Binding Molecules

### 2.1 Preparation of CEA-Targeting 4-1BB Ligand Trimer-Containing Fc Fusion Antigen Binding Molecules

Asymmetric human IgG1 molecules with knob-into hole mutations were prepared as follows: The variable region of heavy and light chain DNA sequences encoding a binder specific for CEA, were subcloned in frame with either the constant heavy chain of the hole or the constant light chain of human IgG1. The DNA sequence encoding part of the ectodomain (amino acid 71-248) of human 4-1BB ligand was synthetized according to the P41273 sequence of Uniprot database.

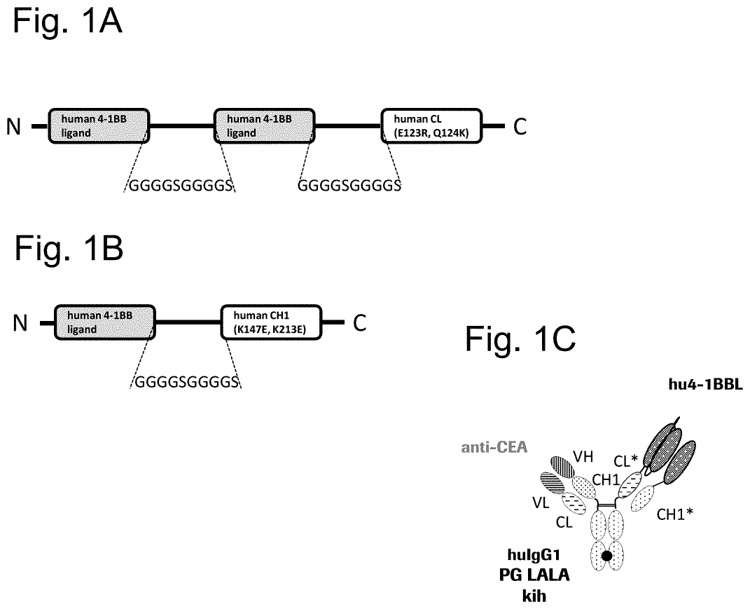

A polypeptide containing two ectodomains of 4-1BB ligand, separated by (G4S)2 linkers, and fused to the human IgG1-CL domain, was cloned as depicted in FIG. 1A: human 4-1BB ligand, (G4S)2 connector, human 4-1BB ligand, (G4S)2 connector, human CL. A polypeptide containing one ectodomain of 4-1BB ligand and fused to the human IgG1-CH domain, was cloned as described in FIG. 1B: human 4-1BB ligand, (G4S)2 connector, human CH.

To improve correct pairing, the following mutations were introduced in the crossed CH-CL. In the dimeric 4-1BB ligand fused to human CL the mutations E123R and Q124K were introduced. In the monomeric 4-1BB ligand fused to human CH1, the mutations K147E and K213E were cloned into the human CH1 domain as described in International Patent Appl. Publ. No. WO 2015/150447.

In the Fc domain the P329G, L234A and L235A mutations were introduced in the constant region of the knob and hole heavy chains to abrogate binding to Fc gamma receptors according to the method described in International Patent Appl. Publ. No. WO 2012/130831. Combination of the dimeric ligand-Fc knob chain containing the S354C/T366W mutations, the monomeric CH1 fusion, the targeted anti-CEA-Fc hole chain containing the Y349C/T366S/L368A/Y407V mutations and the anti-CEA light chain allowed the generation of a heterodimer, which includes an assembled trimeric 4-1BB ligand and a CEA binding Fab (FIG. 1C).

Table 20 and 21 show the amino acid sequences of the monovalent CEA-targeted split trimeric 4-1BB ligand Fc (kih) fusion antigen binding molecule containing CH1-CL crossover and charged residues based on CEA binder A5B7 and its humanized version.

TABLE 20
Amino acid sequences of monovalent CEA(murine A5B7) targeted split
trimeric 4-1B ligand antigen binding molecule with CH-CL
crossover and charged residues (CEA(A5B7)-4-1BBL)

| SEQ ID | | |
|---|---|---|
| NO: | Description | Sequence |
| 99 | Dimeric | REGPELSPDDPAGLLDLRQGMFAQLVAQNVLLIDGPLSWYSDPG |
| | 4-1BBL-CL* | LAGVSLTGGLSYKEDTKELVVAKAGVYYVFFQLELRRVVAGEGS |
| | Fc knob chain | GSVSLALHLQPLRSAAGAAALALTVDLPPASSEARNSAFGFQGR |
| | | LLHLSAGQRLGVHLHTEARARHAWQLTQGATVLGLFRVTPEIPA |
| | | GLGGGGSGGGGSREGPELSPDDPAGLLDLRQGMFAQLVAQNVLL |
| | | IDGPLSWYSDPGLAGVSLTGGLSYKEDTKELVVAKAGVYYVFFQ |
| | | LELRRVVAGEGSGSVSLALHLQPLRSAAGAAALALTVDLPPASS |
| | | EARNSAFGFQGRLLHLSAGQRLGVHLHTEARARHAWQLTQGATV |
| | | LGLFRVTPEIPAGLGGGGSGGGGSRTVAAPSVFIFPPSDRKLKS |
| | | GTASVVCLLNNFYPREAKVQWKVDNALQSGNSQESVTEQDSKDS |
| | | TYSLSSTLTLSKADYEKHKVYACEVTHQGLSSPVTKSFNRGECD |
| | | KTHTCPPCPAPEAAGGPSVFLFPPKPKDTLMISRTPEVTCVVVD |
| | | VSHEDPEVKFNWYVDGVEVHNAKTKPREEQYNSTYRVVSVLTVL |
| | | HQDWLNGKEYKCKVSNKALGAPIEKTISKAKGQPREPQVYTLPP |
| | | CRDELTKNQVSLWCLVKGFYPSDIAVEWESNGQPENNYKTTPPV |
| | | LDSDGSFFLYSKLTVDKSRWQQGNVFSCSVMHEALHNHYTQKSL |
| | | SLSP |
| 100 | Monomeric | REGPELSPDDPAGLLDLRQGMFAQLVAQNVLLIDGPLSWYSDPG |
| | 4-1BBL-CH1* | LAGVSLTGGLSYKEDTKELVVAKAGVYYVFFQLELRRVVAGEGS |
| | | GSVSLALHLQPLRSAAGAAALALTVDLPPASSEARNSAFGFQGR |
| | | LLHLSAGQRLGVHLHTEARARHAWQLTQGATVLGLFRVTPEIPA |
| | | GLGGGGSGGGGSASTKGPSVFPLAPSSKSTSGGTAALGCLVEDY |
| | | FPEPVTVSWNSGALTSGVHTFPAVLQSSGLYSLSSVVTVPSSSL |
| | | GTQTYICNVNHKPSNTKVDEKVEPKSC |
| 236 | anti-CEA | EVKLVESGGGLVQPGGSLRLSCATSGETFTDYYMNWVRQPPGKA |
| | (A5B7) | LEWLGFIGNKANGYTTEYSASVKGRFTISRDKSQSILYLQMNTL |
| | Fc hole chain | RAEDSATYYCTRDRGLRFYFDYWGQGTTLTVSSASTKGPSVFPL |
| | | APSSKSTSGGTAALGCLVKDYFPEPVTVSWNSGALTSGVHTFPA |
| | | VLQSSGLYSLSSVVTVPSSSLGTQTYICNVNHKPSNTKVDKKVE |
| | | PKSCDKTHTCPPCPAPEAAGGPSVFLFPPKPKDTLMISRTPEVT |
| | | CVVVDVSHEDPEVKFNWYVDGVEVHNAKTKPREEQYNSTYRVVS |
| | | VLTVLHQDWLNGKEYKCKVSNKALGAPIEKTISKAKGQPREPQV |
| | | CTLPPSRDELTKNQVSLSCAVKGFYPSDIAVEWESNGQPENNYK |
| | | TTPPVLDSDGSFELVSKLTVDKSRWQQGNVESCSVMHEALHNHY |
| | | TQKSLSLSP |
| 237 | anti- | QTVLSQSPAILSASPGEKVTMTCRASSSVTYIHWYQQKPGSSPK |
| | CEA(A5B7) | SWIYATSNLASGVPARFSGSGSGTSYSLTISRVEAEDAATYYCQ |
| | light chain | HWSSKPPTFGGGTKLEIKRTVAAPSVFIFPPSDEQLKSGTASVV |
| | | CLLNNFYPREAKVQWKVDNALQSGNSQESVTEQDSKDSTYSLSS |
| | | TLTLSKADYEKHKVYACEVTHQGLSSPVTKSFNRGEC |

*stands for charged residues

TABLE 21
Amino acid sequences of monovalent CEA(A5H1EL1D) targeted split
trimeric 4-1BB ligand antigen binding molecule with CH-CL
crossover and charged residues (CEA(A5H1EL1D)-4-1BBL)

| SEQ ID | | |
|---|---|---|
| NO: | Description | Sequence |
| 99 | Dimeric | See Table 20 |
| | 4-1BBL-CL* | |
| | Fc knob chain | |
| 100 | Monomeric | See Table 20 |
| | 4-1BBL-CH1* | |
| 238 | anti-CEA | EVQLLESGGGLVQPGGSLRLSCAASGFTFTDYYMNWVRQAPGKG |
| | (A5H1EL1D)- | LEWLGFIGNKANAYTTEYSASVKGRFTISRDKSKNTLYLQMNSL |
| | Fc hole | RAEDTATYYCTRDRGLRFYFDYWGQGTTVTVSSASTKGPSVFPL |
| | chain | APSSKSTSGGTAALGCLVKDYFPEPVTVSWNSGALTSGVHTFPA |
| | | VLQSSGLYSLSSVVTVPSSSLGTQTYICNVNHKPSNTKVDKKVE |
| | | PKSCDKTHTCPPCPAPEAAGGPSVFLEPPKPKDTLMISRTPEVT |
| | | CVVVDVSHEDPEVKFNWYVDGVEVHNAKTKPREEQYNSTYRVVS |
| | | VLTVLHQDWLNGKEYKCKVSNKALGAPIEKTISKAKGQPREPQV |
| | | CTLPPSRDELTKNQVSLSCAVKGFYPSDIAVEWESNGQPENNYK |
| | | TTPPVLDSDGSFELVSKLTVDKSRWQQGNVESCSVMHEALHNHY |
| | | TQKSLSLSP |
| 239 | anti-CEA | EIVLTQSPATLSLSPGERATLSCRASSSVTYIHWYQQKPGQAPR |
| | (A5H1EL1D) | SWIYATSNLASGIPARFSGSGSGTDFTLTISSLEPEDFAVYYCQ |
| | light chain | HWSSKPPTFGQGTKLEIKRTVAAPSVFIFPPSDEQLKSGTASVV |
| | | CLLNNFYPREAKVQWKVDNALQSGNSQESVTEQDSKDSTYSLSS |
| | | TLTLSKADYEKHKVYACEVTHQGLSSPVTKSFNRGEC |

Tables 22 to 33 show the amino acid sequences of monovalent CEA-targeted split trimeric 4-1BB ligand Fc (kih) fusion antigen binding molecules containing CH1-CL crossover and charged residues based on affinity matured variants of CEA binder A5H1EL1D.

TABLE 22
Amino acid sequences of monovalent CEA(P006.038) targeted split
trimeric 4-1BB ligand antigen binding molecule with CH-CL
crossover and charged residues (CEA(P006.038)-4-1BBL)

| SEQ ID | | |
|---|---|---|
| NO: | Description | Sequence |
| 99 | Dimeric | See Table 20 |
| | 4-1BBL-CL* Fc | |
| | knob chain | |
| 100 | Monomeric | See Table 20 |
| | 4-1BBL-CH1* | |
| 240 | anti-CEA | EVQLLESGGGLVQPGGSLRLSCAASGFTFTDYYMNWVRQAPGKG |
| | (P006.038) | LEWLGFIGNKANAYTTEYSASVKGRFTISRDKSKNTLYLQMNSL |
| | Fc hole chain | RAEDTATYYCTRDRGIREGFDYWGQGTTVTVSSASTKGPSVFPL |
| | | APSSKSTSGGTAALGCLVKDYFPEPVTVSWNSGALTSGVHTFPA |
| | | VLQSSGLYSLSSVVTVPSSSLGTQTYICNVNHKPSNTKVDKKVE |
| | | PKSCDKTHTCPPCPAPEAAGGPSVFLEPPKPKDTLMISRTPEVT |
| | | CVVVDVSHEDPEVKFNWYVDGVEVHNAKTKPREEQYNSTYRVVS |
| | | VLTVLHQDWLNGKEYKCKVSNKALGAPIEKTISKAKGQPREPQV |
| | | CTLPPSRDELTKNQVSLSCAVKGFYPSDIAVEWESNGQPENNYK |
| | | TTPPVLDSDGSFELVSKLTVDKSRWQQGNVESCSVMHEALHNHY |
| | | TQKSLSLSP |
| 241 | anti-CEA | EIVLTQSPATLSLSPGERATLSCRASSSVTYIHWYQQKPGQAPR |
| | (P006.038) | SWIYATSNLASGIPARFSGSGSGTDFTLTISSLEPEDFAVYYCQ |
| | light chain | HWSSVPPTFGQGTKLEIKRTVAAPSVFIFPPSDEQLKSGTASVV |
| | | CLLNNFYPREAKVQWKVDNALQSGNSQESVTEQDSKDSTYSLSS |
| | | TLTLSKADYEKHKVYACEVTHQGLSSPVTKSFNRGEC |

TABLE 23
Amino acid sequences of monovalent CEA(P005.097) targeted split
trimeric 4-1BB ligand antigen binding molecule with CH-CL
crossover and charged residues (CEA(P005.097)-4-1BBL)

| SEQ ID | | |
|---|---|---|
| NO: | Description | Sequence |
| 99 | Dimeric | See Table 20 |
| | 4-1BBL-CL* Fc | |
| | knob chain | |
| 100 | Monomeric | See Table 20 |
| | 14-BBL-CH1* | |
| 242 | anti-CEA | EVQLLESGGGLVQPGGSLRLSCAASGFTFTDYYMNWVRQAPGKG |
| | (P005.097) | LEWLGFIGNKANAYTTEYSASVKGRFTISRDKSKNTLYLQMNSL |
| | Fc hole chain | RAEDTATYYCTRDRGLRFSFDYWGQGTTVTVSSASTKGPSVFPL |
| | | APSSKSTSGGTAALGCLVKDYFPEPVTVSWNSGALTSGVHTFPA |
| | | VLQSSGLYSLSSVVTVPSSSLGTQTYICNVNHKPSNTKVDKKVE |
| | | PKSCDKTHTCPPCPAPEAAGGPSVFLEPPKPKDTLMISRTPEVT |
| | | CVVVDVSHEDPEVKFNWYVDGVEVHNAKTKPREEQYNSTYRVVS |
| | | VLTVLHQDWLNGKEYKCKVSNKALGAPIEKTISKAKGQPREPQV |
| | | CTLPPSRDELTKNQVSLSCAVKGFYPSDIAVEWESNGQPENNYK |
| | | TTPPVLDSDGSFELVSKLTVDKSRWQQGNVESCSVMHEALHNHY |
| | | TQKSLSLSP |
| 243 | anti-CEA | EIVLTQSPATLSLSPGERATLSCRASSSVTYIHWYQQKPGQAPR |
| | (P005.097) | SWIYATSNLASGIPARFSGSGSGTDFTLTISSLEPEDFAVYYCQ |
| | light chain | HWSSQPPTFGQGTKLEIKRTVAAPSVFIFPPSDEQLKSGTASVV |
| | | CLLNNFYPREAKVQWKVDNALQSGNSQESVTEQDSKDSTYSLSS |
| | | TLTLSKADYEKHKVYACEVTHQGLSSPVTKSFNRGEC |

TABLE 24
Amino acid sequences of monovalent CEA(P005.103) targeted split
trimeric 4-1BB ligand antigen binding molecule with CH-CL
crossover and charged residues (CEA(P005.103)-4-1BBL)

| SEQ ID | | |
|---|---|---|
| NO: | Description | Sequence |
| 99 | Dimeric 4- | See Table 20 |
| | 1BBL-CL* Fc | |
| | knob chain | |
| 100 | Monomeric 4- | See Table 20 |
| | 1BBL-CH1* | |
| 244 | anti-CEA | EVQLLESGGGLVQPGGSLRLSCAASGFTFTDYYMNWVRQAPGKG |
| | (P005.103) | LEWLGFIGNKANAYTTEYSASVKGRFTISRDKSKNTLYLQMNSL |
| | Fc hole chain | RAEDTATYYCTRDRGIRFYFDYWGQGTTVTVSSASTKGPSVFPL |
| | | APSSKSTSGGTAALGCLVKDYFPEPVTVSWNSGALTSGVHTFPA |
| | | VLQSSGLYSLSSVVTVPSSSLGTQTYICNVNHKPSNTKVDKKVE |
| | | PKSCDKTHTCPPCPAPEAAGGPSVFLEPPKPKDTLMISRTPEVT |
| | | CVVVDVSHEDPEVKFNWYVDGVEVHNAKTKPREEQYNSTYRVVS |
| | | VLTVLHQDWLNGKEYKCKVSNKALGAPIEKTISKAKGQPREPQV |
| | | CTLPPSRDELTKNQVSLSCAVKGFYPSDIAVEWESNGQPENNYK |
| | | TTPPVLDSDGSFELVSKLTVDKSRWQQGNVESCSVMHEALHNHY |
| | | TQKSLSLSP |
| 245 | anti-CEA | EIVLTQSPATLSLSPGERATLSCRASSSVTYIHWYQQKPGQAPR |
| | (P005.103) | SWIYATSNLASGIPARFSGSGSGTDFTLTISSLEPEDFAVYYCQ |
| | light chain | HWSSISPTFGQGTKLEIKRTVAAPSVFIFPPSDEQLKSGTASVV |
| | | CLLNNFYPREAKVQWKVDNALQSGNSQESVTEQDSKDSTYSLSS |
| | | TLTLSKADYEKHKVYACEVTHQGLSSPVTKSFNRGEC |

TABLE 25
Amino acid sequences of monovalent CEA(P002.139) targeted split
trimeric 4-1BB ligand antigen binding molecule with CH-CL
crossover and charged residues (CEA(P002.139)-4-1BBL)

| SEQ ID | | |
|---|---|---|
| NO: | Description | Sequence |
| 99 | Dimeric 4- | See Table 20 |
| | 1BBL-CL* Fc | |
| | knob chain | |
| 100 | Monomeric 4- | See Table 20 |
| | 1BBL-CH1* | |
| 246 | anti-CEA | EVQLLESGGGLVQPGGSLRLSCAASGFYFTDYAMNWVRQAPGKG |
| | (P002.139) | LEWLGVISNKANAYTTEYSASVKGRFTISRDKSKNTLYLQMNSL |
| | Fc hole chain | RAEDTATYYCTRDRGLRFYFDYWGQGTTVTVSSASTKGPSVFPL |
| | | APSSKSTSGGTAALGCLVKDYFPEPVTVSWNSGALTSGVHTFPA |
| | | VLQSSGLYSLSSVVTVPSSSLGTQTYICNVNHKPSNTKVDKKVE |
| | | PKSCDKTHTCPPCPAPEAAGGPSVFLEPPKPKDTLMISRTPEVT |
| | | CVVVDVSHEDPEVKFNWYVDGVEVHNAKTKPREEQYNSTYRVVS |
| | | VLTVLHQDWLNGKEYKCKVSNKALGAPIEKTISKAKGQPREPQV |
| | | CTLPPSRDELTKNQVSLSCAVKGFYPSDIAVEWESNGQPENNYK |
| | | TTPPVLDSDGSFELVSKLTVDKSRWQQGNVESCSVMHEALHNHY |
| | | TQKSLSLSP |
| 247 | anti-CEA | EIVLTQSPATLSLSPGERATLSCHASSSVTYIHWYQQKPGQAPR |
| | (P002.139) | SWIYATSNLASGIPARFSGSGSGTDFTLTISSLEPEDFAVYYCQ |
| | light chain | HWSSKPPTFGQGTKLEIKRTVAAPSVFIFPPSDEQLKSGTASVV |
| | | CLLNNFYPREAKVQWKVDNALQSGNSQESVTEQDSKDSTYSLSS |
| | | TLTLSKADYEKHKVYACEVTHQGLSSPVTKSFNRGEC |

TABLE 26
Amino acid sequences of monovalent CEA(P001.177) targeted
split trimeric 4-1BB ligand antigen binding molecule with
CH-CL crossover and charged residues (CEA(P001.177)-4-1BBL)

| SEQ ID | | |
|---|---|---|
| NO: | Description | Sequence |
| 99 | Dimeric 4- | See Table 20 |
| | 1BBL-CL* Fc | |
| | knob chain | |
| 100 | Monomeric 4- | See Table 20 |
| | 1BBL-CH1* | |
| 248 | anti-CEA | EVQLLESGGGLVQPGGSLRLSCAASGFYFTDYYMNWVRQAPGKG |
| | (P001.177) | LEWLGFISNKANAYTTEYSASVKGRFTISRDKSKNTLYLQMNSL |
| | Fc hole chain | RAEDTATYYCTRDRGLRFYFDYWGQGTTVTVSSASTKGPSVFPL |
| | | APSSKSTSGGTAALGCLVKDYFPEPVTVSWNSGALTSGVHTFPA |
| | | VLQSSGLYSLSSVVTVPSSSLGTQTYICNVNHKPSNTKVDKKVE |
| | | PKSCDKTHTCPPCPAPEAAGGPSVFLEPPKPKDTLMISRTPEVT |
| | | CVVVDVSHEDPEVKFNWYVDGVEVHNAKTKPREEQYNSTYRVVS |
| | | VLTVLHQDWLNGKEYKCKVSNKALGAPIEKTISKAKGQPREPQV |
| | | CTLPPSRDELTKNQVSLSCAVKGFYPSDIAVEWESNGQPENNYK |
| | | TTPPVLDSDGSFELVSKLTVDKSRWQQGNVESCSVMHEALHNHY |
| | | TQKSLSLSP |
| 249 | anti-CEA | EIVLTQSPATLSLSPGERATLSCRASSSVTYIHWYQQKPGQAPR |
| | (P001.177) | SWIYATSNLASGIPARFSGSGSGTDFTLTISSLEPEDFAVYYCQ |
| | light chain | HWSSKPPTFGQGTKLEIKRTVAAPSVFIFPPSDEQLKSGTASVV |
| | | CLLNNFYPREAKVQWKVDNALQSGNSQESVTEQDSKDSTYSLSS |
| | | TLTLSKADYEKHKVYACEVTHQGLSSPVTKSFNRGEC |

TABLE 27
Amino acid sequences of monovalent CEA(P005.102) targeted
split trimeric 1BB ligand antigen binding molecule with CH—CL
crossover and charged residues (CEA(P005.102)-4-1BBL)

| SEQ ID NO: | Description | Sequence |
|---|---|---|
| 99 | Dimeric 4- | See Table 20 |
| | 1BBL-CL* Fc | |
| | knob chain | |
| 100 | Monomeric 4- | See Table 20 |
| | 1BBL-CH1* | |
| 250 | anti- | EVQLLESGGGLVQPGGSLRLSCAASGFTFTDYYMNWVRQAPGKG |
| | CEA(P005.102) | LEWLGFIGNKANAYTTEYSASVKGRFTISRDKSKNTLYLQMNSL |
| | Fc hole chain | RAEDTATYYCTRDRGIRFQFDYWGQGTTVTVSSASTKGPSVFPL |
| | | APSSKSTSGGTAALGCLVKDYFPEPVTVSWNSGALTSGVHTFPA |
| | | VLQSSGLYSLSSVVTVPSSSLGTQTYICNVNHKPSNTKVDKKVE |
| | | PKSCDKTHTCPPCPAPEAAGGPSVFLEPPKPKDTLMISRTPEVT |
| | | CVVVDVSHEDPEVKFNWYVDGVEVHNAKTKPREEQYNSTYRVVS |
| | | VLTVLHQDWLNGKEYKCKVSNKALGAPIEKTISKAKGQPREPQV |
| | | CTLPPSRDELTKNQVSLSCAVKGFYPSDIAVEWESNGQPENNYK |
| | | TTPPVLDSDGSFELVSKLTVDKSRWQQGNVESCSVMHEALHNHY |
| | | TQKSLSLSP |
| 251 | anti- | EIVLTQSPATLSLSPGERATLSCRASSSVTYIHWYQQKPGQAPR |
| | CEA(P005.102) | SWIYATSNLASGIPARFSGSGSGTDFTLTISSLEPEDFAVYYCQ |
| | light chain | HWSSKSPTFGQGTKLEIKRTVAAPSVFIFPPSDEQLKSGTASVV |
| | | CLLNNFYPREAKVQWKVDNALQSGNSQESVTEQDSKDSTYSLSS |
| | | TLTLSKADYEKHKVYACEVTHQGLSSPVTKSFNRGEC |

TABLE 28
Amino acid sequences of monovalent CEA(P005.103-combo1) targeted
split trimeric 4-1BB ligand antigen binding molecule with CH—CL
crossover and charged residues (CEA(P005.103-combo1)-4-1BBL)

| SEQ ID NO: | Description | Sequence |
|---|---|---|
| 99 | Dimeric 4- | See Table 20 |
| | 1BBL-CL* Fc | |
| | knob chain | |
| 100 | Monomeric 4- | See Table 20 |
| | 1BBL-CH1* | |
| 252 | anti- | EVQLLESGGGLVQPGGSLRLSCAASGFTFTDYYMNWVRQAPGKG |
| | CEA(P005.103- | LEWLGFIGNKANAYTTEYSASVKGRFTISRDKSKNTLYLQMNSL |
| | combo1) Fc hole | RAEDTATYYCTRDRGIRFSFDYWGQGTTVTVSSASTKGPSVFPL |
| | chain | APSSKSTSGGTAALGCLVKDYFPEPVTVSWNSGALTSGVHTFPA |
| | | VLQSSGLYSLSSVVTVPSSSLGTQTYICNVNHKPSNTKVDKKVE |
| | | PKSCDKTHTCPPCPAPEAAGGPSVFLEPPKPKDTLMISRTPEVT |
| | | CVVVDVSHEDPEVKFNWYVDGVEVHNAKTKPREEQYNSTYRVVS |
| | | VLTVLHQDWLNGKEYKCKVSNKALGAPIEKTISKAKGQPREPQV |
| | | CTLPPSRDELTKNQVSLSCAVKGFYPSDIAVEWESNGQPENNYK |
| | | TTPPVLDSDGSFELVSKLTVDKSRWQQGNVESCSVMHEALHNHY |
| | | TQKSLSLSP |
| 253 | anti- | EIVLTQSPATLSLSPGERATLSCRASSSVTYIHWYQQKPGQAPR |
| | CEA(P005.103- | SWIYATSNLASGIPARFSGSGSGTDFTLTISSLEPEDFAVYYCQ |
| | combo1) light | HWSSISPTFGQGTKLEIKRTVAAPSVFIFPPSDEQLKSGTASVV |
| | chain | CLLNNFYPREAKVQWKVDNALQSGNSQESVTEQDSKDSTYSLSS |
| | | TLTLSKADYEKHKVYACEVTHQGLSSPVTKSFNRGEC |

TABLE 29
Amino acid sequences of monovalent CEA(P005.103-combo2) targeted
split trimeric 4-1BB ligand antigen binding molecule with CH—CL
crossover and charged residues (CEA(P005.103-combo2)-4-1BBL)

| SEQ ID NO: | Description | Sequence |
|---|---|---|
| 99 | Dimeric 4- | See Table 20 |
| | 1BBL-CL* Fc | |
| | knob chain | |
| 100 | Monomeric 4- | See Table 20 |
| | 1BBL-CH1* | |
| 254 | anti- | EVQLLESGGGLVQPGGSLRLSCAASGFYFTDYYMNWVRQAPGKG |
| | CEA(P005.103- | LEWLGVISNKANAYTTEYSASVKGRFTISRDKSKNTLYLQMNSL |
| | combo2) Fc hole | RAEDTATYYCTRDRGIRFSFDYWGQGTTVTVSSASTKGPSVFPL |
| | chain | APSSKSTSGGTAALGCLVKDYFPEPVTVSWNSGALTSGVHTFPA |
| | | VLQSSGLYSLSSVVTVPSSSLGTQTYICNVNHKPSNTKVDKKVE |
| | | PKSCDKTHTCPPCPAPEAAGGPSVFLEPPKPKDTLMISRTPEVT |
| | | CVVVDVSHEDPEVKFNWYVDGVEVHNAKTKPREEQYNSTYRVVS |
| | | VLTVLHQDWLNGKEYKCKVSNKALGAPIEKTISKAKGQPREPQV |
| | | CTLPPSRDELTKNQVSLSCAVKGFYPSDIAVEWESNGQPENNYK |
| | | TTPPVLDSDGSFELVSKLTVDKSRWQQGNVESCSVMHEALHNHY |
| | | TQKSLSLSP |
| 255 | anti- | EIVLTQSPATLSLSPGERATLSCRASSSVTYIHWYQQKPGQAPR |
| | CEA(P005.103- | SWIYATSNLASGIPARFSGSGSGTDFTLTISSLEPEDFAVYYCQ |
| | combo2) light | HWSSISPTFGQGTKLEIKRTVAAPSVFIFPPSDEQLKSGTASVV |
| | chain | CLLNNFYPREAKVQWKVDNALQSGNSQESVTEQDSKDSTYSLSS |
| | | TLTLSKADYEKHKVYACEVTHQGLSSPVTKSFNRGEC |

TABLE 30
Amino acid sequences of monovalent CEA(P005.102-combo1) targeted
split trimeric 4-1BB ligand antigen binding molecule with CH—CL
crossover and charged residues (CEA(P005.102-combo1)-4-1BBL)

| SEQ ID NO: | Description | Sequence |
|---|---|---|
| 99 | Dimeric 4- | See Table 20 |
| | 1BBL-CL* Fc | |
| | knob chain | |
| 100 | Monomeric 4- | See Table 20 |
| | 1BBL-CH1* | |
| 256 | anti- | EVQLLESGGGLVQPGGSLRLSCAASGFYFTDYYMNWVRQAPGKG |
| | CEA(P005.102- | LEWLGVISNKANAYTTEYSASVKGRFTISRDKSKNTLYLQMNSL |
| | combo1) Fc hole | RAEDTATYYCTRDRGIRFQFDYWGQGTTVTVSSASTKGPSVFPL |
| | chain | APSSKSTSGGTAALGCLVKDYFPEPVTVSWNSGALTSGVHTFPA |
| | | VLQSSGLYSLSSVVTVPSSSLGTQTYICNVNHKPSNTKVDKKVE |
| | | PKSCDKTHTCPPCPAPEAAGGPSVFLEPPKPKDTLMISRTPEVT |
| | | CVVVDVSHEDPEVKFNWYVDGVEVHNAKTKPREEQYNSTYRVVS |
| | | VLTVLHQDWLNGKEYKCKVSNKALGAPIEKTISKAKGQPREPQV |
| | | CTLPPSRDELTKNQVSLSCAVKGFYPSDIAVEWESNGQPENNYK |
| | | TTPPVLDSDGSFELVSKLTVDKSRWQQGNVESCSVMHEALHNHY |
| | | TQKSLSLSP |
| 257 | anti- | EIVLTQSPATLSLSPGERATLSCRASSSVTYIHWYQQKPGQAPR |
| | CEA(P005.102- | SWIYATSNLASGIPARFSGSGSGTDFTLTISSLEPEDFAVYYCQ |
| | combo1) light | HWSSKSPTFGQGTKLEIKRTVAAPSVFIFPPSDEQLKSGTASVV |
| | chain | CLLNNFYPREAKVQWKVDNALQSGNSQESVTEQDSKDSTYSLSS |
| | | TLTLSKADYEKHKVYACEVTHQGLSSPVTKSFNRGEC |

TABLE 31
Amino acid sequences of monovalent CEA(P005.102-combo2) targeted
split trimeric 4-1BB ligand antigen binding molecule with CH—CL
crossover and charged residues (CEA(P005.102-combo2)-4-1BBL)

| SEQ ID NO: | Description | Sequence |
|---|---|---|
| 99 | Dimeric 4- | See Table 20 |
| | 1BBL-CL* Fc | |
| | knob chain | |
| 100 | Monomeric 4- | See Table 20 |
| | 1BBL-CH1* | |
| 258 | anti- | EVQLLESGGGLVQPGGSLRLSCAASGFYFSDYYMNWVRQAPGKG |
| | CEA(P005.102- | LEWLGVISNKANAYTTEYSASVKGRFTISRDKSKNTLYLQMNSL |
| | combo2) Fc hole | RAEDTATYYCTRDRGIRFQFDYWGQGTTVTVSSASTKGPSVFPL |
| | chain | APSSKSTSGGTAALGCLVKDYFPEPVTVSWNSGALTSGVHTFPA |
| | | VLQSSGLYSLSSVVTVPSSSLGTQTYICNVNHKPSNTKVDKKVE |
| | | PKSCDKTHTCPPCPAPEAAGGPSVFLEPPKPKDTLMISRTPEVT |
| | | CVVVDVSHEDPEVKFNWYVDGVEVHNAKTKPREEQYNSTYRVVS |
| | | VLTVLHQDWLNGKEYKCKVSNKALGAPIEKTISKAKGQPREPQV |
| | | CTLPPSRDELTKNQVSLSCAVKGFYPSDIAVEWESNGQPENNYK |
| | | TTPPVLDSDGSFELVSKLTVDKSRWQQGNVESCSVMHEALHNHY |
| | | TQKSLSLSP |
| 259 | anti- | EIVLTQSPATLSLSPGERATLSCRASSSVTYIHWYQQKPGQAPR |
| | CEA(P005.102- | SWIYATSNLASGIPARFSGSGSGTDFTLTISSLEPEDFAVYYCQ |
| | combo2) light | HWSSKSPTFGQGTKLEIKRTVAAPSVFIFPPSDEQLKSGTASVV |
| | chain | CLLNNFYPREAKVQWKVDNALQSGNSQESVTEQDSKDSTYSLSS |
| | | TLTLSKADYEKHKVYACEVTHQGLSSPVTKSFNRGEC |

TABLE 32
Amino acid sequences of monovalent CEA(P006.038-combo1) targeted
split trimeric 4-1BB ligand antigen binding molecule with CH—CL
crossover and charged residues (CEA(P006.038-combo1)-4-1BBL)

| SEQ ID NO: | Description | Sequence |
|---|---|---|
| 99 | Dimeric 4- | See Table 20 |
| | 1BBL-CL* Fc | |
| | knob chain | |
| 100 | Monomeric 4- | See Table 20 |
| | 1BBL-CH1* | |
| 260 | anti- | EVQLLESGGGLVQPGGSLRLSCAASGFYFTDYAMNWVRQAPGKG |
| | CEA(P006.038- | LEWLGVISNKANAYTTEYSASVKGRFTISRDKSKNTLYLQMNSL |
| | combo1) Fc hole | RAEDTATYYCTRDRGIREGFDYWGQGTTVTVSSASTKGPSVFPL |
| | chain | APSSKSTSGGTAALGCLVKDYFPEPVTVSWNSGALTSGVHTFPA |
| | | VLQSSGLYSLSSVVTVPSSSLGTQTYICNVNHKPSNTKVDKKVE |
| | | PKSCDKTHTCPPCPAPEAAGGPSVFLEPPKPKDTLMISRTPEVT |
| | | CVVVDVSHEDPEVKFNWYVDGVEVHNAKTKPREEQYNSTYRVVS |
| | | VLTVLHQDWLNGKEYKCKVSNKALGAPIEKTISKAKGQPREPQV |
| | | CTLPPSRDELTKNQVSLSCAVKGFYPSDIAVEWESNGQPENNYK |
| | | TTPPVLDSDGSFELVSKLTVDKSRWQQGNVESCSVMHEALHNHY |
| | | TQKSLSLSP |
| 261 | anti- | EIVLTQSPATLSLSPGERATLSCRASSSVTYIHWYQQKPGQAPR |
| | CEA(P006.038- | SWIYATSNLASGIPARFSGSGSGTDFTLTISSLEPEDFAVYYCQ |
| | combo1) light | HWSSVPPTFGQGTKLEIKRTVAAPSVFIFPPSDEQLKSGTASVV |
| | chain | CLLNNFYPREAKVQWKVDNALQSGNSQESVTEQDSKDSTYSLSS |
| | | TLTLSKADYEKHKVYACEVTHQGLSSPVTKSFNRGEC |

TABLE 33
Amino acid sequences of monovalent CEA(P006.038-combo2) targeted
split trimeric 4-1BB ligand antigen binding molecule with CH—CL
crossover and charged residues (CEA(P006.038.combo2)-4-1BBL)

| SEQ ID NO: | Description | Sequence |
|---|---|---|
| 99 | Dimeric 4- | See Table 20 |
| | 1BBL-CL* Fc | |
| | knob chain | |
| 100 | Monomeric 4- | See Table 20 |
| | 1BBL-CH1* | |
| 262 | anti- | EVQLLESGGGLVQPGGSLRLSCAASGFTFSDYEMNWVRQAPGKG |
| | CEA(P006.038- | LEWLGFISNKANAYTTEYSASVKGRFTISRDKSKNTLYLQMNSL |
| | combo2) Fc hole | RAEDTATYYCTRDRGIREGFDYWGQGTTVTVSSASTKGPSVFPL |
| | chain | APSSKSTSGGTAALGCLVKDYFPEPVTVSWNSGALTSGVHTFPA |
| | | VLQSSGLYSLSSVVTVPSSSLGTQTYICNVNHKPSNTKVDKKVE |
| | | PKSCDKTHTCPPCPAPEAAGGPSVFLEPPKPKDTLMISRTPEVT |
| | | CVVVDVSHEDPEVKFNWYVDGVEVHNAKTKPREEQYNSTYRVVS |
| | | VLTVLHQDWLNGKEYKCKVSNKALGAPIEKTISKAKGQPREPQV |
| | | CTLPPSRDELTKNQVSLSCAVKGFYPSDIAVEWESNGQPENNYK |
| | | TTPPVLDSDGSFELVSKLTVDKSRWQQGNVESCSVMHEALHNHY |
| | | TQKSLSLSP |
| 263 | anti- | EIVLTQSPATLSLSPGERATLSCRASSSVTYIHWYQQKPGQAPR |
| | CEA(P006.038- | SWIYATSNLASGIPARFSGSGSGTDFTLTISSLEPEDFAVYYCQ |
| | combo2) light | HWSSVPPTFGQGTKLEIKRTVAAPSVFIFPPSDEQLKSGTASVV |
| | chain | CLLNNFYPREAKVQWKVDNALQSGNSQESVTEQDSKDSTYSLSS |
| | | TLTLSKADYEKHKVYACEVTHQGLSSPVTKSFNRGEC |

Table 34 shows the amino acid sequences of the monovalent CEA-targeted split trimeric 4-1BB ligand Fc (kih) fusion antigen binding molecule containing CH1-CL crossover and charged residues based on CEA binder MFE23 and Tables 35 to 41 show the amino acid sequences of the monovalent CEA-targeted split trimeric 4-1BB ligand Fc (kih) fusion antigen binding molecule based on its humanized versions.

TABLE 34
Amino acid sequences of monovalent CEA(MFE23) targeted split
trimeric 4-1BB ligand antigen binding molecule with CH—CL
crossover and charged residues (CEA(MFE23)-4-1BBL)

| SEQ ID NO: | Description | Sequence |
|---|---|---|
| 99 | Dimeric 4- | See Table 20 |
| | 1BBL-CL* | |
| | Fc knob chain | |
| 100 | Monomeric 4- | See Table 20 |
| | 1BBL-CH1* | |
| 264 | anti- | QVKLQQSGAELVRSGTSVKLSCTASGFNIKDSYMHWLRQGPEQG |
| | CEA(MFE23) | LEWIGWIDPENGDTEYAPKFQGKATFTTDTSSNTAYLQLSSLTS |
| | Fc hole chain | EDTAVYYCNEGTPTGPYYFDYWGQGTTVTVSSASTKGPSVFPLA |
| | | PSSKSTSGGTAALGCLVKDYFPEPVTVSWNSGALTSGVHTFPAV |
| | | LQSSGLYSLSSVVTVPSSSLGTQTYICNVNHKPSNTKVDKKVEP |
| | | KSCDKTHTCPPCPAPEAAGGPSVFLEPPKPKDTLMISRTPEVTC |
| | | VVVDVSHEDPEVKFNWYVDGVEVHNAKTKPREEQYNSTYRVVSV |
| | | LTVLHQDWLNGKEYKCKVSNKALGAPIEKTISKAKGQPREPQVC |
| | | TLPPSRDELTKNQVSLSCAVKGFYPSDIAVEWESNGQPENNYKT |
| | | TPPVLDSDGSFELVSKLTVDKSRWQQGNVESCSVMHEALHNHYT |
| | | QKSLSLSP |
| 265 | anti- | ENVLTQSPAIMSASPGEKVTITCSASSSVSYMHWFQQKPGTSPK |
| | CEA(MFE23) | LWIYSTSNLASGVPARFSGSGSGTSYSLTISRMEAEDAATYYCQ |
| | light chain | QRSSYPLTFGAGTKLELKRTVAAPSVFIFPPSDEQLKSGTASVV |
| | | CLLNNFYPREAKVQWKVDNALQSGNSQESVTEQDSKDSTYSLSS |
| | | TLTLSKADYEKHKVYACEVTHQGLSSPVTKSFNRGEC |

TABLE 35
Amino acid sequences of monovalent CEA(huMFE23-L28-1124) targeted
split trimeric 4-1BB ligand antigen binding molecule with CH—CL
crossover and charged residues (CEA(MFE23-L28-1124)-4-1BBL)
CEA binder huMFE23-L28-H24 corresponds to the clone used in P1AE3101.

| SEQ ID NO: | Description | Sequence |
|---|---|---|
| 99 | Dimeric 4- | See Table 20 |
| | 1BBL-CL* | |
| | Fc knob chain | |
| 100 | Monomeric 4- | See Table 20 |
| | 1BBL-CH1* | |
| 266 | anti- | QVQLVQSGAEVKKPGASVKVSCKASGFNIKDSYMHWVRQAPGQG |
| | CEA(huMFE2 | LEWMGWIDPENGDTEYAPKFQGRVTMTTDTSISTAYMELSRLRS |
| | 3-L28-H24) Fc | DDTAVYYCNEGTPTGPYYFDYWGQGTLVTVSSASTKGPSVFPLA |
| | hole chain | PSSKSTSGGTAALGCLVKDYFPEPVTVSWNSGALTSGVHTFPAV |
| | | LQSSGLYSLSSVVTVPSSSLGTQTYICNVNHKPSNTKVDKKVEP |
| | | KSCDKTHTCPPCPAPEAAGGPSVFLEPPKPKDTLMISRTPEVTC |
| | | VVVDVSHEDPEVKFNWYVDGVEVHNAKTKPREEQYNSTYRVVSV |
| | | LTVLHQDWLNGKEYKCKVSNKALGAPIEKTISKAKGQPREPQVC |
| | | TLPPSRDELTKNQVSLSCAVKGFYPSDIAVEWESNGQPENNYKT |
| | | TPPVLDSDGSFELVSKLTVDKSRWQQGNVESCSVMHEALHNHYT |
| | | QKSLSLSP |
| 267 | anti- | EIQMTQSPSSLSASVGDRVTITCRASSSVPYMHWLQQKPGKAPK |
| | CEA(huMFE2 | LLIYSTSNLASGVPSRFSGSGSGTDFTLTISSVQPEDFATYYCQ |
| | 3-L28-H24) | QRSSYPLTEGGGTKLEIKRTVAAPSVFIFPPSDEQLKSGTASVV |
| | light chain | CLLNNFYPREAKVQWKVDNALQSGNSQESVTEQDSKDSTYSLSS |
| | | TLTLSKADYEKHKVYACEVTHQGLSSPVTKSFNRGEC |

TABLE 36
Amino acid sequences of monovalent CEA(huMFE23-L28-1128) targeted
split trimeric 4-1BB ligand antigen binding molecule with CH—CL
crossover and charged residues (CEA(MFE23-L28-1128)-4-1BBL)
CEA binder huMFE23-L28-H28 corresponds to the clone used in P1AE3097.

| SEQ ID NO: | Description | Sequence |
|---|---|---|
| 99 | Dimeric 4- | See Table 20 |
| | 1BBL-CL* | |
| | Fc knob chain | |
| 100 | Monomeric 4- | See Table 20 |
| | 1BBL-CH1* | |
| 268 | anti- | QVQLVQSGAEVKKPGASVKVSCKASGFNIKDSYMHWVRQAPGQG |
| | CEA(huMFE2 | LEWMGWIDPENGDTEYAPKFQGRVTMTRDTSISTAYMELSRLRS |
| | 3-L28-H28) Fc | DDTAVYYCNEGTPTGPYYFDYWGQGTLVTVSSASTKGPSVFPLA |
| | hole chain | PSSKSTSGGTAALGCLVKDYFPEPVTVSWNSGALTSGVHTFPAV |
| | | LQSSGLYSLSSVVTVPSSSLGTQTYICNVNHKPSNTKVDKKVEP |
| | | KSCDKTHTCPPCPAPEAAGGPSVFLEPPKPKDTLMISRTPEVTC |
| | | VVVDVSHEDPEVKFNWYVDGVEVHNAKTKPREEQYNSTYRVVSV |
| | | LTVLHQDWLNGKEYKCKVSNKALGAPIEKTISKAKGQPREPQVC |
| | | TLPPSRDELTKNQVSLSCAVKGFYPSDIAVEWESNGQPENNYKT |
| | | TPPVLDSDGSFELVSKLTVDKSRWQQGNVESCSVMHEALHNHYT |
| | | QKSLSLSP |
| 267 | anti- | See Table 35 |
| | CEA(huMFE2 | |
| | 3-L28-H28) | |
| | light chain | |

TABLE 37
Amino acid sequences of monovalent CEA(huMFE23-L28-1125) targeted
split trimeric 4-1BB ligand antigen binding molecule with CH—CL
crossover and charged residues (CEA(MFE23-L28-1125)-4-1BBL)
CEA binder huMFE23-L28-H25 corresponds to the clone used in P1AE3100.

| SEQ ID NO: | Description | Sequence |
|---|---|---|
| 99 | Dimeric 4- | See Table 20 |
| | 1BBL-CL* | |
| | Fc knob chain | |
| 100 | Monomeric 4- | See Table 20 |
| | 1BBL-CH1* | |
| 269 | anti- | QVQLVQSGAEVKKPGASVKVSCKASGYTFKDSYMHWVRQAPGQG |
| | CEA(huMFE2 | LEWMGWIDPENGDTEYAPKFQGRVTMTTDTSISTAYMELSRLRS |
| | 3-L28-H25) Fc | DDTAVYYCNEGTPTGPYYFDYWGQGTLVTVSSASTKGPSVFPLA |
| | hole chain | PSSKSTSGGTAALGCLVKDYFPEPVTVSWNSGALTSGVHTFPAV |
| | | LQSSGLYSLSSVVTVPSSSLGTQTYICNVNHKPSNTKVDKKVEP |
| | | KSCDKTHTCPPCPAPEAAGGPSVFLEPPKPKDTLMISRTPEVTC |
| | | VVVDVSHEDPEVKFNWYVDGVEVHNAKTKPREEQYNSTYRVVSV |
| | | LTVLHQDWLNGKEYKCKVSNKALGAPIEKTISKAKGQPREPQVC |
| | | TLPPSRDELTKNQVSLSCAVKGFYPSDIAVEWESNGQPENNYKT |
| | | TPPVLDSDGSFELVSKLTVDKSRWQQGNVESCSVMHEALHNHYT |
| | | QKSLSLSP |
| 267 | anti- | See Table 35 |
| | CEA(huMFE2 | |
| | 3-L28-H25) | |
| | light chain | |

TABLE 38
Amino acid sequences of monovalent CEA(huMFE23-L27-1129) targeted
split trimeric 4-1BB ligand antigen binding molecule with CH—CL
crossover and charged residues (CEA(MFE23-L27-H29)-4-1BBL)
CEA binder huMFE23-L27-H29 corresponds to the clones used in P1AE3102.

| SEQ ID NO: | Description | Sequence |
|---|---|---|
| 99 | Dimeric 4- | See Table 20 |
| | 1BBL-CL* | |
| | Fc knob chain | |
| 100 | Monomeric 4- | See Table 20 |
| | 1BBL-CH1* | |
| 270 | anti- | QVQLVQSGAEVKKPGSSVKVSCKASGFNIKDSYMHWVRQAPGQG |
| | CEA(huMFE2 | LEWMGWIDPENGDTEYAPKFQGRVTITTDESTSTAYMELSSLRS |
| | 3-L27-H29) Fc | EDTAVYYCNEGTPTGPYYFDYWGQGTLVTVSSASTKGPSVFPLA |
| | hole chain | PSSKSTSGGTAALGCLVKDYFPEPVTVSWNSGALTSGVHTFPAV |
| | | LQSSGLYSLSSVVTVPSSSLGTQTYICNVNHKPSNTKVDKKVEP |
| | | KSCDKTHTCPPCPAPEAAGGPSVFLEPPKPKDTLMISRTPEVTC |
| | | VVVDVSHEDPEVKFNWYVDGVEVHNAKTKPREEQYNSTYRVVSV |
| | | LTVLHQDWLNGKEYKCKVSNKALGAPIEKTISKAKGQPREPQVC |
| | | TLPPSRDELTKNQVSLSCAVKGFYPSDIAVEWESNGQPENNYKT |
| | | TPPVLDSDGSFELVSKLTVDKSRWQQGNVESCSVMHEALHNHYT |
| | | QKSLSLSP |
| 271 | anti- | EIQMTQSPSSLSASVGDRVTITCRASSSVPYMHWYQQKPGKAPK |
| | CEA(huMFE2 | LLIYSTSNLASGVPSRFSGSGSGTDFTLTISSVQPEDFATYYCQ |
| | 3-L27-H29) | QRSSYPLTEGGGTKLEIKRTVAAPSVFIFPPSDEQLKSGTASVV |
| | light chain | CLLNNFYPREAKVQWKVDNALQSGNSQESVTEQDSKDSTYSLSS |
| | | TLTLSKADYEKHKVYACEVTHQGLSSPVTKSFNRGEC |

TABLE 39
Amino acid sequences of monovalent CEA(huMFE23-L27-1128) targeted
split trimeric 4-1BB ligand antigen binding molecule with CH—CL
crossover and charged residues (CEA(MFE23-L27-1128)-4-1BBL)
CEA binder huMFE23-L27-H28 corresponds to the clone used in P1AE3103.

| SEQ ID NO: | Description | Sequence |
|---|---|---|
| 99 | Dimeric 4- | See Table 20 |
| | 1BBL-CL* | |
| | Fc knob chain | |
| 100 | Monomeric 4- | See Table 20 |
| | 1BBL-CH1* | |
| 272 | anti- | QVQLVQSGAEVKKPGASVKVSCKASGFNIKDSYMHWVRQAPGQG |
| | CEA(huMFE2 | LEWMGWIDPENGDTEYAPKFQGRVTMTRDTSISTAYMELSRLRS |
| | 3-L27-H28) Fc | DDTAVYYCNEGTPTGPYYFDYWGQGTLVTVSSASTKGPSVFPLA |
| | hole chain | PSSKSTSGGTAALGCLVKDYFPEPVTVSWNSGALTSGVHTFPAV |
| | | LQSSGLYSLSSVVTVPSSSLGTQTYICNVNHKPSNTKVDKKVEP |
| | | KSCDKTHTCPPCPAPEAAGGPSVFLEPPKPKDTLMISRTPEVTC |
| | | VVVDVSHEDPEVKFNWYVDGVEVHNAKTKPREEQYNSTYRVVSV |
| | | LTVLHQDWLNGKEYKCKVSNKALGAPIEKTISKAKGQPREPQVC |
| | | TLPPSRDELTKNQVSLSCAVKGFYPSDIAVEWESNGQPENNYKT |
| | | TPPVLDSDGSFELVSKLTVDKSRWQQGNVESCSVMHEALHNHYT |
| | | QKSLSLSP |
| 271 | anti- | See Table 38 |
| | CEA(huMFE2 | |
| | 3-L27-H28) | |
| | light chain | |

TABLE 40
Amino acid sequences of monovalent CEA(huMFE23-L27-1126) targeted
split trimeric 4-1BB ligand antigen binding molecule with CH—CL
crossover and charged residue (CEA(MFE23-L27-1126)-4-1BBL)
CEA binder huMFE23-L27-H26 corresponds to the clone used in P1AE3105.

| SEQ ID NO: | Description | Sequence |
|---|---|---|
| 99 | Dimeric 4- | See Table 20 |
| | 1BBL-CL* | |
| | Fc knob chain | |
| 100 | Monomeric 4- | See Table 20 |
| | 1BBL-CH1* | |
| 273 | anti- | QVQLVQSGAEVKKPGASVKVSCKASGFNIKDSYMHWVRQAPGQG |
| | CEA(huMFE2 | LEWMGWIDPENGGTNYAQKFQGRVTMTTDTSISTAYMELSRLRS |
| | 3-L27-H26) Fc | DDTAVYYCNEGTPTGPYYFDYWGQGTLVTVSSASTKGPSVFPLA |
| | hole chain | PSSKSTSGGTAALGCLVKDYFPEPVTVSWNSGALTSGVHTFPAV |
| | | LQSSGLYSLSSVVTVPSSSLGTQTYICNVNHKPSNTKVDKKVEP |
| | | KSCDKTHTCPPCPAPEAAGGPSVFLEPPKPKDTLMISRTPEVTC |
| | | VVVDVSHEDPEVKFNWYVDGVEVHNAKTKPREEQYNSTYRVVSV |
| | | LTVLHQDWLNGKEYKCKVSNKALGAPIEKTISKAKGQPREPQVC |
| | | TLPPSRDELTKNQVSLSCAVKGFYPSDIAVEWESNGQPENNYKT |
| | | TPPVLDSDGSFELVSKLTVDKSRWQQGNVESCSVMHEALHNHYT |
| | | QKSLSLSP |
| 271 | anti- | See Table 38 |
| | CEA(huMFE2 | |
| | 3-L27-H26) | |
| | light chain | |

TABLE 41
Amino acid sequences of monovalent CEA(huMFE23-L27-1124) targeted
split trimeric 4-1BB ligand antigen binding molecule with CH—CL
crossover and charged residues (CEA(MFE23-L27-1124)-4-1BBL)
CEA binder huMFE23-L27-H24 corresponds to the clone used in P1AE3107.

| SEQ ID NO: | Description | Sequence |
|---|---|---|
| 99 | Dimeric 4- | See Table 20 |
| | 1BBL-CL* | |
| | Fc knob chain | |
| 100 | Monomeric 4- | See Table 20 |
| | 1BBL-CH1* | |
| 274 | anti- | QVQLVQSGAEVKKPGASVKVSCKASGFNIKDSYMHWVRQAPGQG |
| | CEA(huMFE2 | LEWMGWIDPENGDTEYAPKFQGRVTMTTDTSISTAYMELSRLRS |
| | 3-L27-H24) Fc | DDTAVYYCNEGTPTGPYYFDYWGQGTLVTVSSASTKGPSVFPLA |
| | hole chain | PSSKSTSGGTAALGCLVKDYFPEPVTVSWNSGALTSGVHTFPAV |
| | | LQSSGLYSLSSVVTVPSSSLGTQTYICNVNHKPSNTKVDKKVEP |
| | | KSCDKTHTCPPCPAPEAAGGPSVFLEPPKPKDTLMISRTPEVTC |
| | | VVVDVSHEDPEVKFNWYVDGVEVHNAKTKPREEQYNSTYRVVSV |
| | | LTVLHQDWLNGKEYKCKVSNKALGAPIEKTISKAKGQPREPQVC |
| | | TLPPSRDELTKNQVSLSCAVKGFYPSDIAVEWESNGQPENNYKT |
| | | TPPVLDSDGSFELVSKLTVDKSRWQQGNVESCSVMHEALHNHYT |
| | | QKSLSLSP |
| 271 | anti- | See Table 38 |
| | CEA(huMFE2 | |
| | 3-L27-H24) | |
| | light chain | |

The bispecific constructs were produced by transfecting mammalian cells with the corresponding expression vectors in a 1:1:1:1 (“vector 4-1BBL Fc-knob chain”: “vector 4-1BBL light chain”: “vector Fc-hole chain”: “vector light chain”).

### Production of IgG-Like Proteins in HEK293 EBNA or CHO EBNA Cells

Antibodies and bispecific antibodies were generated by transient transfection of HEK293 EBNA cells or CHO EBNA cells. Cells were centrifuged and, medium was replaced by pre-warmed CD CHO medium (Thermo Fisher, Cat No 10743029). Expression vectors were mixed in CD CHO medium, PEI (Polyethylenimine, Polysciences, Inc, Cat No 23966-1) was added, the solution vortexed and incubated for 10 minutes at room temperature. Afterwards, cells (2 Mio/ml) were mixed with the vector/PEI solution, transferred to a flask and incubated for 3 hours at 37° C. in a shaking incubator with a 5% CO₂ atmosphere. After the incubation, Excell medium with supplements (80% of total volume) was added (W. Zhou and A. Kantardjieff, Mammalian Cell Cultures for Biologics Manufacturing, DOI: 10.1007/978-3-642-54050-9; 2014). One day after transfection, supplements (Feed, 12% of total volume) were added. Cell supernatants were harvested after 7 days by centrifugation and subsequent filtration (0.2 μm filter), and proteins were purified from the harvested supernatant by standard methods as indicated below.

### Production of IgG-Like Proteins in CHO K1 Cells

Alternatively, the antibodies and bispecific antibodies described herein were prepared by Evitria using their proprietary vector system with conventional (non-PCR based) cloning techniques and using suspension-adapted CHO K1 cells (originally received from ATCC and adapted to serum-free growth in suspension culture at Evitria). For the production, Evitria used its proprietary, animal-component free and serum-free media (eviGrow and eviMake2) and its proprietary transfection reagent (eviFect). Supernatant was harvested by centrifugation and subsequent filtration (0.2 μm filter) and, proteins were purified from the harvested supernatant by standard methods.

### Purification of IgG-Like Proteins

Proteins were purified from filtered cell culture supernatants referring to standard protocols. In brief, Fc containing proteins were purified from cell culture supernatants by Protein A-affinity chromatography (equilibration buffer: 20 mM sodium citrate, 20 mM sodium phosphate, pH 7.5; elution buffer: 20 mM sodium citrate, pH 3.0). Elution was achieved at pH 3.0 followed by immediate pH neutralization of the sample. The protein was concentrated by centrifugation (Millipore Amicon® ULTRA-15 (Art.Nr.: UFC903096), and aggregated protein was separated from monomeric protein by size exclusion chromatography in 20 mM histidine, 140 mM sodium chloride, pH 6.0.

### Analytics of IgG-Like Proteins

The concentrations of purified proteins were determined by measuring the absorption at 280 nm using the mass extinction coefficient calculated on the basis of the amino acid sequence according to Pace, et al., Protein Science, 1995, 4, 2411-1423. Purity and molecular weight of the proteins were analyzed by CE-SDS in the presence and absence of a reducing agent using a LabChipGXII (Perkin Elmer). Determination of the aggregate content was performed by HPLC chromatography at 25° C. using analytical size-exclusion column (TSKgel G3000 SW XL or UP-SW3000) equilibrated in running buffer (25 mM K₂HPO₄, 125 mM NaCl, 200 mM L-Arginine Monohydrocloride, pH 6.7 or 200 mM KH₂PO₄, 250 mM KCl at pH 6.2, respectively).

TABLE 42
Biochemical analysis of exemplary CEA-targeted 4-1BB ligand
trimer-containing Fc (kih) fusion antigen binding molecules

| | Monomer | Yield | CE-SDS |
|---|---|---|---|
| Molecule | [%] (SEC) | [mg/l] | (non-red) |
| CEA(A5H1EL1D)-4-1-BBL | 100 | 2.6 | 84 |
| CEA(A5B7)-4-1BBL | 92 | 1.5 | 92 |
| CEA(MFE23)-4-1BBL | 96 | 24 | 93 |
| CEA(MFE23-L28-H24)-4-1BBL | 92 | 13 | 97 |
| CEA(MFE23-L28-H28)-4-1BBL | 93 | 14 | 98 |
| CEA(P001.177)-4-1BBL | 91 | 4.8 | 99 |
| CEA(P005.102)-4-1BBL | 94 | 5.8 | 99 |

### 2.2 Functional Characterization of CEA-Targeting 4-1BB Ligand Trimer-Containing Fc Fusion Antigen Binding Molecules by Surface Plasmon Resonance

The capacity to bind simultaneously to human 4-1BB Fc(kih) and human CEA, in form of NABA construct, was assessed by surface plasmon resonance (SPR). All SPR experiments were performed on a Biacore T200 at 25° C. with HBS-EP as running buffer (0.01 M HEPES pH 7.4, 0.15 M NaCl, 3 mM EDTA, 0.005% Surfactant P20, Biacore, Freiburg/Germany). Human N(A2B2)A protein was directly coupled to a flow cell of a CM5 chip by amine coupling. Immobilization level of approx. 600 RU was used.

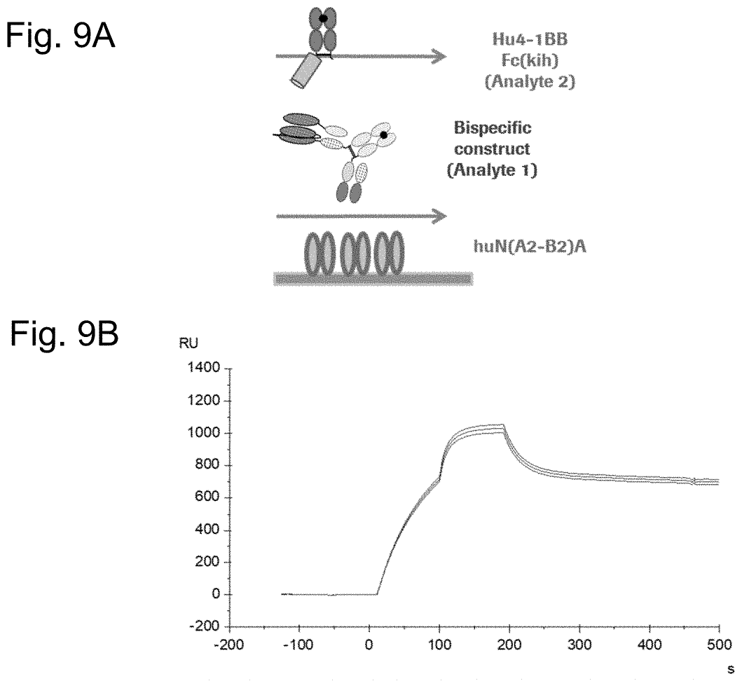

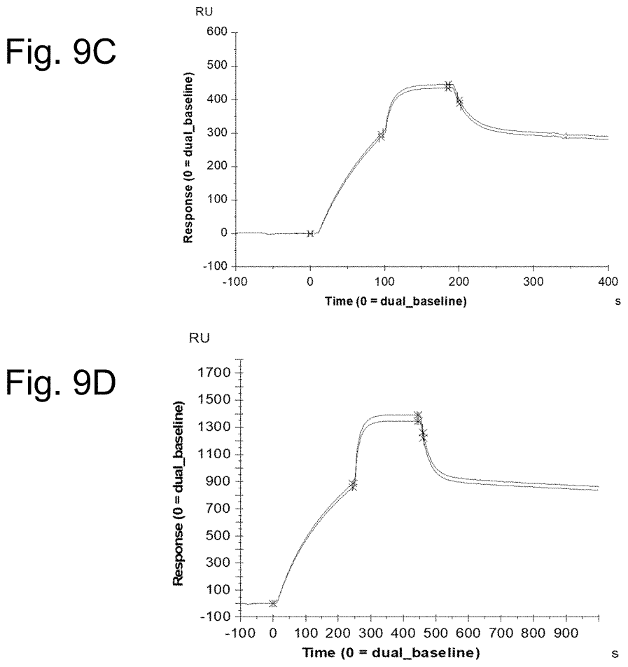

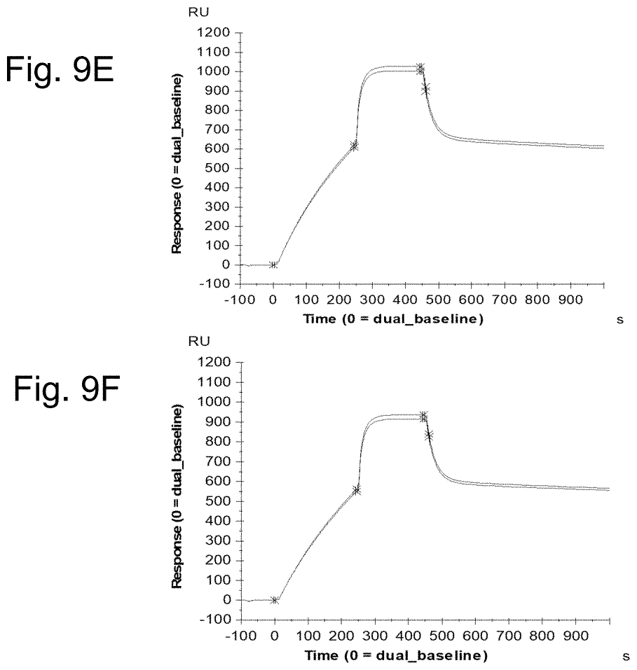

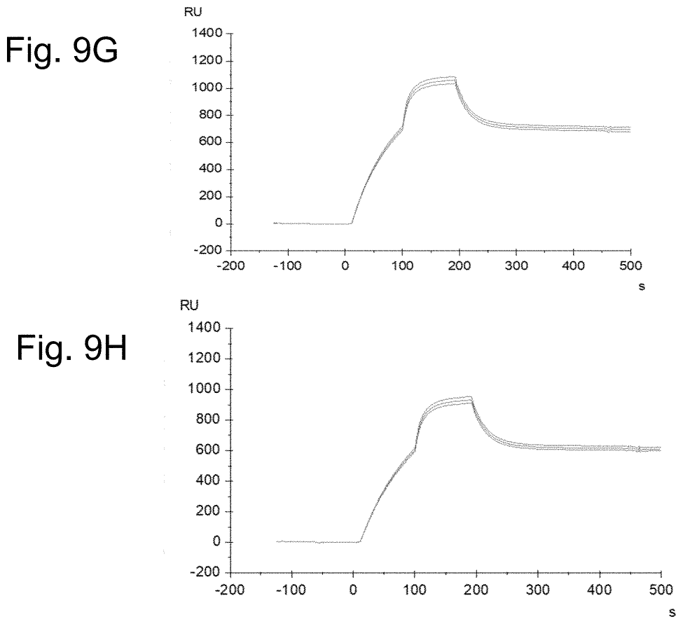

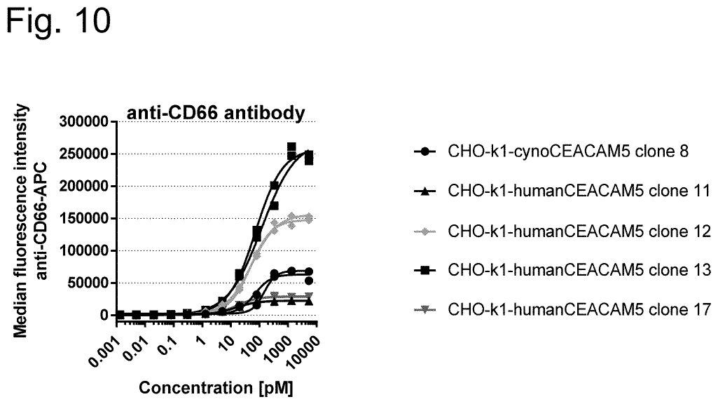

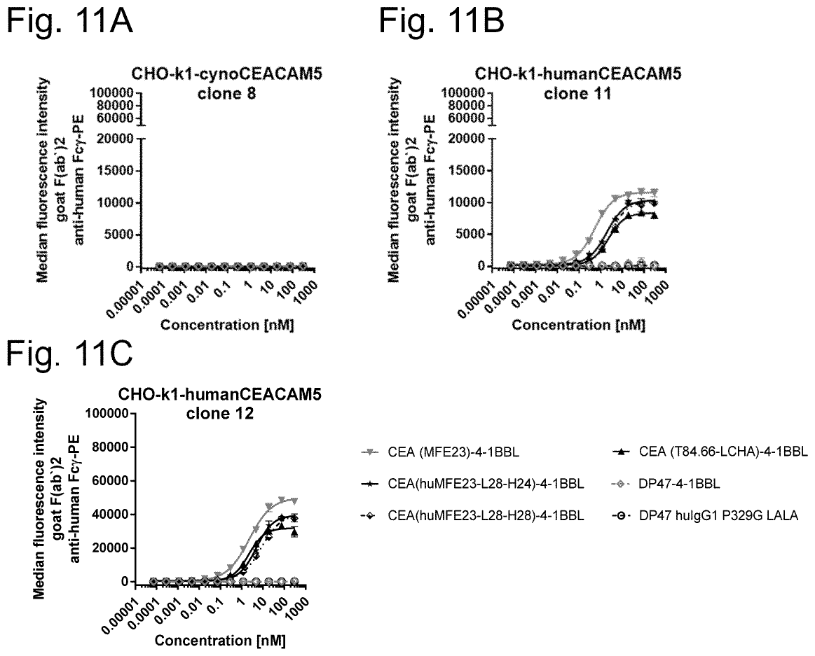

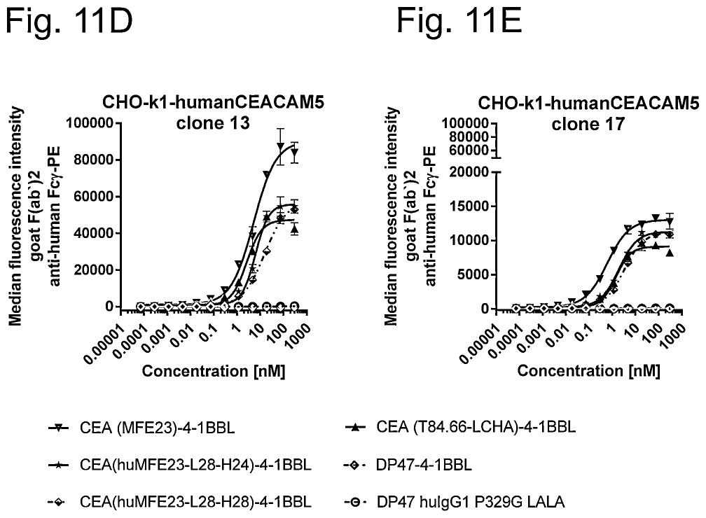

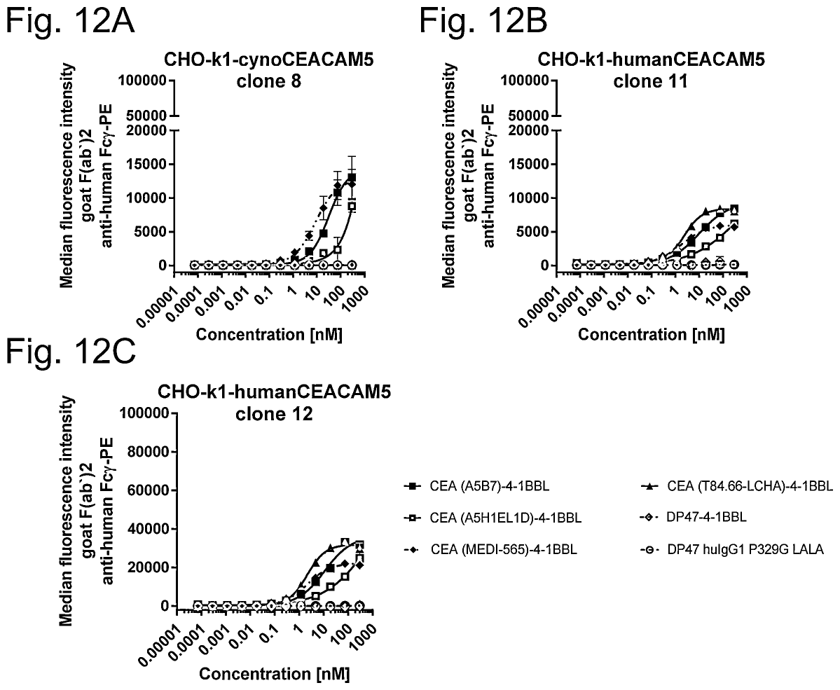

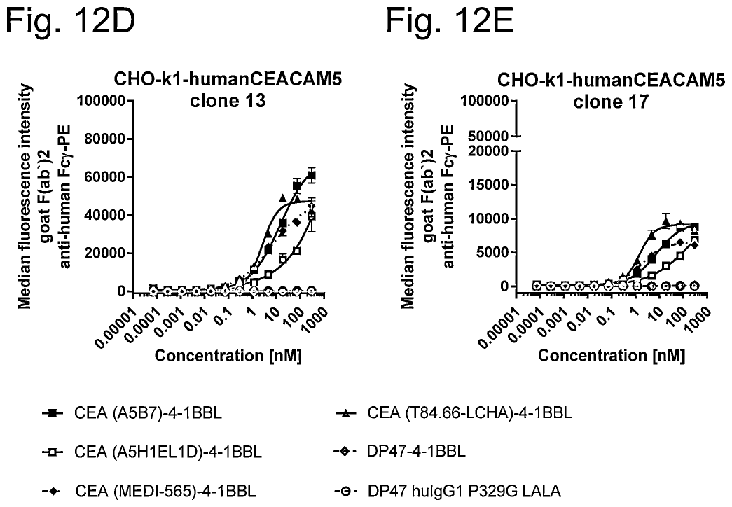

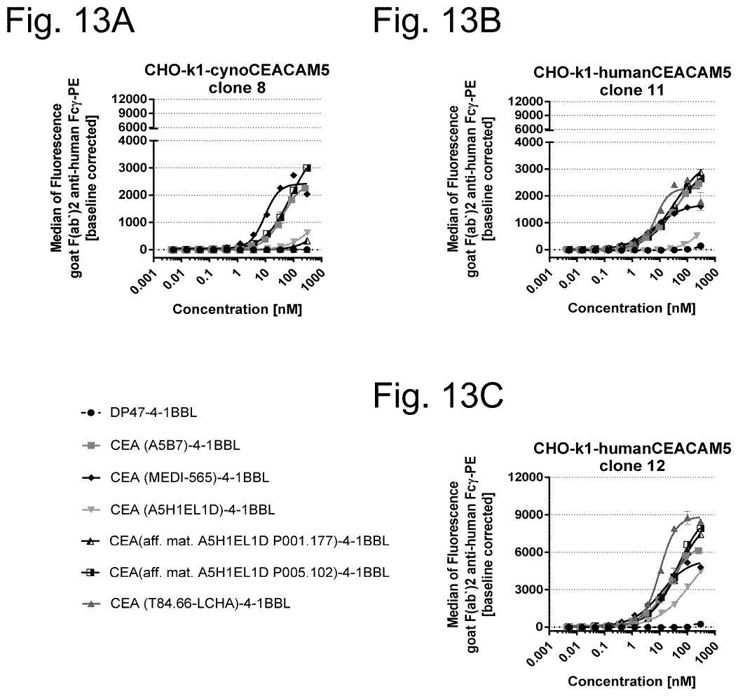

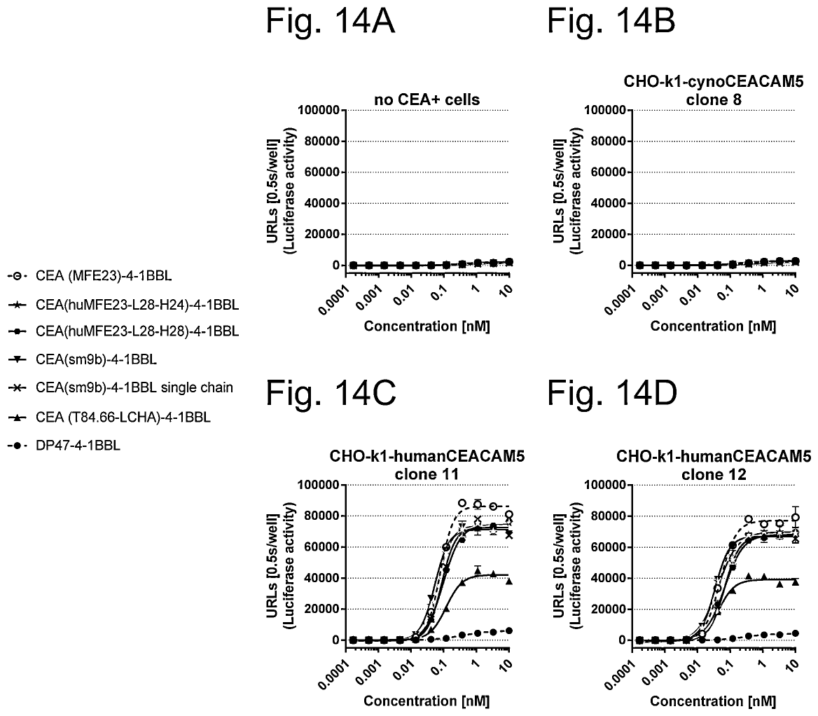

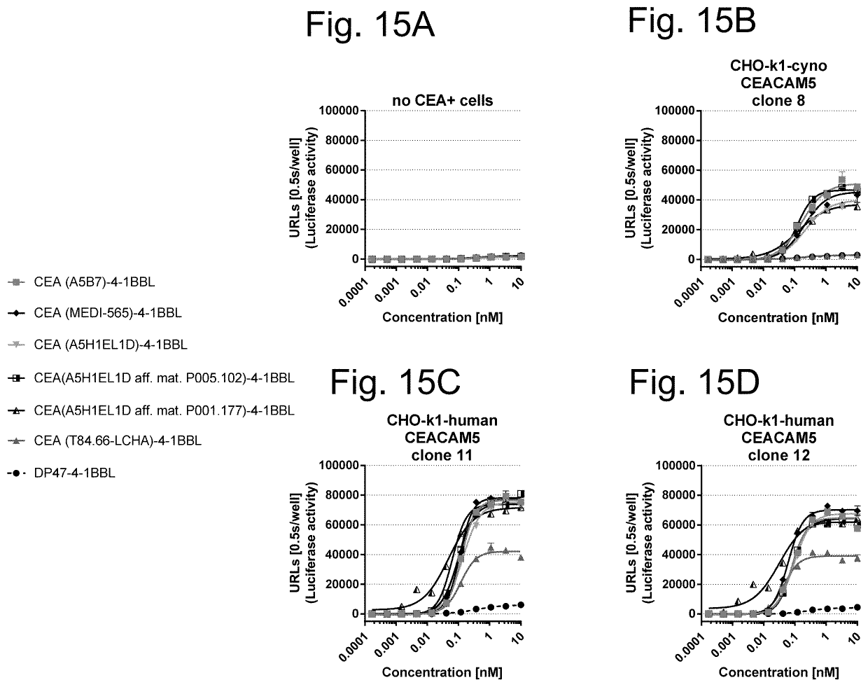

The CEA targeting trimeric split 4-1BBL construct was passed at a concentration range of 200 or 500 nM with a flow of 30 μL/minute through the flow cells over 90 or 240 seconds and dissociation was set to zero sec. Human 4-1BB Fc(kih) was injected as second analyte with a flow of 30 μL/minute through the flow cells over 90 or 200 seconds at a concentration of 500 nM (FIG. 9A). The dissociation was monitored for 120 or 600 sec. Bulk refractive index differences were corrected for by subtracting the response obtained in a reference flow cell, where no protein was immobilized.

As can be seen in FIGS. 9B to 911, the CEA targeting-4-1BBL molecules can bind simultaneously human CEA (in form of N(A2B2)A or hu(NA1)BA construct), and human 4-1BB.

### Example 3

### Functional Characterization of the CEA-Targeting Split Trimeric 4-1BB Ligand Fc Fusion Antigen Binding Molecules

### 3.1 Binding Assays to CEACAM5 Expressing Cells

First, cell lines expressing cynomolgus monkey CEACAM5 or human CEACAM5 were generated. Full-length cDNAs encoding human and cynomolgus CEACAM5 were subcloned into mammalian expression vector. The plasmids were transfected into CHO-K1 (ATCC CRL-9618) cells using Lipofectamine LTX Reagent (Invitrogen, #15338100) according to the manufacturer's protocol. Stably transfected CEACAM5-positive CHO cells were maintained in DMEM/F-12 medium (GIBCO by Lifetechnologies, #11320033) supplemented with 10% fetal bovine serum (FBS, GIBCO by Life Technologies, Cat.-No. 16000-044, Lot 941273, gamma irradiated mycoplasma free, heat inactivated) and 2 mM L-alanyl-L-glutamine dipeptide (Gluta-MAX-I, GIBCO by Life Technologies, Cat.-No. 35050-038). Two days after transfection, puromycin (Invivogen; #ant-pr-1) was added to 6 μg/mL and the cells were cultured for several passages. After initial selection, the cells with high cell surface expression of human and cynomolgus CEACAM5 (detection antibody anti-CD66 clone CD66AB.1.1) were sorted by BD FACSAria II cell sorter (BD Biosciences) and cultured to establish stable cell clones. The expression level and stability was confirmed by flow cytometry analysis over a period of 4 weeks (see FIG. 10).

For the binding assay, CHO-k1-cynoCEACAM5 clone 8, CHO-k1-huCEACAM5 clone 11, CHO-k1-huCEACAM5 clone 12 CHO-k1-huCEACAM5 clone 13 or CHO-k1-huCEACAM5 clone 17 were harvested, washed with DPBS (GIBCO by life technologies, #14190-136) stained in DPBS containing fixable viability dye eF450 (eBioscience #65-0863-18) for 30 min at 4° C. Cells were washed and seeded to 384 well plates (Corning #3830) to 3×10⁴ cells/well. Cells were centrifuged (350×g, 5 min), supernatant was removed and cells were resuspended in 10 μL/well FACS-buffer (DPBS supplied with 2% FBS, 5 nM EDTA, 7.5 mM sodium azide) containing titrated concentrations of CEA-4-1BBL antibodies or controls (start concentration 300 nM). Cells were incubated for 30 minutes at 4° C. and then washed twice with 80 μL/well DPBS. Cells were resuspended in 10 μL/well FACS-buffer containing 2.5 μg/mL PE conjugated AffiniPure anti-human IgG Fcγ-fragment-specific goat F(ab′)2 fragment (Jackson ImmunoResearch, Cat.-No. 109-116-098) for 30 minutes at 4° C. Cells were washed twice with 80 μL/well DPBS and then fixed in 30 μL/well DPBS containing 1% Formaldehyde for at least 15 minutes. The same or the next day cells were resuspended in 50 μL/well FACS-buffer and acquired using MACSQuant Analyzer X (Miltenyi Biotec).

As shown in FIGS. 11A-11E and FIGS. 12A-12E and FIG. 13A-13C, the CEA-4-1BBL antibodies bind efficiently to human CEACAM5-expressing CHO-k1 clone 11, 12, 13 and 17. In contrast only CEA(A5B7)-4-1BBL, CEA (MEDI-565)-4-1BBL and CEA (A5H1EL1D)-4-1BBL as well as CEA (aff. mat. A5H1EL1D P001.117)-4-1BBL and CEA (aff. mat. A5H1EL1D P005.102)-4-1BBL bound to cynomolgus monkey CEACAM5 expressing CHO-k1-cynoCEACAM5 clone 8 cell line as well (FIG. 11A and FIG. 12A and FIG. 13A). Therefore, MFE23, huMFE23-L28-H24, huMFE23-L28-H28, T84.66-LCHA are not cross-reactive whereas A5B7, MEDI565, A5H1EL1D and aff. mat. A5H1EL1D based on clone P001.117 or P005.102 are cynomolgus monkey/human cross-reactive. However, A5H1EL1D and A5H1EL1D based on clone P001.117 show less cyno cross-reactivity than the parental clone A5B7.

The fitting EC₅₀ values and the values of area under the curve are listed in Table 43, Table 44, Table 45 and Table 46.

TABLE 43
EC₅₀ values of binding curves of different humanized CEA-4-
1BBL molecules displayed in FIGS. 11A-11E and FIGS. 12A-12E

| | CHO-k1- | CHO-k1- | CHO-k1- | CHO-k1- | CHO-k1- |
|---|---|---|---|---|---|
| | cyno | human | human | human | human |
| | CEACAM5 | CEACAM5 | CEACAM5 | CEACAM5 | CEACAM5 |
| EC50 [nM] | clone 8 | clone 11 | clone 12 | clone 13 | clone 17 |
| CEA (T84.66- | does not | 2.23 | 2.13 | 2.6 | 1.44 |
| LCHA)-4- | bind to | | | | |
| 1BBL | cynoCECAM | | | | |
| | 5 | | | | |
| CEA (A5B7)- | 31.4 | 10.03 | 10.98 | 15.17 | 7.38 |
| 4-1BBL | | | | | |
| CEA (MEDI- | 8.82 | 1.75 | 2.18 | 6.39 | 1.84 |
| 565)-4-1BBL | | | | | |
| CEA | not | 296 | 691 | not | 190 |
| (A5H1EL1D)- | determined as | | | determined as | |
| 4-1BBL | not in plateau | | | not in plateau | |
| CEA | does not | 0.54 | 2.25 | 5.17 | 0.6 |
| (MFE23)-4- | bind to | | | | |
| 1BBL | cynoCECAM | | | | |
| | 5 | | | | |
| CEA(huMFE2 | | 1.93 | 4.83 | 6.2 | 1.93 |
| 3-L28-H24)-4- | | | | | |
| 1BBL | | | | | |
| CEA(huMFE2 | | 2.98 | 8.32 | 15.44 | 3.26 |
| 3-L28-H28)-4- | | | | | |
| 1BBL | | | | | |
| DP47-4-1BBL | n.d. | n.d. | n.d. | n.d. | n.d. |

TABLE 44
Area under the curve (AUC) of binding curves of different humanized
CEA-4-1BBL molecules displayed in in FIGS. 11A-11E and FIGS. 12A-12E

| | CHO-k1-cyno | CHO-k1-human | CHO-k1-human | CHO-k1-human | CHO-k1-human |
|---|---|---|---|---|---|
| | CEACAM5 | CEACAM5 | CEACAM5 | CEACAM5 | CEACAM5 |
| AUC | clone 8 | clone 11 | clone 12 | clone 13 | clone 17 |
| CEA (T84.66- | 322 | 18769 | 71116 | 102929 | 22327 |
| LCHA)-4-1BBL | | | | | |
| CEA (A5B7)-4- | 15610 | 15032 | 55691 | 98387 | 16251 |
| 1BBL | | | | | |
| CEA (MEDI- | 20630 | 14283 | 49726 | 82088 | 15254 |
| 565)-4-lBBL | | | | | |
| (A5H1EL1D)-4- | 6192 | 8764 | 30539 | 48463 | 9084 |
| 1BBL | | | | | |
| CEA (MFE23)- | 430 | 33204 | 109307 | 168076 | 36340 |
| 4-1BBL | | | | | |
| CEA(huMFE23- | 275 | 24142 | 74663 | 100563 | 26002 |
| L28-H24)-4- | | | | | |
| 1BBL | | | | | |
| CEA(huMFE23- | 270 | 21714 | 65889 | 78487 | 23077 |
| L28-H28)-4- | | | | | |
| 1BBL | | | | | |
| DP47-4-1BBL | 520 | 1297 | 700 | 752 | 430 |

TABLE 45
EC₅₀ values of binding curves of different humanized
CEA-4-1BBL molecules displayed in FIGS. 13A-13C

| | CHO-k1-cyno | CHO-k1-human | CHO-k1-human |
|---|---|---|---|
| EC50 [nM] | CEACAM5 clone 8 | CEACAM5 clone 11 | CEACAM5 clone 12 |
| CEA (T84.66-LCHA)- | n.d. | 6.93 | 10.02 |
| 4-1BBL | | | |
| CEA (A5B7)-4-1BBL | 41.9 | 31.09 | 21.69 |
| CEA (MEDI-565)-4- | 9.43 | 6.68 | 10.88 |
| 1BBL | | | |
| CEA (A5HlELlD)-4- | not determined as | 61.68 | 148.1 |
| 1BBL | not in plateau | | |
| CEA(P001.177)-4- | not determined as | 33.67 | 43.96 |
| 1BBL | not in plateau | | |
| CEA(P005.102)-4- | 79.52 | 40.91 | 43.39 |
| 1BBL | | | |
| DP47-4-1BBL | n.d. | n.d. | n.d. |

TABLE 46
Area under the curve (AUC) of binding curves of different humanized
CEA-4-1BBL molecules displayed in in FIGS. 13A-13C

| | CHO-k1-cyno | CHO-k1-human | CHO-k1-human |
|---|---|---|---|
| AUC | CEACAM5 clone 8 | CEACAM5 clone 11 | CEACAM5 clone 12 |
| CEA (T84.66-LCHA)- | 5 | 3984 | 13576 |
| 4-1BBL | | | |
| CEA (A5B7)-4-1BBL | 2140 | 3008 | 7903 |
| CEA (MEDI-565)-4- | 3903 | 2851 | 8310 |
| 1BBL | | | |
| CEA (A5H1EL1D)-4- | 368 | 1532 | 4421 |
| 1BBL | | | |
| CEA(P001.177)-4- | 164 | 3630 | 8348 |
| 1BBL | | | |
| CEA(P005.102)-4- | 2767 | 3181 | 8992 |
| 1BBL | | | |
| DP47-4-1BBL | 11 | 148 | 140 |

### 3.2 NFκB Activation in Human 4-1BB and NFκB-Luciferase Reporter Gene Expressing Reporter Cell Line Jurkat-Hu4-1BB-NFκB-Luc2

Agonistic binding of the 4-1BB (CD137) receptor to its ligand (4-1BBL) induces 4-1BB-downstream signaling via activation of nuclear factor kappa B (NFκB) and promotes survival and activity of CD8 T cells (Lee H W, Park S J, Choi B K, Kim H H, Nam K O, Kwon B S, J Immunol 2002; 169, 4882-4888). To monitor this NFκB-activation mediated by CEA-4-1BBL antigen binding molecules, Jurkat-hu4-1BB-NFκB-luc2 reporter cell line was purchased from Promega (Germany). The cells were cultured as described above. For the assay, cells were harvested and resuspended in assay medium RPMI 1640 medium supplied with 10% (v/v) FBS and 1% (v/v) GlutaMAX-I. 10 μL containing 2×10³ Jurkat-hu4-1BB-NFκB-luc2 reporter cells were transferred to each well of a sterile white 384-well flat bottom tissue culture plate with lid (Corning, Cat.-No.:3826). 10 μL of assay medium containing titrated concentrations of CEA-4-1BBL antibodies or control molecules were added. Finally, 10 μL of assay medium alone or containing 1×10⁴ cells of different CHO-k1 cells transfected with cynomolgus monkey or human CEACAM5 were supplied and plates were incubated for 6 hours at 37° C. and 5% CO₂ in a cell incubator. 6 μl freshly thawed One-Glo Luciferase assay detection solution (Promega, Cat.-No.: E6110) were added to each well and Luminescence light emission were measured immediately using Tecan microplate reader (500 ms integration time, no filter collecting all wavelength).

As shown in FIGS. 14A-14D and FIGS. 15A-15D, in the absence of CEACAM5 expressing cells none of the molecules was able to induce strong human 4-1BB receptor activation in the Jurkat-hu4-1BB-NFκB-luc2 reporter cell line, leading to NFκB-activation and therefore Luciferase expression. In the presence of human CEACAM5-expressing cells like CHO-k1-human CEACAM5 clone 11 and CHO-k1-human CEACAM5 clone 12 crosslinking of CEA-4-1BBL antibodies led to a strong increase of NFκB-activated Luciferase activity in the Jurkat-hu4-1BB-NFκB-luc2 reporter cell line, which was above the activation mediated by the untargeted control DP47-4-1BBL. In the presence of CHO-k1-cyno CEACAM5 clone 8 activation induction depends on the used CEACAM5-binding clone. CEA-4-1BBL antibodies containing clones MFE23 (parental) or humanized MFE23 clones huMFE23-L28-H24 or huMFE23-L28-H28 or humanized MFE23 clone sm9b (disclosed in US 2005/0147614) or reference clone T84.66-LCHA do not bind to cynomolgus monkey CEACAM5 and therefore do not induce Jurkat-hu4-1BB-NFκB-luc2 reporter cell activation (FIG. 14B). On the other hand, CEA-4-1BBL antibodies containing cynomolgus monkey/human-crossreactive clones A5B7 (parental), humanized A5B7 clones MEDI-565 or A5H1EL1D or affinity maturated A5H1EL1D based clones P005-102 or P001-177 induce Jurkat-hu4-1BB-NFκB-luc2 reporter cell activation also in the presence of CHO-k1-cyno CEACAM5 clone 8 (FIG. 15B).

EC₅₀ values and area under the curve (AUC) of activation curves are listed in Table 47 and Table 48, respectively.

TABLE 47
EC₅₀ values of activation curves of different humanized CEA-
4-1BBL molecules displayed in FIGS. 14A-14D and FIGS. 15A-15D

| | | plate 1 CHO-k1- | plate 1 CHO-k1- | plate 1 CHO-k1- |
|---|---|---|---|---|
| | | cyno | human | human |
| | | CEACAM5 | CEACAM5 | CEACAM5 |
| EC₅₀ [nM] | no CEA⁺ cells | clone 8 | clone 11 | clone 12 |
| DP47-4-1BBL | n.d. | Not cyno- | n.d. | n.d. |
| CEA (MFE23)-4-1BBL | n.d. | crossreactive | 0.08 | 0.05 |
| CEA(huMFE23-L28- | n.d. | | 0.06 | 0.04 |
| H24)-4-1BBL | | | | |
| CEA(huMFE23-L28- | n.d. | | 0.10 | 0.07 |
| H28)-4-1BBL | | | | |
| CEA(sm9b)-4-1BBL | n.d. | | 0.05 | 0.03 |
| CEA(sm9b)-4-1BBL | n.d. | | 0.09 | 0.07 |
| single chain | | | | |
| CEA (A5B7)-4-1BBL | n.d. | 0.17 | 0.12 | 0.08 |
| CEA (MEDI-565)-4- | n.d. | 0.20 | 0.11 | 0.06 |
| 1BBL | | | | |
| CEA (A5H1EL1D)-4- | n.d. | 0.53 | 0.16 | 0.09 |
| 1BBL | | | | |
| CEA(P001.177)-4-lBBL | n.d. | 0.13 | 0.05 | 0.03 |
| CEA(P005.102)-4-lBBL | n.d. | 0.12 | 0.10 | 0.08 |
| CEA (T84.66-LCHA)-4- | n.d. | Not cyno- | 0.12 | 0.04 |
| 1BBL | | crossreactive | | |

TABLE 48
Area under the curve (AUC) of activation curves of different humanized
CEA-4-1BBL molecules displayed in in FIGS. 14A-14D and FIGS. 15A-15D

| | | plate 1 CHO-k1- | plate 1 CHO-k1- | plate 1 CHO-k1- |
|---|---|---|---|---|
| | | cyno | human | human |
| | | CEACAM5 | CEACAM5 | CEACAM5 |
| AUC | no CEA⁺ cells | clone 8 | clone 11 | clone 12 |
| DP47-4-1BBL | 2929 | 4713 | 8636 | 6635 |
| CEA (MFE23)-4-lBBL | 2664 | 3940 | 183090 | 175535 |
| CEA(huMFE23-L28-H24)- | 2561 | 3600 | 161174 | 162747 |
| 4-1BBL | | | | |
| CEA(huMFE23-L28-H28)- | 2528 | 3418 | 147450 | 145894 |
| 4-1BBL | | | | |
| CEA(sm9b)-4-lBBL | 2811 | 3968 | 162068 | 164198 |
| CEA(sm9b)-4-lBBL single | 1976 | 2815 | 150488 | 145930 |
| chain | | | | |
| CEA (A5B7)-4-lBBL | 2038 | 88745 | 145776 | 136594 |
| CEA (MEDI-565)-4-lBBL | 2267 | 76333 | 154791 | 154767 |
| CEA (A5HlELlD)-4-lBBL | 2565 | 61720 | 130920 | 136891 |
| CEA(P001.177)-4-lBBL | 2870 | 72145 | 173531 | 171049 |
| CEA(P005.102)-4-lBBL | 2507 | 88789 | 151541 | 130963 |
| CEA (T84.66-LCHA)-4- | 3511 | 4978 | 82607 | 92372 |
| 1BBL | | | | |

1. A 4-1BBL trimer-containing antigen binding molecule comprising an antigen binding domain capable of specific binding to CEA,
a first and a second polypeptide that are linked to each other by a disulfide bond,
wherein the antigen binding molecule is characterized in that the first polypeptide comprises two ectodomains of 4-1BBL or a fragment thereof that are connected to each other by a peptide linker and in that the second polypeptide comprises one ectodomain of 4-1BBL or a fragment thereof, and an Fc domain composed of a first and a second subunit capable of stable association,
wherein the antigen binding domain capable of specific binding to CEA comprises
(a) a variable heavy chain domain (VH) comprising (i) CDR-H1 comprising the amino acid sequence of SEQ ID NO:17, (ii) CDR-H2 comprising the amino acid sequence of SEQ ID NO:18, and (iii) CDR-H3 comprising the amino acid sequence of SEQ ID NO:19, and a variable light chain domain (VL) comprising (iv) CDR-L1 comprising the amino acid sequence of SEQ ID NO:20, (v) CDR-L2 comprising the amino acid sequence of SEQ ID NO:21, and (vi) CDR-L3 comprising the amino acid sequence of SEQ ID NO:22, or
(b) a VH domain comprising (i) CDR-H1 comprising the amino acid sequence of SEQ ID NO:25, (ii) CDR-H2 comprising the amino acid sequence of SEQ ID NO:26, and (iii) CDR-H3 comprising the amino acid sequence of SEQ ID NO:27, and a VL domain comprising (iv) CDR-L1 comprising the amino acid sequence of SEQ ID NO:28, (v) CDR-L2 comprising the amino acid sequence of SEQ ID NO:29, and (vi) CDR-L3 comprising the amino acid sequence of SEQ ID NO:30, or
(c) a VH domain comprising (i) CDR-H1 comprising the amino acid sequence of SEQ ID NO:65, (ii) CDR-H2 comprising the amino acid sequence of SEQ ID NO:66 or SEQ ID NO:67, and (iii) CDR-H3 comprising the amino acid sequence of SEQ ID NO:68, and a VL domain comprising (iv) CDR-L1 comprising the amino acid sequence of SEQ ID NO:69 or SEQ ID NO:70 or SEQ ID NO:313, (v) CDR-L2 comprising the amino acid sequence of SEQ ID NO:71 or SEQ ID NO:72 or SEQ ID NO:73, and (vi) CDR-L3 comprising the amino acid sequence of SEQ ID NO:74.

2. The 4-1BBL trimer-containing antigen binding molecule of claim 1, wherein the ectodomain of 4-1BBL or a fragment thereof comprises the amino acid sequence selected from the group consisting of SEQ ID NO:87, SEQ ID NO:88, SEQ ID NO:89, SEQ ID NO:90, SEQ ID NO:91, SEQ ID NO:92, SEQ ID NO:93 and SEQ ID NO:94, particularly the amino acid sequence of SEQ ID NO:91.

3. The 4-1BBL trimer-containing antigen binding molecule of claim 1, comprising
(a) an antigen binding domain capable of specific binding to CEA,
(b) a first and a second polypeptide that are linked to each other by a disulfide bond, wherein the antigen binding molecule is characterized in that the first polypeptide comprises the amino acid sequence selected from the group consisting of SEQ ID NO:95, SEQ ID NO:96, SEQ ID NO:97 and SEQ ID NO:98 and in that the second polypeptide comprises the amino acid sequence selected from the group consisting of SEQ ID NO:87, SEQ ID NO:91, SEQ ID NO:89 and SEQ ID NO:94, and
(c) an Fc domain composed of a first and a second subunit capable of stable association,
wherein the antigen binding domain capable of specific binding to CEA comprises
(a) a variable heavy chain domain (VH) comprising (i) CDR-H1 comprising the amino acid sequence of SEQ ID NO:17, (ii) CDR-H2 comprising the amino acid sequence of SEQ ID NO:18, and (iii) CDR-H3 comprising the amino acid sequence of SEQ ID NO:19, and a variable light chain domain (VL) comprising (iv) CDR-L1 comprising the amino acid sequence of SEQ ID NO:20, (v) CDR-L2 comprising the amino acid sequence of SEQ ID NO:21, and (vi) CDR-L3 comprising the amino acid sequence of SEQ ID NO:22, or
(b) a VH domain comprising (i) CDR-H1 comprising the amino acid sequence of SEQ ID NO:25, (ii) CDR-H2 comprising the amino acid sequence of SEQ ID NO:26, and (iii) CDR-H3 comprising the amino acid sequence of SEQ ID NO:27, and a VL domain comprising (iv) CDR-L1 comprising the amino acid sequence of SEQ ID NO:28, (v) CDR-L2 comprising the amino acid sequence of SEQ ID NO:29, and (vi) CDR-L3 comprising the amino acid sequence of SEQ ID NO:30, or
(c) a VH domain comprising (i) CDR-H1 comprising the amino acid sequence of SEQ ID NO:65, (ii) CDR-H2 comprising the amino acid sequence of SEQ ID NO:66 or SEQ ID NO:67, and (iii) CDR-H3 comprising the amino acid sequence of SEQ ID NO:68, and a VL domain comprising (iv) CDR-L1 comprising the amino acid sequence of SEQ ID NO:69 or SEQ ID NO:70 or SEQ ID NO:313, (v) CDR-L2 comprising the amino acid sequence of SEQ ID NO:71 or SEQ ID NO:72 or SEQ ID NO:73, and (vi) CDR-L3 comprising the amino acid sequence of SEQ ID NO:74.

4. The 4-1BBL trimer-containing antigen binding molecule of claim 1, wherein the Fc domain comprises knob-into-hole modifications promoting association of the first and the second subunit of the Fc domain.

5. The 4-1BBL trimer-containing antigen binding molecule of claim 1, wherein the Fc domain comprises one or more amino acid substitution that reduces binding to an Fc receptor, in particular towards Fcγ receptor.

6. The 4-1BBL trimer-containing antigen binding molecule of claim 1, wherein the Fc domain is an IgG1 Fc domain comprising the amino acid substitutions the amino acid substitutions L234A, L235A and P329G (numbering according to Kabat EU numbering).

7. The 4-1BBL trimer-containing antigen binding molecule of claim 1, wherein the antigen binding domain capable of specific binding to CEA is a Fab molecule capable of specific binding to CEA.

8. The 4-1BBL trimer-containing antigen binding molecule of claim 1, wherein the antigen binding domain capable of specific binding to CEA comprises
(a) a variable heavy chain domain (VH) comprising (i) CDR-H1 comprising the amino acid sequence of SEQ ID NO:17, (ii) CDR-H2 comprising the amino acid sequence of SEQ ID NO:18, and (iii) CDR-H3 comprising the amino acid sequence of SEQ ID NO:19, and a variable light chain domain (VL) comprising (iv) CDR-L1 comprising the amino acid sequence of SEQ ID NO:20, (v) CDR-L2 comprising the amino acid sequence of SEQ ID NO:21, and (vi) CDR-L3 comprising the amino acid sequence of SEQ ID NO:22, or
(b) a VH domain comprising (i) CDR-H1 comprising the amino acid sequence of SEQ ID NO:25, (ii) CDR-H2 comprising the amino acid sequence of SEQ ID NO:26, and (iii) CDR-H3 comprising the amino acid sequence of SEQ ID NO:27, and a VL domain comprising (iv) CDR-L1 comprising the amino acid sequence of SEQ ID NO:28, (v) CDR-L2 comprising the amino acid sequence of SEQ ID NO:29, and (vi) CDR-L3 comprising the amino acid sequence of SEQ ID NO:30.

9. The 4-1BBL trimer-containing antigen binding molecule of claim 1, wherein the antigen binding domain capable of specific binding to CEA comprises
(a) a VH domain comprising the amino acid sequence of SEQ ID NO:23 and a VL domain comprising the amino acid sequence of SEQ ID NO:24, or
(b) a VH domain comprising the amino acid sequence of SEQ ID NO:31 and a VL domain comprising the amino acid sequence of SEQ ID NO:32, or
(c) a VH domain comprising the amino acid sequence of SEQ ID NO:33 and a VL domain comprising the amino acid sequence of SEQ ID NO:34, or
(d) a VH domain comprising the amino acid sequence of SEQ ID NO:35 and a VL domain comprising the amino acid sequence of SEQ ID NO:36, or
(e) a VH domain comprising the amino acid sequence of SEQ ID NO:37 and a VL domain comprising the amino acid sequence of SEQ ID NO:38, or
(f) a VH domain comprising the amino acid sequence of SEQ ID NO:39 and a VL domain comprising the amino acid sequence of SEQ ID NO:40, or
(g) a VH domain comprising the amino acid sequence of SEQ ID NO:41 and a VL domain comprising the amino acid sequence of SEQ ID NO:42, or
(h) a VH domain comprising the amino acid sequence of SEQ ID NO:43 and a VL domain comprising the amino acid sequence of SEQ ID NO:44, or
(i) a VH domain comprising the amino acid sequence of SEQ ID NO:45 and a VL domain comprising the amino acid sequence of SEQ ID NO:46, or
(j) a VH domain comprising the amino acid sequence of SEQ ID NO:47 and a VL domain comprising the amino acid sequence of SEQ ID NO:48, or
(k) a VH domain comprising the amino acid sequence of SEQ ID NO:49 and a VL domain comprising the amino acid sequence of SEQ ID NO:50,
(l) a VH domain comprising the amino acid sequence of SEQ ID NO:51 and a VL domain comprising the amino acid sequence of SEQ ID NO:52, or
(m) a VH domain comprising the amino acid sequence of SEQ ID NO:53 and a VL domain comprising the amino acid sequence of SEQ ID NO:54.

10. The 4-1BBL trimer-containing antigen binding molecule of claim 1, wherein the antigen binding domain capable of specific binding to CEA comprises
a VH domain comprising (i) CDR-H1 comprising the amino acid sequence of SEQ ID NO:65, (ii) CDR-H2 comprising the amino acid sequence of SEQ ID NO:66 or SEQ ID NO:67, and (iii) CDR-H3 comprising the amino acid sequence of SEQ ID NO:68, and
a VL domain comprising (iv) CDR-L1 comprising the amino acid sequence of SEQ ID NO:69 or SEQ ID NO:70 or SEQ ID NO:313, (v) CDR-L2 comprising the amino acid sequence of SEQ ID NO:71 or SEQ ID NO:72 or SEQ ID NO:73, and (vi) CDR-L3 comprising the amino acid sequence of SEQ ID NO:74.

11. The 4-1BBL trimer-containing antigen binding molecule of claim 1, wherein the antigen binding domain capable of specific binding to CEA comprises
a heavy chain variable region (VH) comprising the amino acid sequence of SEQ ID NO:75, SEQ ID NO:76, SEQ ID NO:77, SEQ ID NO:78, SEQ ID NO:79 or SEQ ID NO:80, and
a light chain variable region (VL) comprising the amino acid sequence of SEQ ID NO:81, SEQ ID NO:82, SEQ ID NO:83, SEQ ID NO:84, SEQ ID NO:85 or SEQ ID NO:86.

12. The 4-1BBL trimer-containing antigen binding molecule of claim 1, wherein the antigen binding domain capable of specific binding to CEA comprises
(a) a VH domain comprising the amino acid sequence of SEQ ID NO:75 and a VL domain comprising the amino acid sequence of SEQ ID NO:85, or
(b) a VH domain comprising the amino acid sequence of SEQ ID NO:79 and a VL domain comprising the amino acid sequence of SEQ ID NO:85, or
(c) a VH domain comprising the amino acid sequence of SEQ ID NO:76 and a VL domain comprising the amino acid sequence of SEQ ID NO:85, or
(d) a VH domain comprising the amino acid sequence of SEQ ID NO:80 and a VL domain comprising the amino acid sequence of SEQ ID NO:84, or
(e) a VH domain comprising the amino acid sequence of SEQ ID NO:79 and a VL domain comprising the amino acid sequence of SEQ ID NO:84, or
(f) a VH domain comprising the amino acid sequence of SEQ ID NO:77 and a VL domain comprising the amino acid sequence of SEQ ID NO:84, or
(g) a VH domain comprising the amino acid sequence of SEQ ID NO:75 and a VL domain comprising the amino acid sequence of SEQ ID NO:84.

13. The 4-1BBL trimer-containing antigen binding molecule of claim 1, wherein the first peptide comprising two ectodomains of 4-1BBL or a fragment thereof connected to each other by a first peptide linker is fused at its C-terminus by a second peptide linker to a CL domain that is part of a heavy chain, and the second peptide comprising one ectodomain of said 4-1BBL or a fragment thereof is fused at its C-terminus by a third peptide linker to a CH1 domain that is part of a light chain.

14. The 4-1BBL trimer-containing antigen binding molecule of claim 1, wherein the antigen binding molecule comprises
(i) a first heavy chain and a first light chain, both comprising a Fab molecule capable of specific binding to CEA,
(ii) a second heavy chain comprising the amino acid sequence selected from the group consisting of SEQ ID NO:99, SEQ ID NO:101, SEQ ID NO:103 and SEQ ID NO:105, and
(iii) a second light chain comprising the amino acid sequence selected from the group consisting of SEQ ID NO:100, SEQ ID NO:102, SEQ ID NO:104 and SEQ ID NO:106.

15. The 4-1BBL trimer-containing antigen binding molecule of claim 1, wherein the antigen binding molecule comprises
(a) a first heavy chain comprising the amino acid sequence of SEQ ID NO:99, a first light chain comprising the amino acid sequence of SEQ ID NO:100, a second heavy chain comprising the amino acid sequence of SEQ ID NO:238 and a second light chain comprising the amino acid sequence of SEQ ID NO:239, or
(b) a first heavy chain comprising the amino acid sequence of SEQ ID NO:99, a first light chain comprising the amino acid sequence of SEQ ID NO:100, a second heavy chain comprising the amino acid sequence of SEQ ID NO:240 and a second light chain comprising the amino acid sequence of SEQ ID NO:241, or
(c) a first heavy chain comprising the amino acid sequence of SEQ ID NO:99, a first light chain comprising the amino acid sequence of SEQ ID NO:100, a second heavy chain comprising the amino acid sequence of SEQ ID NO:242 and a second light chain comprising the amino acid sequence of SEQ ID NO:243, or
(d) a first heavy chain comprising the amino acid sequence of SEQ ID NO:99, a first light chain comprising the amino acid sequence of SEQ ID NO:100, a second heavy chain comprising the amino acid sequence of SEQ ID NO:244 and a second light chain comprising the amino acid sequence of SEQ ID NO:245, or
(e) a first heavy chain comprising the amino acid sequence of SEQ ID NO:99, a first light chain comprising the amino acid sequence of SEQ ID NO:100, a second heavy chain comprising the amino acid sequence of SEQ ID NO:246 and a second light chain comprising the amino acid sequence of SEQ ID NO:247, or
(f) a first heavy chain comprising the amino acid sequence of SEQ ID NO:99, a first light chain comprising the amino acid sequence of SEQ ID NO:100, a second heavy chain comprising the amino acid sequence of SEQ ID NO:248 and a second light chain comprising the amino acid sequence of SEQ ID NO:249, or
(g) a first heavy chain comprising the amino acid sequence of SEQ ID NO:99, a first light chain comprising the amino acid sequence of SEQ ID NO:100, a second heavy chain comprising the amino acid sequence of SEQ ID NO:250 and a second light chain comprising the amino acid sequence of SEQ ID NO:251, or
(h) a first heavy chain comprising the amino acid sequence of SEQ ID NO:99, a first light chain comprising the amino acid sequence of SEQ ID NO:100, a second heavy chain comprising the amino acid sequence of SEQ ID NO:252 and a second light chain comprising the amino acid sequence of SEQ ID NO:253, or
(i) a first heavy chain comprising the amino acid sequence of SEQ ID NO:99, a first light chain comprising the amino acid sequence of SEQ ID NO:100, a second heavy chain comprising the amino acid sequence of SEQ ID NO:254 and a second light chain comprising the amino acid sequence of SEQ ID NO:255, or
(j) a first heavy chain comprising the amino acid sequence of SEQ ID NO:99, a first light chain comprising the amino acid sequence of SEQ ID NO:100, a second heavy chain comprising the amino acid sequence of SEQ ID NO:256 and a second light chain comprising the amino acid sequence of SEQ ID NO:257, or
(k) a first heavy chain comprising the amino acid sequence of SEQ ID NO:99, a first light chain comprising the amino acid sequence of SEQ ID NO:100, a second heavy chain comprising the amino acid sequence of SEQ ID NO:258 and a second light chain comprising the amino acid sequence of SEQ ID NO:259, or
(l) a first heavy chain comprising the amino acid sequence of SEQ ID NO:99, a first light chain comprising the amino acid sequence of SEQ ID NO:100, a second heavy chain comprising the amino acid sequence of SEQ ID NO:260 and a second light chain comprising the amino acid sequence of SEQ ID NO:261, or
(m) a first heavy chain comprising the amino acid sequence of SEQ ID NO:49, a first light chain comprising the amino acid sequence of SEQ ID NO:50, a second heavy chain comprising the amino acid sequence of SEQ ID NO:262 and a second light chain comprising the amino acid sequence of SEQ ID NO:263.

16. The 4-1BBL trimer-containing antigen binding molecule of claim 1, wherein the antigen binding molecule comprises
(a) a first heavy chain comprising the amino acid sequence of SEQ ID NO:99, a first light chain comprising the amino acid sequence of SEQ ID NO:100, a second heavy chain comprising the amino acid sequence of SEQ ID NO:266 and a second light chain comprising the amino acid sequence of SEQ ID NO:267, or
(b) a first heavy chain comprising the amino acid sequence of SEQ ID NO:99, a first light chain comprising the amino acid sequence of SEQ ID NO:100, a second heavy chain comprising the amino acid sequence of SEQ ID NO:268 and a second light chain comprising the amino acid sequence of SEQ ID NO:267, or
(c) a first heavy chain comprising the amino acid sequence of SEQ ID NO:99, a first light chain comprising the amino acid sequence of SEQ ID NO:100, a second heavy chain comprising the amino acid sequence of SEQ ID NO:269 and a second light chain comprising the amino acid sequence of SEQ ID NO:267, or
(d) a first heavy chain comprising the amino acid sequence of SEQ ID NO:99, a first light chain comprising the amino acid sequence of SEQ ID NO:100, a second heavy chain comprising the amino acid sequence of SEQ ID NO:270 and a second light chain comprising the amino acid sequence of SEQ ID NO:271, or
(e) a first heavy chain comprising the amino acid sequence of SEQ ID NO:99, a first light chain comprising the amino acid sequence of SEQ ID NO:100, a second heavy chain comprising the amino acid sequence of SEQ ID NO:272 and a second light chain comprising the amino acid sequence of SEQ ID NO:271, or
(f) a first heavy chain comprising the amino acid sequence of SEQ ID NO:99, a first light chain comprising the amino acid sequence of SEQ ID NO:100, a second heavy chain comprising the amino acid sequence of SEQ ID NO:273 and a second light chain comprising the amino acid sequence of SEQ ID NO:271, or
(g) a first heavy chain comprising the amino acid sequence of SEQ ID NO:99, a first light chain comprising the amino acid sequence of SEQ ID NO:100, a second heavy chain comprising the amino acid sequence of SEQ ID NO:274 and a second light chain comprising the amino acid sequence of SEQ ID NO:271.

17. A humanized antibody that binds to carcinoembryonic antigen (CEA), comprising
(a) a variable heavy chain domain (VH) comprising (i) CDR-H1 comprising the amino acid sequence of SEQ ID NO:17, (ii) CDR-H2 comprising the amino acid sequence of SEQ ID NO:18, and (iii) CDR-H3 comprising the amino acid sequence of SEQ ID NO:19, and a variable light chain domain (VL) comprising (iv) CDR-L1 comprising the amino acid sequence of SEQ ID NO:20, (v) CDR-L2 comprising the amino acid sequence of SEQ ID NO:21, and (vi) CDR-L3 comprising the amino acid sequence of SEQ ID NO:22, or
(b) a VH domain comprising (i) CDR-H1 comprising the amino acid sequence of SEQ ID NO:25, (ii) CDR-H2 comprising the amino acid sequence of SEQ ID NO:26, and (iii) CDR-H3 comprising the amino acid sequence of SEQ ID NO:27, and a VL domain comprising (iv) CDR-L1 comprising the amino acid sequence of SEQ ID NO:28, (v) CDR-L2 comprising the amino acid sequence of SEQ ID NO:29, and (vi) CDR-L3 comprising the amino acid sequence of SEQ ID NO:30.

18. The humanized antibody of claim 17, wherein the antibody comprises
(a) a VH domain comprising the amino acid sequence of SEQ ID NO:23 and a VL domain comprising the amino acid sequence of SEQ ID NO:24, or
(b) a VH domain comprising the amino acid sequence of SEQ ID NO:31 and a VL domain comprising the amino acid sequence of SEQ ID NO:32, or
(c) a VH domain comprising the amino acid sequence of SEQ ID NO:33 and a VL domain comprising the amino acid sequence of SEQ ID NO:34, or
(d) a VH domain comprising the amino acid sequence of SEQ ID NO:35 and a VL domain comprising the amino acid sequence of SEQ ID NO:36, or
(e) a VH domain comprising the amino acid sequence of SEQ ID NO:37 and a VL domain comprising the amino acid sequence of SEQ ID NO:38, or
(f) a VH domain comprising the amino acid sequence of SEQ ID NO:39 and a VL domain comprising the amino acid sequence of SEQ ID NO:40, or
(g) a VH domain comprising the amino acid sequence of SEQ ID NO:41 and a VL domain comprising the amino acid sequence of SEQ ID NO:42, or
(h) a VH domain comprising the amino acid sequence of SEQ ID NO:43 and a VL domain comprising the amino acid sequence of SEQ ID NO:44, or
(i) a VH domain comprising the amino acid sequence of SEQ ID NO:45 and a VL domain comprising the amino acid sequence of SEQ ID NO:46, or
(j) a VH domain comprising the amino acid sequence of SEQ ID NO:47 and a VL domain comprising the amino acid sequence of SEQ ID NO:48, or
(k) a VH domain comprising the amino acid sequence of SEQ ID NO:49 and a VL domain comprising the amino acid sequence of SEQ ID NO:50,
(l) a VH domain comprising the amino acid sequence of SEQ ID NO:51 and a VL domain comprising the amino acid sequence of SEQ ID NO:52, or
(m) a VH domain comprising the amino acid sequence of SEQ ID NO:53 and a VL domain comprising the amino acid sequence of SEQ ID NO:54.

19. A humanized antibody that binds to carcinoembryonic antigen (CEA), comprising
a VH domain comprising (i) CDR-H1 comprising the amino acid sequence of SEQ ID NO:65, (ii) CDR-H2 comprising the amino acid sequence of SEQ ID NO:66 or SEQ ID NO:67, and (iii) CDR-H3 comprising the amino acid sequence of SEQ ID NO:68, and
a VL domain comprising (iv) CDR-L1 comprising the amino acid sequence of SEQ ID NO:69 or SEQ ID NO:70 or SEQ ID NO:313, (v) CDR-L2 comprising the amino acid sequence of SEQ ID NO:71 or SEQ ID NO:72 or SEQ ID NO:73, and (vi) CDR-L3 comprising the amino acid sequence of SEQ ID NO:74.

20. The humanized antibody of claim 19, wherein the antibody comprises a heavy chain variable region (VH) comprising the amino acid sequence of SEQ ID NO:75, SEQ ID NO:76, SEQ ID NO:77, SEQ ID NO:78, SEQ ID NO:79 or SEQ ID NO:80, and a light chain variable region (VL) comprising the amino acid sequence of SEQ ID NO:81, SEQ ID NO:82, SEQ ID NO:83, SEQ ID NO:84, SEQ ID NO:85 or SEQ ID NO:86.

21. The humanized antibody of claim 19, wherein the antigen binding domain capable of specific binding to CEA comprises
(a) a VH domain comprising the amino acid sequence of SEQ ID NO:75 and a VL domain comprising the amino acid sequence of SEQ ID NO:85, or
(b) a VH domain comprising the amino acid sequence of SEQ ID NO:79 and a VL domain comprising the amino acid sequence of SEQ ID NO:85, or
(c) a VH domain comprising the amino acid sequence of SEQ ID NO:76 and a VL domain comprising the amino acid sequence of SEQ ID NO:85, or
(d) a VH domain comprising the amino acid sequence of SEQ ID NO:80 and a VL domain comprising the amino acid sequence of SEQ ID NO:84, or
(e) a VH domain comprising the amino acid sequence of SEQ ID NO:79 and a VL domain comprising the amino acid sequence of SEQ ID NO:84, or
(f) a VH domain comprising the amino acid sequence of SEQ ID NO:77 and a VL domain comprising the amino acid sequence of SEQ ID NO:84, or
(g) a VH domain comprising the amino acid sequence of SEQ ID NO:75 and a VL domain comprising the amino acid sequence of SEQ ID NO:84.

22. The antibody of claim 17, wherein the antibody is an antibody fragment, in particular a Fab molecule, that specifically binds to CEA.

23. The antibody of claim 17, wherein the antibody is a full length IgG1 antibody.

24. An isolated nucleic acid encoding the 4-1BBL trimer-containing antigen binding molecule of claim 1.

25. A host cell comprising the nucleic acid of claim 24.

26. A method of producing the 4-1BBL trimer-containing antigen binding molecule comprising culturing the host cell of claim 25 under conditions suitable for expression of the 4-1BBL trimer-containing antigen binding molecule.

27-28. (canceled)

29. A pharmaceutical composition comprising the 4-1BBL trimer-containing antigen binding molecule of claim 1 and at least one pharmaceutically acceptable excipient.

30. The pharmaceutical composition of claim 31, further comprising an additional therapeutic agent.

31. The pharmaceutical composition of claim 30, further comprising a T-cell activating anti-CD3 bispecific antibody.

32-36. (canceled)

37. A method of treating an individual having cancer comprising administering to the individual an effective amount of the 4-1BBL trimer-containing antigen binding molecule of claim 1.

38. The method of claim 37, wherein the method further comprises administering to the subject an effective amount of a T-cell activating anti-CD3 bispecific antibody.

39. A method of up-regulating or prolonging cytotoxic T cell activity in an individual having cancer, comprising administering to the individual an effective amount of the 4-1BBL trimer-containing antigen binding molecule of claim 1.

40. An isolated nucleic acid encoding the antibody of claim 17.

41. A host cell comprising the nucleic acid of claim 40.

42. A method of producing an antibody comprising culturing the host cell of claim 41 under conditions suitable for expression of the antibody.

43. A pharmaceutical composition comprising the antibody of claim 17 and at least one pharmaceutically acceptable excipient.

44. A method of treating an individual having cancer comprising administering to the individual an effective amount of the antibody of claim 17.

45. A method of up-regulating or prolonging cytotoxic T cell activity in an individual having cancer, comprising administering to the individual an effective amount of the antibody of claim 17.

46. An isolated nucleic acid encoding the antibody of claim 19.

47. A host cell comprising the nucleic acid of claim 46.

48. A method of producing an antibody comprising culturing the host cell of claim 47 under conditions suitable for expression of the antibody.

49. A pharmaceutical composition comprising the antibody of claim 19 and at least one pharmaceutically acceptable excipient.

50. A method of treating an individual having cancer comprising administering to the individual an effective amount of the antibody of claim 19.

51. A method of up-regulating or prolonging cytotoxic T cell activity in an individual having cancer, comprising administering to the individual an effective amount of the antibody of claim 19.


SEQUENCE LISTING (313 sequences)

SEQ ID NO: 1 - protein, 5 aa, Artificial Sequence
  CEA (A5B7)- CDR-H1
DYYMN

SEQ ID NO: 2 - protein, 19 aa, Artificial Sequence
  CEA (A5B7)- CDR-H2
FIGNKANGYTTEYSASVKG

SEQ ID NO: 3 - protein, 10 aa, Artificial Sequence
  CEA (A5B7)- CDR-H3
DRGLRFYFDY

SEQ ID NO: 4 - protein, 10 aa, Artificial Sequence
  CEA (A5B7)- CDR-L1
RASSSVTYIH

SEQ ID NO: 5 - protein, 7 aa, Artificial Sequence
  CEA (A5B7)- CDR-L2
ATSNLAS

SEQ ID NO: 6 - protein, 9 aa, Artificial Sequence
  CEA (A5B7)- CDR-L3
QHWSSKPPT

SEQ ID NO: 7 - protein, 121 aa, Artificial Sequence
  CEA (A5B7) VH (parental)
EVKLVESGGGLVQPGGSLRLSCATSGFTFTDYYMNWVRQPPGKALEWLGFIGNKANGYTTEYSASVKGRFTISRDKSQSILYLQMNTLRAEDSATYYCTRDRGLRFYFDYWGQGTTLTVSS

SEQ ID NO: 8 - protein, 106 aa, Artificial Sequence
  CEA (A5B7) VL (parental)
QTVLSQSPAILSASPGEKVTMTCRASSSVTYIHWYQQKPGSSPKSWIYATSNLASGVPARFSGSGSGTSYSLTISRVEAEDAATYYCQHWSSKPPTFGGGTKLEIK

SEQ ID NO: 9 - protein, 5 aa, Artificial Sequence
  CEA CDR-H1
SYWMH

SEQ ID NO: 10 - protein, 19 aa, Artificial Sequence
  CEA CDR-H2
FIRNKANGGTTEYAASVKG

SEQ ID NO: 11 - protein, 10 aa, Artificial Sequence
  CEA CDR-H3
DRGLRFYFDY

SEQ ID NO: 12 - protein, 14 aa, Artificial Sequence
  CEA CDR-L1
TLRRGINVGAYSIY

SEQ ID NO: 13 - protein, 13 aa, Artificial Sequence
  CEA CDR-L2
YKSDSDKQQGSGV

SEQ ID NO: 14 - protein, 10 aa, Artificial Sequence
  CEA CDR-L3
MIWHSGASAV

SEQ ID NO: 15 - protein, 121 aa, Artificial Sequence
  CEA VH
EVQLVESGGGLVQPGRSLRLSCAASGFTVSSYWMHWVRQAPGKGLEWVGFIRNKANGGTTEYAASVKGRFTISRDDSKNTLYLQMNSLRAEDTAVYYCARDRGLRFYFDYWGQGTTVTVSS

SEQ ID NO: 16 - protein, 116 aa, Artificial Sequence
  CEA VL
QAVLTQPASLSASPGASASLTCTLRRGINVGAYSIYWYQQKPGSPPQYLLRYKSDSDKQQGSGVSSRFSASKDASANAGILLISGLQSEDEADYYCMIWHSGASAVFGGGTKLTVL

SEQ ID NO: 17 - protein, 10 aa, Artificial Sequence
  CEA (A5H1EL1D)- CDR-H1
GFTFTDYYMN

SEQ ID NO: 18 - protein, 19 aa, Artificial Sequence
  CEA (A5H1EL1D)- CDR-H2
FIGNKANAYTTEYSASVKG

SEQ ID NO: 19 - protein, 10 aa, Artificial Sequence
  CEA (A5H1EL1D)- CDR-H3
DRGLRFYFDY

SEQ ID NO: 20 - protein, 10 aa, Artificial Sequence
  CEA (A5H1EL1D)- CDR-L1
RASSSVTYIH

SEQ ID NO: 21 - protein, 7 aa, Artificial Sequence
  CEA (A5H1EL1D)- CDR-L2
ATSNLAS

SEQ ID NO: 22 - protein, 9 aa, Artificial Sequence
  CEA (A5H1EL1D)- CDR-L3
QHWSSKPPT

SEQ ID NO: 23 - protein, 121 aa, Artificial Sequence
  CEA (A5H1EL1D) VH (3-23A5-1E)
EVQLLESGGGLVQPGGSLRLSCAASGFTFTDYYMNWVRQAPGKGLEWLGFIGNKANAYTTEYSASVKGRFTISRDKSKNTLYLQMNSLRAEDTATYYCTRDRGLRFYFDYWGQGTTVTVSS

SEQ ID NO: 24 - protein, 106 aa, Artificial Sequence
  CEA (A5H1EL1D) VL (A5-L1D)
EIVLTQSPATLSLSPGERATLSCRASSSVTYIHWYQQKPGQAPRSWIYATSNLASGIPARFSGSGSGTDFTLTISSLEPEDFAVYYCQHWSSKPPTFGQGTKLEIK

SEQ ID NO: 25 - protein, 10 aa, Artificial Sequence
  CEA (A5H1EL1D aff. mat.) CDR-H1 consensus
  VARIANT (3)..(3): X is Thr or Tyr
  VARIANT (5)..(5): X is Thr or Ser
  VARIANT (8)..(8): X is Tyr or Ala or Glu
GFXFXDYXMN

SEQ ID NO: 26 - protein, 19 aa, Artificial Sequence
  CEA (A5H1EL1D aff. mat.) CDR-H2 consensus
  VARIANT (1)..(1): X is Phe or Val
  VARIANT (3)..(3): X is Gly or Ser
XIXNKANAYTTEYSASVKG

SEQ ID NO: 27 - protein, 10 aa, Artificial Sequence
  CEA (A5H1EL1D aff. mat.) CDR-H3 consensus
  VARIANT (4)..(4): X is Leu or Ile
  VARIANT (7)..(7): X is Tyr or Gly or Gln or Ser
DRGXRFXFDY

SEQ ID NO: 28 - protein, 10 aa, Artificial Sequence
  CEA (A5H1EL1D aff. mat.) CDR-L1 consensus
  VARIANT (1)..(1): X is Arg or His
XASSSVTYIH

SEQ ID NO: 29 - protein, 7 aa, Artificial Sequence
  CEA (A5H1EL1D aff. mat.) CDR-L2 consensus
ATSNLAS

SEQ ID NO: 30 - protein, 9 aa, Artificial Sequence
  CEA (A5H1EL1D aff. mat.) CDR-L3 consensus
  VARIANT (6)..(6): X is Lys or Val or Gln or Ile
  VARIANT (7)..(7): X is Pro or Ser
QHWSSXXPT

SEQ ID NO: 31 - protein, 121 aa, Artificial Sequence
  CEA (P006.038) VH
EVQLLESGGGLVQPGGSLRLSCAASGFTFTDYYMNWVRQAPGKGLEWLGFIGNKANAYTTEYSASVKGRFTISRDKSKNTLYLQMNSLRAEDTATYYCTRDRGIRFGFDYWGQGTTVTVSS

SEQ ID NO: 32 - protein, 106 aa, Artificial Sequence
  CEA (P006.038) VL
EIVLTQSPATLSLSPGERATLSCRASSSVTYIHWYQQKPGQAPRSWIYATSNLASGIPARFSGSGSGTDFTLTISSLEPEDFAVYYCQHWSSVPPTFGQGTKLEIK

SEQ ID NO: 33 - protein, 121 aa, Artificial Sequence
  CEA (P005.097) VH
EVQLLESGGGLVQPGGSLRLSCAASGFTFTDYYMNWVRQAPGKGLEWLGFIGNKANAYTTEYSASVKGRFTISRDKSKNTLYLQMNSLRAEDTATYYCTRDRGLRFSFDYWGQGTTVTVSS

SEQ ID NO: 34 - protein, 106 aa, Artificial Sequence
  CEA (P005.097) VL
EIVLTQSPATLSLSPGERATLSCRASSSVTYIHWYQQKPGQAPRSWIYATSNLASGIPARFSGSGSGTDFTLTISSLEPEDFAVYYCQHWSSQPPTFGQGTKLEIK

SEQ ID NO: 35 - protein, 121 aa, Artificial Sequence
  CEA (P005.103) VH
EVQLLESGGGLVQPGGSLRLSCAASGFTFTDYYMNWVRQAPGKGLEWLGFIGNKANAYTTEYSASVKGRFTISRDKSKNTLYLQMNSLRAEDTATYYCTRDRGIRFYFDYWGQGTTVTVSS

SEQ ID NO: 36 - protein, 106 aa, Artificial Sequence
  CEA (P005.103) VL
EIVLTQSPATLSLSPGERATLSCRASSSVTYIHWYQQKPGQAPRSWIYATSNLASGIPARFSGSGSGTDFTLTISSLEPEDFAVYYCQHWSSISPTFGQGTKLEIK

SEQ ID NO: 37 - protein, 121 aa, Artificial Sequence
  CEA (P002.139) VH
EVQLLESGGGLVQPGGSLRLSCAASGFYFTDYAMNWVRQAPGKGLEWLGVISNKANAYTTEYSASVKGRFTISRDKSKNTLYLQMNSLRAEDTATYYCTRDRGLRFYFDYWGQGTTVTVSS

SEQ ID NO: 38 - protein, 106 aa, Artificial Sequence
  CEA (P002.139) VL
EIVLTQSPATLSLSPGERATLSCHASSSVTYIHWYQQKPGQAPRSWIYATSNLASGIPARFSGSGSGTDFTLTISSLEPEDFAVYYCQHWSSKPPTFGQGTKLEIK

SEQ ID NO: 39 - protein, 121 aa, Artificial Sequence
  CEA (P001.177) VH
EVQLLESGGGLVQPGGSLRLSCAASGFYFTDYYMNWVRQAPGKGLEWLGFISNKANAYTTEYSASVKGRFTISRDKSKNTLYLQMNSLRAEDTATYYCTRDRGLRFYFDYWGQGTTVTVSS

SEQ ID NO: 40 - protein, 106 aa, Artificial Sequence; identical to SEQ ID NO: 24
  CEA (P001.177) VL

SEQ ID NO: 41 - protein, 121 aa, Artificial Sequence
  CEA (P005.102) VH
EVQLLESGGGLVQPGGSLRLSCAASGFTFTDYYMNWVRQAPGKGLEWLGFIGNKANAYTTEYSASVKGRFTISRDKSKNTLYLQMNSLRAEDTATYYCTRDRGIRFQFDYWGQGTTVTVSS

SEQ ID NO: 42 - protein, 106 aa, Artificial Sequence
  CEA (P005.102) VL
EIVLTQSPATLSLSPGERATLSCRASSSVTYIHWYQQKPGQAPRSWIYATSNLASGIPARFSGSGSGTDFTLTISSLEPEDFAVYYCQHWSSKSPTFGQGTKLEIK

SEQ ID NO: 43 - protein, 121 aa, Artificial Sequence
  CEA (P005.102 combo1) VH
EVQLLESGGGLVQPGGSLRLSCAASGFYFTDYYMNWVRQAPGKGLEWLGVISNKANAYTTEYSASVKGRFTISRDKSKNTLYLQMNSLRAEDTATYYCTRDRGIRFQFDYWGQGTTVTVSS

SEQ ID NO: 44 - protein, 106 aa, Artificial Sequence; identical to SEQ ID NO: 42
  CEA (P005.102 combo1) VL

SEQ ID NO: 45 - protein, 121 aa, Artificial Sequence
  CEA (P005.102 combo2) VH
EVQLLESGGGLVQPGGSLRLSCAASGFYFSDYYMNWVRQAPGKGLEWLGVISNKANAYTTEYSASVKGRFTISRDKSKNTLYLQMNSLRAEDTATYYCTRDRGIRFQFDYWGQGTTVTVSS

SEQ ID NO: 46 - protein, 106 aa, Artificial Sequence; identical to SEQ ID NO: 42
  CEA (P005.102 combo2) VL

SEQ ID NO: 47 - protein, 121 aa, Artificial Sequence
  CEA (P005.103 combo1) VH
EVQLLESGGGLVQPGGSLRLSCAASGFTFTDYYMNWVRQAPGKGLEWLGFIGNKANAYTTEYSASVKGRFTISRDKSKNTLYLQMNSLRAEDTATYYCTRDRGIRFSFDYWGQGTTVTVSS

SEQ ID NO: 48 - protein, 106 aa, Artificial Sequence; identical to SEQ ID NO: 36
  CEA (P005.103 combo1) VL

SEQ ID NO: 49 - protein, 121 aa, Artificial Sequence
  CEA (P005.103 combo2) VH
EVQLLESGGGLVQPGGSLRLSCAASGFYFTDYYMNWVRQAPGKGLEWLGVISNKANAYTTEYSASVKGRFTISRDKSKNTLYLQMNSLRAEDTATYYCTRDRGIRFSFDYWGQGTTVTVSS

SEQ ID NO: 50 - protein, 106 aa, Artificial Sequence; identical to SEQ ID NO: 36
  CEA (P005.103 combo2) VL

SEQ ID NO: 51 - protein, 121 aa, Artificial Sequence
  CEA (P006.038 combo1) VH
EVQLLESGGGLVQPGGSLRLSCAASGFYFTDYAMNWVRQAPGKGLEWLGVISNKANAYTTEYSASVKGRFTISRDKSKNTLYLQMNSLRAEDTATYYCTRDRGIRFGFDYWGQGTTVTVSS

SEQ ID NO: 52 - protein, 106 aa, Artificial Sequence; identical to SEQ ID NO: 32
  CEA (P006.038 combo1) VL

SEQ ID NO: 53 - protein, 121 aa, Artificial Sequence
  CEA (P006.038 combo2) VH
EVQLLESGGGLVQPGGSLRLSCAASGFTFSDYEMNWVRQAPGKGLEWLGFISNKANAYTTEYSASVKGRFTISRDKSKNTLYLQMNSLRAEDTATYYCTRDRGIRFGFDYWGQGTTVTVSS

SEQ ID NO: 54 - protein, 106 aa, Artificial Sequence; identical to SEQ ID NO: 32
  CEA (P006.038 combo2) VL

SEQ ID NO: 55 - protein, 5 aa, Artificial Sequence
  CEA (MFE23)- CDR-H1
DSYMH

SEQ ID NO: 56 - protein, 17 aa, Artificial Sequence
  CEA (MFE23)- CDR-H2
WIDPENGDTEYAPKFQG

SEQ ID NO: 57 - protein, 11 aa, Artificial Sequence
  CEA (MFE23)- CDR-H3
GTPTGPYYFDY

SEQ ID NO: 58 - protein, 10 aa, Artificial Sequence
  CEA (MFE23)- CDR-L1
SASSSVSYMH

SEQ ID NO: 59 - protein, 7 aa, Artificial Sequence
  CEA (MFE23)- CDR-L2
STSNLAS

SEQ ID NO: 60 - protein, 9 aa, Artificial Sequence
  CEA (MFE23)- CDR-L3
QQRSSYPLT

SEQ ID NO: 61 - protein, 120 aa, Artificial Sequence
  CEA (MFE23) VH
QVKLQQSGAELVRSGTSVKLSCTASGFNIKDSYMHWLRQGPEQGLEWIGWIDPENGDTEYAPKFQGKATFTTDTSSNTAYLQLSSLTSEDTAVYYCNEGTPTGPYYFDYWGQGTTVTVSS

SEQ ID NO: 62 - protein, 106 aa, Artificial Sequence
  CEA (MFE23) VL
ENVLTQSPAIMSASPGEKVTITCSASSSVSYMHWFQQKPGTSPKLWIYSTSNLASGVPARFSGSGSGTSYSLTISRMEAEDAATYYCQQRSSYPLTFGAGTKLELK

SEQ ID NO: 63 - protein, 121 aa, Artificial Sequence
  CEA (T84.66-LCHA) VH
QVQLVQSGAEVKKPGSSVKVSCKASGFNIKDTYMHWVRQAPGQGLEWMGRIDPANGNSKYVPKFQGRVTITADTSTSTAYMELSSLRSEDTAVYYCAPFGYYVSDYAMAYWGQGTLVTVSS

SEQ ID NO: 64 - protein, 111 aa, Artificial Sequence
  CEA (T84.66-LCHA) VL
EIVLTQSPATLSLSPGERATLSCRAGESVDIFGVGFLHWYQQKPGQAPRLLIYRASNRATGIPARFSGSGSGTDFTLTISSLEPEDFAVYYCQQTNEDPYTFGQGTKLEIK

SEQ ID NO: 65 - protein, 5 aa, Artificial Sequence
  CEA (MFE-H24 to H29)- CDR-H1
DSYMH

SEQ ID NO: 66 - protein, 17 aa, Artificial Sequence
  CEA (MFE-H24, H25, H27, H28, H29)- CDR-H2
WIDPENGDTEYAPKFQG

SEQ ID NO: 67 - protein, 17 aa, Artificial Sequence
  CEA (MFE-H26)- CDR-H2
WIDPENGGTNYAQKFQG

SEQ ID NO: 68 - protein, 11 aa, Artificial Sequence
  CEA (MFE-H24 to H29)- CDR-H3
GTPTGPYYFDY

SEQ ID NO: 69 - protein, 10 aa, Artificial Sequence
  CEA (MFE-L24, L25)- CDR-L1
RASSSVSYMH

SEQ ID NO: 70 - protein, 10 aa, Artificial Sequence
  CEA (MFE-H26)- CDR-L1
RASQSISSYM

SEQ ID NO: 71 - protein, 7 aa, Artificial Sequence
  CEA (MFE-L24, L25, L27, L28)- CDR-L2
STSNLAS

SEQ ID NO: 72 - protein, 7 aa, Artificial Sequence
  CEA (MFE-L26)- CDR-L2
YTSNLAS

SEQ ID NO: 73 - protein, 7 aa, Artificial Sequence
  CEA (MFE-L29)- CDR-L2
STSSLQS

SEQ ID NO: 74 - protein, 9 aa, Artificial Sequence
  CEA (MFE-L24, L25, L27, L26, L28, L29)- CDR-L3
QQRSSYPLT

SEQ ID NO: 75 - protein, 120 aa, Artificial Sequence
  MFE-H24
QVQLVQSGAEVKKPGASVKVSCKASGFNIKDSYMHWVRQAPGQGLEWMGWIDPENGDTEYAPKFQGRVTMTTDTSISTAYMELSRLRSDDTAVYYCNEGTPTGPYYFDYWGQGTLVTVSS

SEQ ID NO: 76 - protein, 120 aa, Artificial Sequence
  MFE-H25
QVQLVQSGAEVKKPGASVKVSCKASGYTFKDSYMHWVRQAPGQGLEWMGWIDPENGDTEYAPKFQGRVTMTTDTSISTAYMELSRLRSDDTAVYYCNEGTPTGPYYFDYWGQGTLVTVSS

SEQ ID NO: 77 - protein, 120 aa, Artificial Sequence
  MFE-H26
QVQLVQSGAEVKKPGASVKVSCKASGFNIKDSYMHWVRQAPGQGLEWMGWIDPENGGTNYAQKFQGRVTMTTDTSISTAYMELSRLRSDDTAVYYCNEGTPTGPYYFDYWGQGTLVTVSS

SEQ ID NO: 78 - protein, 120 aa, Artificial Sequence
  MFE-H27
QVQLVQSGAEVKKPGASVKVSCKASGFNIKDSYMHWVRQAPGQGLEWMGWIDPENGDTEYAPKFQGRVTMTTDTSISTAYMELSRLRSDDTAVYYCARGTPTGPYYFDYWGQGTLVTVSS

SEQ ID NO: 79 - protein, 120 aa, Artificial Sequence
  MFE-H28
QVQLVQSGAEVKKPGASVKVSCKASGFNIKDSYMHWVRQAPGQGLEWMGWIDPENGDTEYAPKFQGRVTMTRDTSISTAYMELSRLRSDDTAVYYCNEGTPTGPYYFDYWGQGTLVTVSS

SEQ ID NO: 80 - protein, 120 aa, Artificial Sequence
  MFE-H29
QVQLVQSGAEVKKPGSSVKVSCKASGFNIKDSYMHWVRQAPGQGLEWMGWIDPENGDTEYAPKFQGRVTITTDESTSTAYMELSSLRSEDTAVYYCNEGTPTGPYYFDYWGQGTLVTVSS

SEQ ID NO: 81 - protein, 106 aa, Artificial Sequence
  MFE-L24
DIQMTQSPSSLSASVGDRVTITCRASSSVSYMHWYQQKPGKAPKLLIYSTSNLASGVPSRFSGSGSGTDFTLTISSLQPEDFATYYCQQRSSYPLTFGGGTKLEIK

SEQ ID NO: 82 - protein, 106 aa, Artificial Sequence
  MFE-L25
EIQMTQSPSSLSASVGDRVTITCRASSSVSYMHWYQQKPGKAPKLLIYSTSNLASGVPSRFSGSGSGTDFTLTISSLQPEDFATYYCQQRSSYPLTFGGGTKLEIK

SEQ ID NO: 83 - protein, 107 aa, Artificial Sequence
  MFE-L26
EIQMTQSPSSLSASVGDRVTITCRASQSISSYMHWYQQKPGKAPKLLIYSTSNLASGVPSRFSGSGSGTDFTLTISSLQPEDFATYYCQQRSSYPLTFGGGTKLEIK

SEQ ID NO: 84 - protein, 106 aa, Artificial Sequence
  MFE-L27
EIQMTQSPSSLSASVGDRVTITCRASSSVPYMHWYQQKPGKAPKLLIYSTSNLASGVPSRFSGSGSGTDFTLTISSVQPEDFATYYCQQRSSYPLTFGGGTKLEIK

SEQ ID NO: 85 - protein, 106 aa, Artificial Sequence
  MFE-L28
EIQMTQSPSSLSASVGDRVTITCRASSSVPYMHWLQQKPGKAPKLLIYSTSNLASGVPSRFSGSGSGTDFTLTISSVQPEDFATYYCQQRSSYPLTFGGGTKLEIK

SEQ ID NO: 86 - protein, 106 aa, Artificial Sequence
  MFE-L29
EIQMTQSPSSLSASVGDRVTITCRASSSVPYMHWLQQKPGKAPKLLIYSTSSLQSGVPSRFSGSGSGTDFTLTISSVQPEDFATYYCQQRSSYPLTFGGGTKLEIK

SEQ ID NO: 87 - protein, 184 aa, Homo sapiens
REGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 88 - protein, 170 aa, Homo sapiens
LDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 89 - protein, 175 aa, Homo sapiens
DPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 90 - protein, 203 aa, Homo sapiens
PWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 91 - protein, 178 aa, Homo sapiens
REGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGL

SEQ ID NO: 92 - protein, 164 aa, Homo sapiens
LDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGL

SEQ ID NO: 93 - protein, 169 aa, Homo sapiens
DPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGL

SEQ ID NO: 94 - protein, 197 aa, Homo sapiens
PWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGL

SEQ ID NO: 95 - protein, 378 aa, Artificial Sequence
  dimeric hu 4-1BBL (71-254) connected by (G4S)2 linker
REGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 96 - protein, 366 aa, Artificial Sequence
  dimeric hu 4-1BBL (71-248) connected by (G4S)2 linker
REGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGL

SEQ ID NO: 97 - protein, 360 aa, Artificial Sequence
  dimeric hu 4-1BBL (80-254) connected by (G4S)2 linker
DPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 98 - protein, 416 aa, Artificial Sequence
  dimeric hu 4-1BBL (52-254) connected by (G4S)2 linker
PWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 99 - protein, 708 aa, Artificial Sequence
  Dimeric 4-1BBL - CL* Fc knob chain
REGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLGGGGSGGGGSRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 100 - protein, 291 aa, Artificial Sequence
  Monomeric 4-1BBL -CH1*
REGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLGGGGSGGGGSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSC

SEQ ID NO: 101 - protein, 708 aa, Artificial Sequence
  Dimeric 4-1BB ligand (71-248) - CL Fc knob chain
REGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLGGGGSGGGGSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 102 - protein, 291 aa, Artificial Sequence
  Monomeric 4-1BB ligand (71-248)-CH1
REGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLGGGGSGGGGSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 103 - protein, 720 aa, Artificial Sequence
  Dimeric 4-1BB ligand (71-254) - CL* Fc knob chain
REGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 104 - protein, 297 aa, Artificial Sequence
  Monomeric 4-1BB ligand (71-254)-CH1*
REGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSC

SEQ ID NO: 105 - protein, 720 aa, Artificial Sequence
  Dimeric 4-1BB ligand (71-254) - CL Fc knob chain
REGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 106 - protein, 297 aa, Artificial Sequence
  Monomeric 4-1BB ligand (71-254) -CH1
REGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 107 - protein, 328 aa, Artificial Sequence
  Fc hole chain
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 108 - protein, 702 aa, Homo sapiens
MESPSAPPHRWCIPWQRLLLTASLLTFWNPPTTAKLTIESTPFNVAEGKEVLLLVHNLPQHLFGYSWYKGERVDGNRQIIGYVIGTQQATPGPAYSGREIIYPNASLLIQNIIQNDTGFYTLHVIKSDLVNEEATGQFRVYPELPKPSISSNNSKPVEDKDAVAFTCEPETQDATYLWWVNNQSLPVSPRLQLSNGNRTLTLFNVTRNDTASYKCETQNPVSARRSDSVILNVLYGPDAPTISPLNTSYRSGENLNLSCHAASNPPAQYSWFVNGTFQQSTQELFIPNITVNNSGSYTCQAHNSDTGLNRTTVTTITVYAEPPKPFITSNNSNPVEDEDAVALTCEPEIQNTTYLWWVNNQSLPVSPRLQLSNDNRTLTLLSVTRNDVGPYECGIQNKLSVDHSDPVILNVLYGPDDPTISPSYTYYRPGVNLSLSCHAASNPPAQYSWLIDGNIQQHTQELFISNITEKNSGLYTCQANNSASGHSRTTVKTITVSAELPKPSISSNNSKPVEDKDAVAFTCEPEAQNTTYLWWVNGQSLPVSPRLQLSNGNRTLTLFNVTRNDARAYVCGIQNSVSANRSDPVTLDVLYGPDTPIISPPDSSYLSGANLNLSCHSASNPSPQYSWRINGIPQQHTQVLFIAKITPNNNGTYACFVSNLATGRNNSIVKSITVSASGTSPGLSAGATVGIMIGVLVGVALI

SEQ ID NO: 109 - protein, 255 aa, Homo sapiens
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 110 - protein, 256 aa, Mus musculus
MGNNCYNVVVIVLLLVGCEKVGAVQNSCDNCQPGTFCRKYNPVCKSCPPSTFSSIGGQPNCNICRVCAGYFRFKKFCSSTHNAECECIEGFHCLGPQCTRCEKDCRPGQELTKQGCKTCSLGTFNDQNGTGVCRPWTNCSLDGRSVLKTGTTEKDVVCGPPVVSFSPSTTISVTPEGGPGGHSLQVLTLFLALTSALLLALIFITLLFSVLKWIRKKFPHIFKQPFKKTTGAAQEEDACSCRCPQEEEGGGGGYEL

SEQ ID NO: 111 - protein, 254 aa, Macaca fascicularis
MGNSCYNIVATLLLVLNFERTRSLQDLCSNCPAGTFCDNNRSQICSPCPPNSFSSAGGQRTCDICRQCKGVFKTRKECSSTSNAECDCISGYHCLGAECSMCEQDCKQGQELTKKGCKDCCFGTFNDQKRGICRPWTNCSLDGKSVLVNGTKERDVVCGPSPADLSPGASSATPPAPAREPGHSPQIIFFLALTSTVVLFLLFFLVLRFSVVKRSRKKLLYIFKQPFMRPVQTTQEEDGCSCRFPEEEEGGCEL

SEQ ID NO: 112 - protein, 5 aa, Artificial Sequence
  Peptide linker G4S
GGGGS

SEQ ID NO: 113 - protein, 10 aa, Artificial Sequence
  Peptide linker (G4S)2
GGGGSGGGGS

SEQ ID NO: 114 - protein, 10 aa, Artificial Sequence
  Peptide linker (SG4)2
SGGGGSGGGG

SEQ ID NO: 115 - protein, 14 aa, Artificial Sequence
  Peptide linker G4(SG4)2
GGGGSGGGGSGGGG

SEQ ID NO: 116 - protein, 10 aa, Artificial Sequence
  Peptide linker GSPGSSSSGS
GSPGSSSSGS

SEQ ID NO: 117 - protein, 15 aa, Artificial Sequence
  Peptide linker (G4S)3
GGGGSGGGGSGGGGS

SEQ ID NO: 118 - protein, 20 aa, Artificial Sequence
  Peptide linker (G4S)4
GGGGSGGGGSGGGGSGGGGS

SEQ ID NO: 119 - protein, 8 aa, Artificial Sequence
  Peptide linker GSGSGSGS
GSGSGSGS

SEQ ID NO: 120 - protein, 8 aa, Artificial Sequence
  Peptide linker GSGSGNGS
GSGSGNGS

SEQ ID NO: 121 - protein, 8 aa, Artificial Sequence
  Peptide linker GGSGSGSG
GGSGSGSG

SEQ ID NO: 122 - protein, 6 aa, Artificial Sequence
  Peptide linker GGSGSG
GGSGSG

SEQ ID NO: 123 - protein, 4 aa, Artificial Sequence
  Peptide linker GGSG
GGSG

SEQ ID NO: 124 - protein, 8 aa, Artificial Sequence
  GGSGNGSG
GGSGNGSG

SEQ ID NO: 125 - protein, 8 aa, Artificial Sequence
  Peptide inker GGNGSGSG
GGNGSGSG

SEQ ID NO: 126 - protein, 6 aa, Artificial Sequence
  Peptide linker GGNGSG
GGNGSG

SEQ ID NO: 127 - protein, 98 aa, Artificial Sequence
  IGHV3-23-02
EVQLLESGGGLVQPGGSLRLSCAASGFTFSSYAMSWVRQAPGKGLEWVSAISGSGGSTYYGDSVKGRFTISRDNSKNTLYLQMNSLRAEDTAVYYCAK

SEQ ID NO: 128 - protein, 100 aa, Artificial Sequence
  IGHV3-15*01
EVQLVESGGGLVKPGGSLRLSCAASGFTFSNAWMSWVRQAPGKGLEWVGRIKSKTDGGTTDYAAPVKGRFTISRDDSKNTLYLQMNSLKTEDTAVYYCTT

SEQ ID NO: 129 - protein, 121 aa, Artificial Sequence
  3-23A5-1
EVQLLESGGGLVQPGGSLRLSCAASGFTFTDYYMNWVRQAPGKGLEWVGFIGNKANGYTTEYSASVKGRFTISRDNSKNTLYLQMNSLRAEDTAVYYCARDRGLRFYFDYWGQGTTVTVSS

SEQ ID NO: 130 - protein, 121 aa, Artificial Sequence
  3-23A5-2
EVQLLESGGGLVQPGGSLRLSCAASGFTFTDYYMNWVRQAPGKGLEWVGFIGNKANGYTTYYGDSVKGRFTISRDNSKNTLYLQMNSLRAEDTAVYYCARDRGLRFYFDYWGQGTTVTVSS

SEQ ID NO: 131 - protein, 119 aa, Artificial Sequence
  3-23A5-3
EVQLLESGGGLVQPGGSLRLSCAASGFTFTDYYMNWVRQAPGKGLEWVGFIGNKGYTTEYSASVKGRFTISRDNSKNTLYLQMNSLRAEDTAVYYCARDRGLRFYFDYWGQGTTVTVSS

SEQ ID NO: 132 - protein, 121 aa, Artificial Sequence
  3-23A5-4
EVQLLESGGGLVQPGGSLRLSCAASGFTFTDYYMSWVRQAPGKGLEWVGFIGNKANGYTTEYSASVKGRFTISRDNSKNTLYLQMNSLRAEDTAVYYCARDRGLRFYFDYWGQGTTVTVSS

SEQ ID NO: 133 - protein, 121 aa, Artificial Sequence
  3-23A5-1A (all_backmutations)
EVQLLESGGGLVQPGGSLRLSCAASGFTFTDYYMNWVRQAPGKGLEWLGFIGNKANGYTTEYSASVKGRFTISRDKSKNTLYLQMNSLRAEDTATYYCTRDRGLRFYFDYWGQGTTVTVSS

SEQ ID NO: 134 - protein, 121 aa, Artificial Sequence
  3-23A5-1C (A93T)
EVQLLESGGGLVQPGGSLRLSCAASGFTFTDYYMNWVRQAPGKGLEWVGFIGNKANGYTTEYSASVKGRFTISRDNSKNTLYLQMNSLRAEDTAVYYCTRDRGLRFYFDYWGQGTTVTVSS

SEQ ID NO: 135 - protein, 121 aa, Artificial Sequence
  3-23A5-1D (K73)
EVQLLESGGGLVQPGGSLRLSCAASGFTFTDYYMNWVRQAPGKGLEWVGFIGNKANGYTTEYSASVKGRFTISRDKSKNTLYLQMNSLRAEDTAVYYCARDRGLRFYFDYWGQGTTVTVSS

SEQ ID NO: 136 - protein, 121 aa, Artificial Sequence
  3-15A5-1
EVQLVESGGGLVKPGGSLRLSCAASGFTFTDYYMNWVRQAPGKGLEWVGFIGNKANGYTTEYSASVKGRFTISRDDSKNTLYLQMNSLKTEDTAVYYCTRDRGLRFYFDYWGQGTTVTVSS

SEQ ID NO: 137 - protein, 121 aa, Artificial Sequence
  3-15A5-2
EVQLVESGGGLVKPGGSLRLSCAASGFTFTDYYMNWVRQAPGKGLEWVGFIGNKANGYTTEYAAPVKGRFTISRDDSKNTLYLQMNSLKTEDTAVYYCTRDRGLRFYFDYWGQGTTVTVSS

SEQ ID NO: 138 - protein, 121 aa, Artificial Sequence
  3-15A5-3
EVQLVESGGGLVKPGGSLRLSCAASGFTFTDYYMNWVRQAPGKGLEWVGFIGNKANGGTTDYAAPVKGRFTISRDDSKNTLYLQMNSLKTEDTAVYYCTRDRGLRFYFDYWGQGTTVTVSS

SEQ ID NO: 139 - protein, 95 aa, Artificial Sequence
  IGKV3-11
EIVLTQSPATLSLSPGERATLSCRASQSVSSYLAWYQQKPGQAPRLLIYDASNRATGIPARFSGSGSGTDFTLTISSLEPEDFAVYYCQQRSNWP

SEQ ID NO: 140 - protein, 106 aa, Artificial Sequence
  A5-L1
EIVLTQSPATLSLSPGERATLSCRASSSVTYIHWYQQKPGQAPRLLIYATSNLASGIPARFSGSGSGTDFTLTISSLEPEDFAVYYCQHWSSKPPTFGQGTKLEIK

SEQ ID NO: 141 - protein, 107 aa, Artificial Sequence
  A5-L2
EIVLTQSPATLSLSPGERATLSCRASQSVSSYIHWYQQKPGQAPRLLIYATSNLASGIPARFSGSGSGTDFTLTISSLEPEDFAVYYCQHWSSKPPTFGQGTKLEIK

SEQ ID NO: 142 - protein, 106 aa, Artificial Sequence
  A5-L3
EIVLTQSPATLSLSPGERATLSCRASSSVTYIHWYQQKPGQAPRLLIYDASNRATGIPARFSGSGSGTDFTLTISSLEPEDFAVYYCQHWSSKPPTFGQGTKLEIK

SEQ ID NO: 143 - protein, 106 aa, Artificial Sequence
  A5-L4
EIVLTQSPATLSLSPGERATLSCRASSSVTYIHWYQQKPGQAPRLLIYATSNLASGIPARFSGSGSGTDFTLTISSLEPEDFAVYYCQQWSSKPPTFGQGTKLEIK

SEQ ID NO: 144 - protein, 106 aa, Artificial Sequence
  A5-L1A (all_backmutations)
QTVLTQSPATLSLSPGERATLSCRASSSVTYIHWYQQKPGSSPKSWIYATSNLASGIPARFSGSGSGTDYTLTISSLEPEDFAVYYCQHWSSKPPTFGQGTKLEIK

SEQ ID NO: 145 - protein, 106 aa, Artificial Sequence
  A5-L1B (Q1T2)
QTVLTQSPATLSLSPGERATLSCRASSSVTYIHWYQQKPGQAPRLLIYATSNLASGIPARFSGSGSGTDFTLTISSLEPEDFAVYYCQHWSSKPPTFGQGTKLEIK

SEQ ID NO: 146 - protein, 106 aa, Artificial Sequence
  A5-L1C (FR2)
EIVLTQSPATLSLSPGERATLSCRASSSVTYIHWYQQKPGSSPKSWIYATSNLASGIPARFSGSGSGTDFTLTISSLEPEDFAVYYCQHWSSKPPTFGQGTKLEIK

SEQ ID NO: 147 - protein, 428 aa, Artificial Sequence
  NABA-avi-His
QLTTESMPFNVAEGKEVLLLVHNLPQQLFGYSWYKGERVDGNRQIVGYAIGTQQATPGPANSGRETIYPNASLLIQNVTQNDTGFYTLQVIKSDLVNEEATGQFHVYPELPKPSISSNNSNPVEDKDAMAFTCEPETQDTTYLWWINNQSLPVSPRLQLSNGNRTLTLLSVTRNDTGPYECEIQNPVSANRSDPVTLNVTYGPDTPTISPSDTYYRPGANLSLSCYAASNPPAQYSWLINGTFQQSTQELFIPNITVNNSGSYTCHANNSVTGCNRTTVKTIIVTELSPVVAKPQIKASKTTVTGDKDSVNLTCSTNDTGISIRWFFKNQSLPSSERMKLSQGNITLSINPVKREDAGTYWCEVFNPISKNQSDPIMLNVNYNALPQENLINVDLEVLFQGPGSGLNDIFEAQKIEWHEARAHHHHHH

SEQ ID NO: 148 - protein, 420 aa, Artificial Sequence
  N(A2B2)A-avi-His
QLTTESMPFNVAEGKEVLLLVHNLPQQLFGYSWYKGERVDGNRQIVGYAIGTQQATPGPANSGRETIYPNASLLIQNVTQNDTGFYTLQVIKSDLVNEEATGQFHVYPELPKPFITSNNSNPVEDEDAVALTCEPEIQNTTYLWWVNNQSLPVSPRLQLSNDNRTLTLLSVTRNDVGPYECGIQNKLSVDHSDPVILNVLYGPDDPTISPSYTYYRPGVNLSLSCHAASNPPAQYSWLIDGNIQQHTQELFISNITEKNSGLYTCQANNSASGHSRTTVKTITVSALSPVVAKPQIKASKTTVTGDKDSVNLTCSTNDTGISIRWFFKNQSLPSSERMKLSQGNITLSINPVKREDAGTYWCEVFNPISKNQSDPIMLNVNYNALPQENLINVDGSGLNDIFEAQKIEWHEARAHHHHHH

SEQ ID NO: 149 - protein, 420 aa, Artificial Sequence
  NA(B2)A-avi-His
QLTTESMPFNVAEGKEVLLLVHNLPQQLFGYSWYKGERVDGNRQIVGYAIGTQQATPGPANSGRETIYPNASLLIQNVTQNDTGFYTLQVIKSDLVNEEATGQFHVYPELPKPSISSNNSNPVEDKDAMAFTCEPETQDTTYLWWINNQSLPVSPRLQLSNGNRTLTLLSVTRNDTGPYECEIQNPVSANRSDPVTLNVTYGPDDPTISPSYTYYRPGVNLSLSCHAASNPPAQYSWLIDGNIQQHTQELFISNITEKNSGLYTCQANNSASGHSRTTVKTITVSALSPVVAKPQIKASKTTVTGDKDSVNLTCSTNDTGISIRWFFKNQSLPSSERMKLSQGNITLSINPVKREDAGTYWCEVFNPISKNQSDPIMLNVNYNALPQENLINVDGSGLNDIFEAQKIEWHEARAHHHHHH

SEQ ID NO: 150 - DNA, 90 bp, Artificial Sequence
  A5H1EL1D_H1_rev_TN
  misc_feature (40)..(42): n is a, c, g, or t and codes for 50% N; 20% S; 3% D/E/Q/G/Y/V/T/H/A/L
  misc_feature (46)..(48): n is a, c, g, or t and codes for  50% Y; 20% A; 3.75% G/V/T/H/L/I/R/F
  misc_feature (49)..(51): n is a, c, g, or t and codes for  60% Y; 4% G/V/H/S/E/Q/N/D/R/F
  misc_feature (52)..(54): n is a, c, g, or t and codes for  50% D; 20% S; 4.3% G/Y/T/N/A/E/Q
  misc_feature (55)..(57): n is a, c, g, or t and codes for  50% T; 20% S; 4.3% A/G/Y/N/D/E/Q
  misc_feature (61)..(62): n is a, c, g, or t and codes for  60% T; 5% A/S/G/Y/N/D/E/Q
  misc_feature (63)..(63): n is a, c, g, or t
cagccactcgaggcctttacccggtgcttggcgtacccannncatnnnnnnnnnnnngaannngaagccagaagccgcgcagctgagacg

SEQ ID NO: 151 - DNA, 84 bp, Artificial Sequence
  A5H1EL1D_H2_for_TN
  misc_feature (37)..(39): n is a, c, g, or t and codes for  60% F; 10% A; 6 % Y/V/L/I/G
  misc_feature (43)..(45): n is a, c, g, or t and codes for  50% G; 20% S; 3% A/K/T/V/N/D/E/Q/L/I
  misc_feature (46)..(48): n is a, c, g, or t and codes for  650% N; 20% G; 3.75 % D/E/Q/S/Y/T/H/A
  misc_feature (49)..(51): n is a, c, g, or t and codes for  60% K; 5% A/T/Y/N/D/E/Q/R
  misc_feature (52)..(54): n is a, c, g, or t and codes for  60% A; 4% V/G/D/P/H/N/E/Q/L/I
  misc_feature (55)..(57): n is a, c, g, or t and codes for  60% N; 5% D/E/Q; 4.17% G/T/H/S/A/R
cgccaagcaccgggtaaaggcctcgagtggctgggtnnnatcnnnnnnnnnnnnnnngcgtacaccacggaatactccgcctcc

SEQ ID NO: 152 - DNA, 26 bp, Artificial Sequence
  LMB3 long
caggaaacagctatgaccatgattac

SEQ ID NO: 153 - DNA, 53 bp, Artificial Sequence
  HCDR3-rev-constant
aacggtcaccgtggtaccctggccccagtagtcgaaatagaagcgcagaccac

SEQ ID NO: 154 - DNA, 84 bp, Artificial Sequence
  A5H1EL1D _L1_rev_TN
  misc_feature (37)..(39): n is a, c, g, or t and codes for 50% H; 20% A; 3.33% R/K/G/S/T/Q/Y/N/V
  misc_feature (40)..(42): n is a, c, g, or t and codes for 70% I; 30% L
  misc_feature (43)..(45): n is a, c, g, or t and codes for  60% Y; 4% F/G/A/V/T/H/S/N/Q/R
  misc_feature (46)..(48): n is a, c, g, or t and codes for  50% T; 20% S; 2.72% A/G/Y/V/P/H/N/D/E/Q/R
  misc_feature (49)..(51): n is a, c, g, or t and codes for 50% V; 20% S; 3.33% T/A/G/N/Q/F/Y/P/H
  misc_feature (52)..(54): n is a, c, g, or t and codes for 50% S; 20% V; 3.33% T/A/G/N/D/E/Q/Y/H
ggaacgcggggcctggcctggtttttgctgataccannnnnnnnnnnnnnnnnngctggatgcgcggcaagacagggtagcacg

SEQ ID NO: 155 - DNA, 84 bp, Artificial Sequence
  A5H1EL1D _L2_for_TN
  misc_feature (37)..(39): n is a, c, g, or t and codes for  60% Y; 10% F; 7.5% H/K/N/S
  misc_feature (40)..(42): n is a, c, g, or t and codes for  50% A; 20% D; 3.33% V/G/S/T/Y/H/N/E/Q
  misc_feature (43)..(45): n is a, c, g, or t and codes for  50% T; 20% A; 3.33% S/G/V/P/H/N/D/E/Q
  misc_feature (46)..(48): n is a, c, g, or t and codes for  60% S; 4% T/A/G/N/D/E/Q/Y/V/H
  misc_feature (49)..(51): n is a, c, g, or t and codes for  60% N; 4% D/E/Q/Y/K/T/H/S/A/R
cagcaaaaaccaggccaggccccgcgttcctggatcnnnnnnnnnnnnnnnctcgcttctggtatcccggcacgtttctccggc

SEQ ID NO: 156 - DNA, 84 bp, Artificial Sequence
  A5H1EL1D _L3_for_TN
  misc_feature (31)..(33): n is a, c, g, or t and codes for  90% Q; 10% H
  misc_feature (34)..(36): n is a, c, g, or t and codes for  60% H; 5% R/K/Q/E/Y/F/N/D
  misc_feature (37)..(39): n is a, c, g, or t and codes for 65% W; 7% F/Y/V/L/I
  misc_feature (40)..(42): n is a, c, g, or t and codes for  58% S; 4% T/A/G/N/D/E/Q; 2% Y/V/P/H/L/I/R
  misc_feature (43)..(45): n is a, c, g, or t and codes for  58% S; 4% T/A/G/N/D/E/Q; 2% Y/V/P/H/L/I/R
  misc_feature (46)..(48): n is a, c, g, or t and codes for  60% K; 5% R/H; 2.72% A/V/T/P/Y/N/D/E/Q/L/I
  misc_feature (49)..(51): n is a, c, g, or t and codes for  70% P; 5% A/S/T/R/S/L
  misc_feature (52)..(54): n is a, c, g, or t and codes for 60% P; 5% L/G/R/M; 2.86% A/V/L/I/F/S/R
gagcctgaagattttgccgtatactattgtnnnnnnnnnnnnnnnnnnnnnnnnactttcggtcagggcaccaagctggaaatc

SEQ ID NO: 157 - DNA, 90 bp, Artificial Sequence
  A5H1EL1D _H3_rev_TN
  misc_feature (34)..(36): n is a, c, g, or t and codes for  80% F; 10% I/L
  misc_feature (37)..(39): n is a, c, g or t and codes for  60% Y; 5% F/W; 2.14% G/A/V/T/P/H/S/N/D/E/Q/L/I/R
  misc_feature (40)..(42): n is a, c, g, or t and codes for  65% F; 5% Y/W/A/V/L/I/G
  misc_feature (43)..(45): n is a, c, g or t and codes for 60% R; 5% K/H; 2.72% A/V/T/P/Y/N/D/E/Q/L/I
  misc_feature (46)..(48): n is a, c, g, or t and codes for 60% L; 4% I/V/A/F; 2.4% G/Y/T/P/H/S/N/D/E/Q
  misc_feature (49)..(51): n is a, c, g, or t and codes for 60% G; 5% A/S/T; 2.5% Y/V/P/H/N/D/E/Q/L/I
  misc_feature (52)..(54): n is a, c, g, or t and codes for  60% R; 5% K/H; 2.72% A/V/T/P/Y/N/D/E/Q/L/I
  misc_feature (55)..(57): n is a, c, g, or t
  misc_feature (58)..(60): n is a, c, g, or t and codes for  80% F; 10% I/L
  misc_feature (58)..(60): n is a, c, g, or t and codes for  60% R; 10% K; 2.72% A/V/T/P/Y/N/D/E/Q/L/H
aacggtcaccgtggtaccctggccccagtagtcnnnnnnnnnnnnnnnnnnnnnnnnnnnagtacagtagtaggtggcggtgtcttctgc

SEQ ID NO: 158 - DNA, 30 bp, Artificial Sequence
  LCDR3-rev-constant
acaatagtatacggcaaaatcttcaggctc

SEQ ID NO: 159 - DNA, 36 bp, Artificial Sequence
  HCDR3 amplification
agaaacggtcaccgtggtaccctggccccagtagtc

SEQ ID NO: 160 - protein, 10 aa, Artificial Sequence
  CEA (P006.038)- CDR-H1
GFTFTDYYMN

SEQ ID NO: 161 - protein, 19 aa, Artificial Sequence
  CEA (P006.038)- CDR-H2
FIGNKANAYTTEYSASVKG

SEQ ID NO: 162 - protein, 10 aa, Artificial Sequence
  CEA (P006.038)- CDR-H3
DRGIRFGFDY

SEQ ID NO: 163 - protein, 10 aa, Artificial Sequence
  CEA (P006.038)- CDR-L1
RASSSVTYIH

SEQ ID NO: 164 - protein, 7 aa, Artificial Sequence
  CEA (P006.038)- CDR-L2
ATSNLAS

SEQ ID NO: 165 - protein, 9 aa, Artificial Sequence
  CEA (P006.038)- CDR-L3
QHWSSVPPT

SEQ ID NO: 166 - protein, 10 aa, Artificial Sequence
  CEA (P005.097)- CDR-H1
GFTFTDYYMN

SEQ ID NO: 167 - protein, 19 aa, Artificial Sequence
  CEA (P005.097)- CDR-H2
FIGNKANAYTTEYSASVKG

SEQ ID NO: 168 - protein, 10 aa, Artificial Sequence
  CEA (P005.097)- CDR-H3
DRGLRFSFDY

SEQ ID NO: 169 - protein, 10 aa, Artificial Sequence
  CEA (P005.097)- CDR-L1
RASSSVTYIH

SEQ ID NO: 170 - protein, 7 aa, Artificial Sequence
  CEA (P005.097)- CDR-L2
ATSNLAS

SEQ ID NO: 171 - protein, 9 aa, Artificial Sequence
  CEA (P005.097)- CDR-L3
QHWSSQPPT

SEQ ID NO: 172 - protein, 10 aa, Artificial Sequence
  CEA (P005.103)- CDR-H1
GFTFTDYYMN

SEQ ID NO: 173 - protein, 19 aa, Artificial Sequence
  CEA (P005.103)- CDR-H2
FIGNKANAYTTEYSASVKG

SEQ ID NO: 174 - protein, 10 aa, Artificial Sequence
  CEA (P005.103)- CDR-H3
DRGIRFYFDY

SEQ ID NO: 175 - protein, 10 aa, Artificial Sequence
  CEA (P005.103)- CDR-L1
RASSSVTYIH

SEQ ID NO: 176 - protein, 7 aa, Artificial Sequence
  CEA (P005.103)- CDR-L2
ATSNLAS

SEQ ID NO: 177 - protein, 9 aa, Artificial Sequence
  CEA (P005.103)- CDR-L3
QHWSSISPT

SEQ ID NO: 178 - protein, 10 aa, Artificial Sequence
  CEA (P002.139)- CDR-H1
GFYFTDYAMN

SEQ ID NO: 179 - protein, 19 aa, Artificial Sequence
  CEA (P002.139)- CDR-H2
VISNKANAYTTEYSASVKG

SEQ ID NO: 180 - protein, 10 aa, Artificial Sequence
  CEA (P002.139)- CDR-H3
DRGLRFYFDY

SEQ ID NO: 181 - protein, 10 aa, Artificial Sequence
  CEA (P002.139)- CDR-L1
HASSSVTYIH

SEQ ID NO: 182 - protein, 7 aa, Artificial Sequence
  CEA (P002.139)- CDR-L2
ATSNLAS

SEQ ID NO: 183 - protein, 9 aa, Artificial Sequence
  CEA (P002.139)- CDR-L3
QHWSSKPPT

SEQ ID NO: 184 - protein, 10 aa, Artificial Sequence
  CEA (P001.177)- CDR-H1
GFYFTDYYMN

SEQ ID NO: 185 - protein, 19 aa, Artificial Sequence
  CEA (P001.177)- CDR-H2
FISNKANAYTTEYSASVKG

SEQ ID NO: 186 - protein, 10 aa, Artificial Sequence
  CEA (P001.177)- CDR-H3
DRGLRFYFDY

SEQ ID NO: 187 - protein, 10 aa, Artificial Sequence
  CEA (P001.177)- CDR-L1
RASSSVTYIH

SEQ ID NO: 188 - protein, 7 aa, Artificial Sequence
  CEA (P001.177)- CDR-L2
ATSNLAS

SEQ ID NO: 189 - protein, 9 aa, Artificial Sequence
  CEA (P001.177)- CDR-L3
QHWSSKPPT

SEQ ID NO: 190 - protein, 10 aa, Artificial Sequence
  CEA (P005.102)- CDR-H1
GFTFTDYYMN

SEQ ID NO: 191 - protein, 19 aa, Artificial Sequence
  CEA (P005.102)- CDR-H2
FIGNKANAYTTEYSASVKG

SEQ ID NO: 192 - protein, 10 aa, Artificial Sequence
  CEA (P005.102)- CDR-H3
DRGIRFQFDY

SEQ ID NO: 193 - protein, 10 aa, Artificial Sequence
  CEA (P005.102)- CDR-L1
RASSSVTYIH

SEQ ID NO: 194 - protein, 7 aa, Artificial Sequence
  CEA (P005.102)- CDR-L2
ATSNLAS

SEQ ID NO: 195 - protein, 9 aa, Artificial Sequence
  CEA (P005.102)- CDR-L3
QHWSSKSPT

SEQ ID NO: 196 - protein, 10 aa, Artificial Sequence
  CEA (P005.102-combo1)- CDR-H1
GFYFTDYYMN

SEQ ID NO: 197 - protein, 19 aa, Artificial Sequence
  CEA (P005.102-combo1)- CDR-H2
VISNKANAYTTEYSASVKG

SEQ ID NO: 198 - protein, 10 aa, Artificial Sequence
  CEA (P005.102-combo1)- CDR-H3
DRGIRFQFDY

SEQ ID NO: 199 - protein, 10 aa, Artificial Sequence
  CEA (P005.102-combo1)- CDR-L1
RASSSVTYIH

SEQ ID NO: 200 - protein, 7 aa, Artificial Sequence
  CEA (P005.102-combo1)- CDR-L2
ATSNLAS

SEQ ID NO: 201 - protein, 9 aa, Artificial Sequence
  CEA (P005.102-combo1)- CDR-L3
QHWSSKSPT

SEQ ID NO: 202 - protein, 10 aa, Artificial Sequence
  CEA (P005.102-combo2)- CDR-H1
GFYFSDYYMN

SEQ ID NO: 203 - protein, 19 aa, Artificial Sequence
  CEA (P005.102-combo2)- CDR-H2
VISNKANAYTTEYSASVKG

SEQ ID NO: 204 - protein, 10 aa, Artificial Sequence
  CEA (P005.102-combo2)- CDR-H3
DRGIRFQFDY

SEQ ID NO: 205 - protein, 10 aa, Artificial Sequence
  CEA (P005.102-combo2)- CDR-L1
RASSSVTYIH

SEQ ID NO: 206 - protein, 7 aa, Artificial Sequence
  CEA (P005.102-combo2)- CDR-L2
ATSNLAS

SEQ ID NO: 207 - protein, 9 aa, Artificial Sequence
  CEA (P005.102-combo2)- CDR-L3
QHWSSKSPT

SEQ ID NO: 208 - protein, 10 aa, Artificial Sequence
  CEA (P005.103-combo1)- CDR-H1
GFTFTDYYMN

SEQ ID NO: 209 - protein, 19 aa, Artificial Sequence
  CEA (P005.103-combo1)- CDR-H2
FIGNKANAYTTEYSASVKG

SEQ ID NO: 210 - protein, 10 aa, Artificial Sequence
  CEA (P005.103-combo1)- CDR-H3
DRGIRFSFDY

SEQ ID NO: 211 - protein, 10 aa, Artificial Sequence
  CEA (P005.103-combo1)- CDR-L1
RASSSVTYIH

SEQ ID NO: 212 - protein, 7 aa, Artificial Sequence
  CEA (P005.103-combo1)- CDR-L2
ATSNLAS

SEQ ID NO: 213 - protein, 9 aa, Artificial Sequence
  CEA (P005.103-combo1)- CDR-L3
QHWSSISPT

SEQ ID NO: 214 - protein, 10 aa, Artificial Sequence
  CEA (P005.103-combo2)- CDR-H1
GFYFTDYYMN

SEQ ID NO: 215 - protein, 19 aa, Artificial Sequence
  CEA (P005.103-combo2)- CDR-H2
VISNKANAYTTEYSASVKG

SEQ ID NO: 216 - protein, 10 aa, Artificial Sequence
  CEA (P005.103-combo2)- CDR-H3
DRGIRFSFDY

SEQ ID NO: 217 - protein, 10 aa, Artificial Sequence
  CEA (P005.103-combo2)- CDR-L1
RASSSVTYIH

SEQ ID NO: 218 - protein, 7 aa, Artificial Sequence
  CEA (P005.103-combo2)- CDR-L2
ATSNLAS

SEQ ID NO: 219 - protein, 9 aa, Artificial Sequence
  CEA (P005.103-combo2)- CDR-L3
QHWSSISPT

SEQ ID NO: 220 - protein, 10 aa, Artificial Sequence
  CEA (P006.038-combo1)- CDR-H1
GFYFTDYAMN

SEQ ID NO: 221 - protein, 19 aa, Artificial Sequence
  CEA (P006.038-combo1)- CDR-H2
VISNKANAYTTEYSASVKG

SEQ ID NO: 222 - protein, 10 aa, Artificial Sequence
  CEA (P006.038-combo1)- CDR-H3
DRGIRFGFDY

SEQ ID NO: 223 - protein, 10 aa, Artificial Sequence
  CEA (P006.038-combo1)- CDR-L1
RASSSVTYIH

SEQ ID NO: 224 - protein, 7 aa, Artificial Sequence
  CEA (P006.038-combo1)- CDR-L2
ATSNLAS

SEQ ID NO: 225 - protein, 9 aa, Artificial Sequence
  CEA (P006.038-combo1)- CDR-L3
QHWSSVPPT

SEQ ID NO: 226 - protein, 10 aa, Artificial Sequence
  CEA (P006.038-combo2)- CDR-H1
GFTFSDYEMN

SEQ ID NO: 227 - protein, 19 aa, Artificial Sequence
  CEA (P006.038-combo2)- CDR-H2
FISNKANAYTTEYSASVKG

SEQ ID NO: 228 - protein, 10 aa, Artificial Sequence
  CEA (P006.038-combo2)- CDR-H3
DRGIRFGFDY

SEQ ID NO: 229 - protein, 10 aa, Artificial Sequence
  CEA (P006.038-combo2)- CDR-L1
RASSSVTYIH

SEQ ID NO: 230 - protein, 7 aa, Artificial Sequence
  CEA (P006.038-combo2)- CDR-L2
ATSNLAS

SEQ ID NO: 231 - protein, 9 aa, Artificial Sequence
  CEA (P006.038-combo2)- CDR-L3
QHWSSVPPT

SEQ ID NO: 232 - protein, 96 aa, Artificial Sequence
  IGHV1-2-02 human acceptor sequence
QLVQSGAEVKKPGASVKVSCKASGYTFTGYYMHWVRQAPGQGLEWMGWINPNSGGTNYAQKFQGRVTMTRDTSISTAYMELSRLRSDDTAVYYCAR

SEQ ID NO: 233 - protein, 98 aa, Artificial Sequence
  IGHV1-69-01 human acceptor sequence
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCAR

SEQ ID NO: 234 - protein, 98 aa, Artificial Sequence
  IGHV1-69-05 human acceptor sequence
QVQLVQSGAEVKKPGSSVKVSCKASGGTFSSYAISWVRQAPGQGLEWMGGIIPIFGTANYAQKFQGRVTITTDESTSTAYMELSSLRSEDTAVYYCAR

SEQ ID NO: 235 - protein, 95 aa, Artificial Sequence
  IGKV1-39-01
DIQMTQSPSSLSASVGDRVTITCRASQSISSYLNWYQQKPGKAPKLLIYAASSLQSGVPSRFSGSGSGTDFTLTISSLQPEDFATYYCQQSYSTP

SEQ ID NO: 236 - protein, 449 aa, Artificial Sequence
  anti- CEA(A5B7) Fc hole chain
EVKLVESGGGLVQPGGSLRLSCATSGFTFTDYYMNWVRQPPGKALEWLGFIGNKANGYTTEYSASVKGRFTISRDKSQSILYLQMNTLRAEDSATYYCTRDRGLRFYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 237 - protein, 213 aa, Artificial Sequence
  anti-CEA(A5B7) light chain
QTVLSQSPAILSASPGEKVTMTCRASSSVTYIHWYQQKPGSSPKSWIYATSNLASGVPARFSGSGSGTSYSLTISRVEAEDAATYYCQHWSSKPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 238 - protein, 449 aa, Artificial Sequence
  anti- CEA(A5H1EL1D)-Fc hole chain
EVQLLESGGGLVQPGGSLRLSCAASGFTFTDYYMNWVRQAPGKGLEWLGFIGNKANAYTTEYSASVKGRFTISRDKSKNTLYLQMNSLRAEDTATYYCTRDRGLRFYFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 239 - protein, 213 aa, Artificial Sequence
  anti-CEA(A5H1EL1D) light chain
EIVLTQSPATLSLSPGERATLSCRASSSVTYIHWYQQKPGQAPRSWIYATSNLASGIPARFSGSGSGTDFTLTISSLEPEDFAVYYCQHWSSKPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 240 - protein, 449 aa, Artificial Sequence
  anti- CEA(P006.038) Fc hole chain
EVQLLESGGGLVQPGGSLRLSCAASGFTFTDYYMNWVRQAPGKGLEWLGFIGNKANAYTTEYSASVKGRFTISRDKSKNTLYLQMNSLRAEDTATYYCTRDRGIRFGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 241 - protein, 213 aa, Artificial Sequence
  anti-CEA(P006.038) light chain
EIVLTQSPATLSLSPGERATLSCRASSSVTYIHWYQQKPGQAPRSWIYATSNLASGIPARFSGSGSGTDFTLTISSLEPEDFAVYYCQHWSSVPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 242 - protein, 449 aa, Artificial Sequence
  anti- CEA(P005.097) Fc hole chain
EVQLLESGGGLVQPGGSLRLSCAASGFTFTDYYMNWVRQAPGKGLEWLGFIGNKANAYTTEYSASVKGRFTISRDKSKNTLYLQMNSLRAEDTATYYCTRDRGLRFSFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 243 - protein, 213 aa, Artificial Sequence
  anti-CEA(P005.097) light chain
EIVLTQSPATLSLSPGERATLSCRASSSVTYIHWYQQKPGQAPRSWIYATSNLASGIPARFSGSGSGTDFTLTISSLEPEDFAVYYCQHWSSQPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 244 - protein, 449 aa, Artificial Sequence
  anti- CEA(P005.103) Fc hole chain
EVQLLESGGGLVQPGGSLRLSCAASGFTFTDYYMNWVRQAPGKGLEWLGFIGNKANAYTTEYSASVKGRFTISRDKSKNTLYLQMNSLRAEDTATYYCTRDRGIRFYFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 245 - protein, 213 aa, Artificial Sequence
  anti-CEA(P005.103) light chain
EIVLTQSPATLSLSPGERATLSCRASSSVTYIHWYQQKPGQAPRSWIYATSNLASGIPARFSGSGSGTDFTLTISSLEPEDFAVYYCQHWSSISPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 246 - protein, 449 aa, Artificial Sequence
  anti- CEA(P002.139) Fc hole chain
EVQLLESGGGLVQPGGSLRLSCAASGFYFTDYAMNWVRQAPGKGLEWLGVISNKANAYTTEYSASVKGRFTISRDKSKNTLYLQMNSLRAEDTATYYCTRDRGLRFYFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 247 - protein, 213 aa, Artificial Sequence
  anti-CEA(P002.139) light chain
EIVLTQSPATLSLSPGERATLSCHASSSVTYIHWYQQKPGQAPRSWIYATSNLASGIPARFSGSGSGTDFTLTISSLEPEDFAVYYCQHWSSKPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 248 - protein, 449 aa, Artificial Sequence
  anti- CEA(P001.177) Fc hole chain
EVQLLESGGGLVQPGGSLRLSCAASGFYFTDYYMNWVRQAPGKGLEWLGFISNKANAYTTEYSASVKGRFTISRDKSKNTLYLQMNSLRAEDTATYYCTRDRGLRFYFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 249 - protein, 213 aa, Artificial Sequence; identical to SEQ ID NO: 239
  anti-CEA(P001.177) light chain

SEQ ID NO: 250 - protein, 449 aa, Artificial Sequence
  anti- CEA(P005.102) Fc hole chain
EVQLLESGGGLVQPGGSLRLSCAASGFTFTDYYMNWVRQAPGKGLEWLGFIGNKANAYTTEYSASVKGRFTISRDKSKNTLYLQMNSLRAEDTATYYCTRDRGIRFQFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 251 - protein, 213 aa, Artificial Sequence
  anti-CEA(P005.102) light chain
EIVLTQSPATLSLSPGERATLSCRASSSVTYIHWYQQKPGQAPRSWIYATSNLASGIPARFSGSGSGTDFTLTISSLEPEDFAVYYCQHWSSKSPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 252 - protein, 449 aa, Artificial Sequence
  anti- CEA(P005.103-combo1) Fc hole chain
EVQLLESGGGLVQPGGSLRLSCAASGFTFTDYYMNWVRQAPGKGLEWLGFIGNKANAYTTEYSASVKGRFTISRDKSKNTLYLQMNSLRAEDTATYYCTRDRGIRFSFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 253 - protein, 213 aa, Artificial Sequence; identical to SEQ ID NO: 245
  anti-CEA(P005.103-combo1) light chain

SEQ ID NO: 254 - protein, 449 aa, Artificial Sequence
  anti- CEA(P005.103-combo2) Fc hole chain
EVQLLESGGGLVQPGGSLRLSCAASGFYFTDYYMNWVRQAPGKGLEWLGVISNKANAYTTEYSASVKGRFTISRDKSKNTLYLQMNSLRAEDTATYYCTRDRGIRFSFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 255 - protein, 213 aa, Artificial Sequence; identical to SEQ ID NO: 245
  anti-CEA(P005.103-combo2) light chain

SEQ ID NO: 256 - protein, 449 aa, Artificial Sequence
  anti- CEA(P005.102-combo1) Fc hole chain
EVQLLESGGGLVQPGGSLRLSCAASGFYFTDYYMNWVRQAPGKGLEWLGVISNKANAYTTEYSASVKGRFTISRDKSKNTLYLQMNSLRAEDTATYYCTRDRGIRFQFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 257 - protein, 213 aa, Artificial Sequence; identical to SEQ ID NO: 251
  anti-CEA(P005.102-combo1) light chain

SEQ ID NO: 258 - protein, 449 aa, Artificial Sequence
  anti- CEA(P005.102-combo2) Fc hole chain
EVQLLESGGGLVQPGGSLRLSCAASGFYFSDYYMNWVRQAPGKGLEWLGVISNKANAYTTEYSASVKGRFTISRDKSKNTLYLQMNSLRAEDTATYYCTRDRGIRFQFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 259 - protein, 213 aa, Artificial Sequence; identical to SEQ ID NO: 251
  anti-CEA(P005.102-combo2) light chain

SEQ ID NO: 260 - protein, 449 aa, Artificial Sequence
  anti- CEA(P006.038-combo1) Fc hole chain
EVQLLESGGGLVQPGGSLRLSCAASGFYFTDYAMNWVRQAPGKGLEWLGVISNKANAYTTEYSASVKGRFTISRDKSKNTLYLQMNSLRAEDTATYYCTRDRGIRFGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 261 - protein, 213 aa, Artificial Sequence; identical to SEQ ID NO: 241
  anti-CEA(P006.038-combo1) light chain

SEQ ID NO: 262 - protein, 449 aa, Artificial Sequence
  anti- CEA(P006.038-combo2) Fc hole chain
EVQLLESGGGLVQPGGSLRLSCAASGFTFSDYEMNWVRQAPGKGLEWLGFISNKANAYTTEYSASVKGRFTISRDKSKNTLYLQMNSLRAEDTATYYCTRDRGIRFGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 263 - protein, 213 aa, Artificial Sequence; identical to SEQ ID NO: 241
  anti-CEA(P006.038-combo2) light chain

SEQ ID NO: 264 - protein, 448 aa, Artificial Sequence
  anti- CEA(MFE23) Fc hole chain
QVKLQQSGAELVRSGTSVKLSCTASGFNIKDSYMHWLRQGPEQGLEWIGWIDPENGDTEYAPKFQGKATFTTDTSSNTAYLQLSSLTSEDTAVYYCNEGTPTGPYYFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 265 - protein, 213 aa, Artificial Sequence
  anti-CEA(MFE23) light chain
ENVLTQSPAIMSASPGEKVTITCSASSSVSYMHWFQQKPGTSPKLWIYSTSNLASGVPARFSGSGSGTSYSLTISRMEAEDAATYYCQQRSSYPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 266 - protein, 448 aa, Artificial Sequence
  anti- CEA(huMFE23-L28-H24) Fc hole chain
QVQLVQSGAEVKKPGASVKVSCKASGFNIKDSYMHWVRQAPGQGLEWMGWIDPENGDTEYAPKFQGRVTMTTDTSISTAYMELSRLRSDDTAVYYCNEGTPTGPYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 267 - protein, 213 aa, Artificial Sequence
  anti-CEA(huMFE23-L28-H24) light chain
EIQMTQSPSSLSASVGDRVTITCRASSSVPYMHWLQQKPGKAPKLLIYSTSNLASGVPSRFSGSGSGTDFTLTISSVQPEDFATYYCQQRSSYPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 268 - protein, 448 aa, Artificial Sequence
  anti- CEA(huMFE23-L28-H28) Fc hole chain
QVQLVQSGAEVKKPGASVKVSCKASGFNIKDSYMHWVRQAPGQGLEWMGWIDPENGDTEYAPKFQGRVTMTRDTSISTAYMELSRLRSDDTAVYYCNEGTPTGPYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 269 - protein, 448 aa, Artificial Sequence
  anti- CEA(huMFE23-L28-H25) Fc hole chain
QVQLVQSGAEVKKPGASVKVSCKASGYTFKDSYMHWVRQAPGQGLEWMGWIDPENGDTEYAPKFQGRVTMTTDTSISTAYMELSRLRSDDTAVYYCNEGTPTGPYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 270 - protein, 448 aa, Artificial Sequence
  anti- CEA(huMFE23-L27-H29) Fc hole chain
QVQLVQSGAEVKKPGSSVKVSCKASGFNIKDSYMHWVRQAPGQGLEWMGWIDPENGDTEYAPKFQGRVTITTDESTSTAYMELSSLRSEDTAVYYCNEGTPTGPYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 271 - protein, 213 aa, Artificial Sequence
  anti-CEA(huMFE23-L27-H29) light chain
EIQMTQSPSSLSASVGDRVTITCRASSSVPYMHWYQQKPGKAPKLLIYSTSNLASGVPSRFSGSGSGTDFTLTISSVQPEDFATYYCQQRSSYPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 272 - protein, 448 aa, Artificial Sequence; identical to SEQ ID NO: 268
  anti- CEA(huMFE23-L27-H28) Fc hole chain

SEQ ID NO: 273 - protein, 448 aa, Artificial Sequence
  anti- CEA(huMFE23-L27-H26) Fc hole chain
QVQLVQSGAEVKKPGASVKVSCKASGFNIKDSYMHWVRQAPGQGLEWMGWIDPENGGTNYAQKFQGRVTMTTDTSISTAYMELSRLRSDDTAVYYCNEGTPTGPYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 274 - protein, 448 aa, Artificial Sequence; identical to SEQ ID NO: 266
  anti- CEA(huMFE23-L27-H24) Fc hole chain

SEQ ID NO: 275 - protein, 5 aa, Artificial Sequence
  CD3-HCDR1
TYAMN

SEQ ID NO: 276 - protein, 19 aa, Artificial Sequence
  CD3-HCDR2
RIRSKYNNYATYYADSVKG

SEQ ID NO: 277 - protein, 14 aa, Artificial Sequence
  CD3-HCDR3
HGNFGNSYVSWFAY

SEQ ID NO: 278 - protein, 14 aa, Artificial Sequence
  CD3-LCDR1
GSSTGAVTTSNYAN

SEQ ID NO: 279 - protein, 7 aa, Artificial Sequence
  CD3-LCDR2
GTNKRAP

SEQ ID NO: 280 - protein, 9 aa, Artificial Sequence
  CD3-LCDR3
ALWYSNLWV

SEQ ID NO: 281 - protein, 125 aa, Artificial Sequence
  CD3 VH
EVQLLESGGGLVQPGGSLRLSCAASGFTFSTYAMNWVRQAPGKGLEWVSRIRSKYNNYATYYADSVKGRFTISRDDSKNTLYLQMNSLRAEDTAVYYCVRHGNFGNSYVSWFAYWGQGTLVTVSS

SEQ ID NO: 282 - protein, 109 aa, Artificial Sequence
  CD3 VL
QAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVL

SEQ ID NO: 283 - protein, 5 aa, Artificial Sequence
  CEA (CH1A1A 98/99)- CDR-H1
EFGMN

SEQ ID NO: 284 - protein, 17 aa, Artificial Sequence
  CEA (CH1A1A 98/99)- CDR-H2
WINTKTGEATYVEEFKG

SEQ ID NO: 285 - protein, 12 aa, Artificial Sequence
  CEA (CH1A1A 98/99)- CDR-H3
WDFAYYVEAMDY

SEQ ID NO: 286 - protein, 11 aa, Artificial Sequence
  CEA (2F1)- CDR-L1
KASAAVGTYVA

SEQ ID NO: 287 - protein, 7 aa, Artificial Sequence
  CEA (2F1)- CDR-L2
SASYRKR

SEQ ID NO: 288 - protein, 10 aa, Artificial Sequence
  CEA (2F1)- CDR-L3
HQYYTYPLFT

SEQ ID NO: 289 - protein, 121 aa, Artificial Sequence
  VH (CEA CH1A1A 98/99)
QVQLVQSGAEVKKPGASVKVSCKASGYTFTEFGMNWVRQAPGQGLEWMGWINTKTGEATYVEEFKGRVTFTTDTSTSTAYMELRSLRSDDTAVYYCARWDFAYYVEAMDYWGQGTTVTVSS

SEQ ID NO: 290 - protein, 108 aa, Artificial Sequence
  VL (CEA 2F1)
DIQMTQSPSSLSASVGDRVTITCKASAAVGTYVAWYQQKPGKAPKLLIYSASYRKRGVPSRFSGSGSGTDFTLTISSLQPEDFATYYCHQYYTYPLFTFGQGTKLEIK

SEQ ID NO: 291 - protein, 5 aa, Artificial Sequence
  CEA (T84.66-LCHA)- CDR-H1
DTYMH

SEQ ID NO: 292 - protein, 17 aa, Artificial Sequence
  CEA (T84.66-LCHA)- CDR-H1
RIDPANGNSKYVPKFQG

SEQ ID NO: 293 - protein, 12 aa, Artificial Sequence
  CEA (T84.66-LCHA)- CDR-H1
FGYYVSDYAMAY

SEQ ID NO: 294 - protein, 15 aa, Artificial Sequence
  CEA (T84.66-LCHA)- CDR-L1
RAGESVDIFGVGFLH

SEQ ID NO: 295 - protein, 7 aa, Artificial Sequence
  CEA (T84.66-LCHA)- CDR-L2
RASNRAT

SEQ ID NO: 296 - protein, 9 aa, Artificial Sequence
  CEA (T84.66-LCHA)- CDR-L3
QQTNEDPYT

SEQ ID NO: 297 - protein, 254 aa, Homo sapiens
MEYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 298 - protein, 199 aa, Homo sapiens
ACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGL

SEQ ID NO: 299 - protein, 107 aa, Homo sapiens
APELLGGPSVFLFPPKPKDTLMISRTPEVTCVWDVSHEDPEVKFNWYVDGVEVHNAKTKPREEQESTYRWSVLTVLHQDWLNGKEYKCKVSNKALPAPIEKTISKAK

SEQ ID NO: 300 - protein, 106 aa, Homo sapiens [sequence omitted]

SEQ ID NO: 301 - protein, 98 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 302 - protein, 5 aa, Homo sapiens
EPKSC

SEQ ID NO: 303 - protein, 10 aa, Homo sapiens
  MISC_FEATURE (8)..(8): X is S or P
DKTHTCPXCP

SEQ ID NO: 304 - protein, 7 aa, Homo sapiens
  MISC_FEATURE (5)..(5): X is S or P
HTCPXCP

SEQ ID NO: 305 - protein, 5 aa, Homo sapiens
  MISC_FEATURE (3)..(3): X is S or P
CPXCP

SEQ ID NO: 306 - protein, 330 aa, Homo sapiens [sequence omitted]

SEQ ID NO: 307 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 308 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 309 - protein, 377 aa, Homo sapiens
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 310 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 311 - protein, 88 aa, Homo sapiens
PKPFITSNNSNPVEDEDAVALTCEPEIQNTTYLWWVNNQSLPVSPRLQLSNDNRTLTLLSVTRNDVGPYECGIQNKLSVDHSDPVILN

SEQ ID NO: 312 - protein, 88 aa, Homo sapiens
PKPSISSNNSKPVEDKDAVAFTCEPETQDATYLWWVNNQSLPVSPRLQLSNGNRTLTLFNVTRNDTASYKCETQNPVSARRSDSVILN

SEQ ID NO: 313 - protein, 10 aa, Artificial Sequence
  CEA (MFE-L27, L28, L29)- CDR-L1
RASSSVPYMH